SEQUENCE LISTING (276 sequences)

SEQ ID NO: 1 - protein, 457 aa, Unknown
  Synthesized or naturally derived
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVSSGGSGGSGGSGGSGGSSGEWDREINNYTSLIHSLIEESQNQQEKNEQELLELDKWASLWNWF

SEQ ID NO: 2 - protein, 449 aa, Unknown
  Synthesized or naturally derived
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVSSGGSGGSGGSGGSGGSSGSGIVQQQNNLLRAIEAQQHLLQLTVWGIKQLQARIL

SEQ ID NO: 3 - protein, 435 aa, Unknown
  Synthesized or naturally derived
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVSSGGSGGSGGSGGSGGSSGLQARILAVERYLKDQQLLGIWG

SEQ ID NO: 4 - protein, 465 aa, Unknown
  Synthesized or naturally derived
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVSSGGSGGSGGSGGSGGSSGLQARILAVERYLKDQQLLGIWGCSGKLICTTAVPWNASWSNKSLEQIWNHTT

SEQ ID NO: 5 - protein, 465 aa, Unknown
  Synthesized or naturally derived
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVSSGGSGGSGGSGGSGGSSGLQARILAVERYLKDQQLLGIWGSSGKLISTTAVPWNASWSNKSLEQIWNHTT

SEQ ID NO: 6 - protein, 436 aa, Unknown
  Synthesized or naturally derived
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVSSGGSGGSGGSGGSGGSSGTTAVPWNASWSNKSLEQIWNHTT

SEQ ID NO: 7 - protein, 444 aa, Unknown
  Synthesized or naturally derived
MNRGVPFRHLLLVLQLALLPAATQGKKVHHHHHHKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVSSGGSGGSGGSGGSGGSSGTTAVPWNASWSNKSLEQIWNHTT

SEQ ID NO: 8 - protein, 465 aa, Unknown
  Synthesized or naturally derived
MNRGVPFRHLLLVLQLALLPAATQGKKVHHHHHHKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVSSGGSGGSGGSGGSGGSSGEWDREINNYTSLIHSLIEESQNQQEKNEQELLELDKWASLWNWF

SEQ ID NO: 9 - protein, 394 aa, Unknown
  Synthesized or naturally derived
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPV

SEQ ID NO: 10 - protein, 402 aa, Unknown
  Synthesized or naturally derived
MNRGVPFRHLLLVLQLALLPAATQGKKVHHHHHHKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPV

SEQ ID NO: 11 - protein, 44 aa, Unknown
  Synthesized or naturally derived
EWDREINNYTSLIHSLIEESQNQQEKNEQELLELDKWASLWNWF

SEQ ID NO: 12 - protein, 36 aa, Unknown
  Synthesized or naturally derived
SGIVQQQNNLLRAIEAQQHLLQLTVWGIKQLQARIL

SEQ ID NO: 13 - protein, 22 aa, Unknown
  Synthesized or naturally derived
LQARILAVERYLKDQQLLGIWG

SEQ ID NO: 14 - protein, 52 aa, Unknown
  Synthesized or naturally derived
LQARILAVERYLKDQQLLGIWGCSGKLICTTAVPWNASWSNKSLEQIWNHTT

SEQ ID NO: 15 - protein, 52 aa, Unknown
  Synthesized or naturally derived
LQARILAVERYLKDQQLLGIWGSSGKLISTTAVPWNASWSNKSLEQIWNHTT

SEQ ID NO: 16 - protein, 23 aa, Unknown
  Synthesized or naturally derived
TTAVPWNASWSNKSLEQIWNHTT

SEQ ID NO: 17 - protein, 23 aa, Unknown
  Synthesized or naturally derived
TTAVPWNASWSNKSLEQIWNHTT

SEQ ID NO: 18 - protein, 44 aa, Unknown; identical to SEQ ID NO: 11
  Synthesized or naturally derived

SEQ ID NO: 19 - protein, 19 aa, Unknown
  Synthesized or naturally derived
SSGGSGGSGGSGGSGGSSG

SEQ ID NO: 20 - protein, 156 aa, Unknown
  Synthesized or naturally derived
STMGAASITLTVQARQLLSGIVQQQSNLLRAIEAQQHMLKLTVWGIKQLQARVLALERYLKDQQLLGIWGCSGKLICTTNVPWNNTWSNKNKSEIWDKMTWLQWDKEISNYTQIIYNLIEESQTQQEINEQELLALDKWANLWNWFDISQWLWYIK

SEQ ID NO: 21 - protein, 156 aa, Unknown
  Synthesized or naturally derived
STMGATSITLTVQARQLLSGIVQQQNNLLRAIEAQQHLLKLTVWGIKQLQARVLAVERYLRDQQLLGIWGCSGKLICTTNVPWNSSWSNKSLDEIWNNMTWLQWDKEINNYTQLIYRLIEESQNQQEKNEKELLELDKWANLWSWFDISNWLWYIK

SEQ ID NO: 22 - protein, 156 aa, Unknown
  Synthesized or naturally derived
STMGAASMTLTVQARQLLSGIVQQQSNLLRAIEAQQHLLKLTVWGIKQLQARVLAVESYLRDQQLLGIWGCSGKLICTTTVPWNASWSNKSYSEIWENMTWLQWDKEISNYTNLIYGLIEESQNQQEKNEQDLLALDKWANLWSWFEISNWLWYIK

SEQ ID NO: 23 - protein, 156 aa, Unknown
  Synthesized or naturally derived
STMGAASITLTVQARKLLSGIVQQQSNLLRAIEAQQHLLKLTVWGIKQLQARVLAVERYLRDQQLLGIWGCSGKLICPTNVPWNSSWSNKSLDEIWENMTWLQWDKEISNYTIKIYELIEESQIQQERNEKDLLELDKWASLWNWFDISKWLWYIK

SEQ ID NO: 24 - protein, 156 aa, Unknown
  Synthesized or naturally derived
STMGAASITLTVQARQLLSGIVQQQSNLLKAIEAQQHLLKLTVWGIKQLQARVLALERYLQDQQLLGIWGCSGKLICTTTVPWNSSWSNKTYEEIWNNMTWLQWDREIDNYTNIIYNLLEESQNQQEKNEQDLLALDKWASLWNWFSITNWLWYIR

SEQ ID NO: 25 - protein, 156 aa, Unknown
  Synthesized or naturally derived
STMGAASITLTVQARQLLTGIVQQQSNLLKAIEAQQQMLRLTVWGIKQLQARVLALERYLQDQQLLGIWGCSGKLICATDVRWNSSWSNKTQEQIWKNMTWLQWDKEISTYTDIIYMLLEESQNQQEKNEQDLLALDKWANLWNWFDITRWLWYIK

SEQ ID NO: 26 - protein, 156 aa, Unknown
  Synthesized or naturally derived
STMGAASLTLTVQARQLLSGIVQQQSNLLQAIEAQQHLLKLTVWGIKQLQARVLAVERYLKDQQLLGIWGCSGKLICATTVPWNTSWSNKSQDEIWDNMTWLQWDKEISNYTNIIYRLLEESQNQQEKNEQDLLALDKWADLWSWFNISHWLWYIR

SEQ ID NO: 27 - protein, 156 aa, Unknown
  Synthesized or naturally derived
STMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIK

SEQ ID NO: 28 - protein, 156 aa, Unknown
  Synthesized or naturally derived
STMGAASMALTVQARQLLSGIVQQQNNLLRAIEAQQHLLQLTVWGIKQLQARVLAIERYLQDQQLLGIWGCSGKLICTTTVPWNASWSNKSLDQIWENMTWMQWEREIDNYTSLIYTLIEDSQKQQEKNEQELLALDTWASLWNWFSITNWLWYIK

SEQ ID NO: 29 - protein, 156 aa, Unknown
  Synthesized or naturally derived
STMGAASVTLTVQARLLLSGIVQQQNNLLRAIEAQQHLLQLTVWGIKQLQARVLAVERYLKDQQLLGIWGCSGKLICTTAVPWNASWSNKSLDEIWNNMTWMQWEREINNYTGLIYTLIEESQNQQEKNELDLLQLDKWASLWNWFDITNWLWYIK

SEQ ID NO: 30 - protein, 156 aa, Unknown
  Synthesized or naturally derived
STMGAASMTLTVQARLLLSGIVQQQNNLLRAIEAQQHLLQLTVWGIKQLQARVLAVERYLEDQQLLGIWGCSGKLICTTAVPWNASWSNKSRSEIWNNMTWMQWDKEIHNYTNLIYTLIGESQIQQEKNEQELLGLDKWASLWNWFDITKWLWYIK

SEQ ID NO: 31 - protein, 156 aa, Unknown
  Synthesized or naturally derived
STMGAASIALTVQTRHLLSGIVQQQNNLLRAIEAQQHLLQLTVWGIKQLQARILAVERYLRDQQLLGIWGCSGKLICPTAVPWNASWSNKSLEEIWENMTWREWEREIDNYTGKIYDLLAKSQNQREMNEQELLKLDKWADLWNWFDITQWLWYIK

SEQ ID NO: 32 - protein, 156 aa, Unknown
  Synthesized or naturally derived
STMGAASITLTVQVRQLLSGIVQQQSNLLRAIEAQQHMLQLTVWGIKQLQTRVLAIERYLRDQQLLGIWGCSGKLICTTAVPWNSSWSNRSQEDIWNNMTWMQWDREISNYTNTIYRLLEDSQNQQEKNEQDLLALDKWQNLWTWFGITNWLWYIK

SEQ ID NO: 33 - protein, 156 aa, Unknown
  Synthesized or naturally derived
STMGAASITLTVQARQLLSGIVQQQSNLLKAIEAQQHMLQLTVWGIKQLQTRVLAIERHLRDQQLLGIWGCSGKLICTTAVPWNSSWSNKSQEEIWDNMTWMQWDREISNYTDIIYNLLEVSQNQQDKNEKDLLALDKWENLWNWFNITNWLWYIK

SEQ ID NO: 34 - protein, 156 aa, Unknown
  Synthesized or naturally derived
STMGAASITLTVQARQLLSGIVQQQSNLLRAIEAQQHLLQLTVWGIKQLQTRVLAIERYLKDQQLLGIWGCSGKLICTTAVPWNSSWSNRTQKEIWDNMTWMQWDREINNYTNTIYRLLEESQNQQEENEKDLLALDSWKNLWNWFDITKWLWYIK

SEQ ID NO: 35 - protein, 178 aa, Unknown
  Synthesized or naturally derived
STMGAASITLTVQARQLLSGIVQQQSNLLRAIEAQQHMLQLTVWGIKQLQARVLAIERYLKDQQLLGLWGCSGKLICTTAVHWNSSWSNKSQDYIWGNMTWMQWDREINNYTDIIYTLLEESQSQQEKNEKDLLALDSWNNLWNWFSITKWLWYIKIFIMIVGGLIGLRIILGVLSIV

SEQ ID NO: 36 - protein, 156 aa, Unknown
  Synthesized or naturally derived
STMGARSVTLTVQARQLMSGIVQQQNNLLRAIEAQQHLLQLTVWGIKQLQARILAVERYLKDQQLLGIWGCSGKHICTTNVPWNSSWSNRSLNEIWQNMTWMEWEREIDNYTGLIYSLIEESQTQQEKNEKELLELDKWASLWNWFSITQWLWYIK

SEQ ID NO: 37 - protein, 156 aa, Unknown
  Synthesized or naturally derived
STMGAASVTLTVQARQLLSGIVQQQNNLLRAIEAQQHLLQLTVWGIKQLQARVLAVERYLKDQQLLGIWGCSGKHICTTNVPWNSSWSNRSLDDIWQNMTWMQWEREIENYTGVIYSLIEESQIQQEKNEKELLELDKWASLWNWFSISNWLWYIR

SEQ ID NO: 38 - protein, 156 aa, Unknown
  Synthesized or naturally derived
STMGAASLTLTVQARQLLSGIVQQQNNLLRAIEAQQHLLQLTVWGIKQLQARVLAVESYLKDQQLLGIWGCSGKHICTTAVPWNSSWSNKSLDDIWNNMTWMEWEKEIDNYTGVIYSLIEESQVQQEKNEKELLELDKWASLWNWFSITKWLWYIK

SEQ ID NO: 39 - protein, 156 aa, Unknown
  Synthesized or naturally derived
STMGAVSLTLTVQARQVLSGIVQQQNNLLRAIEAQQHLLQLTVWGIKQLQARILAVESYLKDQQLLGIWGCSGKHICTTNVPWNSSWSNRSVDEIWNNMTWMEWEREIDNYTELVYSLLEVSQIQQEKNEQELLKLDTWASLWNWFSITQWLWYIK

SEQ ID NO: 40 - protein, 156 aa, Unknown
  Synthesized or naturally derived
EHMGAASITLTVQARQLLSGIVQQQNNLLRAIEAQQHLLQLTVWGIKQLQARVLAVERYLKDQQLLGIWGCSGKLICTTNVPWNSSWSNKSQEEIWNNMTWMEWEKEISNYSNIIYKLIEESQNQQEKNEQELLALDKWASLWNWFDISNWLWYIK

SEQ ID NO: 41 - protein, 156 aa, Unknown
  Synthesized or naturally derived
STMGAASITLTVQARQLLSGIVQQQSNLLRAIEAQQHLLQLTVWGIKQLQARVLAVERYLKDQQLLGLWGCSGKLICTTNVPWNSSWSNKSLEEIWGNMTWMEWEKEVSNYSKEIYRLIEDSQNQQEKNEQELLALDKWASLWNWFDITQWLWYIK

SEQ ID NO: 42 - protein, 156 aa, Unknown
  Synthesized or naturally derived
STMGAASLTLTVQARQLLSGIVQQQNNLLQAIEAQQHMLQLTVWGIKQLQARVLAVERYLKDQQLLGLWGCSGKLICTTNVPWNSSWSNKSQDEIWNNMTWMQWEKEISNYSKTIYMLIEKSQSQQERNEQELLELDKWDSLWSWFDITNWLWYIK

SEQ ID NO: 43 - protein, 156 aa, Unknown
  Synthesized or naturally derived
SNIGAASITLTVQARQLLSGIVQQQNNLLRAIEAQQHLLQLTVWGIKQLQARVLAVERYLKDQQLLGIWGCSGKLICTTNVPWNTSWSNKSHDEIWNNMTWMQWEKEINNYSNTIYRLIEESQNQQEKNEQELLALDKWASLWSWFDISNWLWYIK

SEQ ID NO: 44 - protein, 156 aa, Unknown
  Synthesized or naturally derived
STMGAASITLTVQARQLLSGIVQQQNNLLKAIEAQQHLLQLTVWGIKQLQARILAVERYLKDQQLLGIWGCSGKLICTTNVPWNSSWSNKSQEEIWGNMTWMQWEKEIDNYTDTIYRLIEEAQNQQEKNEQDLLALDKWDSLWSWFTITNWLWYIR

SEQ ID NO: 45 - protein, 156 aa, Unknown
  Synthesized or naturally derived
STMGAAAITLTAQARQLLSGIVQQQSNLLKAIEAQQHLLQLTVWGIKQLQARILAVERYLKDQQLLGIWGCSGKLICTTNVRWNSSWSNKSYDDIWDNMTWMQWEKEIDNYTKTIYSLIEDAQNQQERNEQELLALDKWDSLWSWFSITNWLWYIK

SEQ ID NO: 46 - protein, 156 aa, Unknown
  Synthesized or naturally derived
STMGAASITLTVQARNLLSGIVQQQSNLLKAIEAQQHLLQLTVWGIKQLQARILAVERYLKDQQLLGIWGCSGKLICPTTVPWNLSWSNKSQDEIWGNMTWMEWEKEIGNYTDTIYRLIESAQNQQEKNEQDLLALDKWDNLWNWFSITRWLWYIE

SEQ ID NO: 47 - protein, 156 aa, Unknown
  Synthesized or naturally derived
STMGAASMTLTVQARQLLSGIVQQQNNLLKAIEAQQHLLQLTVWGIKQLQARILAVERYLKDQQLLGIWGCSGKLICTTNVPWNSSWSNKSQNEIWENMTWMQWEKEISNYTGTIYKLIENAQNQQEKNEQDLLALDKWDNLWSWFTITNWLWYIK

SEQ ID NO: 48 - protein, 156 aa, Unknown
  Synthesized or naturally derived
STMGAASITLTVQVRQLLSGIVQQQSNLLRAIEAQQHLLQLTVWGIKQLRARVLALERYLKDQQLLGIWGCSGKLICTTNVPWNTSWSNKSYNEIWENMTWIEWEREIDNYTYHIYSLIEQSQIQQEKNEQDLLALDQWASLWSWFSISNWLWYIR

SEQ ID NO: 49 - protein, 156 aa, Unknown
  Synthesized or naturally derived
STMGAASITLTVQVRQLLSGIVQQQSNLLRAIEAQQHLLQLTVWGIKQLQARVLALERYLRDQQLLGIWGCSGKLICTTNVPWNASWSNKTYNDIWDNMTWIQWDREISNYTQQIYSLIEESQNQQEKNEQDLLALDNWASLWTWFDITKWLWYIK

SEQ ID NO: 50 - protein, 156 aa, Unknown
  Synthesized or naturally derived
STMGAASITLTAQVRQLLSGIVQQQSNLLRAIEAQQHLLQLTVWGIKQLQSRVLAIERYLKDQQLLGIWGCSGKLICTTNVPWNTSWSNKSYNEIWDNMTWLEWEREIHNYTQHIYSLIEESQNQQEKNEQDLLALDKWASLWNWFDISNWLWYIR

SEQ ID NO: 51 - protein, 156 aa, Unknown
  Synthesized or naturally derived
STMGAASITLTVQVRQLLSGIVQQQSNLLRAIEAQQHLLQLTVWGIKQLQARVLAVERYLKDQQLLGIWGCSGRLICTTNVPWNASWSNKSYNQIWDNLTWVQWEREISNYTQQIYTLLEESQNQQEKNEQDLLALDKWADLWNWFDISRWLWYIK

SEQ ID NO: 52 - protein, 156 aa, Unknown
  Synthesized or naturally derived
STMGAASITLTVQARQLLSGIVQQQSNLLRAIQAQQHMLQLTVWGIKQLQARVLAVERYLKDQQLLGIWGCSGKLICTTNVPWNSSWSNKSLDEIWDNMTWMEWDKQINNYTDEIYRLLEVSQNQQEKNEQDLLALDKWANLWNWFSITNWLWYIR

SEQ ID NO: 53 - protein, 156 aa, Unknown
  Synthesized or naturally derived
STMGAASITLTVQARQLLSGIVQQQSNLLRAIQAQQHMLQLTVWGVKQLQARVLAVERYLKDQQLLGIWGCSGKLICTTNVPWNSTWSNKSLAEIWDNMTWMEWDRQIDNYTEVIYRLLELSQTQQEQNEQDLLALDKWDSLWNWFSITNWLWYIK

SEQ ID NO: 54 - protein, 156 aa, Unknown
  Synthesized or naturally derived
STMGAASITLTVQARQLLSGIVQQQSNLLRAIQARQHMLQLTVWGIKQLQARVLAVERYLRDQQLLGIWGCSGKLICTTNVPWNSSWSNKSQSEIWDNMTWMEWDKQISNYTEEIYRLLEVSQTQQEKNEQDLLALDKWASLWTWFDISHWLWYIK

SEQ ID NO: 55 - protein, 156 aa, Unknown
  Synthesized or naturally derived
STMGAASIALTVQARQLLSGIVQQQSNLLKAIEAQQHLLRLTVWGIKQLQARILAVERYLKDQQLLGIWGCSGKLICTTNVPWNSSWSNKSHDEIWNNMTWVEWEREIDNYTRIIYNLIEVSQNQQEKNEQDLLALDKWTSLWSWFKISNWLWYIR

SEQ ID NO: 56 - protein, 156 aa, Unknown
  Synthesized or naturally derived
STMGAASITLTVQVRQLLSGIVQQQSNLLKAIEAQQHLLKLTVWGIKQLQARVLAVERYLKDQQLLGIWGCSGKLICTTNVPWNASWSNKSYEDIWENMTWIQWEREINNYTGIIYSLIEEAQNQQENNEKDLLALDKWTNLWNWFNISNWLWYIK

SEQ ID NO: 57 - protein, 156 aa, Unknown
  Synthesized or naturally derived
STMGAASITLTVQVRQLLSGIVQQQSNLLKAIXAQQHLLKLTVWGIKQLQARVLAVERYLKDQQLLGIWGCSGKLICTTNVPWNASWSNKSYEDIWENMTWIQWEREINNYTGIIYSLIEEAQNQQETNEKDLLALDKWTNLWNWFNISNWLWYIK

SEQ ID NO: 58 - protein, 156 aa, Unknown
  Synthesized or naturally derived
STMGAASITLTVQARQLLSGIVQQQNNLLRAIEAQQQMLQLTVWGIKQLRARVLAVERYLRDQQLLGIWGCSGKLICTTNVPWNSSWSNKSQSEIWENMTWMQWEKEISNHTSTIYRLIEESQIQQEKNEQDLLALDKWASLWNWFDISNWLWYIK

SEQ ID NO: 59 - protein, 156 aa, Unknown
  Synthesized or naturally derived
STMGAASITLTVQARQLLSGIVQQQSNLLRAIEAQQHLLQLTVWGIKQLRARILAVERYLKDQQLLGIWGCSGKLICTTNVPWNSSWSNKSWEEIWNNMTWMEWEKEIGNYSDTIYKLIEESQTQQEKNEQDLLALDKWASLWNWFDITKWLWYIK

SEQ ID NO: 60 - protein, 157 aa, Unknown
  Synthesized or naturally derived
STMGAASITLTVQARTLLSGIVQQQNNLVRAIEAQQHLLQLSIWGIKQLRAKVLAIERYLRDQQILSLWGCSGKTICYTTVPWNETWSNNTSYDXIWGNLTWQQWDRKVRNYSGVIFELIXKAQEQQNTNEKSLLELDQWASLWNWFSITNWLWYIK

SEQ ID NO: 61 - protein, 157 aa, Unknown
  Synthesized or naturally derived
STMGAASITLTVQARTLLSGIVQQQNILLRAIEAQQHLLQLSIWGIKQLQAKVLAIERYLRDQQILSLWGCSGKTICYTTVPWNETWSNNTSYDTIWNNLTWQQWDEKVRNYSGVIFGLIEQAQEQQNTNEKSLLELDQWDSLWSWFGITKWLWYIK

SEQ ID NO: 62 - protein, 157 aa, Unknown
  Synthesized or naturally derived
STMGAASITLTVQARTLLSGIVQQQNNLLRAIEAQQHLLQLSIWGIKQLRAKVLAIERYLRDQQILSLWGCSGKTICYTTVPWNDXWSSNTSYDTIWXNLTWQQWDRKVRNYSGVIFDLIEQAQEQQNTNEKALLELDQWASLWNWFDITKWLWYIK

SEQ ID NO: 63 - protein, 154 aa, Unknown
  Synthesized or naturally derived
STMGAAATTLAVQTHTLLKGIVQQQDNLLRAIQAQQQLLRLSXWGIRQLRARLLALETLLQNQQLLSLWGCKGKLVCYTSVKWNRTWIGNESIWDTLTWQEWDRQISNISSTIYEEIQKAQVQQEQNEKKLLELDEWASIWNWLDITKWLWYIK

SEQ ID NO: 64 - protein, 157 aa, Unknown
  Synthesized or naturally derived
STMGAAATALTVRTHSVLKGIVQQQDNLLRAIQAQQHLLRLSVWGIRQLRARLQALETLIQNQQRLNLWGCKGKLICYTSVKWNTSWSGRYNDDSIWDNLTWQQWDQHINNVSSIIYDEIQAAQDQQEKNVKALLELDEWASLWNWFDITKWLWYIK

SEQ ID NO: 65 - protein, 158 aa, Unknown
  Synthesized or naturally derived
STMGAAATTLAVQTHTLMKGIVQQQDNLLRAIQAQQQLLRLSVWGIRQLRARLLALETLIQNQQLLNLWGCKGRLVCYTSVKWNRTWTNNNTDLDTIWGNLTWQEWDQQISNISATIYDEIQKAQVQQEHNEKKLLELDEWASIWNWLDITKWLWYIK

SEQ ID NO: 66 - protein, 167 aa, Unknown
  Synthesized or naturally derived
SAMGTAALTLSAQSRTLLAGIVQQQQQLLDVVKRQQEMLRLTVWGTKNLQARVTAIEKYLKDQARLNSWGCAFRQVCHTTVPWVNNSLKPDWDNMTWQEWEQQVRYLEANISEQLERAQIQQEKNTYELQKLNSWDVFTNWLDLTAWVKYIQYGVYIIVGIVALRIV

SEQ ID NO: 67 - protein, 162 aa, Unknown
  Synthesized or naturally derived
SAMGAASLTLSAQSRTLLAGIVQQQQQLLDVVKRQQEMLRLTVWGTKNLQARVTAIEKYLKDQAQLNSWGCAFRQVCHTTVPWVNDTLTPEWNNMTWQEWEGKIRDLEANISQQLEQAQIQQEKNMYELQKLNSWDVFSNWFDLTSWIKYIQYGVYIIIGIV

SEQ ID NO: 68 - protein, 158 aa, Unknown
  Synthesized or naturally derived
VAMGTASLTLSAQSRTLLAGIVQQQQQLLDVVKRQQELLRLTVWGTKILQARVTAIEKYLKDQAQLNSWGCAFRQVCHTTVPWANESLTPDWNNMTWQEWEQKVRYLEANISQSLEEAQLQQEKNMYELQKLNNWDVFTNWFDLTSWISYIQYGVYIV

SEQ ID NO: 69 - protein, 162 aa, Unknown
  Synthesized or naturally derived
SAMGAASLTLSAQSRTLLAGIVQQQQQLLDVVKRQQEMLRLTVWGTKNLQARVTAIEKYLKDQAQLNSWGCAFRQVCHTSVPWVNDTLTPDWNNMTWQEWEQKVRYLEANISQSLEQAQIQQEKNMYELQKLNSWDVFTNWLDFTSWVRYIQYGVYVVVGIV

SEQ ID NO: 70 - protein, 167 aa, Unknown
  Synthesized or naturally derived
SAMGAASLTVSAQSRTLLAGIVQQQQQLLDVVKRQQELLRLTVWGTKNLQARVTAIEKYLQDQARLNSWGCAFRQVCHTTVPWVNDSLAPDWDNMTWQEWEKQVRYLEANISKSLEQAQIQQEKNMYELQKLNSWDIFGNWFDLTSWVKYIQYGVLIIVAVIALRIV

SEQ ID NO: 71 - protein, 162 aa, Unknown
  Synthesized or naturally derived
AAMGAASLTLSAQSRTLFRGIVQQQQQLLDVVKRQQEMLRLTVWGTKNLQARVTAIEKYLADQARLNSWGCAFRQVCHTTVPWVNDTLTPEWNNMTWQEWEHKIRFLEANISESLEQAQIQQEKNMYELQKLNSWDVFGNWFDLTSWIKYIQYGVMIVVGIV

SEQ ID NO: 72 - protein, 167 aa, Unknown
  Synthesized or naturally derived
SAMGAASLTLSAHPGLYWAGIVQQQQQLLDVVKRQQEMLRLTVWGTKNLQTRVTAIEKYLRDQARLNSWGCAFRQVCYTTVLWENNSIVPDWNNMTWQEWEQQTRDLEANISRSLEQAQIQQEKNMYELQKLNSWDVFGNWFDLTSWIKYIQYGVYVIIGIIALRIV

SEQ ID NO: 73 - protein, 171 aa, Unknown
  Synthesized or naturally derived
SAMGGASLTLSAQSRTLLAGIVQQQQQLLDVVKRQQEMLRLTVWGTKNLQARVTAIEKYLKDQAQLNSWGCAFRQVCHTTVPWVNDSLTPDWNNMTWQEWEKRVHYLEANISQSLEQAQIQQEKNMYELQKLNSWDVFGNWFDLTSWIKYIQYGVYIVVGIIGLRIAIYIV

SEQ ID NO: 74 - protein, 171 aa, Unknown
  Synthesized or naturally derived
SAMGARSLTLSAQSRTLLAGIVQQQQQLLDVVKRQQEMLRLTVWGTKNLQARVTAIEKYLKHQAQLNSWGCAFRQVCHTTVPWVNDSLSPDWKNMTWQEWEKQVRYLEANISQSLEEAQIQQEKNMYELQKLNSWDILGNWFDLTSWVKYIQYGVHIVVGIIALRIAIYVV

SEQ ID NO: 75 - protein, 171 aa, Unknown
  Synthesized or naturally derived
SAMGAASLTLSAQSRTLLAGIVQQQQQLLDVVKRQQEMLRLTVWGTKNLQARVTAIEKYLKDQAQLNSWGCAFRQVCHTTVPWVNDSLSPDWNNMTWQEWEKQVRYLEANISQSLEQAQIQQEKNMYELQKLNSWDVFGNWFDLTSWIKYIQYGVYIVVGVIVLRIAIYIV

SEQ ID NO: 76 - protein, 171 aa, Unknown
  Synthesized or naturally derived
SAMGAASLTLSAQSRTLLAGIVQQQQQLLDIVKRQQELLRLTVWGTKNLQARVTAIEKYLKDQAQLNSWGCTFRQVCHTTVPWVNDSLTPRWNNMTWQEWEKQVRYLEANISQSLEEAQIQQEKNMYELQKLNSWDVFGNWFDLTSWIKYIQYGVYIVVGIIALRIAIYVV

SEQ ID NO: 77 - protein, 171 aa, Unknown
  Synthesized or naturally derived
AAMGAASLTLSAQSRTLLAGIVQQQQQLLDVVKRQQEMLRLTVWGTKNLQARVTAIEKYLKDQAQLNSWGCAFRQVCHTTVPWVNSSLEPDWENMTWQEWEQKVRYLEANISQKLEEAQIQQEQNMYELQKLNSWDIFGNWFDLTSWIKYIQYGVYIVVGIIVLRIVIYVV

SEQ ID NO: 78 - protein, 171 aa, Unknown
  Synthesized or naturally derived
SAMGAASLTLSAQSRTLLAGIVQQQQQLLDVVKRQQELLRLTVWGTKNLQARVTAIEKYLKDQALLNSWGCAFRQVCHTTVPWPNKNFTPNWDNMTWQQWENQVRFLDENITKLLEVAQIQQEENMYKLQKLNQWDVFSNWFDFTSWIAYIQIGLYVIVGLVVLRIVIYIL

SEQ ID NO: 79 - protein, 171 aa, Unknown; identical to SEQ ID NO: 78
  Synthesized or naturally derived

SEQ ID NO: 80 - protein, 171 aa, Unknown
  Synthesized or naturally derived
SAMGAASLTLSAQSRTLLAGIVQQQQQLVDVVKRQQELLRLTVWGTKNLQARVTAIEKYLKDQAQLNSWGCAFRQVCHTTVPWVNESLKPDWNNMTWQQWERQVRFLDANITKLLEEAQIQQEKNMYELQKLNQWDIFSNWFDFTSWMAYIRLGLYIVIGIVVLRIAIYII

SEQ ID NO: 81 - protein, 171 aa, Unknown
  Synthesized or naturally derived
SAMGATSLTLSAQSRTLLAGIVQQQQQPVDVVKRQQELLRLTVWGTKNLQARVTAIEKYLKDQAQLNSWGCAFRQVCHTTVPWPNETLTPNWNNMTWQQWEKQVHFLDANITALLEEAQIQQEKNMYELQKINSWDVFGNWFDLTSWIKYIHLGLYIVAGLVVLRIVVYIV

SEQ ID NO: 82 - protein, 171 aa, Unknown
  Synthesized or naturally derived
AAMGAASLTLSAQSRTLLAGIVQQQQQLLDVVKRQQEMLRLTVWGTKNLQARVTAIEKYLKDQAQLNSWGCAFRQVCHTTVPWVNDSLTPDWDNMTWQQWEKQIRDLEANISESLEQAQIQQEKNMYELQKLNSWDVFGNWFDLASWVKYIQYGVYIVVGIVALRVIIYVV

SEQ ID NO: 83 - protein, 171 aa, Unknown
  Synthesized or naturally derived
SAMGAASLTLSAQSRTLLAGIVQQQQQLLDVVKRQQEMLRLTVWGTKNLQTRVTAIEKYLKDQASLNAWGCAFRQVCHTTVPWINDTLTPNWDNMTWQEWEEKVNYLEENITQLLEAAQIQQEKNMYELQKLNNWDIFGNWFDLTSWVKYVYLGLYVVAGIIILRIVIYVV

SEQ ID NO: 84 - protein, 171 aa, Unknown
  Synthesized or naturally derived
SAMGAASLTLTAQSRTLLAGIVQQQQQLLDVVKRQQELLRLTVWGTKNLQTRVTAIEKYLKDQAQLNAWGCAFRQVCHTTVPWPNASLTPKWNNETWQEWERKVDFLEENITALLEEAQIQQEKNMYELQKLNSWDVFGNWFDLASWIKYIQYGVYIVVGVILLRIVIYIV

SEQ ID NO: 85 - protein, 171 aa, Unknown; identical to SEQ ID NO: 84
  Synthesized or naturally derived

SEQ ID NO: 86 - protein, 171 aa, Unknown; identical to SEQ ID NO: 84
  Synthesized or naturally derived

SEQ ID NO: 87 - protein, 170 aa, Unknown
  Synthesized or naturally derived
SAMGAASLTLTAQSRTLLAGIVQQQQQLLDVVKRQQELLRLTVWGTKNLQTRVTAIEKYLKDQAQLNAWGCAFRQVCHTTVPWPNASLTPKWNNETWQEWERKVDFLEENITALLEEAQIQQEKNMYELQKLNSWDVFGNWFLASWIKYIQYGVYIVVGVILLRIVIYIV

SEQ ID NO: 88 - protein, 171 aa, Unknown; identical to SEQ ID NO: 84
  Synthesized or naturally derived

SEQ ID NO: 89 - protein, 171 aa, Unknown
  Synthesized or naturally derived
SAMGAASLTLTAQSRTLLAGIVQQQQQLLDVVKRQQELLRLTVWGTKNLQTRVTAIEKYLKDQAQLNAWGCAFRQVCHTTVPWPNASLTPKWSNETWQEWERKVDFLEENITALLEEAQIQQEKNMYELQKLNSWDVFGNWFDLASWIKYIQYGVYIVVGVILLRIVIYIV

SEQ ID NO: 90 - protein, 171 aa, Unknown; identical to SEQ ID NO: 84
  Synthesized or naturally derived

SEQ ID NO: 91 - protein, 171 aa, Unknown; identical to SEQ ID NO: 84
  Synthesized or naturally derived

SEQ ID NO: 92 - protein, 171 aa, Unknown; identical to SEQ ID NO: 84
  Synthesized or naturally derived

SEQ ID NO: 93 - protein, 171 aa, Unknown
  Synthesized or naturally derived
SAMGAASLTLTAQSRTLLAGIVQQQQQLLDVVKRQQELLRLTVWGTKNLQTRVTAIEKYLKDQAQLNAWGCAFRQVCHTTVPWPNANLTPKWNNETWQEWERKVDFLEENITALLEEAQIQQEKNMYELQKLNSWDVFGNWFDLASWIKYIQYGVYIVVGVILLRIVIYIV

SEQ ID NO: 94 - protein, 171 aa, Unknown; identical to SEQ ID NO: 84
  Synthesized or naturally derived

SEQ ID NO: 95 - protein, 171 aa, Unknown; identical to SEQ ID NO: 84
  Synthesized or naturally derived

SEQ ID NO: 96 - protein, 171 aa, Unknown; identical to SEQ ID NO: 84
  Synthesized or naturally derived

SEQ ID NO: 97 - protein, 171 aa, Unknown
  Synthesized or naturally derived
SAMGAASLTLTAQSRTLLAGIVQQQQQLLDVVKRQQELLRLTVWGTKNLQTRVTAIEKYLKDQAQLNAWGCAFRQVCHTTVPWPNASLTPEWNNETWQEWERKVDFLEENITALLEEAQIQQEKNMYELQKLNSWDVFGNWFDLTSWIKYIQYGVYIVVGVILLRIVIYIV

SEQ ID NO: 98 - protein, 171 aa, Unknown
  Synthesized or naturally derived
SAMGAASLTLTAQSRTLLAGIVQQQQQLLDVVKRQQELLRLTVWGTKNLQTRVTAIEKYLKDQAQLNAWGCAFRQVCHTTVPWPNASLTPKWDNETWQEWERKVDFLEENITALLEEAQIQQEKNMYELQKLNSWDVFGNWFDLTSWIKYIQYGVYIVVGVILLRIVIYIV

SEQ ID NO: 99 - protein, 171 aa, Unknown
  Synthesized or naturally derived
SAMGAASLTLTAQSRTLLAGIVQQQQQLLDVVKRQQELLRLTVWGTKNLQTRVTAIEKYLKDQAQLNAWGCAFRQVCHTTVPWPNASLTPKWDNETWQEWERKVDFLEENITALLEEAQIQQEKNMYELQKLNSWDVFGNWFDLXSWIKYIQYGVYIVVGVILLRIVIYIV

SEQ ID NO: 100 - protein, 171 aa, Unknown
  Synthesized or naturally derived
SAMGAASLTLTAQSRTLLAGIVQQQQQLLDVVKRQQELLRLTVWGTKNLQTRVTAIEKYLKDQAQLNAWGCAFRQVCHTTVPWPNANLTPNWNNETWQEWERKVDFLEENITALLEEAQIQQEKNMYELQKLNSWDVFGNWFDLASWIRYIQYGVYIVVGVILLRIVIYIV

SEQ ID NO: 101 - protein, 171 aa, Unknown
  Synthesized or naturally derived
SAMGAASLTLTAQSRTLLAGIVQQQQQLLDVVKRQQELLRLTVWGTKNLQTRVTAIEKYLKDQAQLNSWGCAFRQVCHTTVLWPNDSLVPDWNNMTWQEWEKKVEFLEANITQMLEEARLQQEKNMYELQKLNSWDVFGNWFDLTSWVRYIQYGVFLVIGIVLLRIVIYVV

SEQ ID NO: 102 - protein, 171 aa, Unknown
  Synthesized or naturally derived
SAMGAASLTLSAQSRTLLAGIVQQQQQLLDVVKRQHELLRLTVWGTKNLQTRVTAIEKYLKDQAQLNSWGCAFRQVCHTTVPWPNDSLVPNWDNMTWQEWEGKVDFLEANITQLLEEAQIQQEKNMYELQKLNSWDIFGNWFDLTSWIRYIQYGVLIVLGVVGLRIVIYVV

SEQ ID NO: 103 - protein, 171 aa, Unknown
  Synthesized or naturally derived
SAMGAASLTLTAQSRTLLAGIVQQQQQLLDVVKRQQELLRLTVWGTKNLQTRVTAIEKYLKDQAQLNTWGCAFRQVCHTTVPWPNASLIPKWNNETWQEWERKVDFLEENITALLEEAQIQQEKNMYELQKLNSWNVFGNWFDLASWIKYIQYGVYIVVGVILLRIVIYIV

SEQ ID NO: 104 - protein, 171 aa, Unknown; identical to SEQ ID NO: 103
  Synthesized or naturally derived

SEQ ID NO: 105 - protein, 171 aa, Unknown
  Synthesized or naturally derived
SAMGAASLTLTAQSRTLLAGIVQQQQQLLDVVKRQQELLRLTVWGTKNLQTRVTAIEKYLKDQAQLNAWGCAFRQVCHTTVPWPNASLTPDWNNDTWQEWERKVDFLEENITALLEEAQIQQEKNMYELQKLNSWDVFGNWFDLASWIKYIQYGVYIVVGVILLRIVIYIV

SEQ ID NO: 106 - protein, 171 aa, Unknown; identical to SEQ ID NO: 84
  Synthesized or naturally derived

SEQ ID NO: 107 - protein, 171 aa, Unknown
  Synthesized or naturally derived
SAMGAASLTLTAQSRTLLAGIVQQQQQLLDVVKRQQELLRLTVWGTKNLQTRVTAIEKYLKDQAQLNAWGCAFRQVCHTTVPWPNASLTPNWNNETWQEWERKVDFLEENITALLEEAQIQQEKNMYELQKLNSWDVFGNWFDLASWIRYIQYGVYIVVGVILLRIVIYIV

SEQ ID NO: 108 - protein, 171 aa, Unknown
  Synthesized or naturally derived
SAMGAASVTRSAQSRTLLAGIVQQQQQLLDVVKRQQELLRLTVWGTKNLQTRVTAIEKYRKDQAQLNSWGCAFRQVCHTTVPWPNASLVPNWNNMTWQEWERQVDDLEANITQALEEAQIQQEKNMYELQKLNSWDIFGNWFDLTSWIKYIQYGVLIVLGVVGLRIVIYVV

SEQ ID NO: 109 - protein, 171 aa, Unknown
  Synthesized or naturally derived
SAMGAASVTLSAQSRTLLAGIVQQQQQLLDVVKRQQELLRLTVWGAKNLQTRVTAIEKYLKDQAQLNSWGCAFRQVCHTTVPWPNDTLTPNWNNMTWQEWEKQVNFLEANITQSLEEAQIQQEKNTYELQKLNSWDIFGNWFDLTSWIKYIQYGVLIVLGVIGLRIVIYVV

SEQ ID NO: 110 - protein, 171 aa, Unknown; identical to SEQ ID NO: 109
  Synthesized or naturally derived

SEQ ID NO: 111 - protein, 171 aa, Unknown
  Synthesized or naturally derived
SAMSAASVTLSAQSRTLLAGIVQQQQQLLDVVKRQQELLRLTVWGAKNLQTRVTAIEKYLKDQAQLNSWGCAFRQVCHTTVPRPNDTLTPNWNNMTWQEWEKQVNFLEANITQSLEEAQIQQEKNTYELQKLNSWDIFGNWFDLTSWIKYIQYGVLIVLGVIGLRIVIYVV

SEQ ID NO: 112 - protein, 171 aa, Unknown
  Synthesized or naturally derived
SAMGAASVTLSAQSRTLLAGIVQQQQQLLDVVKRQQELLRLTVWGTKNLQTRVTAIEKYLKDQAQLNSWGCAFRQVCHTTVPWPNETLVPNWNNMTWQEWERQVDFLEANITQLLEEAQIQQEKNMYELQKLNSWDIFGNWFDLTSWIRYIQYGVLIVLGVIGLRIVIYVV

SEQ ID NO: 113 - protein, 170 aa, Unknown
  Synthesized or naturally derived
SAMGAASLTLTAQSRTLLTGIVQQQQQLLDVVKRQQELLRLTVWGTKNLQTRVTAIEKYLKDQAQLNSWGCAFRQVCHTTVPWPNDSLVPDWNNMTWQEWERKVDFLEANITQLLEEAQVQQEKNMYELQKLNSWDVFGNWFDLTSWVRYIQYGVYLVIGLVMLRVAIYI

SEQ ID NO: 114 - protein, 171 aa, Unknown
  Synthesized or naturally derived
STMGAASITLTVQARKLLSGIVQQQNNLLRAIEAQQHLLQLSVWGIKQLQARVLAIERYLRDQQILGLWGCSGKSVCYTNVPWNTTWSNNNSYDTIWGNMTWQNWDEQVRNYSGVIFGLLEQAQEQQSINEKSLLELDQWSSLWNWFDITKWLWYIKIFIMVVAGIVGIRI

SEQ ID NO: 115 - protein, 178 aa, Unknown
  Synthesized or naturally derived
STMGAASLTLTVQARQLLTGIVQQQSNLLRAIEAQQHLLQLSVWGIKQLQARVLAIERYLKDQQLLGIWGCSGKLICTTSVPWNRTWSNKTYNEIWDNMTWMEWDREVRNYTEIIYGLIEQAQDQQENNEKKLLELDHWTSLWNWFDISHWLWYIKIFIMIIGGLIVCRIIFAVLAIV

SEQ ID NO: 116 - protein, 179 aa, Unknown
  Synthesized or naturally derived
STMGAAAVTLTVQARQLLSGIVQQQNNLLRAIEAQQHLLQLTVWGVKQLQARLLAVERYLQDQQILGLWGCSGKSICYTTVPWNKTWSGKSMSDIWNNLTWQQWDKLITNYTGTIFGLLEEAQSQQEKNEKDLLELDQWASLWNWFDITNWLWYIKIFLMAVGGIIGLRIIMSVVSVIR

SEQ ID NO: 117 - protein, 178 aa, Unknown
  Synthesized or naturally derived
STMGAASLTLTVQARQLLSGIVQQQSNLLRAIEAQQHLLQLSVWGIKQLQARVLAVERYLKDQQLLGLWGCSGKLICTTSVPWNTTWTNKSYDDIWYNMTWMQWDKEVSNYTDVIYNLLEKAQTQQENNEKELLELDKWASLWNWFDITSWLWYIKIFIIIVGGLIGLRIVFALLSIV

SEQ ID NO: 118 - protein, 179 aa, Unknown
  Synthesized or naturally derived
STMGAASVVLTVQARQLLTGIVQQQNNLLRAIEAQQHLLQLSVWGIKQLQARVLAVERYLRDQQLLGLWGCTGKTICPTAVRWNKTWGNISDYQVIWNNYTWQQWDREVNNYTGLIYTLLEEANTQQEKNEKELLELDSWANLWSWFDITNWLWYIKMFLIVVGGIIGLRICFAIGSLI

SEQ ID NO: 119 - protein, 176 aa, Unknown
  Synthesized or naturally derived
STMGAASVVLTVQARQLLSGIVQQQNNLLRAIEAQQHLLQLSVWGIKQLQARVLAVERYLRDQQILGLWGCSGKAICYTTVPWNNTWSANTSFDEIWNNLTWQDWDKRVKNYSGVIFSLIEQAQEQQNTNEKSLLELDQWSSLWNWFDITRWLWYIKLFIMIVAGLVGIRIVGAII

SEQ ID NO: 120 - protein, 175 aa, Unknown
  Synthesized or naturally derived
STMGAASVVLTVQARHLLSGIVQQQNNLLRAIEAQQHLLQLSVWGIKQLQARVLAVERYLKDQQILSLWGCSGKAICYTTVPWNATWSANTSYDEIWNNLTWQDWDKKVKNYSGVIFSLIEQAQEQQNTNEKDLLELDQWSSLWSWFNITQWLWYIKIFLIVVAGLIGFRLIGIV

SEQ ID NO: 121 - protein, 172 aa, Unknown
  Synthesized or naturally derived
STMGAAAVTLTVQARQLLSGIVQQQNNLLKAIEAQQHLLQLSIWGVKQLQARLLAVERYLQDQQILGLWGCSGKAVCYTTVPWNNSWPGSNSTDDIWGNLTWQQWDKLVSNYTGKIFGLLEEAQSQQEKNERDLLELDQWASLWNWFDITKWLWYIKIFLMAVGGIIGLRII

SEQ ID NO: 122 - protein, 172 aa, Unknown; identical to SEQ ID NO: 121
  Synthesized or naturally derived

SEQ ID NO: 123 - protein, 172 aa, Unknown
  Synthesized or naturally derived
STMGAASVVLTVQARQLLSGIVQQQNNLLRAIEAQQHLLQLSVWGIKQLQARVLAVERYLKDQQILGLWGCSGKTICYTTVPWNDTWSNNLSYDAIWGNLTWQEWDRKVRNYSGTIFSLIEQAQEQQNTNEKSLLELDQWSSLWNWFDITNWLWYIKIFLIVVASLVGIRIV

SEQ ID NO: 124 - protein, 175 aa, Unknown
  Synthesized or naturally derived
STMGAASIALTAQTRNLXHGIVQQQANLLQAIETQQHLLQLSVWGVKQLQARMLAVEKYLRDQQLLSLWGCADKVTCHTTVPWNNSWVNFTQTCAKNSSDIQCIWENMTWQEWDRLVQNSTGQIYNILQIAHEQQERNKKELYELDKWSSLWNWFDITQWLWYIKIFIMIVGAIV

SEQ ID NO: 125 - protein, 172 aa, Unknown
  Synthesized or naturally derived
STMGAASIALTAQARGLLSGIVQQQQNLLQAIEAQQHLLQLSVWGIKQLQARMLAVEKYIRDQQLLSLWGCANKLVCHSSVPWNLTWAEDSTKCNHSDAKYYDCIWNNLTWQEWDRLVENSTGTIYSLLEKAQTQQEKNKQELLELDKWSSLWDWFDITQWLWYIKIAIIIV

SEQ ID NO: 126 - protein, 173 aa, Unknown
  Synthesized or naturally derived
AMGSASVALTIQAQSLNGRASASSNRMLLKLVETQSALLQLTVWGVKNLQVRVATIEGYLEEQAKLASIGCANMQICRTIVPWNKTWGEEDPWQNMTWKQWHERVRNYTDIIEADLVEAYDLQEENEKKLAELGDWTNWFSGFGLFNIFKYVLYAAYVVGGLIGLRIIMVVIA

SEQ ID NO: 127 - protein, 173 aa, Unknown
  Synthesized or naturally derived
AAMGAASTALTVQSRSLLSGIVQQQQELLKAVEAHGQLLTLTAWGVRNLNTRLTAIEKYLKDQAKLNEWGCAFKQICHTTVPWNNSLEDPDWDNMTWQEWEMKVANYTDEWEGALQRAQEQQERNVHALQSLQDWDSLWNWFDLSRWFWWIRLVVYIIAALILLRIAMFGVNI

SEQ ID NO: 128 - protein, 166 aa, Unknown
  Synthesized or naturally derived
AAMGAASTALTVQSRSLLSGIVQQQQELLKAVEAHGHLLSLTAWGVRNLNTRLTAIEKYLKDQSKLNEWGCAFKQICHTTVPWNHTWGEPDWNNMTWQEWERKVANYTDEWEGALQRAQEQQERNVHALQSLTDWDSLWNWFDLSRWFWWIRLVVYIIAALILLRI

SEQ ID NO: 129 - protein, 170 aa, Unknown
  Synthesized or naturally derived
TTMGAAATALTEQSRSLLAGIMQQQENLLRAVEAQQSLLQPSVWGIKQLQTRLSSLEKYLRDQTILQAWGCANRPICHTIVPWNTSWANGSLPDWENMTWQKWSMLVENDTYTIQQLLEQANQQQASNLNELMKLSKWDSLWSWFDISDWQRYIKIFVIVVAALIALRIV

SEQ ID NO: 130 - protein, 170 aa, Unknown
  Synthesized or naturally derived
ATMGAAATALTVQSRSLLAGIVQQQENLLRAVEAQQSLLQLSVWGIKQLQARLSSLEKYLRDQTILQAWGCANQPICHTIVPWNDSWAKNSTPDWEHMTWQEWSKLIENDTYTIQQLLENANHQQSKNMNDLLKLSKWDSLWSWFDISNWLWYIKIFIMVVAALVALRII

SEQ ID NO: 131 - protein, 171 aa, Unknown
  Synthesized or naturally derived
SAMGTAALTLSAQSRTLLAGIVQQQQQLLDVVKRQQEMLRLTVWGTKNLQARVTAIEKYLKDQARLNSWGCAFRQVCHTTVPWVNNSLKPDWDNMTWQEWEQQVRYLEANISEQLERAQIQQEKNTYELQKLNSWDVFTNWLDLTAWVKYIQYGVYIIVGIVALRIVIYVV

SEQ ID NO: 132 - protein, 171 aa, Unknown; identical to SEQ ID NO: 74
  Synthesized or naturally derived

SEQ ID NO: 133 - protein, 169 aa, Unknown
  Synthesized or naturally derived
AAMGAASLTLSAQSRTLLAGIVQQQQQLLDVVKRQQEMLRLTVWGTKNLQARVTAIEKYLKDQAQLNSWGCAFRQVCHTTVPWVNDTLTPDWNNMTWQEWEQRIRNLEANISESLEQAQIQQEKNMYELQKLNSWDVFGNWFDLTSWIKYIQYGVYIVVGIIVLRIVIY

SEQ ID NO: 134 - protein, 171 aa, Unknown; identical to SEQ ID NO: 81
  Synthesized or naturally derived

SEQ ID NO: 135 - protein, 171 aa, Unknown; identical to SEQ ID NO: 80
  Synthesized or naturally derived

SEQ ID NO: 136 - protein, 175 aa, Unknown
  Synthesized or naturally derived
SAMGTASLTLSAQSRTLLAGIVQQQQQLLDVVKRQQEMLRLTVWGTKNLQTRVTAIEKYLKDQARLNSWGCAFRQVCHTTVPWDALGANKTLEPQWNNMTWQEWEKQINFLEDNITRLLEEAQIQQEKNMYELQKLNSWDVFGNWFDLTSWVKYVYLGLYVVAGVIVLRIVIYVV

SEQ ID NO: 137 - protein, 171 aa, Unknown; identical to SEQ ID NO: 83
  Synthesized or naturally derived

SEQ ID NO: 138 - protein, 171 aa, Unknown
  Synthesized or naturally derived
SAMGAASFRLTAQSRTLLAGIVQQQQQLLGVVKRQQELLRLTVWGTKNLQTRVTAIEKYLEDQAQLNAWGCAFRQVCHTTVPWPNASLTPDWNNDTWQEWERKVDFLEENITALLEEAQIQQEKNMYELQKLNSWDVFGNWFDLASWIKYIQYGIYVVVGVILLRIVIYIV

SEQ ID NO: 139 - protein, 171 aa, Unknown; identical to SEQ ID NO: 84
  Synthesized or naturally derived

SEQ ID NO: 140 - protein, 171 aa, Unknown; identical to SEQ ID NO: 102
  Synthesized or naturally derived

SEQ ID NO: 141 - protein, 171 aa, Unknown
  Synthesized or naturally derived
SAMGAASLTLSAQSRTLLAGIVQQQQQLLDVVKRQQELLRLTVWGTKNLQTRVTAIEKYLKDQAPXNSWGCAFRQVCHTTVPWPNDTLTPXWNNMXWQEWEKQVNFLEANITZXLEEAQIQQEXNMYELQKLNXXDXFGNWXDLTXWIKYIQYGVLIVLGVIGLRIVIYVV

SEQ ID NO: 142 - protein, 170 aa, Unknown; identical to SEQ ID NO: 113
  Synthesized or naturally derived

SEQ ID NO: 143 - protein, 171 aa, Unknown; identical to SEQ ID NO: 101
  Synthesized or naturally derived

SEQ ID NO: 144 - protein, 171 aa, Unknown; identical to SEQ ID NO: 108
  Synthesized or naturally derived

SEQ ID NO: 145 - protein, 171 aa, Unknown; identical to SEQ ID NO: 107
  Synthesized or naturally derived

SEQ ID NO: 146 - protein, 171 aa, Unknown
  Synthesized or naturally derived
TAMGAAATALTVQSRHLLAGILQQQKKLLDIVEQQQELLKLTVWGTKNLQARVTAIEKYLADQSLLNTFGCAWRQVCHTTVEWIYSQTPEWNKQTWLEWERNISRLEGNISVALQDAQEQHERNVHDLEKLNSWGDMLSWLNMDWWLKYIRIGIFIILGIIGLRIIFLLWS

SEQ ID NO: 147 - protein, 169 aa, Unknown
  Synthesized or naturally derived
TAMGAAATALTVQSRHLLAGILQQQKNLLDIVKRQQNLLKLTVWGTKNLQARVTAIEKYLADQSLLNTFGCAWRQVCHTVVPWTFNKTPEWQKESWLQWERNISYLEANITIALQEAQDQHEKNVHELEKLSNWGDAFSWLNLDWWMQYIKIGFFIVIGIIGLRVAWLL

SEQ ID NO: 148 - protein, 170 aa, Unknown
  Synthesized or naturally derived
SAMCSVATAMTVQSQALLTGMVEQQKQLLRLVEQQQELLKLTIWGVKNLQARLTALEEYIGDQAMLSLWGCSFAQVCHTNVVWPNESVTPNWTSETWMEWQKRVDSISNNITLDLQKAYEQEQKNIFELQKLGDLTSWANWFDFTWWSKYIKIGFFIVMAIIGLRILAAL

SEQ ID NO: 149 - protein, 170 aa, Unknown
  Synthesized or naturally derived
SAMGSVSVALTVQSQSLVTGIVEQQKQLLKLIEQQSELLKLTIWGVKNLQTRLTSLENYIKDQALLSQWGCSWAQVCHTSVEWTNTSITPNWTSETWKEWETRTDYLQQNITEMLKQAYDREQRNTYELQKLGDLTSWASWFDFTWWVQYLKWGVFLVLGIIGLRILLAL

SEQ ID NO: 150 - protein, 171 aa, Unknown
  Synthesized or naturally derived
SAMGSVAVALTVQSQTLLNGIVEQQKVLLSLIDQHSELLKLTIWGVKNLQVRLTALEEYVADQSRLSVWGCSFSQVCHTSVKWPNNSIVPNWTSETWLEWDRRVNSIVTNMTIDLQRAYELEQRNIFELQKLGDLNFHGLTGFDLTWWLKYVKIGLLVVVVIIGLRMLACL

SEQ ID NO: 151 - protein, 169 aa, Unknown
  Synthesized or naturally derived
SAMGSVAVALTVQSQALLNGIVEQQKILLSLIDQHSELLKLTIWGVKNLQARLTALEDYVADQSRLAVWGCSFSQVCHTNVPWPNESITPNWTSETWLEWDRRVTAITNNMTIDLQRAYELEQKNMYELQKLGDLTSWASWFDLTWWLKYVKIGILIIMVVIGLRILAC

SEQ ID NO: 152 - protein, 169 aa, Unknown
  Synthesized or naturally derived
STMGSVAVALTVQSQALLNGIVEQQKVLLSLIDQHSELLKLTIWGVKNLQARLTALEDYVADQARLSMWGCSFAQVCHTHVPWPNDSITPNWTSETWLEWDKRVTALTDNMTVNLQKAYELEQKNIYELEKLGDWTSWASWFDFTWWLKYVKIGLLIVIVIIVLRILAC

SEQ ID NO: 153 - protein, 172 aa, Unknown
  Synthesized or naturally derived
TAMGAAATTLTVQSRHLLAGILQQQKNLLAAVEQQQQLLKLTIWGVKNLNARVTALEKYLEDQARLNSWGCAWKQVCHTTVPWKYNNTPKWDNMTWLEWERQINALEGNITQLLEEAQNQESKNLDLYQKLDDWSGFWSWFSLSTWLGYVKIGFLVIVIILGLRFAWVLWGC

SEQ ID NO: 154 - protein, 172 aa, Unknown
  Synthesized or naturally derived
AAMGATATALTVQSQQLLAGILQQQKNLLAAVEQQQQMLKLTIWGVKNLNARVTALEKYLEDQTRLNLWGCAFKQVCHTTVPWTFNNTPDWDNMTWQEWESQITALEGNISTTLVKAYEQEQKNMDTYQKLGDWTSWWNIFDVSSWFWWIKWGFYIVIGLILFRMAWLIWGC

SEQ ID NO: 155 - protein, 176 aa, Unknown
  Synthesized or naturally derived
TAMGLVSTILTVQAQVVIQGILQQQKQLLVLVEKQQELLRLTIWGVKNLQARLTAIEEYLKDQTLLASWGCQWKQVCHTNVEWNYNITPNWTRDTWIEWDRQVGVLEANISTLLQEAYTTELENRNAFKKLQEFNFWNWLDILSWFQYIKYAVLIIIGIIVLRVVSFIVQNIVKMC

SEQ ID NO: 156 - protein, 176 aa, Unknown
  Synthesized or naturally derived
TAMGLVSTILTVQAQVVIQGILQQQKQLLVLVEKQQELLRLTIWGVKNLQARLTAIEEYLKDQALLASWGCQWKQICHTNVEWNYNITPNWTRDTWIEWDRRVGVLEANISTLLQEAYTTELENRNAFKKLQEFNFWSWLDILSWFQYIKYAVLIIIGIIVLRIVSFIVQNIVKMC

SEQ ID NO: 157 - protein, 176 aa, Unknown
  Synthesized or naturally derived
TAMGLVSTILTVQAQVVIQGILQQQKQLLVLVEKQQELLRLTIWGVKNLQARLTAIEEYLKDQALLASWGCQWKQVCHTNVPWNYNVTPNWTRDTWIEWERQVGSLEANITTLLQEAYTTELENRNNFKKLQDFNFWSWMDLTTWFQYIKYAVLIIIGIIILRILSFIIQSVVKMC

SEQ ID NO: 158 - protein, 176 aa, Unknown
  Synthesized or naturally derived
TAMGLVSTILTVQAQAVLQGILQQQKQLLVLVEKQQELLRLTIWGVKNLQARLTALEEYVKHQALLASWGCQWKQVCHTNVEWTYNITPNWTKDTWREWESKVAIYDKNITSLLQEAYTTELENQNKFKKLQEFNFWSWLDISHWFTYVKYAVLIILVIIGLRVLSFIIQNVVKMC

SEQ ID NO: 159 - protein, 177 aa, Unknown
  Synthesized or naturally derived
GTMGAAATALTVQSRSLLAGIVQQQENLLRAVTAQQSLLQLTVWGVKQLQARLTAVEKFIKDQTLLNAWGCANKAVCHTTVPWNNSWAKGHFPEWDNMTWQQWSELVDNDTMTIQQLLEAAQEQQGKNQHELMKPGQWDFLWNWFDISKWLWYIKIFIIVVAALIGLRILMFILGVI

SEQ ID NO: 160 - protein, 177 aa, Unknown
  Synthesized or naturally derived
GTMGAAATALTVQSRSLLAGIVQQQENLLRAVTAQQSLLQLSVWGIKQLQARLTAVEKFIKDQTLLNSWGCANRAVCHTQVLWNNTWAKGHFPEWDNMTWQQWSMLVDNDTALIQXLLEEAQEQQGKNAHELMKLGQWDWLWNWFDISKWLWYIKIFIIVVAALVGLRVLMFILGII

SEQ ID NO: 161 - protein, 174 aa, Unknown
  Synthesized or naturally derived
TAMGAVATALTVQSRSLLSGIVQQQEHLLRAIEHQQHLLQLTVWGIKNLNARLTALEKYLEDQARLNSWGCAWKQICYTSVPWNKTWTNSTNPDWQNMTWQEWEKLVDNASDTITVLLQEAQEQQERNVHELQKLNDWDSLWSWFNLSAWFRWLRIAVIVVASLILLRIVMYII

SEQ ID NO: 162 - protein, 174 aa, Unknown
  Synthesized or naturally derived
TAMGAVATALTVQSRSLLSGIVQQQEHLLRAIEHQQHLLQLTVWGIKNLNARLTALEKYLEDQAKLNSWGCAWKQICYTSVPWNKTWSNYTDPQWQNMTWQEWEMKVDNHTGLISQLLQEAQEQQERNVHELQKLNDWDSLWSWFNLSAWFRWLRIAVIVVASLILLRIVMYIV

SEQ ID NO: 163 - protein, 177 aa, Unknown
  Synthesized or naturally derived
GTMGAAATALTVQSRSLLAGIVQQQANLLRAVEAQQHLLQLSVWGIKQLQARLTALEKFIKDQALLNLWGCANRQICHTRVPWNDSWANHTQPGWENMTWQQWSRLVDNDTTTIQELLELAQRQQEENQHKLQKLLEWDSLWEWFDISKWLWYIKIFCMVVAGLVLFRLVMFVLGIL

SEQ ID NO: 164 - protein, 173 aa, Unknown
  Synthesized or naturally derived
AAMGAAATALTVQSQQLLAGILQQQKNLLAAVEQQQQMLKLTIWGVKNLNARVTALEKYLEDQARLNIWGCAFRQVCHTTVLWKYNNTPDWENMTWQEWERQIEKYEANISRILEQAHEQEQKNLDSYQKLVSWSDFWSWFDLTKWFGWMKIAIMVIAGIIVARVLLVIIGIL

SEQ ID NO: 165 - protein, 173 aa, Unknown
  Synthesized or naturally derived
TAMGAAATALTVQSQHLLAGILQQQKNLLAAVEAQQQMLKLTIWGVKNLNARVTALEKYLEDQARLNAWGCAWKQVCHTTVPWQWNNRTPDWNNMTWLEWERQISYLEGNITTQLEEARAQEEKNLDAYQKLSSWSDFWSWFDFSKWLNILKIGFLDVLGIIGLRLLYTVYSC

SEQ ID NO: 166 - protein, 173 aa, Unknown
  Synthesized or naturally derived
TAMGAAATALTVQSQHLLAGIMQQQKNLLAAVEAQQQMLKLTIWGVKNLNARVTALEKYLEDQARLNVWGCAWKQVCHTTVPWQWQNMTPNWQNMTWLEWERQIGELEGNITEQLVKAREQEEKNLDAYQRLTSWSNFWSWFDFSKWLNILKIGFLVVVGIIGLRLLYTIYSC

SEQ ID NO: 167 - protein, 172 aa, Unknown
  Synthesized or naturally derived
TAMGAAATALTVQSQHLLAGILQQQKNLLAAVGAQQQMLKLTIWGVKNLNARVTALEKYLADQARLNAWGCAWKQVCHTTVPWTWNNTPEWNNMTWLEWEKQIEGLEGNITKQLEQAREQEEKNLDAYQKLSDWSSFWSWFDFSKWLNILKIGFLAVIGVIGLRLLYTLYTC

SEQ ID NO: 168 - protein, 175 aa, Unknown
  Synthesized or naturally derived
TAMGAAASSLTVQSRHLLAGILQQQKNLLAAVEAQQQMLKLTIWGVKNLNARVTALEKYLEDQARLNSWGCAWKQVCHTTVEWPWTNRTPDWQNMTWLEWERQIADLESNITGQLVKAREQEEKNLDAYQKLTSWSDFWSWFDFSKWLNILKMGFLVIVGIIGLRLLYTVYGCIV

SEQ ID NO: 169 - protein, 229 aa, Unknown
  Synthesized or naturally derived
TAMGLVSTILTVQAQAVLQGILQQQKQLLVLVEKQQELLRLTIWGVKNLQARLTALEEYVQDQSLLASWGCQWKQVCHTNVPWNYNITPNWTKDTWMEWDRQVKMYDDNITALLQEAYVTELENQNKFKQLQEFNFWSWLDLSQWFLYIKYAVLIIGIIIAARILSFIIQQIYRMCQGYRVLSPSAYVEQDWLQETCPKPTDKEEEEETEKERIYINLEQSKKESLPPP

SEQ ID NO: 170 - protein, 169 aa, Unknown
  Synthesized or naturally derived
TAMGGAATALTLQSQTLLAGIVQQQQKLLEAVEAQQHLLGLTVWGVKNLNARLTALETYLRDQAILSNWGCAFKQICHTAVTWEKACGNNSNFCPKPQWKNMTWHRWEQEVDNLTDHIDGLLREAQEQQERNVHDLTKLQEWDSLWSWFDLSKWFFYLKIGFYVIGALV

SEQ ID NO: 171 - protein, 168 aa, Unknown
  Synthesized or naturally derived
TAMGSAATALTLQSQTLLAGIVQQQQKLLEAVEAQQHLLGLTVWGVKNLNARLTALETYLRDQAIMSNWGCAFKQICHTAVTWQQACGNNSRCPTPQWENMTWHTWERQVDNLTDHIDNLLREAQEQQEKNVHDLTKLQEWDSLWSWFDLSKWFQYLKIGFFAIAAIV

SEQ ID NO: 172 - protein, 101 aa, Unknown
  Synthesized or naturally derived
LKEVEKSITNLEKSLTSLSEVVLQNRRGLDLLFLKEGGLCAALKEECCFYADHTGLVRDSMAKLRERLSQRQKLFESQQGWFEGLFNKSPWFTTLISTIMG

SEQ ID NO: 173 - protein, 135 aa, Unknown
  Synthesized or naturally derived
TKVMGTQEDIDKKIEDRLSALYDVVRVLGEQVQSINFRMKIQCHANYKWICVTKKPYNTSDFPWDKVKKHLQGIWFNTNLSLDLLQLHNEILDIENSPKATLNIADTVDNFLQNLFSNFPSLHSLWKTLIGLGIF

SEQ ID NO: 174 - protein, 101 aa, Unknown
  Synthesized or naturally derived
IQALEESISALEKSLTSLSEVVLQNRRGLDILFLQEGGLCAALKEECCFYADHTGLVRDNMAKLRERLKQRQQLFDSQQGWFEGWFNKSPWFTTLISSIMG

SEQ ID NO: 175 - protein, 103 aa, Unknown
  Synthesized or naturally derived
LEQDQQRLITAINQTHYNLLNVASVVAQNRRGLDWLYIRLGFQSLCPTINEPCCFLRIQNDSIIRLGDLQPLSQRVSTDWQWPWNWDLGLTAWVRETIHSVLS

SEQ ID NO: 176 - protein, 135 aa, Unknown
  Synthesized or naturally derived
STQKAIDGVTNKVNSIIDKMNTQFEAVGREFNNLERRIENLNKQMEDGFLDVWTYNAELLVLMENEHDSNVKNLYDKVRLQLRDNAKELGNGCFEFYHKCDNECMESVKNGTYDYPQYSEEARLNREEISGVKLE

SEQ ID NO: 177 - protein, 140 aa, Unknown
  Synthesized or naturally derived
STQNAINGITNKVNSVIEKMNTQFTAVGKEFNKLERRMEYLNKKVDDGFLDIWTYNAELLVLLENERTLDFHDSNVKNLYEKVKSQLKNNAKEIGYGCFEFYHKCNNECMESVKNGTYDYPKYSEESKLNREKIDGVKLE

SEQ ID NO: 178 - protein, 140 aa, Unknown
  Synthesized or naturally derived
STQNAIDGITNKVNSVIEKMNTKFTAVGKEFNNLERRIENLNKKVDDGFLDVWTYNAELLVLLENERTLDFHDSNVRNLYEKVKSQLRNNAKELGNGCFEFYHKCDDECIESVKNGTYDYPKYSEESKLNREEIDGVKLE

SEQ ID NO: 179 - protein, 140 aa, Unknown
  Synthesized or naturally derived
STQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHNVYRDEALNNRFQIKGVELK

SEQ ID NO: 180 - protein, 140 aa, Unknown
  Synthesized or naturally derived
STQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEIKGVKLS

SEQ ID NO: 181 - protein, 140 aa, Unknown
  Synthesized or naturally derived
STQKAIDGITNKVNSIIDKMNTQFEAVEHEFSNLERRIGNLNKRMEDGFLDVWTYNAELLVLLENERTLDMHDANVKNLHEKVKSQLKDNAKDLGNGCFEFWHKCDNDCIKSVKNGTYDYPKYQEESRLNRQEIKSVMLE

SEQ ID NO: 182 - protein, 140 aa, Unknown
  Synthesized or naturally derived
STQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESIRNGTYNYPQYSEEARLKREEISGVKLE

SEQ ID NO: 183 - protein, 140 aa, Unknown
  Synthesized or naturally derived
STQKAIDGITNKVNSIIDKMNTQFEAVEHEFSSLERRIDNLNKRMEDGFLDVWTYNAELLVLLENERTLDMHDANVKNLHEKVKSQLKDNAKDLGNGCFEFWHKCDDECINSVKNGTYDYPKYQEESRLNRQEIKSVMLE

SEQ ID NO: 184 - protein, 140 aa, Unknown
  Synthesized or naturally derived
STQSAINQITGKLNRLIEKTNQQFELIDNEFNEIEKQIGNVINWTRDSIIEVWSYNAEFLVAVENQHTIDLTDSEMNKLYEKVRRQLRENAEEDGNGCFEIFHQCDNDCMASIRNNTYDHKKYRKEAIQNRIQIDAVKLS

SEQ ID NO: 185 - protein, 140 aa, Unknown
  Synthesized or naturally derived
STQEAIDKITNKVNNIVDKMNREFEVVNHEFSEVEKRINMINDKIDDQIEGLWAYNAELLVLLENQKTLDEHDSNVKNLFDEVKRRLSTNAMDAGNGCFDILHKCNNECMETIKNGTYNHKEYEEEAKLERSKINGVKLE

SEQ ID NO: 186 - protein, 140 aa, Unknown
  Synthesized or naturally derived
STQRAIDKITSKVNNIVDKMNKQYEIIDHEFSEVETRLNMINNKIDDQIQDIWAYNAELLVLLENQKTLDEHDANVNNLYNKVKRALGSNAVEDGKGCFELYHKCDDQCMETIRNGTYNKRKYKEESRLERQKIEGVKLE

SEQ ID NO: 187 - protein, 140 aa, Unknown
  Synthesized or naturally derived
STQTAIDQITGKLNRLIEKTNTEFESIESEFSQIEHQIGNVINWTKDSITDIWTYQAELLVAMENQHTIDMADSEMLNLYERVRKQLRQNAEEDGKGCFEIYHTCDDSCMESIRNNTYDHSQYREEALLNRLNINPVELS

SEQ ID NO: 188 - protein, 140 aa, Unknown
  Synthesized or naturally derived
STQKAIDQITSKVNNIVDRMNTNFESVQHEFSEIEERINQLSAHVDDSLIDIWSYNAQLLVLLENEKTLDLHDSNVRNLHEKVRRMLKDNAKDEGNGCFTFYHKCDNECIEKVRNGTYDHKEFEEESKLNRQEIEGVKLD

SEQ ID NO: 189 - protein, 140 aa, Unknown
  Synthesized or naturally derived
STQKAIDNMQNKLNNVIDKMNKQFEVVKHEFSEVESRINMINSKIDDQITDIWAYNAELLVLLENQKTLDEHDANVRNLHDRVRRVLKENAIDTGDGCFEILHKCDDGCMDTIKNGTYNHQDYEEESKLERQRINGVKLE

SEQ ID NO: 190 - protein, 140 aa, Unknown
  Synthesized or naturally derived
STQKAIDQITTKINNIIDKMNGNYDSIRGEFSQVERRINMLADRIDDAVTDVWSYNAKLLVLLENDKTLDMHDANVRNLHEQVRRTLKANAINEGNGCFELLHKCNDSCMETIRNGTYNHAEYAEESKLKRQEIEGIKLK

SEQ ID NO: 191 - protein, 140 aa, Unknown
  Synthesized or naturally derived
STQAAIDQINGKLNRLIEKTNEKYHQIEKEFEQVEGRIQDLEKYVEDTKIDLWSYNAELLVALENQHTIDVTDSEMNKLFERVRRQLRENAEDQGNGCFEIFHQCDNNCIESIRNGTYDHNIYRDEAINNRIKINPVTLT

SEQ ID NO: 192 - protein, 140 aa, Unknown
  Synthesized or naturally derived
STQAAIDQITGKLNRLIEKTNKQFELIDNEFTEVEQQIGNVINWTRDSLTEIWSYNAELLVAMENQHTIDLADSEMNKLYERVRRQLRENAEEDGTGCFEIFHRCDDQCMESIRNNTYNHTEYRQEALQNRIMINPVKLS

SEQ ID NO: 193 - protein, 140 aa, Unknown
  Synthesized or naturally derived
STQKAIDEITTKINNIIEKMNGNYDSIRGEFNQVEKRINMLADRVDDAVTDIWSYNAKLLVLLENGRTLDLHDANVRNLHDQVKRILKSNAIDEGDGCFNLLHKCNDSCMETIRNGTYNHEDYREESQLKRQEIEGIKLK

SEQ ID NO: 194 - protein, 140 aa, Unknown
  Synthesized or naturally derived
STQAAIDQINGKLNRLIEKTNEKYHQIEKEFEQVEGRIQDLEKYVEDTKIDLWSYNAELLVALENQHTIDVTDSEMNKLFERVRRQLRENAEDKGNGCFEIFHQCDNNCIESIRNGTYDHDIYRDEAINNRFQIQGVKLT

SEQ ID NO: 195 - protein, 140 aa, Unknown
  Synthesized or naturally derived
STQSAIDQVTGKLNRLIEKTNQQFKLIDNEFTEVEKQIGNVINWTRDSMTEVWSYNAELLVAMENQHTIDLADSEMNKLYERVKRQLRENAEEDGTGCFEIFHKCDDDCMASIRNNTYDHSKYREEAMQNRIQIDPVKLS

SEQ ID NO: 196 - protein, 140 aa, Unknown; identical to SEQ ID NO: 180
  Synthesized or naturally derived

SEQ ID NO: 197 - protein, 140 aa, Unknown
  Synthesized or naturally derived
STQAAIDQINGKLNRVIEKTNEKFHQIEKEFSEVEGRIQDLEKYVEDTKIDLWSYNAELLVALENQHTIDLTDSEMNKLFEKTRRQLRENAEEMGNGCFKIYHKCDNACIESIRNGTYDHDVYRDEALNNRFQIKGVELK

SEQ ID NO: 198 - protein, 140 aa, Unknown
  Synthesized or naturally derived
STQNAIDGITNKVNSVIEKMNTQFTAVGKEFNNLERRIENLNKKVDDGFLDIWTYNAELLVLLENERTLDFHDSNVRNLYEKVKSQLKNNAKEIGNGCFEFYHKCDDACMESVRNGTYDYPKYSEESKLNREEIDGVKLE

SEQ ID NO: 199 - protein, 140 aa, Unknown
  Synthesized or naturally derived
STQNAIDGITNKVNSVIEKMNTQFTAVGKEFNHLEKRIENLNKKVDDGFLDVWTYNAELLVLLENERTLDFHDSNVKNLYEKVRSQLRNNAKEIGNGCFEFYHKCDDTCMESVKNGTYDYSKYSEESKLNREVIDGVKLD

SEQ ID NO: 200 - protein, 140 aa, Unknown
  Synthesized or naturally derived
STQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLE

SEQ ID NO: 201 - protein, 140 aa, Unknown
  Synthesized or naturally derived
STQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLND

SEQ ID NO: 202 - protein, 140 aa, Unknown
  Synthesized or naturally derived
STQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLND

SEQ ID NO: 203 - protein, 142 aa, Unknown
  Synthesized or naturally derived
SAEKGFEKIGNDIQILKSSINIAIEKLNDRISHDEQAIRDLTLEIENARSEALLGELGIIRALLVGNISIGLQESLWELASEITNRAGDLAVEVSPGCWIIDNNICDQSCQNFIFKFNETAPVPTIPPLDTKIDLQSDPFYW

SEQ ID NO: 204 - protein, 142 aa, Unknown
  Synthesized or naturally derived
SAEKGFEKIGNDIQILRSSTNIAIEKLNDRISHDEQAIRDLTLEIENARSEALLGELGIIRALLVGNISIGLQESLWELASEITNRAGDLAVEVSPGCWVIDNNICDQSCQNFIFKFNETAPVPTIPPLDTKIDLQSDPFYW

SEQ ID NO: 205 - DNA, 1371 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctacaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagtttgaaactggagaacaaggaggcaaaggtctcgaagcgggagaaggcggtgtgggtgctgaacccagaagcggggatgtggcagtgtctgctgagtgactcgggacaggtcctgctggaatccaacatcaaggttctgcccacatggtccaccccggtctcgagtgggggatccggaggttcaggtgggtctggaggctcggggggctcctcaggtgaatgggatagagaaattaataactatacttctctgatccacagccttatagaggaatcgcaaaaccaacaggagaagaacgaacaggagcttctggaactggataaatgggcatcgctttggaattggttc

SEQ ID NO: 206 - DNA, 1350 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctacaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagtttgaaactggagaacaaggaggcaaaggtctcgaagcgggagaaggcggtgtgggtgctgaacccagaagcggggatgtggcagtgtctgctgagtgactcgggacaggtcctgctggaatccaacatcaaggttctgcccacatggtccaccccggtctcgagtgggggatccggaggttcaggtgggtctggaggctcggggggctcctcaggttctggtatagtgcagcagcagaacaatttgctgagggctattgaggcgcaacagcatctgttgcaactcacagtctggggcatcaagcagctccaggcaagaatcctgtaa

SEQ ID NO: 207 - DNA, 1308 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctacaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagtttgaaactggagaacaaggaggcaaaggtctcgaagcgggagaaggcggtgtgggtgctgaacccagaagcggggatgtggcagtgtctgctgagtgactcgggacaggtcctgctggaatccaacatcaaggttctgcccacatggtccaccccggtctcgagtgggggatccggaggttcaggtgggtctggaggctcggggggctcctcaggtctccaggcaagaatcctggctgtggaaagatacctaaaggatcaacagctcctggggatttggggttaa

SEQ ID NO: 208 - DNA, 1398 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctacaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagtttgaaactggagaacaaggaggcaaaggtctcgaagcgggagaaggcggtgtgggtgctgaacccagaagcggggatgtggcagtgtctgctgagtgactcgggacaggtcctgctggaatccaacatcaaggttctgcccacatggtccaccccggtctcgagtgggggatccggaggttcaggtgggtctggaggctcggggggctcctcaggtctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctaa

SEQ ID NO: 209 - DNA, 1398 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctacaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagtttgaaactggagaacaaggaggcaaaggtctcgaagcgggagaaggcggtgtgggtgctgaacccagaagcggggatgtggcagtgtctgctgagtgactcgggacaggtcctgctggaatccaacatcaaggttctgcccacatggtccaccccggtctcgagtgggggatccggaggttcaggtgggtctggaggctcggggggctcctcaggtctccaggcaagaatcctggctgtggaaagatacctaaaggatcaacagctcctggggatttggggttcctctggaaaactcatttccaccactgctgtgccttggaatgctagttggagtaataaatctctggaacagatttggaatcacacgacctaa

SEQ ID NO: 210 - DNA, 1311 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctacaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagtttgaaactggagaacaaggaggcaaaggtctcgaagcgggagaaggcggtgtgggtgctgaacccagaagcggggatgtggcagtgtctgctgagtgactcgggacaggtcctgctggaatccaacatcaaggttctgcccacatggtccaccccggtctcgagtgggggatccggaggttcaggtgggtctggaggctcggggggctcctcaggtaccactgctgtgccttggaatgctagttggagtaataaatctctggaacagatttggaatcacacgacctaa

SEQ ID NO: 211 - DNA, 1335 bp, Artificial sequence
  Synthesized
atgaaccggggagtcccttttaggcacttgcttctggtgctgcaactggcgctcctcccagcagccactcagggaaagaaagtgcatcatcaccatcaccacaaagttgtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctacaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagtttgaaactggagaacaaggaggcaaaggtctcgaagcgggagaaggcggtgtgggtgctgaacccagaagcggggatgtggcagtgtctgctgagtgactcgggacaggtcctgctggaatccaacatcaaggttctgcccacatggtccaccccggtctcgagtgggggatccggaggttcaggtgggtctggaggctcggggggctcctcaggtaccactgctgtgccttggaatgctagttggagtaataaatctctggaacagatttggaatcacacgacctaa

SEQ ID NO: 212 - protein, 48 aa, Artificial sequence
  Synthesized
MRAWIFFLLCLAGRALAASEQKLISEEDLNMHTGHHHHHHGENLYFQG

SEQ ID NO: 213 - DNA, 144 bp, Artificial sequence
  Synthesized
atgagggcctggatcttctttctcctttgcctggccgggagggctctggcagctagcgaacaaaaactcatctcagaagaggatctgaatatgcataccggtcatcatcaccatcaccatggtgagaatctttattttcagggt

SEQ ID NO: 214 - DNA, 1395 bp, Artificial sequence
  Synthesized
atgaaccggggagtcccttttaggcacttgcttctggtgctgcaactggcgctcctcccagcagccactcagggaaagaaagtgcatcatcaccatcaccacaaagttgtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctacaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagtttgaaactggagaacaaggaggcaaaggtctcgaagcgggagaaggcggtgtgggtgctgaacccagaagcggggatgtggcagtgtctgctgagtgactcgggacaggtcctgctggaatccaacatcaaggttctgcccacatggtccaccccggtctcgagtgggggatccggaggttcaggtgggtctggaggctcggggggctcctcaggtgaatgggatagagaaattaataactatacttctctgatccacagccttatagaggaatcgcaaaaccaacaggagaagaacgaacaggagcttctggaactggataaatgggcatcgctttggaattggttc

SEQ ID NO: 215 - protein, 394 aa, Artificial sequence
  Synthesized
MNRGVPFRHLLLVLQLALLPAATQGKKVVLGKKGDTVELTCTASQKKSIQFHWKNSNQTKILGNQGSFLTKGPSKLNDRVDSRRSLWDQGNFTLIIKNLKIEDSDTYICEVGDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPV

SEQ ID NO: 216 - protein, 454 aa, Artificial sequence
  Synthesized
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVGDQKEEVQLLVFGLTASDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNKVLPTWSTPVSSGGSGGSGGSGGSGGSSGEWDREINNYTSLIHSLIEESQNQQEKNEQELLELDKWASLWNWF

SEQ ID NO: 217 - protein, 474 aa, Artificial sequence
  Synthesized
MRAWIFFLLCLAGRALAASEQKLISEEDLNMHTGHHHHHHGENLYFQGKKVVLGKKGDTVELTCTASQKKSIQFHWKNSNQIKILGNQGSFLTKGPSKLNDRADSRRSLWDQGNFPLIIKNLKIEDSYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLGGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVNPAGMWQCLLSDSGQVLLESNIKVLPTWSTPVSSGGSGGSGGSGGSGGSSGEWDREINNYTSLIHSLIEESQNQQEKNEQELLELDKWASLWNWF

SEQ ID NO: 218 - protein, 444 aa, Artificial sequence
  Synthesized
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTADTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDKKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNKVLPTWSTPVSSGGSGGSGGSGGSGGSSGSWETWEREIENYTRQIYRILEESQEQQDRNERDLLE

SEQ ID NO: 219 - protein, 461 aa, Artificial sequence
  Synthesized
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTADTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDKKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNKVLPTWSTPVSSGGSGGSGGSGGSGGSSGERYLKDQQLLGIWGCSGKLICTTAVPWNASWSNKSLEQIWNHTTWMEWDREIN

SEQ ID NO: 220 - protein, 461 aa, Artificial sequence
  Synthesized
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTADTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDKKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNKVLPTWSTPVSSGGSGGSGGSGGSGGSSGERYLKDQQLLGIWGSSGKLISTTAVPWNASWSNKSLEQIWNHTTWMEWDREIN

SEQ ID NO: 221 - protein, 490 aa, Artificial sequence
  Synthesized
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTADTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDKKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNKVLPTWSTPVSSGGSGGSGGSGGSGGSSGERYLKDQQLLGIWGCSGKLICTTAVPWNASWSNKSLEQIWNHTTWMEWDREINNYTSLIHSLIEESQNQQEKNEQELLELDK

SEQ ID NO: 222 - protein, 446 aa, Artificial sequence
  Synthesized
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTADTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDKKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNKVLPTWSTPVSSGGSGGSGGSSGEWDREINNYTSLIHSLIEESQNQQEKNEQELLELDKWASLWNWF

SEQ ID NO: 223 - protein, 458 aa, Artificial sequence
  Synthesized
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTADTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDKKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNKVLPTWSTPVSSGGSGGSGGSGGSGGSGGSGGSSGEWDREINNYTSLIHSLIEESQNQQEKNEQELLELDKWASLWNWF

SEQ ID NO: 224 - protein, 270 aa, Artificial sequence
  Synthesized
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSGGSGGSGGSGGSGGSSGEWDREINNYTSLIHSLIEESQNQQEKNEQELLELDKWASLWNWF

SEQ ID NO: 225 - protein, 270 aa, Artificial sequence; identical to SEQ ID NO: 224
  Synthesized

SEQ ID NO: 226 - DNA, 1354 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagtcccagaagaagagcatacaattccactggaaaaactccaaccagataaagattctgggaaatcagggctccttcttaactaaaggtccatccaagctgaatgatcgcgctgactcaagaagaagcttgggaccaaggaaactttcccctgatcatcaagaatcttaagatagaagactcagatacttacatctgtggagtggaggaccagaaggaggaggtgcaattgctagtgttcggattgactgccaatctgacacccacctgcttcaggggcagagcctgaccctgaccttggagagcccccctggtagtagcccctcaggcaatgtaggagtccaaggggtaaaaacatacagggggggaagaccctctccggtctcagctggagctacaggatagtggcacctggacatgcactgtcttgcagaaccagaagaaggtggagttcaaatagacatcgtggtgctagctttccagaaggcctccagcatagtctataagaagagggggaacaggtggagttctccttcccactcgcctttacagttgaaaagctgacgggcagtggcgagctgtgtggcaggcggagagggcttcctcctccaagtcttggatcacctttgacctaagaacaaggaagtgtctgtaaaacgggttacccaggaccctaagctccagatgggcaagaagctcccgctccacccaccctgccccaggccttgcctcagtatgctggctctggaaacctcacccggcccttgaagcgaaaacaggaaagttgcatcaggaagtgaacctggtggtgatgagagccactcagctccagaaaatttgacctgtgaggtgtggggacccacctcccctaagctgatgctgagttgaaactggagaacaaggaggcaaaggtctcgaagcgggagaaggcggtgtgggtgctgaacccagaagcggggatgtgcagtgtctgctgagtgactcgggacaggtcctgctggaatccaacataaggttctgcccacatggtccaccccggtctcgagtgggggatccggaggttcaggtgggtctggaggctcggggggctctcaggtgaatgggatagagaaattaataactatacttctctgatcccagccttatagaggaatcgcaaaaccaacaggagaagaacgaacaggagcttctggaactggataaatgggcatcgctttgaattggttctaa

SEQ ID NO: 227 - DNA, 1419 bp, Artificial sequence
  Synthesized
atgagggcctggatcttctttctcctttgcctggccgggagggctctggcagctagcgaacaaaaactcatctcagaagaggatctgaatatgcataccggtcatcatcaccatcaccatggtgagatctttattttcagggtaagaaagtggtgctgggcaaaaaaggggatacagtggaactgacctgtacagcttcccagaaaagagcatacaattccactggaaaaactccaaccagataaagattctggaaatcagggctccttcttaactaaaggtccatccaagctgaatgatcgcgctgactcaagaagaagcctttgggaccaggaaactttcccctgatcatcaagaatcttaagatagaagactcagaacttacatctgtgaagtggaggaccagaaggaggaggtgcaattgctagtgttcggattgactgccaactctgacacccactgcttcaggggcagagcctgaccctgaccttggagagcccccctgtagtagcccctcagtgcaatgtaggagtccaaggggtaaaaacatacagggggggaagaccctctccgtgtctcagctggactacaggatagtggcacctggacatgcactgtcttgcagaaccagagaaggtggagttcaaaatagacatcgtggtgctagctttccagaaggcctccagcatagtctataagaaagagggggaacagtggagttctccttcccactcgcctttacagttgaaaagctgacggcagtggcgagctgtggtggcaggcggagagggcttcctcctccaagtcttggatcacctttgacctgaagaacaaggaagttctgtaaaacgggttacccaggaccctaagctccagatgggcagaagctcccgctccacctcaccctgccccaggccttgcctcagtatgctggctctggaaacctcaccctggcccttgaagcgaaacaggaaagttgcatcaggaagtgaacctggtggtgatgagaccactcagctccagaaaaatttgacctgtgaggtgtggggacccacctcccctaagctgatgctgagtttgaaactggagaacaagaggcaaaggtctcgaagcgggagaaggcggtgtgggtgctaacccagaagcggggatgtggcagtgtctgctgagtgactcgggacaggtcctgctggaatccaacatcaaggttctgcccacatgtccaccccggtctcgagtgggggatccggaggttcaggtggtctggaggctcggggggctcctcaggtgaatgggatagagaaattaataactatacttctctgatccacagccttatagaggaatccaaaaccaacaggagaagaacgaacaggagcttctggaatggataaatgggcatcgctttggaattggttc

SEQ ID NO: 228 - DNA, 1330 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagtcccagaagaagagcatacaattccactggaaaaactccaaccagataaagattctgggaaatcagggctccttcttaactaaaggtccatccaagctgaatgatcgcgctgactcaagaagaagcttgggaccaaggaaactttcccctgatcatcaagaatcttaagatagaagactcagatacttacatctgtgaagtggaggaccagaaggaggaggtgcaattgctagtgttcggattgactgccaatcgacacccacctgcttcaggggcagagcctgaccctgaccttggagagcccccctggtagtagcccctcagtgcaatgtaggagtccaaggggtaaaaacatacagggggggaagaccctctccggtccagctggagctacaggatagtggcacctggacatgcactgtcttgcagaaccagaagaaggtggagttcaaaatagacatcgtggtgctagctttccagaaggcctccagcatagtctataagaagaggggaacaggtggagttctccttcccactcgcctttacagttgaaaagctgacgggcagtggcgagctgtggtggcaggcggagagggcttcctcctccaagtcttggatcacctttgacctaagaaaaggaagtgtctgtaaaacgggttacccaggaccctaagctccagatgggcaagaagctcccgctccacctcaccctgccccaggccttgcctcagtatgctggctctggaaacctcacccggccctgaagcgaaaacaggaaagttgcatcaggaagtgaacctggtggtgatgagagccactcagctccagaaaaatttgacctgtgaggtgtggggacccacctcccctaagctgatgctgagttgaaactgagaacaaggaggcaaaggtctcgaagcgggagaaggcggtgtgggtgctgaacccagaagcggggatgtggcagtgtctgctgagtgactcgggacaggtcctgctggaatccaacataaggttctgcccacatggtccaccccggtctcgagtgggggatccggaggttcaggtgggtctggaggctcggggggctcctcaggttcttgggaaacttgggagcgggaaattgaaaactatacccgtcaaatttaccggatactagaagagagccaggaacaacaagatcggaacgagagagatctgctcgaa

SEQ ID NO: 229 - DNA, 1380 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagtcccagaagaagagcatacaattccactggaaaaactccaaccagataaagattctgggaaatcagggctccttcttaactaaaggtccatccaagctgaatgatcgcgctgactcaagaagaagcttgggaccaaggaaactttcccctgatcatcaagaatcttaagatagaagactcagatacttacatctgtgaagtggaggaccagaaggaggaggtgcaattgctagtgttcggattgactgccaatcgacacccacctgcttcaggggcagagcctgaccctgaccttggagagcccccctggtagtagcccctcagtgcaatgtaggagtccaaggggtaaaaacatacagggggggaagaccctctccggtccagctggagctacaggatagtggcacctggacatgcactgtcttgcagaaccagaagaaggtggagttcaaaatagacatcgtggtgctagctttccagaaggcctccagcatagtctataagaagaggggaacaggtggagttctccttcccactcgcctttacagttgaaaagctgacgggcagtggcgagctgtggtggcaggcggagagggcttcctcctccaagtcttggatcacctttgacctaagaaaaggaagtgtctgtaaaacgggttacccaggaccctaagctccagatgggcaagaagctcccgctccacctcaccctgccccaggccttgcctcagtatgctggctctggaaacctcacccggccctgaagcgaaaacaggaaagttgcatcaggaagtgaacctggtggtgatgagagccactcagctccagaaaaatttgacctgtgaggtgtggggacccacctcccctaagctgatgctgagttgaaactgagaacaaggaggcaaaggtctcgaagcgggagaaggcggtgtgggtgctgaacccagaagcggggatgtggcagtgtctgctgagtgactcgggacaggtcctgctggaatccaacataaggttctgcccacatggtccaccccggtctcgagtgggggatccggaggttcaggtgggtctggaggctcggggggctcctcaggtgaacgctacctgaaagatcagcaactgctcggcatctgggctgttctgggaagctgatctgtaccaccgcggtcccctggaacgcttcttggtcaaacaaatctctagagcagatctggaaccacacaacttggatggaatgggaccgggagatcaac

SEQ ID NO: 230 - DNA, 1380 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagtcccagaagaagagcatacaattccactggaaaaactccaaccagataaagattctgggaaatcagggctccttcttaactaaaggtccatccaagctgaatgatcgcgctgactcaagaagaagcttgggaccaaggaaactttcccctgatcatcaagaatcttaagatagaagactcagatacttacatctgtgaagtggaggaccagaaggaggaggtgcaattgctagtgttcggattgactgccaatcgacacccacctgcttcaggggcagagcctgaccctgaccttggagagcccccctggtagtagcccctcagtgcaatgtaggagtccaaggggtaaaaacatacagggggggaagaccctctccggtccagctggagctacaggatagtggcacctggacatgcactgtcttgcagaaccagaagaaggtggagttcaaaatagacatcgtggtgctagctttccagaaggcctccagcatagtctataagaagaggggaacaggtggagttctccttcccactcgcctttacagttgaaaagctgacgggcagtggcgagctgtggtggcaggcggagagggcttcctcctccaagtcttggatcacctttgacctaagaaaaggaagtgtctgtaaaacgggttacccaggaccctaagctccagatgggcaagaagctcccgctccacctcaccctgccccaggccttgcctcagtatgctggctctggaaacctcacccggccctgaagcgaaaacaggaaagttgcatcaggaagtgaacctggtggtgatgagagccactcagctccagaaaaatttgacctgtgaggtgtggggacccacctcccctaagctgatgctgagttgaaactgagaacaaggaggcaaaggtctcgaagcgggagaaggcggtgtgggtgctgaacccagaagcggggatgtggcagtgtctgctgagtgactcgggacaggtcctgctggaatccaacataaggttctgcccacatggtccaccccggtctcgagtgggggatccggaggttcaggtgggtctggaggctcggggggctcctcaggtgaacgctacctgaaagatcagcaactgctcggcatctgggctcttctgggaagctgatctctaccaccgcggtcccctggaacgcttcttggtcaaacaaatctctagagcagatctggaaccacacaacttggatggaatgggaccgggagatcaac

SEQ ID NO: 231 - DNA, 1466 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagtcccagaagaagagcatacaattccactggaaaaactccaaccagataaagattctgggaaatcagggctccttcttaactaaaggtccatccaagctgaatgatcgcgctgactcaagaagaagcttgggaccaaggaaactttcccctgatcatcaagaatcttaagatagaagactcagatacttacatctgtgaagtggaggaccagaaggaggaggtgcaattgctagtgttcggattgactgccaatcgacacccacctgcttcaggggcagagcctgaccctgaccttggagagcccccctggtagtagcccctcagtgcaatgtaggagtccaaggggtaaaaacatacagggggggaagaccctctccggtccagctggagctacaggatagtggcacctggacatgcactgtcttgcagaaccagaagaaggtggagttcaaaatagacatcgtggtgctagctttccagaaggcctccagcatagtctataagaagaggggaacaggtggagttctccttcccactcgcctttacagttgaaaagctgacgggcagtggcgagctgtggtggcaggcggagagggcttcctcctccaagtcttggatcacctttgacctaagaaaaggaagtgtctgtaaaacgggttacccaggaccctaagctccagatgggcaagaagctcccgctccacctcaccctgccccaggccttgcctcagtatgctggctctggaaacctcacccggccctgaagcgaaaacaggaaagttgcatcaggaagtgaacctggtggtgatgagagccactcagctccagaaaaatttgacctgtgaggtgtggggacccacctcccctaagctgatgctgagttgaaactgagaacaaggaggcaaaggtctcgaagcgggagaaggcggtgtgggtgctgaacccagaagcggggatgtggcagtgtctgctgagtgactcgggacaggtcctgctggaatccaacataaggttctgcccacatggtccaccccggtctcgagtgggggatccggaggttcaggtgggtctggaggctcggggggctcctcaggtgaacgctacctgaaagatcagcaactgctcggcatctgggctgttctgggaagctgatctgtaccaccgcggtcccctggaacgcttcttggtcaaacaaatctctagagcagatctggaaccacacaacttggatggaatgggaccgggagatcaacaactacaccccctcatccattccctcatcgaggaatcccagaatcaacaggagaaaaacgagcaggaactcctggaactcgataag

SEQ ID NO: 232 - DNA, 1335 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagtcccagaagaagagcatacaattccactggaaaaactccaaccagataaagattctgggaaatcagggctccttcttaactaaaggtccatccaagctgaatgatcgcgctgactcaagaagaagcttgggaccaaggaaactttcccctgatcatcaagaatcttaagatagaagactcagatacttacatctgtgaagtggaggaccagaaggaggaggtgcaattgctagtgttcggattgactgccaatcgacacccacctgcttcaggggcagagcctgaccctgaccttggagagcccccctggtagtagcccctcagtgcaatgtaggagtccaaggggtaaaaacatacagggggggaagaccctctccggtccagctggagctacaggatagtggcacctggacatgcactgtcttgcagaaccagaagaaggtggagttcaaaatagacatcgtggtgctagctttccagaaggcctccagcatagtctataagaagaggggaacaggtggagttctccttcccactcgcctttacagttgaaaagctgacgggcagtggcgagctgtggtggcaggcggagagggcttcctcctccaagtcttggatcacctttgacctaagaaaaggaagtgtctgtaaaacgggttacccaggaccctaagctccagatgggcaagaagctcccgctccacctcaccctgccccaggccttgcctcagtatgctggctctggaaacctcacccggccctgaagcgaaaacaggaaagttgcatcaggaagtgaacctggtggtgatgagagccactcagctccagaaaaatttgacctgtgaggtgtggggacccacctcccctaagctgatgctgagttgaaactgagaacaaggaggcaaaggtctcgaagcgggagaaggcggtgtgggtgctgaacccagaagcggggatgtggcagtgtctgctgagtgactcgggacaggtcctgctggaatccaacataaggttctgcccacatggtccaccccggtctcgagtgggggatccggaggttcggggggctcctcaggtgaatgggatagagaaattaataactatacttctctgatccacagccttatagaggaatgcaaaaccaacaggagaagaacgaacaggagcttctggaactggataaatgggcatcgctttggaattggttc

SEQ ID NO: 233 - DNA, 1371 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagtcccagaagaagagcatacaattccactggaaaaactccaaccagataaagattctgggaaatcagggctccttcttaactaaaggtccatccaagctgaatgatcgcgctgactcaagaagaagcttgggaccaaggaaactttcccctgatcatcaagaatcttaagatagaagactcagatacttacatctgtgaagtggaggaccagaaggaggaggtgcaattgctagtgttcggattgactgccaatcgacacccacctgcttcaggggcagagcctgaccctgaccttggagagcccccctggtagtagcccctcagtgcaatgtaggagtccaaggggtaaaaacatacagggggggaagaccctctccggtccagctggagctacaggatagtggcacctggacatgcactgtcttgcagaaccagaagaaggtggagttcaaaatagacatcgtggtgctagctttccagaaggcctccagcatagtctataagaagaggggaacaggtggagttctccttcccactcgcctttacagttgaaaagctgacgggcagtggcgagctgtggtggcaggcggagagggcttcctcctccaagtcttggatcacctttgacctaagaaaaggaagtgtctgtaaaacgggttacccaggaccctaagctccagatgggcaagaagctcccgctccacctcaccctgccccaggccttgcctcagtatgctggctctggaaacctcacccggccctgaagcgaaaacaggaaagttgcatcaggaagtgaacctggtggtgatgagagccactcagctccagaaaaatttgacctgtgaggtgtggggacccacctcccctaagctgatgctgagttgaaactgagaacaaggaggcaaaggtctcgaagcgggagaaggcggtgtgggtgctgaacccagaagcggggatgtggcagtgtctgctgagtgactcgggacaggtcctgctggaatccaacataaggttctgcccacatggtccaccccggtctcgagtgggggatccggaggttcaggtgggagtggcgggtcaggtggctctggaggctcggggggctcctcaggtgaatgggatagagaaattaatactatacttctctgatccacagccttatagaggaatcgcaaaaccaacaggagaagaacgaacaggagcttctggaactggataaatgggcatcgctttggaattggttc

SEQ ID NO: 234 - DNA, 800 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagtcccagaagaagagcatacaattccactggaaaaactccaaccagataaagattctgggaaatcagggctccttcttaactaaaggtccatccaagctgaatgatcgcgctgactcaagaagaagcttgggaccaaggaaactttcccctgatcatcaagaatcttaagatagaagactcagatacttacatctgtgaagtggaggaccagaaggaggaggtgcaattgctagtgttcggattgactgccaatcgacacccacctgcttcaggggcagagcctgaccctgaccttggagagcccccctggtagtagcccctcagtgcaatgtaggagtccaaggggtaaaaacatacagggggggaagaccctctccggtccagctggagctacaggatagtggcacctggacatgcactgtcttgcagaaccagaagaaggtggagttcaaaatagacatcgtggtgctagctttccagaaggcctccagtgggggatccggagttcaggtgggtctggaggctcggggggctcctcaggtgaatgggatagagaaattaataactatacttctctgatccacagccttatagaggaatcgcaaaaccaacaggagaagaacgaacaggacttctggaactggataaatgggcatcgctttggaattggttc

SEQ ID NO: 235 - DNA, 818 bp, Artificial sequence
  Synthesized
atgaaccggggagtcccttttaggcacttgcttctggtgctgcaactggcgctcctcccagcagccactcagggaaagaaagtggtgctgggcaaaaaaggggatacagtggaactgacctgtacagtcccagaagaagagcatacaattccactggaaaaactccaaccagataaagattctgggaaatcagggctccttcttaactaaaggtccatccaagctgaatgatcgcgctgactcaagaagaagcttgggaccaaggaaactttcccctgatcatcaagaatcttaagatagaagactcagatacttacatctgtgaagtggaggaccagaaggaggaggtgcaattgctagtgttcggattgactgccaatcgacacccacctgcttcaggggcagagcctgaccctgaccttggagagcccccctggtagtagcccctcagtgcaatgtaggagtccaaggggtaaaaacatacagggggggaagaccctctccggtccagctggagctacaggatagtggcacctggacatgcactgtcttgcagaaccagaagaaggtggagttcaaaatagacatcgtggtgctagctttccagaaggcctccagtgggggatccggagttcaggtgggagtggcgggtcaggtggctctggaggctcggggggctcctcaggtgaatgggatagagaaattaataactatacttctctgatccacagccttatagaggaatcgcaaaaccaacagagaagaacgaacaggagcttctggaactggataaatgggcatcgctttggaattggttc

SEQ ID NO: 236 - DNA, 9141 bp, Artificial sequence
  Synthesized
gggctgggctgagacccgcagaggaagacgctctagggatttgtcccggactagcgagatggcaaggctgaggacgggaggctgattgagaggcgaaggtacaccctaatctcaatacaacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcgccgccaccatgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctacaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagtttgaaactggagaacaaggaggcaaaggtctcgaagcgggagaaggcggtgtgggtgctgaacccagaagcggggatgtggcagtgtctgctgagtgactcgggacaggtcctgctggaatccaacatcaaggttctgcccacatggtccaccccggtctcgagtgggggatccggaggttcaggtgggtctggaggctcggggggctcctcaggtgaatgggatagagaaattaataactatacttctctgatccacagccttatagaggaatcgcaaaaccaacaggagaagaacgaacaggagcttctggaactggataaatgggcatcgctttggaattggttctaaccgcggccgctacgtaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgaagatccgggcgcccagcatggaaataaagcacccagcgctgccctgggcccctgcgagactgtgatggttctttccacgggtcaggccgagtctgaggcctgagtggcatgagatctgatatcatcgatgaattaattcctttgcctaatttaaatgaggacttaacctgtggaaatattttgatgtgggaagctgttactgttaaaactgaggttattggggtaactgctatgttaaacttgcattcagggacacaaaaaactcatgaaaatggtgctggaaaacccattcaagggtcaaattttcatttttttgctgttggtggggaacctttggagctgcagggtgtgttagcaaactacaggaccaaatatcctgctcaaactgtaaccccaaaaaatgctacagttgacagtcagcagatgaacactgaccacaaggctgttttggataaggataatgcttatccagtggagtgctgggttcctgatccaagtaaaaatgaaaacactagatattttggaacctacacaggtggggaaaatgtgcctcctgttttgcacattactaacacagcaaccacagtgcttcttgatgagcagggtgttgggcccttgtgcaaagctgacagcttgtatgtttctgctgttgacatttgtgggctgtttaccaacacttctggaacacagcagtggaagggacttcccagatattttaaaattacccttagaaagcggtctgtgaaaaacccctacccaatttcctttttgttaagtgacctaattaacaggaggacacagagggtggatgggcagcctatgattggaatgtcctctcaagtagaggaggttagggtttatgaggacacagaggagcttcctggggatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaagatctaaagccagcaaaagtcccatggtcttataaaaatgcatagctttaggaggggagcagagaacttgaaagcatcttcctgttagtctttcttctcgtagacttcaaacttatacttgatgcctttttcctcctggacctcagagaggacgcctgggtattctgggagaagtttatatttccccaaatcaatttctgggaaaaacgtgtcactttcaaattcctgcatgatccttgtcacaaagagtctgaggtggcctggttgattcatggcttcctggtaaacagaactgcctccgactatccaaaccatgtctactttacttgccaattccggttgttcaataagtcttaaggcatcatccaaacttttggcaagaaaatgagctcctcgtggtggttctttgagttctctactgagaactatattaattctgtcctttaaaggtcgattcttctcaggaatggagaaccaggttttcctacccataatcaccagattctgtttaccttccactgaagaggttgtggtcattctttggaagtacttgaactcgttcctgagcggaggccagggtaggtctccgttcttgccaatccccatattttgggacacggcgacgatgcagttcaatggtcgaaccatgatggcagcggggataaaatcctaccagccttcacgctaggattgccgtcaagtttggcgcgaaatcgcagccctgagctgtccccccccccccccccccaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 237 - protein, 36 aa, Artificial sequence
  Synthesized
YTSLIHSLIEESQNQQEKNEQELLELDKWASLWNWF

SEQ ID NO: 238 - protein, 36 aa, Artificial sequence
  Synthesized
WMEWDREINNYTSLIHSLIEESQNQQEKNEQELLEL

SEQ ID NO: 239 - protein, 33 aa, Artificial sequence
  Synthesized
EWDREINNYTSLIHSLIEESQNQQEKNEQEGGC

SEQ ID NO: 240 - protein, 34 aa, Artificial sequence
  Synthesized
WMEWDREINNYTSLIHSLIEESQNQQEKNEQELL

SEQ ID NO: 241 - protein, 39 aa, Artificial sequence
  Synthesized
WQEWEQKITALLEQAQIQQEKNEYELQKLDKWASLWEWF

SEQ ID NO: 242 - protein, 39 aa, Artificial sequence
  Synthesized
WQEWEQKITALLEQAQIQQEKNEYELRKLDKWASLWEWF

SEQ ID NO: 243 - protein, 36 aa, Artificial sequence
  Synthesized
YTSLIHSLIEEAQNQQEKNEQELLELDKWASLWNWF

SEQ ID NO: 244 - protein, 38 aa, Artificial sequence
  Synthesized
MTWMEWDREINNYTSLIHSLIEESQNQQEKNEQELLEL

SEQ ID NO: 245 - protein, 38 aa, Artificial sequence
  Synthesized
TTWEAWDRAIAEYAARIEALIRAAQEQQEKNEAALREL

SEQ ID NO: 246 - protein, 38 aa, Artificial sequence
  Synthesized
TTWEAWDRAIAEYAARIEALIRAAQEQQEKNEAALQEL

SEQ ID NO: 247 - protein, 36 aa, Artificial sequence
  Synthesized
SWETWEREIENYTRQIYRILEESQEQQDRNERDLLE

SEQ ID NO: 248 - protein, 35 aa, Artificial sequence
  Synthesized
WZEWDRKIEEYTKKIEELIKKSQEQQEKNEKELKI

SEQ ID NO: 249 - protein, 35 aa, Artificial sequence
  Synthesized
WEEWDKKIEEYTKKIEELIKKSEEQQKKNEEELKK

SEQ ID NO: 250 - protein, 29 aa, Artificial sequence
  Synthesized
WEEWDKKIEEYTKKIEELIKKSEEQQKKN

SEQ ID NO: 251 - protein, 22 aa, Artificial sequence
  Synthesized
WEEWDKKIEEYTKKIEELIKKS

SEQ ID NO: 252 - protein, 36 aa, Artificial sequence
  Synthesized
YTSLIEELIKKWEEQQKKNEEELKKLEEWAKKWNWF

SEQ ID NO: 253 - protein, 62 aa, Artificial sequence
  Synthesized
NHTTWMEWDREINNYTSLIHSLIEESQNQQEKNEQELLELDKWASLWNWFNIKIKQIEDKIK

SEQ ID NO: 254 - protein, 55 aa, Artificial sequence
  Synthesized
NHTTWMEWDREINNYTSLIHSLIEESQNLQEKNEQELLELDKWASLWNWFNIKIK

SEQ ID NO: 255 - protein, 32 aa, Artificial sequence
  Synthesized
VEWNEMTWMEWEREIENYTKLIYKILEESQEQ

SEQ ID NO: 256 - protein, 37 aa, Artificial sequence
  Synthesized
WMEWDREIEEYTKKIEEYTKKIEEYTKKIWASLWNWF

SEQ ID NO: 257 - protein, 56 aa, Artificial sequence
  Synthesized
WMEWDREINNYTSLIHSLIEESQNQQEKNEQELLELDKWASLWNWFNITNWLWYIK

SEQ ID NO: 258 - protein, 36 aa, Artificial sequence; identical to SEQ ID NO: 12
  Synthesized

SEQ ID NO: 259 - protein, 36 aa, Artificial sequence
  Synthesized
SGIDQEQNNLTRLIEAQIHELQLTQWKIKQLLARIL

SEQ ID NO: 260 - protein, 38 aa, Artificial sequence
  Synthesized
NNLLRAIEAQQHLLQLTVWGIKQLQARILAVERYLKDQ

SEQ ID NO: 261 - protein, 20 aa, Artificial sequence
  Synthesized
GCNLLQLTVWGIKQLQARIL

SEQ ID NO: 262 - protein, 26 aa, Artificial sequence
  Synthesized
GCNIEAQQHLLQLTVWGIKQLQARIL

SEQ ID NO: 263 - protein, 35 aa, Artificial sequence
  Synthesized
SGIVQQQNNLLRAIEAQQHLLQLTVWGIKQCCGRI

SEQ ID NO: 264 - protein, 18 aa, Artificial sequence
  Synthesized
GICRCICGRGICRCICGR

SEQ ID NO: 265 - protein, 18 aa, Artificial sequence
  Synthesized
GICRCICGKGICRCICGR

SEQ ID NO: 266 - protein, 18 aa, Artificial sequence
  Synthesized
GICYCICGRGICRCICGR

SEQ ID NO: 267 - protein, 18 aa, Artificial sequence
  Synthesized
GICRCICGRYICRCICGR

SEQ ID NO: 268 - protein, 18 aa, Artificial sequence
  Synthesized
RYICRCICGRGICRCICG

SEQ ID NO: 269 - protein, 20 aa, Artificial sequence
  Synthesized
LEAIPMSIPPEVKFNKPFVF

SEQ ID NO: 270 - protein, 20 aa, Artificial sequence
  Synthesized
LEAIPCSIPPCVFFNKPFVF

SEQ ID NO: 271 - protein, 20 aa, Artificial sequence
  Synthesized
LEAIPCSIPPCFAFNKPFVF

SEQ ID NO: 272 - protein, 20 aa, Artificial sequence
  Synthesized
LEAIPMSIPPEFLFGKPFVF

SEQ ID NO: 273 - protein, 20 aa, Artificial sequence
  Synthesized
LEAIPCSIPPCFLFNKPFVF

SEQ ID NO: 274 - protein, 20 aa, Artificial sequence
  Synthesized
LEAIPMGIPPEVLFNKPFVF

SEQ ID NO: 275 - protein, 20 aa, Artificial sequence
  Synthesized
LEAIPCSIPPEFLFGKPFVF

SEQ ID NO: 276 - protein, 171 aa, Artificial sequence
  Synthesized
SAMGAASLTLSAQSRTLLAGIVQQQQQLLDVVKRQQEMLRLTVWGTKNLQARVTAIEKYLKDQAQLNSWGCAFRQVCHTTVPWVNDTLTPEWNNMTWQEWEGKIRDLEANISQQLEQAQIQQEKNMYELQKLNSWDVFGNWFDLTSWIKYIQYGVYIIIGIVVLRIVIYIV

(19) United States
(12) Patent Application Publication
(10) Pub. No.: US 2013/0108653 A1
(43) Pub. Date: May 2, 2013

(54) BIVALENT MOLECULES FOR HIV ENTRY INHIBITION

(71) Applicants: Shervin Bahrami, Artius C (DK); Martin Tolstrup, Risskov (DK); Mogens Duch Ryttergmrd, Risskov (DK); Finn Skou Pedersen, Arhus V (DK); Lars Jørgen Ostergaard, Lystrup (DK)

(72) Inventors: Shervin Bahrami, Artius C (DK); Martin Tolstrup, Risskov (DK); Mogens Duch Ryttergmrd, Risskov (DK); Finn Skou Pedersen, Arhus V (DK); Lars Jørgen Ostergaard, Lystrup (DK)

(21) Appl. No.: 13/517,364

(22) PCT Filed: Dec. 22, 2010
(86) PCT No.: PCT/GB2010/002321

§ 371 Date: Nov. 16, 2012

(30) Foreign Application Priority Data
Dec. 22, 2009 (DK) PA200970296

(51) Int. Cl. C07K 16/10; C07K 14/73

(52) U.S. Cl. CPC C07K 16/1063; C07K 14/70514
USPC 424/178.1; 530/391.7; 530/350; 530/391.9; 530/387.3; 536/23.4; 514/3.7; 514/44 R; 514/3.8

(57) ABSTRACT

The present invention relates to a new class of virus fusion inhibitors or virus entry inhibitors. More specifically the present invention relates to bivalent molecules that are pre-fusion inhibitors of viruses that makes use of the type (1) fusion mechanism belonging to the groups consisting of Othomyxoviridae, Paramyxoviridae, Retroviridae, Filoviridae and Coronaviridae, in particular HIV. The bivalent molecules of the present invention are molecules that comprise a first part capable of mimicking the function of a mammalian cell receptor, and a second part capable of binding to a virus, preferably HIV, resulting in the neutralization of the virus which is thus rendered harmless. Further, the present invention relates to compositions comprising the pre-fusion inhibitors, as well as to methods for obtaining the pre-fusion inhibitors and the use of the pre-fusion inhibitors.

### FIELD OF INVENTION

The present invention relates to a new class of virus entry inhibitors, in particular inhibitors of human immunodeficiency virus (HIV). The entry inhibitors of the present are bivalent molecules, encompassing one part (the first part) with functional and/or structural homology to a mammalian receptor involved in viral fusion, and another part (the second part) with sequence homology to peptides originating from virus. The entry inhibitors of the present invention are in particular useful against viruses that make use of the type 1 fusion mechanism belonging to the groups of viruses consisting of Orthomyxoviridae, Paramyxoviridae, Retroviridae, Filoviridae and Coronaviridae. The two parts of the bivalent molecule are joined by a linker molecule. Moreover the invention relates to methods for obtaining the bivalent molecules as well as uses of the bivalent molecules.

### BACKGROUND OF INVENTION

In order for human immunodeficiency virus (HIV) to replicate, the virus must infect living cells. HIV infect cells via a process known as “fusion”, wherein the virus particle fuses with the cell membrane of the target cell, and thereafter deliver its genetic material for integration into the host cell genome. The integration of the virus' genome into the genome of the host cell results in the production of new virus particles every time the host cell replicates. New virus particles are exported out of the host cell and are thus able to infect new cells in the surroundings.

The HIV fusion process starts with the binding of the HIV protein gp120 to the CD4 receptor protein on the surface of the target cell. Hereafter the virus binds to a co-receptor protein (mainly CRCX4 or CCR5 depending on the HIV strain, but other co-receptors exist) also present on the surface of the target cell. After this dual attachment the virus inserts a harpoon-like protein (gp41), which enables HIV to pull itself very close to the target cell, fuse with the cell membrane of the target cell, and deliver its genetic material inside the now infected cell.

Fusion inhibitors or entry inhibitors are molecules that prevent virus, e.g. human immunodeficiency virus (HIV) from entering healthy T-lymphocytes (T-cells or CD4 cells). Although HIV infects a variety of cells, its main target is the T4-lymphocyte (also called the “T-helper cell”), a type of white blood cell that carry many copies of the CD4 receptor, but also macrophages expressing the CD4 receptor are HIV targets. Entry inhibitors work differently from many of the currently approved anti-HIV drugs, e.g. protease inhibitors, nucleoside reverse transcriptase inhibitors (NRTIs), and non-nucleoside reverse transcriptase inhibitors (NNRTIs)—which all are active against HIV after it has infected a CD4 cell. HIV-positive humans who have become resistant to protease inhibitors, nucleoside reverse transcriptase inhibitors, and non-nucleoside reverse transcriptase inhibitors will most likely benefit from these entry inhibitors since they are of a different class of drugs.

Entry inhibitors work by binding either to the surface of CD4 cells or to proteins on the surface of the virus, e.g. HIV. In order for HIV to bind to CD4 cells, the proteins on HIV's outer surface must bind to the proteins on the surface of CD4 cells. Some entry inhibitors target the gp120 or gp41 proteins on HIV's surface. Other entry inhibitors target the CD4 receptor or the co-receptors on the CD4 cell surface.

Currently, two entry inhibitors have been approved by the U.S. Food and Drug Administration (FDA). Roche's Fuzeon (enfuvirtide), approved in March 2003, targets the gp41 protein on HIV's surface. Pfizer's Selzentry (maraviroc), approved in August 2007, targets the CCR5 co-receptor protein on CD4 cells. Although both entry inhibitors have shown activity against HIV infection, they do have several disadvantages as described below.

Fuzeon (enfurvirtide, T20) targets the gp41 protein on HIV's surface. The gp41 protein is in its resting state embedded in the HIV envelope structure (ENV). Binding of HIV to the CD4 receptor on the surface of CD4+-cells triggers a conformational change in the ENV structure, and gp41 becomes exposed. Only at this point is Fuzeon able to interfere with gp41 and inhibit fusion of the HIV particle with the CD4+ cell membrane. This means that Fuzeon has a very limited window of action. As soon as the HIV particle binds to the CD4 receptor, Fuzeon must be present in the vicinity and be able to quickly bind to the exposed gp41 molecule in order to prevent HIV from completing the fusion process and entering the target cell. As a result hereof, a relatively high concentration of Fuzeon must constantly be maintained within the body in order for Fuzeon to be effective.

Selzentry (maraviroc) targets the CCR5 co-receptor on the surface of the target cells. As described above the HIV particle binds first to the CD4 receptor, and hereafter to the co-receptor CCR5 or CRCX4. Binding of Selzentry to the CCR5 co-receptor will thus prevent HIV strains that utilize CCR5 to bind to this co-receptor. Thus, Selzentry will only prevent HIV strains that are CCR5-tropic, but not CXCR4-tropic or CXC4/CCR5 bitropic (dualtropic) HIV strains. Moreover, binding of Selzentry to the CCR-5 co-receptor may interfere with the normal function of CCR-5 as a chemokine receptor with a putative role in the inflammatory response to infection.

Thus, the entry/fusion inhibitors Fuzeon and Selzentry are both effective only when the HIV particle is already bound to the CD4 receptor of its target cell. Therefore, there is great need for a new class entry/fusion inhibitors that will both inhibit free virus particles not bound to its target cell, and not interfere with the normal functions of mammalian cell receptors.

The bivalent molecules of the present invention are effective on free HIV particles, not bound to the target cell, and thus the bivalent molecules of the present invention are effective before the fusion process has begun. Therefore the bivalent molecules of the present invention represent a new class of entry/fusion inhibitors, which herein are referred to as “pre-fusion inhibitors”. The pre-fusion inhibitors of the present invention are bivalent molecules, encompassing one part that is able to mimic the function and/or structure of the CD4 receptor, and another part that is able to interact with and/or bind to the ENV protein, or part thereof, of the virus particle. The two parts of the bivalent molecule are preferably joined by linker molecule. The pre-fusion inhibitors comprising the bivalent molecules of the present invention work by contacting the free virus particle and then the first part of the bivalent molecules mimic the function of the CD4 receptor, forcing the virus particle to undergo conformational changes, and then the second part of the bivalent molecules will interact with and/or bind to the ENV protein of the virus particle. Hereby, the action of the bivalent molecules of the present invention triggers the virus to undergo the necessary molecular steps of the fusion process, while not being near or in contact with a CD4+ cell. Since the virus only once can perform these molecular steps, it has forever lost its ability to infect CD4+ cells. This means that the virus particle is permanently neutralized and rendered harmless. Therefore, the bivalent molecules of the present invention are particular effective for use as a microbicide.

### SUMMARY OF INVENTION

The present invention relates to a new class of virus entry inhibitors, in particular inhibitors of human immunodeficiency virus (HIV). The entry inhibitors of the present are bivalent molecules, encompassing one part (the first part) with functional and/or structural homology to a mammalian receptor involved in viral fusion, and another part (the second part) with sequence homology to peptides originating from virus. The entry inhibitors of the present invention are in particular useful against viruses that make use of the type 1 fusion mechanism belonging to the groups of viruses consisting of Orthomyxoviridae, Paramyxoviridae, Retroviridae, Filoviridae and Coronaviridae. The two parts of the bivalent molecule are joined by a linker molecule.

In one aspect, the present invention relates to a molecule comprising:

i) a first part that comprises or consists of a first virus binding moiety that binds to a first viral protein; and
ii) a second part that comprises or consists of a second virus binding moiety that binds to a second viral protein.
[NB—the embodiment wherein the first and second parts bind to different domains on the same protein is included in the new second aspect of the invention, below]

Preferably, the first and second parts are linked by a linker.

In one embodiment the first part exhibits the virus binding function of a mammalian membrane receptor or a soluble part thereof.

In one embodiment, the first part comprises structural homology to a mammalian membrane receptor. Hence, it exhibits the 3-dimensional structure and/or charge distribution of a mammalian membrane receptor or a part thereof.

Preferably, the first part comprises or consists of an amino acid sequence. Most preferably, the amino acid sequence corresponds to the amino acid sequence of a mammalian membrane receptor or a soluble part, a fragment, mimic or functional homologue thereof or an amino acid sequence at least 80% identical to a amino acid sequence corresponds to the amino acid sequence of a mammalian membrane receptor or a soluble part, a fragment, mimic or functional homologue thereof.

The mammalian membrane receptor is preferably a receptor that is used by a virus during viral infection. For example, it may be used in a type 1 fusion mechanism. Preferably, it is used by a virus for docking on a target cell.

Examples of viruses that use type 1 fusion mechanisms include Othomyxoviridae, Paramyxoviridae, Retroviridae, Filoviridae and Coronaviridae. Preferably, the virus is selected from the group of viruses consisting of HTLV-1, HTLV-2, HERV, BLV, ELV, FeLV, PuLV, O/CLV, visna/maedi, PrLV, HIV-1, HIV-2, SIV, MLV, JSRV, FeLV A, Influenza HA, and ebola. More preferably, the virus is selected from the group of viruses consisting of HTLV-1, HTLV2, HERV, HIV-1, HIV-2, Sly, MLV, BLV, JSRV and FeLV A. For example, the virus may be selected from the group of viruses consisting of HIV-1, HIV-2 and SIV. However, it is preferred that the virus is Human Immunodeficiency Virus (HIV).

Preferably, the mammalian membrane receptor is selected from the group consisting of CD4, sCD4, ICAM-1, coxsackievirus-adenovirus receptor (CAR), poliovirus receptor (CD155), HAVCr-1, neural cell adhesion molecule (CD56), MHC class I, MHC class II, Nectin 1, Nectin 2, aV integrins, a2b1, a chemokine receptor, c Complement receptor CR2 (CD21), CD46, decay-accelerating factor (CD55), low-density lipoprotein receptor, acetylcholine receptor, epidermal growth factor receptor, herpesvirus entry mediator (HVEM), sialic acid and heparan sulfate.

In one embodiment, the first part comprises or consists of mammalian soluble CD4 (sCD4) or a fragment, mimic, or functional homologue thereof, or an amino acid sequence at least 80% identical to soluble CD4 (sCD4) or a fragment, mimic, or functional homologue thereof.

In a further embodiment, the first part is human soluble CD4 (sCD4) or a fragment, mimic, functional homologue thereof, or an amino acid sequence at least 80% identical to human soluble CD4 (sCD4) or a fragment, mimic, or functional homologue thereof. Preferably, the first part is sCD4.

In an alternative embodiment, the mammalian membrane receptor is a co-receptor. The co-receptor may be is selected from the group consisting of Claudin-1, Occludin (utilized by hepatitis C virus), PILR-α in (utilized by HSV), mannose-binding lectin, FR-alpha, Integrins (utilized by EBOV), AlphaVbeta5 integrin (utilized by Adeno-associated virus type 2), Human Hepatocyte Growth Factor (utilized by AAV3), CCR5, CXCR4, CCR2, CCR3, CCRB, CCR9, CXCR6 (Bonzo/STRL33/TYMSTR), CX3CR1, ChemR23, APJ, Bob/GPR15, GPR1 and RDC1 (utilized by HIV). However, it is preferred that the co-receptor is CCR5 or CXCR4.

In a further alternative embodiment, the first part comprises or consists of an N-phenyl-N′-piperidine-oxalamide derivative, for example, an N-phenyl-N′-piperidine-oxalamide derivative selected from the group of compounds consisting of NBD-556, NBD-557, DN-3186, JRC-II-75 and JRC-II-11.

The molecule may further comprise a purification tag, such as a hexahistidine tag.

In one embodiment the first part of the molecule is a peptide with amino acid sequence selected from the group consisting of SEQ ID NOS: 9-10, and the linker is a peptide with amino acid sequence consisting of SEQ ID NO: 19, and the second part is a peptide with amino acid sequences selected from the group consisting of SEQ ID NOS: 11-18, 20-204.

Thus, in a particular embodiment, the molecule is a peptide with amino acid sequence selected from the group consisting of SEQ ID NOS: 1-8 or 216-225.

In a preferred embodiment, the molecule is a peptide with amino acid sequence selected from the group consisting of SEQ ID NOS: 1, 6-8.

In one embodiment, the second part of the molecule comprises or consists of a peptide with an amino acid sequence selected from the group consisting of any one of SEQ ID NOs: 237-275. Preferably, the second part of the molecule comprises or consists of SEQ ID NO: 237. More preferably, the second part of the molecule consists of SEQ ID NO: 237

In one embodiment the second viral protein is a peptide capable of forming a coiled coil. Preferably, the second viral protein is a heptad repeat structural motif, for example, the protein may be part of the HIV envelope structure (ENV). Preferably, the second viral protein is HIV gp160. More preferably, it is HIV gp41.

In one embodiment, the first and/or second part is an antibody or an antigen-binding fragment. Preferably, the antibody or antigen-binding fragment is selected from the group consisting of intact antibodies, Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]). Most preferably, the antibody or antigen-binding fragment is a single chain Fv (scFv).

Alternatively, the first and/or second part comprises or consists of an antibody-like binding agent, for example an affibody or aptamer.

In one embodiment, the second part is capable of binding to a viral membrane anchored protein such as gp41 of HIV. Preferably, the virus makes use of a type 1 fusion mechanism, such as Othomyxoviridae, Paramyxoviridae, Retroviridae, Filoviridae and Coronaviridae. Preferably, the virus is selected from the group of viruses consisting of HTLV-1, HTLV-2, HERV, BLV, ELV, FeLV, PuLV, O/CLV, visna/maedi, PrLV, HIV-1, HIV-2, SIV, MLV, JSRV, FeLV A, Influenza HA, Marburg, and ebola. However, it is preferred that the virus is Human Immunodeficiency Virus (HIV) and the membrane anchored protein is gp41.

Preferably, the second part comprises or consists of a peptide with amino acid sequence corresponding to the amino acid sequence of a second viral protein, or a part, fragment, mimic, or functional homologue thereof. For example, the second part may comprise or consist of a peptide capable of forming a coiled coil. This may be a heptad repeat structural motif. The second viral protein is capable of forming a triple-helix. Preferably, the second viral protein is part of the viral envelope structure (ENV), preferably, the HIV viral envelope.

In one embodiment, the second viral protein comprises or consists of the HIV gp41 protein, or any part, fragment, mimic or functional homologue thereof.

Thus, in one embodiment, the second part comprises or consists of a peptide having an amino acid sequence according to any one of SEQ ID NOS: 11-18 or 20-204 or a fragment, mimic, or functional homologue thereof, or a peptide having an amino acid sequence at least 80% identical to any one of SEQ ID NOS: 11-18 or 20-204.

Thus, the second part may comprise or consist of a peptide having an amino acid sequence according to any one of SEQ ID NOS: 20-65 or any part, fragment, mimic or functional homologue thereof, or a peptide having an amino acid sequence at least 80% identical to any one of SEQ ID NOS: 20-65.

The second part may comprise or consist of a peptide having an amino acid sequence according to any one of SEQ ID NOS: 66-83 or any part, fragment, mimic or functional homologue thereof, or a peptide having an amino acid sequence at least 80% identical to any one of SEQ ID NOS: 66-83.

The second part may comprise or consist of a peptide having an amino acid sequence according to any one of SEQ ID NOS: 84-175 or any part, fragment, mimic or functional homologue thereof, or a peptide having an amino acid sequence at least 80% identical to any one of SEQ ID NOS: 84-175.

The second part may comprise or consist of a peptide having an amino acid sequence according to any one of SEQ ID NOS: 176-204 or any part, fragment, mimic or functional homologue thereof, or a peptide having an amino acid sequence at least 80% identical to any one of SEQ ID NOS: 176-204.

The second part may comprise or consist of a peptide having an amino acid sequence according to any one of SEQ ID NOS: 11-18 or any part, fragment, mimic or functional homologue thereof, or a peptide having an amino acid sequence at least 80% identical to any one of SEQ ID NOS: 11-18.

The second part may comprise or consist of a peptide having an amino acid sequence according to any one of SEQ ID NOS: 12-15, or any part, fragment, mimic or functional homologue thereof, or a peptide having an amino acid sequence at least 80% identical to any one of SEQ ID NOS: 12-15.

Alternatively, the second part may comprise or consist of a peptide having an amino acid sequence according to any one of SEQ ID NOS: 11, 16, 17 or 18, or any part, fragment, mimic or functional homologue thereof, or a peptide having an amino acid sequence at least 80% identical to any one of SEQ ID NOS: 11, 16, 17 or 18.

It is preferred that the linker is a polymer. The polymer may be selected from the group of polymers consisting of polyamides, polypeptides, polysaccharides and polynucleotides. Preferably the polymer comprises or consists of a peptide with an amino acid sequence according to SEQ ID NO: 19, or any part, fragment, mimic, or functional homologue thereof, or an amino acid sequence at least 80% identical SEQ ID NO: 19. Thus, it is preferred that the linker is a peptide with amino acid sequence consisting of SEQ ID NO: 19.

Where the molecule of the invention is a polypeptide, the first part may located N-terminally relative to the amino acid sequence of the second part. Alternatively, the first part may be located C-terminally relative to the amino acid sequence of the second part.

The molecules of the present invention are suitable for inhibiting viral infection. It is preferred that they are virus pre-fusion inhibitors. Alternatively or additionally they may be virus entry inhibitors and/or virus fusion inhibitors. Thus, the molecule may be able to destabilize the virus envelope structure (ENV) by triggering conformational changes in said envelope structure. Preferably, the molecule is capable of transforming the virus envelope structure (ENV) from the pre-fusion state to the post-fusion state, or any intermediate transition state. Most preferably, the molecule is capable of maintaining the virus in the post-fusion state.

Preferably the molecule is an inhibitor of a virus selected from the group consisting of Othomyxoviridae, Paramyxoviridae, Retroviridae, Filoviridae and Coronaviridae. For example, the molecule may be an inhibitor of a virus selected from the group consisting of HTLV-1, HTLV-2, HERV, BLV, ELV, FeLV, PuLV, O/CLV, visna/maedi, PrLV, HIV-1, HIV-2, SIV, MLV, JSRV, FeLV A, Influenza HA, Marburg, and ebola.

In one embodiment the molecule is an inhibitor of a virus selected from the group consisting of HTLV-1, HTLV2, HERV, HIV-1, HIV-2, SIV, MLV, BLV, JSRV and FeLV A. For example, the molecule may be an inhibitor of a virus selected from the group consisting of HIV-1, HIV-2 and SIV.

Preferably, the molecule is an inhibitor of Human Immunodeficiency Virus (HIV) such as HIV-1 or HIV-2.

Accordingly, it is preferred that the molecule of the invention comprises or consists of a peptide having an amino acid sequence according to any one of SEQ ID NOS: 1-8 or any part, fragment, mimic, or functional homologue thereof, or a peptide having an amino acid sequence at least 80% identical to any one of SEQ ID NOS: 1-8.

Thus, the molecule may comprise or consist of a peptide having an amino acid sequence according to any one of SEQ ID NOS: 2-5 or any part, fragment, mimic, or functional homologue thereof, or a peptide having an amino acid sequence at least 80% identical to any one of SEQ ID NOS: 2-5.

The molecule may comprise or consist of a peptide having an amino acid sequence according to any one of SEQ ID NOS: 1 or 6-8 or any part, fragment, mimic or functional homologue thereof, or a peptide having an amino acid sequence at least 80% identical to any one of SEQ ID NOS: 1 or 6-8.

The molecule may comprise or consist of a peptide having an amino acid sequence according to SEQ ID NO: 1 or any part, fragment, mimic or functional homologue thereof, or a peptide having an amino acid sequence at least 80% identical to SEQ ID NO: 1.

The molecule may comprise or consist of a peptide having an amino acid sequence according to SEQ ID NO: 6 or any part, fragment, mimic or functional homologue thereof, or a peptide having an amino acid sequence at least 80% identical to SEQ ID NO: 6.

The molecule may comprise or consist of a peptide having an amino acid sequence according to SEQ ID NO: 7 or any part, fragment, mimic or functional homologue thereof, or a peptide having an amino acid sequence at least 80% identical to SEQ ID NO: 7.

Alternatively, it may comprise or consist of a peptide having an amino acid sequence according to SEQ ID NO: 8 or any part, fragment, mimic or functional homologue thereof, or a peptide having an amino acid sequence at least 80% identical to SEQ ID NO: 8.

The present invention also pertains to a polynucleotide comprising and/or consisting of a nucleic acid sequence encoding at least one molecule as defined herein or any part thereof, or fragment thereof, or mimic thereof, or functional homologue of said molecule, or a polynucleotide at least 80% identical to said nucleic acid sequence or part thereof, or any polynucleotide that have been modified by codon optimization, encoding at least one molecule as defined herein.

In a preferred embodiment the present invention relates to a polynucleotide comprising and/or consisting of a nucleic acid sequence selected from the group consisting of SEQ ID NOS: 205-212 or 226-235 encoding at least one molecule selected from the group consisting of SEQ ID NOS: 1-8 or 216-225 or any part thereof, or fragment thereof, or mimic thereof, or functional homologue of said molecule, or a polynucleotide at least 80% identical to said nucleic acid sequence or part thereof, or any polynucleotide that have been modified by codon optimization, encoding at least one molecule with SEQ ID NOS: 1-8 or 216-225.

The molecule of the first aspect of the invention may be capable of forming multimers such as dimers or trimers, preferably trimers under physiological conditions. The multimers of the invention may have enhanced viral infection inhibition compared to the monomeric form.

Whilst not wishing to be bound by theory, the molecule of the first aspect of the invention may comprise a second part corresponding to and/or mimicking a part of gp41 that is thought to be able to trimerize. Therefore, the natural conformation of the bivalent inhibitor may be a trimer. Since the envelope protein (gp120) is also a trimer, a trimer of sCD4 may have has a better chance of neutralizing the envelope protein either by binding to and/or inducing irreversible conformational changes in gp41.

Hence, multimerization of sCD4 or other gp120-binding molecules may result in more potent anti-HIV molecules.

In one embodiment, the molecule of the first aspect of the invention comprises a peptide fusion inhibitor such as sifurvitide or enfuvirtide in order to stabilise the helix structure.

A second aspect of the present invention provides a molecule comprising:

i) a first part that comprises or consists of a first virus binding moiety that binds to a viral protein; and
ii) a second part that comprises or consists of a second virus binding moiety that binds to the viral protein at a different site to the first virus binding moiety.

Preferably, the first and second parts are linked by a linker.

In one embodiment the first part exhibits the virus binding function of a mammalian membrane receptor or a soluble part thereof.

In one embodiment the first part corresponds to the first part of the molecule according to the first aspect of the present invention. Preferably, the first part binds to a mammalian membrane receptor-binding domain of the viral protein. Preferably, the mammalian membrane receptor-binding domain of the viral protein overlaps with the site of the viral protein that interacts with and/or binds to the viral membrane anchored protein (or a subunit thereof). In another, also preferred embodiment, the mammalian membrane receptor-binding domain of the viral protein does not overlap with the site of the viral protein that interacts with and/or binds to the viral membrane anchored protein (or a subunit thereof).

In one embodiment, the site of the viral protein that interacts with and/or binds to the viral membrane anchored protein (or a subunit thereof) is responsible for inducing a conformational change in the membrane anchored protein (or a subunit thereof) when the viral protein binds to a mammalian membrane receptor.

Preferably, the virus makes use of the type 1 fusion. Suitable viruses include Orthomyxoviridae, Paramyxoviridae, Retroviridae, Filoviridae and Coronaviridae. Preferably, the virus is selected from the group of viruses consisting of HTLV-1, HTLV-2, HERV, BLV, ELV, FeLV, PuLV, O/CLV, visna/maedi, PrLV, HIV-1, HIV-2, SIV, MLV, JSRV, FeLV A, Influenza HA, Marburg, and ebola.

However, it is especially preferred that the virus is HIV and the viral protein is gp120.

In one embodiment, the second part of the molecule of the second aspect of the invention comprises or consists of a second virus binding moiety that binds to the viral protein at a site that interacts with and/or binds to the viral membrane anchored protein (or a subunit thereof). Preferably, the site of the viral protein that interacts with and/or binds to the viral membrane anchored protein (or a subunit thereof) is responsible for inducing a conformational change in the membrane anchored protein (or a subunit thereof) when the viral protein binds to a mammalian membrane receptor.

In this embodiment, the viral protein, when bound to the molecule of the second aspect of the invention, is prevented from binding to, interacting with, and/or inducing conformational change of the membrane anchored protein (or a subunit thereof) of a/the corresponding membrane anchored protein (or a subunit thereof). Thus, the membrane anchored protein (or subunit thereof) is prevented from assuming its active conformation when the viral protein binds to a mammalian membrane receptor.

However, in another preferred embodiment, the second part of the molecule binds to the viral protein at a site that does not interact with and/or bind to the viral membrane anchored protein (or a subunit thereof).

Preferably, the viral protein is shed following binding by the molecule of the second aspect of the invention, resulting in permanent inactivation of the viral fusion machinery.

Preferably, the membrane anchored protein is gp41.

Preferably, binding of the viral protein by the first part and the second part of the molecule of the second aspect of the invention causes the viral protein to be shed from the virus. Hence, the virus is unable to bind to target cells and rendered non-infectious.

In one embodiment, the second part is an antibody or an antigen-binding fragment. Preferably, the antibody or antigen-binding fragment is selected from the group consisting of intact antibodies, Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]). Most preferably, the antibody or antigen-binding fragment is a single chain Fv (scFv).

Alternatively, the second part comprises or consists of an antibody-like binding agent, for example an affibody or aptamer.

In one embodiment, the molecule of the second aspect of the invention comprises a peptide fusion inhibitor such as sifurvitide or enfuvirtide.

The molecules of the first and second aspects of the invention may irreversibly bind to their target virus. However, it is preferred that they bind reversibly to their target virus so that, following immobilisation of the bound virus particle's ability to bind to and/or infect target cells, the molecule is liberated, allowing them to inactivate further virus particles. Reversible binding of the molecules of the invention can be achieved using, for the first part of the molecule, CD4 or sCD4 with one or more point mutations corresponding to Chimpanzee CD4 or sCD4 (for example, SEQ ID NO 215). SEQ ID NO: 216 corresponds to the amino acid sequence encoding a complete molecule of the invention.

Thus, a third aspect of the present invention relates to a polynucleotide comprising or consisting of a polynucleotide having a nucleic acid sequence encoding a molecule according to the first or second aspects of the present invention. Preferably, the polynucleotide has been codon optimised.

Hence, in one embodiment, the polynucleotide comprises or consists of a polynucleotide having a nucleic acid sequence according to any one of SEQ ID NOS: 205-212 or 226-235 or a part or fragment thereof, or a codon optimised polynucleotide encoding a polypeptide according to any one of SEQ ID NOS: 1-8 or 216-225.

In another embodiment, the polynucleotide comprises or consists of a polynucleotide having a nucleic acid sequence according to any one of SEQ ID NOS: 206-209 or any part or fragment thereof, or a codon optimised polynucleotide encoding a polypeptide according to any one of SEQ ID NOS: 2-5.

In another embodiment, the polynucleotide comprises or consists of a polynucleotide having a nucleic acid sequence according to any one of SEQ ID NOS: 205 or 210-212 or any part or fragment thereof, or a codon optimised polynucleotide encoding a polypeptide according to any one of SEQ ID NOS: 1 or 6-8.

In another embodiment, the polynucleotide comprises or consists of a polynucleotide having a nucleic acid sequence according to SEQ ID NO: 205 or any part or fragment thereof, or a polynucleotide having a nucleic acid sequence at least 80% identical to SEQ ID NO: 205, or a codon optimised polynucleotide encoding a polypeptide according to SEQ ID NO: 1.

In another embodiment, the polynucleotide comprises or consists of a polynucleotide having a nucleic acid sequence according to SEQ ID NO: 210 or a part or fragment thereof, or a codon optimised polynucleotide having a nucleic acid sequence at least 80% identical to SEQ ID NO: 210, or a codon optimised polynucleotide encoding a polypeptide according to SEQ ID NO: 6.

In another embodiment, the polynucleotide comprises or consists of a polynucleotide having a nucleic acid sequence according to SEQ ID NO: 211 or a part or fragment thereof, or a polynucleotide having a nucleic acid sequence at least 80% identical to SEQ ID NO: 211, or a codon optimised polynucleotide encoding a polypeptide according to SEQ ID NO: 7.

Thus, the polynucleotide may comprise or consist of a polynucleotide having a nucleic acid sequence according to SEQ ID NO: 212 or a part or fragment thereof, or a polynucleotide having a nucleic acid sequence at least 80% identical to SEQ ID NO: 212, or a codon optimised polynucleotide encoding a polypeptide according to SEQ ID NO: 8.

The present invention further relates to an isolated expression vector comprising at least one polynucleotide comprising or consisting of at least one nucleic acid sequence as described above coding for at least one molecule as described above.

A fourth aspect of the present invention provides an expression vector comprising a nucleic acid sequence encoding a molecule according to the first or second aspects of the present invention or a polynucleotide according to the second aspect.

Preferably, the vector is a prokaryotic expression vector. The prokaryotic expression vector may be selected from the group consisting of pUC18, pUC19, pBR322, pBR329, pTrc99A, pKK223-3, pKK233-3, pDR540, pRIT5, pBS vectors, Phagescript vectors, Bluescript vectors, pNH8A, pNH16A, pNH18A or pNH46A.

However, it is equally preferred that the vector is a eukaryotic expression vector such as pRS403-406, pRS413-416, pRS403, pRS404, pRS405, pRS406 or pRS413-416. Hence, the vector may be a mammalian expression vector, for example, pSVL or pMSG. In one embodiment the vector is isolated.

A fifth aspect of the invention provides a host cell comprising the fourth aspect of the invention. The molecules of the invention can, in principal, be produced in any type of cells including prokaryotic cells. Especially preferred are insect cells (for example, a baculovirus expression system) and yeast cells as a practical means of production. Preferably, the host cell type is selected from the group consisting of 293T, Vero, HeLa, Jurkat, TE671, 293 and HEK 293. However, it is preferred that the host cell type is 293T.

The invention also relates to pharmaceutical compositions comprising one or more molecules as defined herein for use as a medicament. The present invention also relates to pharmaceutical compositions comprising one or more molecules as defined herein for the prevention and/or amelioration and/or treatment of virus infections caused by viruses belonging to the groups of viruses consisting of Orthomyxoviridae, Paramyxoviridae, Retroviridae, Filoviridae and Coronaviridae, and especially HIV.

Thus, a sixth aspect of the present invention provides a pharmaceutical composition comprising one or more molecules according to the first, second, third, fourth and/or fifth aspects of the present invention. Preferably, the pharmaceutical composition comprises one or more molecules according to the first or second aspects.

Preferably, the pharmaceutical composition further comprises a pharmaceutically and/or physiologically acceptable salt and/or a physiologically acceptable carrier.

The pharmaceutical composition may be for the prevention and/or amelioration and/or treatment of diseases and/or clinical conditions arising from virus infection. Preferably, the virus is Human Immunodeficiency Virus (HIV). Preferably, the disease is Acquired Immune Deficiency Syndrome (AIDS).

A seventh aspect of the present invention provides a method of preparation of the pharmaceutical composition according to the sixth aspect comprising:

a) providing one or more molecules according to the first or second aspects of the invention;
b) optionally, providing a salt and/or a carrier;
c) providing a substance; and
d) mixing the molecules of step (a) and (b) with the substance of step c).

Preferably, the one or more molecules of step (a) are produced by expression of the vector(s) of the invention. Alternatively, the one or more molecules of step (a) are produced by chemical synthesis.

It is preferred that the substance of step (c) is selected from the group of substances consisting of lubricants, creams, lotions, shake lotions, ointments, gels, balms, salves, oils, foams, shampoos, sprays and aerosoloes as well as transdermal patches and bandages.

Most preferably, the substance of step (c) is a lubricant, gel, cream, foam and/or lotion.

An eighth aspect of the invention provides the use of a molecule or a pharmaceutical composition of the invention in medicine. Preferably, the use is as a virus inhibitor, for example, a pre-fusion inhibitor, an entry inhibitor and/or a fusion inhibitor.

Most preferably, the inhibitor is able to destabilize the virus envelope structure (ENV) by triggering conformational changes in said envelope structure.

Thus, the inhibitor may be capable of transforming the virus envelope structure (ENV) from the pre-fusion state to the post-fusion state, or any intermediate transition state and/or maintaining the virus in the post-fusion state.

Preferably, the virus is a virus making use of the type 1 envelope fusion mechanism such as Orthomyxoviridae, Paramyxoviridae, Retroviridae, Filoviridae and Coronaviridae. Preferably, the virus is Human Immunodeficiency Virus (HIV).

A tenth aspect of the present invention provides a molecule or pharmaceutical of the invention for use in medicine.

An eleventh aspect of the present invention provides the use of a molecule or pharmaceutical of the invention for the manufacture of a medicament for the treatment and/or amelioration and/or prevention of a disease and/or a clinical condition.

Preferably, the disease and/or clinical condition belongs to the group of diseases and/conditions arising from viral infection. Most preferably, the virus is Human Immunodeficiency Virus (HIV) and/or the disease and/or clinical condition is Acquired Immune Deficiency Syndrome (AIDS).

A twelfth aspect of the present invention provides a molecule or pharmaceutical of the invention for the treatment and/or amelioration and/or prevention of a disease and/or a clinical condition. Preferably, the disease and/or clinical condition belongs to the group of diseases and/conditions arising from viral infection. Most preferably, the virus is Human Immunodeficiency Virus (HIV) and/or the disease and/or clinical condition is Acquired Immune Deficiency Syndrome (AIDS).

A thirteenth aspect of the present invention provides the use of a molecule or pharmaceutical of the invention for as a microbicide. The use may be as part of a coating composition. For example, the microbicide may be used as a coating of contraceptive devices, medico-technological devices and micro-devices.

A fourteenth aspect of the invention provides the use of a polynucleotide and/or a vector of the present invention in gene therapy. In one embodiment, the one or more polynucleotides and/or vector is expressed in a mammalian cell. In an alternative embodiment, the one or more polynucleotides and/or vector is expressed in a single-cell organism. Preferably, the single-cell organism is selected from the group consisting of bacteria, protozoa, amoebae, moulds, yeast and fungus.

Another aspect of the present invention pertains to a method of preparation of the pharmaceutical composition as defied above comprising the steps of

a. providing one or more molecules as defined herein
b. optionally providing a salt and/or a carrier
c. providing a substance
d. mixing the molecules of step a. or b. with the substance of step c.
e. obtaining the pharmaceutical composition of claims as defined herein

Yet another aspect of the present invention relates to the use of one or more molecules as defined herein, or the pharmaceutical compositions defined herein, as a virus inhibitor, more specifically a virus fusion/entry inhibitor, and preferably a virus pre-fusion inhibitor. The invention is particular useful for inhibiting viruses belonging to the groups of viruses consisting of Orthomyxoviridae, Paramyxoviridae, Retroviridae, Filoviridae and Coronaviridae, and especially HIV.

The invention also relates to the use of one or more molecules as defined herein for the manufacture of a medicament for the treatment and/or amelioration and/or prevention of diseases and/or clinical conditions. Further, the invention relates to the use of one or more molecules as defined herein, or the pharmaceutical compositions as defined herein, as a microbicide.

Another aspect of the present invention pertains to a compound comprising one or more molecules as defined herein for the prevention and/or amelioration and/or treatment of a disease and/or clinical condition belonging to the group of diseases and/or clinical conditions arising from virus infections, in particular infections caused by viruses belonging to the groups of viruses consisting of Orthomyxoviridae, Paramyxoviridae, Retroviridae, Filoviridae and Coronaviridae, and especially HIV.

In a final aspect the present invention relates to a method of treating, preventing and/or ameliorating a disease and/or clinical condition, said method comprising administering to an individual suffering from said disease and/or clinical condition an effective amount of one or more molecules as defined herein, wherein said disease and/or clinical condition belongs to the group of diseases and/or clinical condition arising from virus infections, in particular infections caused by viruses belonging to the groups of viruses consisting of Orthomyxoviridae, Paramyxoviridae, Retroviridae, Filoviridae and Coronaviridae, and especially HIV.

### DESCRIPTION OF THE DRAWINGS

FIG. 1. The viral membrane fusion mechanism. Viral envelopes mediate fusion by undergoing several sequential conformational changes. The envelope protein (ENV) is kinetically arrested in a meta-stable conformation upon synthesis in the producer cells. It is this meta-stable protein that finds its way into virions. In other words, the envelope protein on the surface of the viral particles is not in its thermodynamically most stable conformation. This is necessary, since fusion between the cellular and viral membranes involves overcoming a large activation-energy barrier. The events that lead to membrane fusion benefit from the latent energy stored in the envelope protein. This energy is released when the ENV protein undergoes the conformational changes seen in FIG. 1. The release of this latent energy involves several stepwise conformational changes, the most important of which is binding to the receptor and formation and folding of the extended triple-helix (FIGS. 1A, 1B and 1C).

FIG. 2. Free energy diagram illustration of the ENV protein during fusion. The effect of the bivalent molecule of the present invention on stabilizing the transition states/intermediates is illustrated by the dashed line. The bivalent molecules of the present invention work by lowering the activation energies of at least two of the conformational changes illustrated in FIG. 1. The first (“Receptor binding”) is through binding of the first part of the bivalent molecules, that mimics receptor binding, to the ENV, and the second (“Triple helix formation”) is by stabilizing the coiled coil structures that are formed in the gp41 protein during fusion, through interaction of second part of the bivalent molecules with the alpha-helices of this protein (FIG. 2). One other consequence of the large difference between the free energy of the pre-fusion conformation and the post-fusion conformation in the envelope protein is that there is no equilibrium between the two forms: Once the conformational changes occur, the post-fusion form of the ENV protein can never go back to its meta-stable pre-fusion conformation. This means that the bivalent molecules of the present invention triggers the envelope proteins on the viral surface to undergo the conformational changes towards the thermodynamically stable form of the protein (post-fusion conformation), while not in the vicinity of the target cell membrane, the stored energy that was meant for mediating membrane fusion is wasted and the envelope protein is neutralized as far as fusion activity is concerned, as a direct result of the effect of the bivalent molecules of the present invention.

FIGS. 3-5. Inhibitory effect of the bivalent molecules of the present invention. The bivalent molecule sCD4-T20 corresponds to a polypeptide with SEQ ID NO: 1 of the present invention. Pseudotyped viral particles containing MLV core (gagpol and a neo containing retroviral vector) and truncated HIV envelope protein were incubated with supernatant containing sCD4-T20 for the indicated period of time at 37 degrees C. Subsequently, the infectivity (titer) of the virus was measured on D17 cells that stably express HIV receptor and co-receptor, through serial dilutions. After 10 days of selection with G418, colonies were counted and the titer calculated. Ordinate units are Titer cfu/ml. (See also Example 1 elsewhere herein)

FIGS. 6-8: Inhibitory effect of the bivalent molecules of the present invention on HIV spreading. ELISA measurements of the HIV core protein p24gag in the supernatant of cultured Jurkat cells infected with HIV HXB2 strain as a function of days after infection and addition of the bivalent molecules of the present invention. Ordinate values are p24gag concentration (pg/ml) (See also Example 2+3 elsewhere herein). The indicated concentrations represent the content of the corresponding bivalent molecule in the 20% fraction of the figure. The 20% fraction refers to an experiment where the cells have received 1 part supernatant containing the bivalent molecules of the present invention and 4 parts medium.

FIG. 6: 518 corresponds to a polypeptide with SEQ ID NO: 5 and 519 corresponds to a polypeptide with SEQ ID NO: 6 of the present invention.

FIG. 7: 500 corresponds to a polypeptide with SEQ ID NO: 1 and 517 corresponds to a polypeptide with SEQ ID NO: 3 of the present invention.

FIG. 8: 520 corresponds to a polypeptide with SEQ ID NO: 4 and 521 corresponds to a polypeptide with SEQ ID NO: 2

FIG. 9. Control experiment wherein the HIV core protein p24gag (pg/ml) in the supernatant of cultured Jurkat cells infected with HIV HXB2 are measured as a function of days after the addition of sCD4 alone, T20 (Fuzeon, Enfuvirtude) alone, or both sCD4 and T20 (in 1:1 molar ratio). Only a slight inhibitory effect is seen when sCD4 or sCD4+T20 are added. This result should be compared to the result presented in FIG. 7, wherein one of the bivalent molecules comprising sCD4-linker-T20 (SEQ ID NO: 1) has a dramatically profound inhibitory effect on HIV spreading. Ordinate values are p24gag concentration (pg/ml).

FIGS. 10-14. Inhibitory effect of the bivalent molecules of the present invention on HIV spreading. The experiment is based on the activation of the luciferase gene upon infection of TZM-bl cells. X-axis depict 3 different dilutions of a virus stock (HXB2 (CRCX4-trop, virus 89.6 (Dual-trop) or JRCSF (CCR5-trop)) with unknown titer (1×, 5× and 25×). Y-axis depicts the amount of luminescence. Lower luminescence corresponds to greater inhibitory effect of the bivalent molecules of the present invention. (See also Example 4 elsewhere herein).

FIG. 10: mol 500 corresponds to a polypeptide with SEQ ID NO: 1 of the present invention. HIV HXB2 is incubated in the presence of mol 500 in a concentration of 0.2 micrograms/ml and 0.1 micrograms/ml. HBX2 incubated without mol 500 is shown as a control.

FIG. 11: mol 519 corresponds to a polypeptide with SEQ ID NO: 6 of the present invention. HIV HXB2 is incubated in the presence of mol 519 in a concentration of 0.2 micrograms/ml and 0.1 micrograms/ml. HBX2 incubated without mol 519 is shown as a control.

FIG. 12: mol 500 corresponds to a polypeptide with SEQ ID NO: 1 of the present invention. Virus 89.6 is incubated in the presence of mol 500 in a concentration of 0.2 micrograms/ml and 0.1 micrograms/ml. Virus 89.6 incubated without mol 500 is shown as a control, as well as virus 89.6 incubated with sCD4 in concentrations of 0.2 micrograms/ml and 0.1 micrograms/ml.

FIG. 13: mol 519 corresponds to a polypeptide with SEQ ID NO: 6 of the present invention. Virus 89.6 is incubated in the presence of mol 519 in a concentration of 0.2 micrograms/ml and 0.1 micrograms/ml. Virus 89.6 incubated without mol 519 is shown as a control.

FIG. 14: mol 500 corresponds to a polypeptide with SEQ ID NO: 1 of the present invention. JRCSF is incubated in the presence of mol 500 in a concentration of 0.2 micrograms/ml and 0.1 micrograms/ml. JRCSF incubated without mol 500 is shown as a control.

FIG. 15: A: Entry of the virus is initiated by the binding of the envelope protein to the CD4 receptor. Subsequent interaction with the co-receptor results in shedding of the gp120 and exposure of the fusion mechanism in gp41, which forms a long triple helix and inserts the fusion peptide into the cell membrane. The membranes are pulled together and fuse when the triple helix in gp41 folds back onto itself and forms a six helix bundle. B: the putative mode of action by the bivalent inhibitor. The CD4 moiety of the bivalent molecule (black) binds to the envelope protein positioning the helix (purple) to interact with the gp41. This interaction stabilizes the formation of the extended triple helix in absence of a co-receptor resulting in “firing” of the fusion mechanism, which causes the shedding of gp120 and subsequent inactivation of the envelope protein.

FIG. 16: HXB2 replication in Jurkat T cells. Viral replication is determined by p24 antigen in the supernatant. The legend percentage (%) indicates the percentage of the supernatant from transfected 293T cells (that contains the bivalent inhibitor) in the final virus suspension that was added to the cells. (The concentration of the inhibitor was not determined in this experiment.)

FIG. 17: The effect of the bivalent inhibitor on the replication of the HXB2 virus in Jurkat cells in comparison with sCD4 produced under the same circumstances or the commercially available sCD4.

FIG. 18: Virus subtype 89.6 replication in primary human peripheral blood mononuclear cells (PBMCs), Concentrations used are 50 ng/mL of Bivalent and sCD4 with T20 added at 1:1 molar ratio. Viral replication was measurement as the amount of p24 antigen in supernatant. UT: untreated.

FIG. 19: HXB2 single round infection in TZM-bl luciferase indicator cells. Luciferase signal is dependent upon viral entry and transcription. Three inhibitor concentrations are tested. From left to right: 15, 5 and 2.5 ng/mL. CD4 supernatant control is sCD4 produced under the same conditions as the bivalent inhibitor, while sCD4 is the commercial product. Please note the logarithmic scale. UT: untreated.

FIG. 20: JR-CSF single round infection in TZM-bl luciferase indicator cells. The bivalent inhibitor is able to neutralize this virus albeit at higher concentration compared to HXB2. T20 and sCD4 have very minor effects on this HIV subtype. Please note the logarithmic scale. UT: untreated.

FIG. 21: HIV subtype 89.6 single round infection in TZM-bl luciferase indicator cells. The concentrations used were: Bivalent inhibitor 0.05 ug/mL (2 nM), Control sCD4 0.05 ug/mL (2 nM), sCD4 0.05 ug/mL, T20 25 nM, T20+sCD4 1:1 molar 2 nM, Retrovir 125 nM, Abacavir 25 nM, Ritonavir 35 nM, Saquinavir 45 nM. Results from two independent experiments are shown and the RLUI levels are only comparable within each experiment. UT: Untreated. Note the logarithmic scale.

FIG. 22: HIV isolate 89.6 infection of TZM-bl cells upon four different incubation times of virus and inhibitor prior to seeding on target cells. The time dependency of the effect of the bivalent inhibitor sample is highly statistically significant (ANOVA comparing groups). Control sCD4 supernatant is sCD4 produced under the same conditions as the bivalent inhibitor, while sCD4 is the commercially available product. All compounds were added at a concentration of 0.05 ug/mL.

FIG. 23: Virus was incubated at 37° C. with anti-viral compounds, inc. Nucleotide Reverse Transcriptase Inhibitors (NRTIs) and Protease Inhibitors (PIs). Samples were taken at five different time points and added to target cells. A and B depict two independent experiments, that are representative of four performed, in consistently showing a statistically significant time dependency of the bivalent inhibitor, in contrast to all other anti viral compounds tested. Please note the different scales in the graphs.

FIG. 24: Relative infectivity decrease beyond time=0 in one representative experiment. The different drug classes have been color-coded. Red: Fusion inhibitors. Blue: NRTIs. Yellow: PI; Bivalent inhibitor 0.05 ug/mL (2 nM), Control sCD4 0.05 ug/mL (2 nM), T20 25 nM, T20+sCD4 1:1 molar 2 nM, Retrovir 125 nM, Abacavir 25 nM, Ritonavir 35 nM, Saquinavir 45 nM.

FIG. 25: Stability of the bivalent inhibitor and controls in human serum and PBS at 37° C. The indicated compounds were incubated in either serum or PBS at 37° C. for 24 h and their anti-viral activity were measured using the TZM-bl indicator cells. No incubation indicates that the compounds were mixed with either PBS or human serum and added to virus immediately without any incubation. UT: indicates untreated virus by the active compounds (but still containing either serum or PBS).

FIG. 26: Shedding of gp120 by the bivalent inhibitor. HIV-virus was incubated with either #500 bivalent inhibitor (SEQ ID NO: 1) or with medium for 3 hours. The samples were ultracentrifuged using a SW60 rotor at 25000 rpm for 1.5 hours on a sucrose cushion of either 20%, 25% or 30% in order to separate the shed gp120 from the virus particles. The supernatant on top of the sucrose containing the shed gp120 was removed and the amount of gp120 was measured using ELISA.

FIG. 27: Western blot showing the bivalent inhibitor and sCD4 purified from supernatants of either transfected or stably expressing 293T cells using a polyclonal goat anti human CD4 antibody (RnD systems cat. Nr. BAF379). The molecules were purified from the supernatant using CD4 binding magnetic beads (Dynal Biotech) prior to running on a gel. As can be seen the bivalent inhibitor molecule runs slightly slower than the sCD4 because of its larger size.

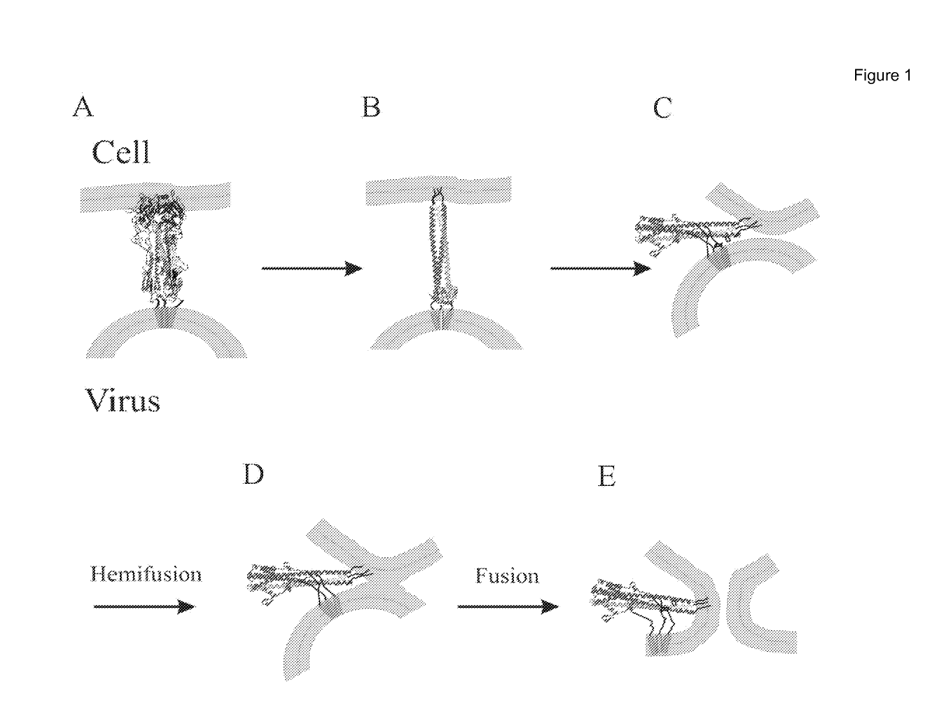

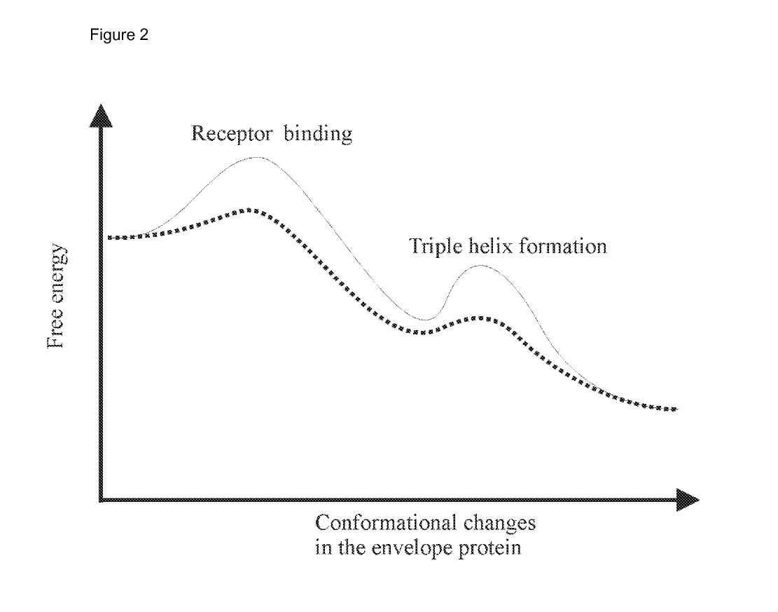

### DETAILED DESCRIPTION OF THE INVENTION

It is a major objective of the present invention of providing a new type of highly efficient viral fusion/entry inhibitor molecules. The viral fusion/entry inhibitor molecules of the present invention are of a new class of fusion/entry inhibitors, herein referred to as “pre-fusion” inhibitors. It is appreciated that the pre-fusion inhibitor molecules of the present invention are able to neutralize free virus particles, and thus render them harmless, even when the virus particles are not in the vicinity of their target cells, or any other cell for that matter. The pre-fusion inhibitors of the present invention are effective against any virus that make use of the type 1 fusion mechanism belonging to the groups of Othomyxoviridae, Paramyxoviridae, Retroviridae, Filoviridae and Coronaviridae, and they are particularly useful for inhibiting and neutralizing HIV virus particles. The pre-fusion inhibitors of the present invention relates to bivalent molecules. Bivalent molecules according to the present invention encompass any molecule that comprise at least two structural and/or functional distinct parts, the at least two parts being able to bind to and/or interact with one or at least two other parts, e.g. other molecular entities.

### TERMS AND DEFINITIONS

By “first viral protein” and “second viral protein” we include a first type of protein from a virus and a second type of protein from the virus, respectively. Preferably, the first and second protein types are from the same virus type. For example, the first viral protein may be a protein used by a virus for docking on a target cell (such as HIV gp120) and the second viral protein may be a protein used by a virus for membrane fusion (such as gp41).

By “co-receptor” we include a further receptor that is bound by a virus in addition to a first receptor bound by that virus. For example, HIV utilizes CD4 as a receptor and CRCX4 or CCR5 as a co-receptor.

The term “polynucleotide” or “nucleic acid sequence” refers to a polymeric form of nucleotides at least 2 bases in length. By “isolated nucleic acid sequence” is meant a polynucleotide that is not immediately contiguous with either of the coding sequences with which it is immediately contiguous (one on the 5′ end and one on the 3′ end) in the naturally occurring genome of the organism from which it is derived. The term therefore includes, for example, a recombinant DNA or RNA which is incorporated into a vector. The nucleotides of the invention can be ribonucleotides, deoxyribonucleotides, or modified forms of either nucleotide. The term includes single and double stranded forms of DNA.

The term “polynucleotide(s)” generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can also refer to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term “polynucleotide” includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

The term “codon optimization” refers to the process of optimizing or substituting nucleotide bases in a given polynucleotide, without changing the amino acid sequence translation product (the polypeptide). Because there are four nucleotides in DNA, adenine (A), guanine (G), cytosine (C) and thymine (T), there are 64 possible triplets encoding 20 amino acids, and three translation termination (nonsense) codons. Because of this degeneracy, all but two amino acids are encoded by more than one triplet. It is within the scope of the present invention that any polynucleotide as disclosed herein may be subjected to codon optimization.

The term “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule. Thus, the term “amino acid” comprises any synthetic or naturally occurring amino carboxylic acid, including any amino acid occurring in peptides and polypeptides including proteins and enzymes synthesized in vivo thus including modifications of the amino acids. The term amino acid is herein used synonymously with the term “amino acid residue” which is meant to encompass amino acids as stated which have been reacted with at least one other species, such as 2, for example 3, such as more than 3 other species. The generic term amino acid comprises both natural and non-natural amino acids any of which may be in the “D” or “L” isomeric form.

| One-letter | Three-letter | |
|---|---|---|
| symbol | symbol | Amino acid |
| A | Ala | alanine |
| B | Asx | aspartic acid or asparagine |
| C | Cys | cysteine |
| D | Asp | aspartic acid |
| E | Glu | glutamic acid |
| F | Phe | phenylalanine |
| G | Gly | glycine |
| H | His | histidine |
| I | Ile | isoleucine |
| K | Lys | lysine |
| L | Leu | leucine |
| M | Met | methionine |
| N | Asn | asparagine |
| P | Pro | proline |
| Q | Gln | glutamine |
| R | Arg | arginine |
| S | Ser | serine |
| T | Thr | threonine |
| U* | Sec | selenocysteine |
| V | Val | valine |
| W | Trp | tryptophan |
| X | Xaa | unknown or other amino acid, i.e. X can be any |
| | | of the conventional amino acids. |
| Y | Tyr | tyrosine |
| Z | Glx | glutamic acid or glutamine (or substances such |
| | | as 4-carboxyglutamic acid and 5-oxoproline |
| | | that yield glutamic acid on acid hydrolysis of |
| | | peptides) |

A “fragment” is a unique portion of the polynucleotide encoding bivalent molecules of the present invention which is identical in sequence to but shorter in length than the parent sequence. Similarly the term ‘fragment’ refers to an HIV-1 envelope polypeptide of the present invention a fragment may comprise up to the entire length of the defined sequence, minus one nucleotide or amino acid residues. For example, a fragment may comprise from 5 to 2000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250, 500, 750, 1000, 1250, 1500, 1750 or at least 2000 contiguous nucleotides or amino acid residues in length. Fragments may be preferentially selected from certain regions of a molecule. For example, a polypeptide fragment may comprise a certain length of contiguous amino acids selected from the first 100 or 250 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

The term “antibody” or “antibodies” as used herein refers to immunoglobulin molecules and active portions of immunoglobulin molecules. Antibodies are for example intact immunoglobulin molecules or fragments thereof retaining the immunologic activity, e.g. single chain antibody fragments (scFv).

The term “structural homology” refers to sequence similarity or, interchangeably, sequence identity, between two or more polynucleotide sequences or two or more polypeptide sequences. The term “structural homology” also refers to similarity or identity between two more molecular entities. Hence, molecular entities comprising structural homology exhibits the 3-dimensional structure and/or charge distribution of another molecular entity (such as a mammalian membrane receptor) or a part thereof.

The term “exhibits the virus binding function of a mammalian membrane receptor” includes the feature referred to having the virus binding capacity (specificity and/or affinity) as a protein from a mammalian membrane. The word “receptor” refers to the mammalian membrane protein's function as a receptor for a virus. Thus, the feature may comprise the virus binding specificity and/or affinity of a mammalian membrane receptor such as CD4 (which binds to gp120 of HIV).

The term “functional homologue” or “functional equivalent” refers to homologues of the molecules according to the present invention is meant to comprise any molecule which is capable of mimicking the function of the first part and/or the second part of the bivalent molecule as described herein. Further the term covers any molecule capable of mimicking the function of the linker molecule of the present invention. Thus, the terms refer to functional similarity or, interchangeably, functional identity, between two or more molecular entities. The term “functional homology” is further used herein to describe that one molecular entity are able to mimic the function of one or more molecular entities. Functional homologues according to the present invention may comprise polypeptides with an amino acid sequence, which are sharing at least some homology with the predetermined polypeptide sequences as outlined herein. For example such polypeptides are at least about 40 percent, such as at least about 50 percent homologous, for example at least about 60 percent homologous, such as at least about 70 percent homologous, for example at least about 75 percent homologous, such as at least about 80 percent homologous, for example at least about 85 percent homologous, such as at least about 90 percent homologous, for example at least 92 percent homologous, such as at least 94 percent homologous, for example at least 95 percent homologous, such as at least 96 percent homologous, for example at least 97 percent homologous, such as at least 98 percent homologous, for example at least 99 percent homologous with the predetermined polypeptide sequences as outlined herein above. The homology between amino acid sequences may be calculated using well known algorithms such as for example any one of BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, and BLOSUM 90.

Functional homologues may comprise an amino acid sequence that comprises at least one substitution of one amino acid for any other amino acid. For example such a substitution may be a conservative amino acid substitution or it may be a non-conservative substitution. A conservative amino acid substitution is a substitution of one amino acid within a predetermined group of amino acids for another amino acid within the same group, wherein the amino acids within predetermined groups exhibit similar or substantially similar characteristics. Within the meaning of the term “conservative amino acid substitution” as applied herein, one amino acid may be substituted for another within groups of amino acids characterized by having
i) hydrophilic (polar) side chains (Asp, Glu, Lys, Arg, His, Asn, Gln, Ser, Thr, Tyr, and Cys,)
ii) hydrophobic (non-polar) side chains (Gly, Ala, Val, Leu, Ile, Phe, Trp, Pro, and Met)
iii) aliphatic side chains (Gly, Ala Val, Leu, Ile)
iv) cyclic side chains (Phe, Tyr, Trp, H is, Pro)
v) aromatic side chains (Phe, Tyr, Trp)
vi) acidic side chains (Asp, Glu)
vii) basic side chains (Lys, Arg, His)
viii) amide side chains (Asn, Gln)
ix) hydroxy side chains (Ser, Thr)
x) sulphor-containing side chains (Cys, Met), and
xi) amino acids being monoamino-dicarboxylic acids or monoamino-monocarboxylic-monoamidocarboxylic acids (Asp, Glu, Asn, Gln).

Non-conservative substitutions are any other substitutions. A non-conservative substitution leading to the formation of a functional homologue would for example i) differ substantially in hydrophobicity, for example a hydrophobic residue (Val, Ile, Leu, Phe or Met) substituted for a hydrophilic residue such as Arg, Lys, Trp or Asn, or a hydrophilic residue such as Thr, Ser, His, Gln, Asn, Lys, Asp, Glu or Trp substituted for a hydrophobic residue; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Functional homologues according to the present invention may comprise more than one such substitution, such as e.g. two amino acid substitutions, for example three or four amino acid substitutions, such as five or six amino acid substitutions, for example seven or eight amino acid substitutions, such as from 10 to 15 amino acid substitutions, for example from 15 to 25 amino acid substitution, such as from 25 to 30 amino acid substitutions, for example from 30 to 40 amino acid substitution, such as from 40 to 50 amino acid substitutions, for example from 50 to 75 amino acid substitution, such as from 75 to 100 amino acid substitutions, for example more than 100 amino acid substitutions. The addition or deletion of an amino acid may be an addition or deletion of from 2 to 5 amino acids, such as from 5 to 10 amino acids, for example from 10 to 20 amino acids, such as from 20 to 50 amino acids. However, additions or deletions of more than 50 amino acids, such as additions from 50 to 200 amino acids, are also comprised within the present invention. The polypeptides according to the present invention, including any variants and functional homologues thereof, may in one embodiment comprise more than 5 amino acid residues, such as more than 10 amino acid residues, for example more than 20 amino acid residues, such as more than 25 amino acid residues, for example more than 50 amino acid residues, such as more than 75 amino acid residues, for example more than 100 amino acid residues, such as more than 150 amino acid residues, for example more than 200 amino acid residues.

In a further embodiment the present invention relates to functional equivalents which comprise substituted amino acids having hydrophilic or hydropathic indices that are within +/−2.5, for example within +/−2.3, such as within +/−2.1, for example within +/−2.0, such as within +/−1.8, for example within +/−1.6, such as within +/−1.5, for example within +/−1.4, such as within +/−1.3 for example within +/−1.2, such as within +/−1.1, for example within +/−1.0, such as within +/−0.9, for example within +/−0.8, such as within +/−0.7, for example within +/−0.6, such as within +/−0.5, for example within +/−0.4, such as within +/−0.3, for example within +/−0.25, such as within +/−0.2 of the value of the amino acid it has substituted. The importance of the hydrophilic and hydropathic amino acid indices in conferring interactive biologic function on a protein is well understood in the art (Kyte & Doolittle, 1982 and Hopp, U.S. Pat. No. 4,554,101, each incorporated herein by reference).

The amino acid hydropathic index values as used herein are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) (Kyte & Doolittle, 1982).

The amino acid hydrophilicity values are: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+-0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4) (U.S. Pat. No. 4,554,101).

Substitution of amino acids can therefore in one embodiment be made based upon their hydrophobicity and hydrophilicity values and the relative similarity of the amino acid side-chain substituents, including charge, size, and the like. Exemplary amino acid substitutions which take several of the foregoing characteristics into consideration are well known to those of skill in the art and include: arginine and lysine; glutamate and aspartate; serine and threonine; glutamine and asparagine; and valine, leucine and isoleucine.

In addition to the polypeptide compounds described herein, sterically similar compounds may be formulated to mimic the key portions of the peptide structure and that such compounds may also be used in the same manner as the peptides of the invention. This may be achieved by techniques of modelling and chemical designing known to those of skill in the art. For example, esterification and other alkylations may be employed to modify the amino terminus of, e.g., a di-arginine peptide backbone, to mimic a tetra peptide structure. It will be understood that all such sterically similar constructs fall within the scope of the present invention.

Peptides with N-terminal alkylations and C-terminal esterifications are also encompassed within the present invention. Functional equivalents also comprise glycosylated and covalent or aggregative conjugates, including dimers or unrelated chemical moieties. Such functional equivalents are prepared by linkage of functionalities to groups which are found in fragment including at any one or both of the N- and C-termini, by means known in the art.

Functional equivalents may thus comprise fragments conjugated to aliphatic or acyl esters or amides of the carboxyl terminus, alkylamines or residues containing carboxyl side chains, e.g., conjugates to alkylamines at aspartic acid residues; O-acyl derivatives of hydroxyl group-containing residues and N-acyl derivatives of the amino terminal amino acid or amino-group containing residues, e.g. conjugates with Met-Leu-Phe. Derivatives of the acyl groups are selected from the group of alkyl-moieties (including C3 to C10 normal alkyl), thereby forming alkanoyl species, and carbocyclic or heterocyclic compounds, thereby forming aroyl species. The reactive groups preferably are bifunctional compounds known per se for use in cross-linking proteins to insoluble matrices through reactive side groups. However, functional equivalents may also encompass antibodies, antibody fragments, or any other molecular entity capable of mimicking the function (or structure) of the bivalent molecules of the present invention.

Homologues of nucleic acid sequences within the scope of the present invention are nucleic acid sequences, which encodes an RNA and/or a protein with similar biological function, and which is either
a) at least 50% identical, such as at least 60% identical, for example at least 70% identical, such as at least 75% identical, for example at least 80% identical, such as at least 85% identical, for example at least 90% identical, such as at least 95% identical
b) or able to hybridise to the complementary strand of said nucleic acid sequence under stringent conditions.

Stringent conditions as used herein shall denote stringency as normally applied in connection with Southern blotting and hybridisation as described e.g. by Southern E. M., 1975, J. Mol. Biol. 98:503-517. For such purposes it is routine practise to include steps of prehybridization and hybridization. Such steps are normally performed using solutions containing 6×SSPE, 5% Denhardt's, 0.5% SDS, 50% formamide, 100 ug/ml denaturated salmon testis DNA (incubation for 18 hrs at 42° C.), followed by washings with 2×SSC and 0.5% SDS (at room temperature and at 37° C.), and a washing with 0.1×SSC and 0.5% SDS (incubation at 68° C. for 30 min), as described by Sambrook et al., 1989, in “Molecular Cloning/A Laboratory Manual”, Cold Spring Harbor), which is incorporated herein by reference.

Homologous of nucleic acid sequences also encompass nucleic acid sequences which comprise additions and/or deletions. Such additions and/or deletions may be internal or at the end. Additions and/or deletions may be of 1-5 nucleotides, such as 5 to 10 nucleotides, for example 10 to 50 nucleotides, such as 50 to 100 nucleotides, for example at least 100 nucleotides.

Methods of alignment of sequences for comparison are well-known in the art. Various programs and alignment algorithms are described and present a detailed consideration of sequence alignment methods and homology calculations, such as VECTOR NTI. The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences will be.

The NCBI Basic Local Alignment Search Tool (BLAST) is available from several sources, including the National Center for Biotechnology Information (NBC', Bethesda, Md.) and on the Internet, for use in connection with the sequence analysis programs blastp, blastn, blastx, tblastn and tblastx. It can be accessed at http://www.ncbi.nlm.nih.gov/BLAST/.

Structural homologues of the disclosed bivalent molecules are typically characterised by possession of at least 80% sequence identity counted over the full length alignment with the disclosed amino acid sequence using the NCBI Basic Blast 2.0, gapped blastp with databases such as the nr or swissprot database. Alternatively, one may manually align the sequences and count the number of identical amino acids. This number divided by the total number of amino acids in your sequence multiplied by 100 results in the percent identity.

However, structural homologues within the scope of the present invention may also refer to similar chemical structures, such as organic chemical molecules and their derivatives.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70, at least 100, at least 150, at least, 200, at least 300, at least 400 or at least 500 contiguous residues. Such lengths are exemplary only, and it is understood that any fragment length supported by the sequences shown herein, in the tables, figures or Sequence Listing, may be used to describe a length over which percentage identity may be measured.

Percent identity may be measured over the length of an entire defined sequence, for example, as defined by a particular SEQ ID number, or may be measured over a shorter length, for example, over the length of a fragment taken from a larger, defined sequence, for instance, a fragment of at least 15, at least 20, at least 30, at least 40, at least 50, at least 70, at least 100, at least 150, at least, 200, at least 300, at least 400, at least 500, at least 750, at least 1000, at least 1250, or at least 1500 contiguous nucleotides. Such lengths are exemplary only, and it is understood that any fragment length supported by the sequences shown herein, in the tables, figures, or Sequence Listing, may be used to describe a length over which percentage identity may be measured.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), other nucleic acid analogue, or to any DNA-like or RNA-like material.

The term “target cell,” when used herein refers to a cell capable of being infected by a virus, preferably HIV. Preferably, the target cell is one or more human cells, and more preferably, human cells capable of being infected by a virus via a process, including membrane fusion as described elsewhere, and in particular viruses that make use of the type 1 membrane fusion mechanism belonging to the groups of Orthomyxoviridae, Paramyxoviridae, Retroviridae, Filoviridae and Coronaviridae.

The term “HIV” refers to Human Immunodeficiency Virus, and more preferably HIV-1 and HIV-2, and/or to any strain of HIV.

The term “tropism” or “tropic” according to the present invention is used to define the tissues or cells of a host which support growth of a particular virus.

The term “linker” when used herein, means a compound or a chemical moiety that may act as a molecular bridge to operably link two different molecules. Additionally the linker may be used to separate two different molecules or molecular entities. The linker may be peptides as in production of a recombinant fusion protein containing one or more copies of the bivalent HIV fusion inhibitor molecule of the present invention. Alternatively, the two different molecules may be linked to the linker in a step-wise manner (e.g., via chemical coupling). In general, there is no particular size or content limitations for the linker so long as it can fulfil its purpose as a molecular bridge, or a molecular separator long enough to introduce flexibility between the two parts of the bivalent molecules of the present invention. Linkers are known to those skilled in the art to include, but are not limited to, polymers of any sort and chemical chains, e.g. hydrocarbons, polypeptides, peptides, polyamides, carbohydrates, polynucleotides etc.

The term “treatment”, as used anywhere herein comprises any type of therapy, which aims at terminating, preventing, ameliorating and/or reducing the susceptibility to a clinical condition as described herein. In a preferred embodiment, the term treatment relates to prophylactic treatment, i.e. a therapy to reduce the susceptibility of a clinical condition, a disorder or condition as defined herein. Hence, the molecules of the invention may be used in the treatment or prevention of viral infection (such as HIV) and may be used in conjunction with other anti-viral molecules (for example, may be part of Highly Active Antiretroviral Therapy (HAART)). However, the molecules of the invention may also be used as an alternative to HAART, for example where it is clinically necessary to withdraw HAART.

Thus, “treatment,” “treating,” and the like, as used herein, refer to obtaining a desired pharmacologic and/or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse affect attributable to the disorder. That is, “treatment” includes (1) preventing the disorder from occurring or recurring in a subject, (2) inhibiting the disorder, such as arresting its development, (3) stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter, such as inflammation, pain, and/or immune deficiency.

The terms “prevent,” “preventing,” and “prevention”, as used herein, refer to a decrease in the occurrence of pathological cells in an animal. The prevention may be complete, e.g., the total absence of pathological cells in a subject. The prevention may also be partial, such that for example the occurrence of pathological cells in a subject is less than that which would have occurred without the present invention. Prevention also refers to reduced susceptibility to a clinical condition.

A “pharmaceutically acceptable carrier,” “pharmaceutically acceptable diluent,” or “pharmaceutically acceptable excipient”, or “pharmaceutically acceptable vehicle,” used interchangeably herein, refer to a non-toxic solid, semisolid or liquid filler, diluent, encapsulating material or formulation auxiliary of any conventional type. A pharmaceutically acceptable carrier is essentially non-toxic to recipients at the dosages and concentrations employed and is compatible with other ingredients of the formulation. For example, the carrier for a formulation containing polypeptides would not normally include oxidizing agents and other compounds that are known to be deleterious to polypeptides. Suitable carriers include, but are not limited to, water, dextrose, glycerol, saline, ethanol, and combinations thereof. The carrier can contain additional agents such as wetting or emulsifying agents, pH buffering agents, or adjuvants which enhance the effectiveness of the formulation. Adjuvants of the invention include, but are not limited to Freunds's, Montanide ISA Adjuvants &Isqb;Seppic, Paris, France&rsqb;, Ribi's Adjuvants (Ribi ImmunoChem Research, Inc., Hamilton, Mont.), I Hunter's TiterMax (CytRx Corp., Norcross, Ga.), Aluminum Salt Adjuvants (Alhydrogel—Superfos of Denmark/Accurate Chemical and Scientific Co., Westbury, N.Y.), Nitrocellulose-Adsorbed Protein, Encapsulated Antigens, and Gerbu Adjuvant (Gerbu Biotechnik GmbH, Gaiberg, Germany/C-C Biotech, Poway, Calif.). Topical carriers include liquid petroleum, isopropyl palmitate, polyethylene glycol, ethanol (95%), polyoxyethylene monolaurate (5%) in water, or sodium lauryl sulfate (5%) in water. Other materials such as anti-oxidants, humectants, viscosity stabilizers, and similar agents can be added as necessary. Percutaneous penetration enhancers such as Azone can also be included.

“Pharmaceutically acceptable salts” include the acid addition salts (formed with the free amino groups of the polypeptide) and which are formed with inorganic acids such as, for example, hydrochloric or phosphoric acids, or such organic acids as acetic, mandelic, oxalic, and tartaric. Salts formed with the free carboxyl groups can also be derived from inorganic bases such as, for example, sodium, potassium, ammonium, calcium, or ferric hydroxides, and such organic bases as isopropylamine, trimethylamine, 2-ethylamino ethanol, and histidine.

The term “unit dosage form” as used herein refers to physically discrete units suitable as unitary dosages for human and animal subjects, each unit containing a predetermined quantity of a composition, alone or in combination with other agents, calculated in an amount sufficient to produce the desired effect in association with a pharmaceutically acceptable diluent, carrier, or vehicle. The specifications for the unit dosage forms of the present invention depend on the particular composition or compositions employed and the effect to be achieved, as well as the pharmacodynamics associated with each composition in the host. The dose administered should be an “effective amount” or an amount necessary to achieve an “effective level” in the individual patient.

The term “fusion” according to the present invention comprises cell-cell fusion as well as virus-cell fusion. Cell-cell Fusion or Syncytia formation is a process by which the plasma membranes of two cells merge to form a single continuous double lipid membrane. This process does not happen spontaneously and is often mediate by the surface proteins of enveloped viruses such as the envelope proteins of retroviruses. Virus cell fusion is process by which an enveloped virus mediates merging of its lipid membrane with that of a target cell through interaction of the viral coat protein with a cellular receptor. The result of viral cell fusion process is entry of the viral core into the cytoplasm of a target cell, which is necessary for productive infection. The bivalent molecules of the present invention are in particular useful for inhibiting viruses that makes use of the type 1 envelope fusion mechanism, wherein these viruses belong to the main groups of Orthomyxoviridae, Paramyxoviridae, Retroviridae, Filoviridae and Coronaviridae.

The term “fusion inhibitor” or “entry inhibitor” according to the present invention encompass any molecule or molecular entity that are able to interfere with the binding, fusion and/or entry of an virus, in particular HIV, into a cell, essentially by blocking the fusion process as described here above.

The term “pre-fusion inhibitor according to the present invention encompass one or more of the bivalent molecules of the present invention, said molecules being able to bind to and inhibit the virus particle, in particular the HIV particle, before the virus contacts its target cells and hence before the fusion process starts.

The term “bivalent molecules” according to the present invention encompass any molecule that comprise at least two structural and/or functional distinct parts, wherein the at least two parts are being able to bind to and/or interact with one or at least two different other parts, e.g. other molecular entities. These different molecular entities may be present on the same molecule or on different molecules. These different molecular entities may further be present on different organisms, such as tow or more different virus particles.

The term “coiled coil” according to the present invention is a structural motif in proteins, in which two or more alpha-helices (most often 2-7 alpha-helices) are coiled together like the strands of a rope (dimers and trimers are the most common types). Many coiled coil type proteins are involved in important biological functions such as the regulation of gene expression e.g. transcription factors. Coiled coils often, but not always, contain a repeated pattern, hpphppp, of hydrophobic (h) and polar (p) amino-acid residues, referred to as a heptad repeat (see herein below). Folding a sequence with this repeating pattern into an alpha-helical secondary structure causes the hydrophobic residues to be presented as a ‘stripe’ that coils gently around the helix in left-handed fashion, forming an amphipathic structure. The most favourable way for two such helices to arrange themselves in a water-filled environment of is to wrap the hydrophobic strands against each other sandwiched between the hydrophilic amino acids. It is thus the burial of hydrophobic surfaces, which provides the thermodynamic driving force for the oligomerization. The packing in a coiled-coil interface is exceptionally tight. The α-helices may be parallel or anti-parallel, and usually adopt a left-handed super-coil. Although disfavored, a few right-handed coiled coils have also been observed in nature and in designed proteins.

The term “heptad repeat” as used herein refers to a structural motif found in some proteins, which contain a repeated stretch of seven amino acids with the following structure
a b c d e f g
H.P P H P P P

wherein “H” represents hydrophobic residues (non-polar) and “P” represents polar (and therefore hydrophilic) residues. The positions in the heptad repeat are usually labelled abcdefg, where a and d are the hydrophobic positions, often being occupied by isoleucine, leucine or valine, with almost complete van der Waals contact between the side chains of the a and d residues.

The term “triple-helix” or triple-helices” according to the present invention refer to structural motif found in some proteins, and has often been associated with collagen. The supramolecular structure of the triple-helix motif is characterized by a rod shaped appearance of parallel, anti-parallel or staggered helices that are able to self-associate in a variety of forms as well, and are able to bind to a wide variety of ligands. The distinctive amino acid features include the presence of glycine at every third position along the polypeptide chain and a high content of imino acids, including both proline and hydroxyproline. This results in a (Gly-X-Y)ₙ, repeating pattern, where Gly-Pro-Hyp is the most common triplet. However, the triple-helices according to the present invention, are not limited to triple-helices containing the (Gly-X-Y), repeating pattern, but may be any amino acid sequence capable of forming a triple-helix, or any other molecular entity capable of forming a triple-helix, or a triple-helix-like structure being a functional and/or structural homologue of the triple-helix motif.

The term “tag for purification” or “purification tag” according to the present invention relates to a stretch of amino acids, or other molecular entities, added to and/or integrated in the bivalent molecules of the invention, which enables the recovery of the labelled (tagged) bivalent molecules by its unique affinity. The tag for purification may be located at either end of the molecule to be purified, or the tag for purification may be located internally in the molecule to be purified. A tag for purification is according to the present invention not limited to a tag suitable only for purification, but may be any tag known to a person skilled in the art, such as, but not limited to, BCCP, Myc-tag (c-myc-tag), Calmodulin-tag, FLAG-tag, HA-tag, His-tag (Hexahistidine-tag, His6, 6H), Maltose binding protein-tag, Nus-tag, Glutathione-S-transferase-tag (GST-tag), Green fluorescent protein-tag (GFP-tag), Thioredoxin-tag, S-tag, Softag 1, Softag 3, Strep-tag, SBP-tag, biotin-tag, streptavidin-tag and V5-tag.

The term “ENV” according to the present invention refers to the viral envelope protein, in particular to the HIV envelope protein. The HIV envelope protein comprises the protein gp120 and the protein gp41. When HIV binds to the CD4-receptor, a conformational change occurs in the gp120 protein which results in the exposure of gp41. ENV is encoded by the gene env, which does not actually code for gp120 and gp41, but for a precursor to both, gp160. During HIV reproduction, the host cell's endogenous enzymes cleave gp160 into gp120 and gp41.

The terms “Pre-fusion state”, “intermediate-fusion state” and “post-fusion state” according to the present invention refers to the different free energy states in which the ENV protein may be present, on the way from before start of the fusion process to after the fusion process has been completed. The pre-fusion state refers to the meta-stable free energy state of the ENV protein before start of the fusion process. The post-fusion state refers to the stable free energy state of the ENV protein after completion of the fusion process. The intermediate-fusion state refers to any transition state or intermediate state between the pre-fusion state and the post-fusion state.

The term “gene therapy” according to present invention relates to the insertion of genes into cells and/or tissues with the aim for alleviating and/or preventing and/treating to treat a disease. The cell of insertion may be any cell or tissue of the individual. The disease to be treated with gene therapy may be any disease, including infections and diseases arising from infections, e.g. HIV infections. However, gene therapy as used herein also relates to the insertion of the gene in question into a single-cell organism, such as bacteria, protozoa, amoebae, viruses, moulds, yeast, fungus and the like, for stable endogenous production of the therapeutic agent, which then is used to alleviate, prevent or treat the disease in question. The bivalent molecules of the present invention may be expressed from any type of viral or non viral expression vector). The cells may be transduced both ex vivo and the cells reinstalled into the patient or in vivo (directly into the cells of interest). The cells to be transduced may originate from a cultured cell line or from another individual or another organism.

The term “microbicide” as used here in refers to any compound or substance whose purpose is to reduce the infectivity of microbes, such as viruses or bacteria. The microbicide may be any form of antibiotic, fungicide, bactericide, in particular a microbicide for any sexually transmitted diseases.

As used herein, “AIDS” refers to the symptomatic phase of HIV infection, and includes both Acquired Immune Deficiency Syndrome (commonly known as AIDS) and “ARC,” or AIDS Related Complex. The immunological and clinical manifestations of AIDS are well known in the art and include, for example, opportunistic infections and cancers resulting from immune deficiency.

### Structure of the Molecules of the Present Invention

One aspect of the present invention relates to bivalent molecules, and the structure of these molecules. Bivalent molecules are molecules that posses two or more distinct functional and/or structural characteristics. The bivalent molecules of the present invention are molecules wherein one part (the first part) of the molecule is able to mimic the function and/or structure of a mammalian receptor (and thus is a functional/structural homologue or functional/structural equivalent) as a virus binding molecule, while the other part (the second part) is able to bind to a viral protein. In a certain aspect, the one or two parts, or both parts, is an antibody or an antibody fragment capable of binding to a virus. The two parts of the molecule may be separated by a linker in order to introduce flexibility between the two parts. The two parts of the bivalent molecules may in one embodiment be directly coupled to each other, and thus not separated by a linker. However, in a preferred embodiment of the present invention, the two parts of the bivalent molecules are separated by a linker. In a further embodiment the two parts of the bivalent molecules are separated by two or more linkers.

### First Part of the Bivalent Molecule

The first part of the bivalent molecules of the present invention is able to mimic the function and/or structure of a mammalian receptor as virus binding molecule. In one embodiment of the present invention, the first part of the bivalent molecules is able to mimic the function and/or structure of a human receptor. In another embodiment, the first part of the bivalent molecules is able to mimic the function and/or structure of a human T-lymphocyte (T-cell) receptor. In a particular embodiment of the present invention, the first part of the bivalent molecules is able to mimic the function and/or structure of the human CD4 receptor present on the surface of CD4+ T-lymphocytes. In a preferred embodiment of the present invention, the first part of the bivalent molecules is able to mimic the function and/or structure of the extracellular, soluble part of the human CD4 protein (sCD4).

It is within the scope of the present invention that the first part of the bivalent molecules is a protein or a peptide. In one embodiment the first part of the bivalent molecules is the complete human CD4 receptor protein (CD4), or any part thereof or fragment thereof. In another embodiment of the present the first part of the bivalent molecules is the extracellular, soluble part of the human CD4 receptor protein (sCD4), or any part thereof or fragment thereof. Thus, in one embodiment the first part of the bivalent molecules of the present invention is a peptide with amino acid sequence selected from the group of amino acid sequences consisting of SEQ ID NOS: 9-10 or a fragment thereof, or a mimic thereof, or functional homologue thereof or any peptide with at least 80% identity to a peptide with amino acid sequence consisting of any of SEQ ID NOS: 9-10.

In a preferred embodiment of the present invention, the first part of the bivalent molecules is a peptide with amino acid sequence consisting of SEQ ID NO: 9 or a fragment thereof, or a mimic thereof, or functional homologue thereof or any peptide with at least 80% identity to a peptide with amino acid sequence consisting of SEQ ID NO: 9, such as at least 81% identity, for example at least 82% identity, at least 83% identity, such as at least 84% identity, for example at least 85% identity, at least 86% identity, such as at least 87% identity, for example at least 88% identity, at least 89% identity, such as at least 90% identity, for example at least 91% identity, at least 92% identity, such as at least 93% identity, for example at least 94% identity, at least 95% identity, such as at least 96% identity, for example at least 97% identity, at least 98% identity, such as at least 99% identity to a peptide with amino acid sequence consisting of SEQ ID NO: 9.

The first part of the bivalent molecules of the present invention may further comprise a tag for purification, such as the GST-tag or the hexahistidine tag (His6, 6H).

Thus, in another preferred embodiment of the present invention, the first part of the bivalent molecules is a peptide with amino acid sequence consisting of SEQ ID NO: 10 or a fragment thereof, or a mimic thereof, or functional homologue thereof or any peptide with at least 80% identity to a peptide with amino acid sequence consisting of SEQ ID NO: 10, such as at least 81% identity, for example at least 82% identity, at least 83% identity, such as at least 84% identity, for example at least 85% identity, at least 86% identity, such as at least 87% identity, for example at least 88% identity, at least 89% identity, such as at least 90% identity, for example at least 91% identity, at least 92% identity, such as at least 93% identity, for example at least 94% identity, at least 95% identity, such as at least 96% identity, for example at least 97% identity, at least 98% identity, such as at least 99% identity to a peptide with amino acid sequence consisting of SEQ ID NO: 10.

The first part of the bivalent molecules of the present invention may in other embodiments comprise other molecular entities than proteins or peptides that are able to mimic the function and/or structure of a mammalian receptor, in particular the human CD4 receptor. Thus the first part of the bivalent molecules of the present invention may comprise molecular entities related to and/or derivatives of N-phenyl-N′-piperidine-oxalamides. The structure of N-phenyl-N′-piperidine-oxalamide is shown here below as structure (A), where the substituent R(R-group) is a phenyl-substituent:

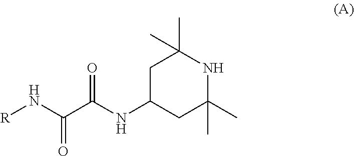

These may be, but certainly not limited to, the compounds known as NBD-556, NBD-557, DN-3186, JRC-II-75 and JRC-II-11:

| Compound | NBD-556 | NBD-557 | DN-3186 | JRC-II-75 | JRC-II-11 |
|---|---|---|---|---|---|
| R-group on | para- | para- | para-iodo- | para- | para-2- |
| structure | chloro- | bromo- | pehnyl | trifluor- | propyl- |
| (A) above | phenyl | phenyl | | methyl- | phenyl |
| | | | | phenyl | |

In one embodiment the first part of the bivalent molecules of the present invention is the compound NBD-556. In another embodiment of the present invention the first part of the bivalent molecules is the compound NBD-557. In another embodiment of the present invention the first part of the bivalent molecules is the compound DN-3186. In further embodiment of the present invention the first part of the bivalent molecules is the compound JRC-II-75. In an even further embodiment of the present invention the first part of the bivalent molecules is the compound NBD-557. However, the first part of the bivalent molecule may not be limited to the above listed compounds. Therefore, the first part of the bivalent molecules of the present invention may in certain embodiments be any functional and/or structural analogues to N-phenyl-N′-piperidine-oxalamides and derivatives thereof.

It is also within the scope of the present invention that the first part of the bivalent molecule may comprise one or more antibodies, and/or antibody fragments, e.g. scFv fragments, capable of binding to a virus, or to a viral antigen. In another embodiment, the one or more antibodies are capable of binding to an ENV protein of a virus. In a particular embodiment, the one or more antibodies are capable of binding to the ENV protein of a HIV virus. In a further particular embodiment, the one or more antibodies are capable of binding to the gp120 and/or the gp41 protein of a HIV virus. The one or more antibodies may in separate embodiments be monoclonal antibodies, polyclonal antibodies or a combination of both monoclonal and polyclonal antibodies.

### Second Part of the Bivalent Molecule

The second part of the bivalent molecules of the present invention comprises one or more peptides that are able bind to a protein from a virus, i.e. a viral protein. It is within the scope of the present invention that this viral protein is a protein from Human Immunodeficiency Virus (HIV) or any virus belonging to the groups of Orthomyxoviridae, Paramyxoviridae, Retroviridae, Filoviridae and Coronaviridae. In one embodiment the second part of the bivalent molecules is any peptide capable of forming a coiled coil. In another embodiment the second part of the bivalent molecules is any peptide comprising the heptad repeat structural motif. In a further embodiment the second part of the bivalent molecules is any peptide capable of forming a triple-helix. Further, the second part of the bivalent molecules may comprise a tag for purification, such as the GST-tag or the hexahistidine tag (His6, 6H).

In one embodiment of the present invention the second part of the bivalent molecules comprises one or more peptides selected from the group of peptides with amino acid sequences consisting of SEQ ID NOS: 11-18, 20-204 or any part thereof or fragment thereof, or mimic thereof, or functional homologue thereof, or an amino acid sequence at least 80% identical to any of SEQ ID NOS: 11-18, 20-204, such as at least 81% identity, for example at least 82% identity, at least 83% identity, such as at least 84% identity, for example at least 85% identity, at least 86% identity, such as at least 87% identity, for example at least 88% identity, at least 89% identity, such as at least 90% identity, for example at least 91% identity, at least 92% identity, such as at least 93% identity, for example at least 94% identity, at least 95% identity, such as at least 96% identity, for example at least 97% identity, at least 98% identity, such as at least 99% identity to a peptide with amino acid sequence consisting of any of SEQ ID NOS: 11-18, 20-204.

Thus in one embodiment of the present invention the second part of the bivalent molecules comprises one or more HIV-1 derived peptides selected from the group of peptides with amino acid sequences consisting of SEQ ID NOS: 20-40: or any part thereof or fragment thereof, or mimic thereof, or functional homologue thereof, or an amino acid sequence at least 80% identical to any of SEQ ID NOS: 20-40.

In another embodiment of the present invention the second part of the bivalent molecules comprises one or more HIV-1 peptides selected from the group of peptides with amino acid sequences consisting of SEQ ID NOS: 41-65: or any part thereof or fragment thereof, or mimic thereof, or functional homologue thereof, or an amino acid sequence at least 80% identical to any of SEQ ID NOS: 41-65.

In another embodiment of the present invention the second part of the bivalent molecules comprises one or more HIV-2 derived peptides selected from the group of peptides with amino acid sequences consisting of SEQ ID NOS: 66-75: or any part thereof or fragment thereof, or mimic thereof, or functional homologue thereof, or an amino acid sequence at least 80% identical to any of SEQ ID NOS: 66-75.

In yet another embodiment of the present invention the second part of the bivalent molecules comprises one or more HIV-2 derived peptides selected from the group of peptides with amino acid sequences consisting of SEQ ID NOS: 76-83: or any part thereof or fragment thereof, or mimic thereof, or functional homologue thereof, or an amino acid sequence at least 80% identical to any of SEQ ID NOS: 76-83.

In a further embodiment of the present invention the second part of the bivalent molecules comprises one or more SIV derived peptides selected from the group of peptides with amino acid sequences consisting of SEQ ID NOS: 84-115: or any part thereof or fragment thereof, or mimic thereof, or functional homologue thereof, or an amino acid sequence at least 80% identical to any of SEQ ID NOS: 84-115.

In yet a further embodiment of the present invention the second part of the bivalent molecules comprises one or more SIV derived peptides selected from the group of peptides with amino acid sequences consisting of SEQ ID NOS: 116-145: or any part thereof or fragment thereof, or mimic thereof, or functional homologue thereof, or an amino acid sequence at least 80% identical to any of SEQ ID NOS: 116-145.

In an even further embodiment of the present invention the second part of the bivalent molecules comprises one or more SIV derived peptides selected from the group of peptides with amino acid sequences consisting of SEQ ID NOS: 146-171: or any part thereof or fragment thereof, or mimic thereof, or functional homologue thereof, or an amino acid sequence at least 80% identical to any of SEQ ID NOS: 146-171.

In another embodiment of the present invention the second part of the bivalent molecules comprises one or more peptides selected from the group of peptides with amino acid sequences consisting of SEQ ID NOS: 172-175: or any part thereof or fragment thereof, or mimic thereof, or functional homologue thereof, or an amino acid sequence at least 80% identical to any of SEQ ID NOS: 172-175.

In a further embodiment of the present invention the second part of the bivalent molecules comprises one or more influenza derived peptides selected from the group of peptides with amino acid sequences consisting of SEQ ID NOS: 176-204: or any part thereof or fragment thereof, or mimic thereof, or functional homologue thereof, or an amino acid sequence at least 80% identical to any of SEQ ID NOS: 176-204.

However, in a particular embodiment of the present invention the second part of the bivalent molecules comprise one or more peptides selected from the group of peptides with amino acid sequences consisting of SEQ ID NOS: 11-18: or any part thereof or fragment thereof, or mimic thereof, or functional homologue thereof, or an amino acid sequence at least 80% identical to any of SEQ ID NOS: 11-18, such as at least 81% identity, for example at least 82% identity, at least 83% identity, such as at least 84% identity, for example at least 85% identity, at least 86% identity, such as at least 87% identity, for example at least 88% identity, at least 89% identity, such as at least 90% identity, for example at least 91% identity, at least 92% identity, such as at least 93% identity, for example at least 94% identity, at least 95% identity, such as at least 96% identity, for example at least 97% identity, at least 98% identity, such as at least 99% identity to a peptide with amino acid sequence consisting of any of SEQ ID NOS: 11-18.

In another particular embodiment of the present invention the second part of the bivalent molecules comprises one or more peptides selected from the group of peptides with amino acid sequences consisting of SEQ ID NOS: 12-15: or any part thereof or fragment thereof, or mimic thereof, or functional homologue thereof, or an amino acid sequence at least 80% identical to any of SEQ ID NOS: 12-15.

In a further particular embodiment of the present invention the second part of the bivalent molecules comprises one or more peptides selected from the group of peptides with amino acid sequences consisting of SEQ ID NOS: 11, 16-18 or any part thereof or fragment thereof, or mimic thereof, or functional homologue thereof, or an amino acid sequence at least 80% identical to any of SEQ ID NOS: 11, 16-18.

In a preferred embodiment of the present invention the second part of the bivalent molecules comprises one or more peptides with amino acid sequences consisting of SEQ ID NO: 11, or any part thereof or fragment thereof, or mimic thereof, or functional homologue thereof, or an amino acid sequence at least 80% identical to any of SEQ ID NO: 11.

In another preferred embodiment of the present invention the second part of the bivalent molecules comprises one or more peptides with amino acid sequences consisting of SEQ ID NO: 16, or any part thereof or fragment thereof, or mimic thereof, or functional homologue thereof, or an amino acid sequence at least 80% identical to any of SEQ ID NO: 16.

In yet another preferred embodiment of the present invention the second part of the bivalent molecules comprises one or more peptides with amino acid sequences consisting of SEQ ID NO: 17, or any part thereof or fragment thereof, or mimic thereof, or functional homologue thereof, or an amino acid sequence at least 80% identical to any of SEQ ID NO: 17.

In another preferred embodiment of the present invention the second part of the bivalent molecules comprises one or more peptides with amino acid sequences consisting of SEQ ID NO: 18, or any part thereof or fragment thereof, or mimic thereof, or functional homologue thereof, or an amino acid sequence at least 80% identical to any of SEQ ID NO: 18.

It is also within the scope of the present invention that the second part of the bivalent molecule may comprise one or more antibodies, and/or antibody fragments, e.g. scFv fragments, capable of binding to a virus, or to a viral antigen. In another embodiment, the one or more antibodies are capable of binding to an ENV protein of a virus. In a particular embodiment, the one or more antibodies are capable of binding to the ENV protein of a HIV virus. In a further particular embodiment, the one or more antibodies are capable of binding to the gp120 and/or the gp41 protein of a HIV virus.

### Linker

The first part of the bivalent molecules may be directly joined to the second part of the bivalent molecules, and thus not separated by a linker molecule. However, it falls within the scope of the present invention that the first part of the bivalent molecules as defined herein above and the second part of the bivalent molecules as defined herein above, are separated, and hence joined by one or more linker molecules. The linker molecule serves to add flexibility to the bivalent molecules of the present invention, as well as ensuring appropriate separation between the two parts of the bivalent molecules. The linker may be any molecular entity capable of joining two or more other molecules. The linker of the bivalent molecules of the present invention may thus in one embodiment be a polymer, such as a hydrocarbon, for example a hydrocarbon selected from the group of hydrocarbons consisting of alkanes, alkenes and alkynes. In another embodiment of the present invention the one or more linkers are polymers selected from the group of different types of polymers consisting of hydrocarbons, polyamides, polypeptides, polysacchrarides and polynucleotides. The one or more linkers may be of the same type of polymer, or the one or more linker may be of different types of polymers. The linker of the present invention may further comprise a tag for purification, such as the GST-tag or the hexahistidine tag (His6, 6H).

In a particular embodiment of the present invention, the linker is polypeptide. The linker may thus be a polypeptide of any length suitable of performing the action of joining the first and the second part of the bivalent molecules of the present invention. Hence, the linker polypeptide may be a peptide comprising at least 2 consecutive amino acid residues, such as at least 3 consecutive amino acid residues, for example at least 4 consecutive amino acid residues, such as at least 5 consecutive amino acid residues, at least 6 consecutive amino acid residues, for example at least 7 consecutive amino acid residues, such as at least 8 consecutive amino acid residues, at least 9 consecutive amino acid residues, such as at least 10 consecutive amino acid residues, at least 11 consecutive amino acid residues, such as at least 12 consecutive amino acid residues, for example at least 13 consecutive amino acid residues, such as at least 14 consecutive amino acid residues, at least 15 consecutive amino acid residues, for example at least 16 consecutive amino acid residues, such as at least 17 consecutive amino acid residues, at least 18 consecutive amino acid residues, such as at least 19 consecutive amino acid residues, at least 20 consecutive amino acid residues, such as at least 21 consecutive amino acid residues, for example at least 22 consecutive amino acid residues, such as at least 23 consecutive amino acid residues, at least 24 consecutive amino acid residues, for example at least 25 consecutive amino acid residues, such as at least 26 consecutive amino acid residues, at least 27 consecutive amino acid residues, such as at least 28 consecutive amino acid residues, at least 29 consecutive amino acid residues, such as at least 30 consecutive amino acid residues, for example at least 31 consecutive amino acid residues, such as at least 32 consecutive amino acid residues, at least 33 consecutive amino acid residues, for example at least 34 consecutive amino acid residues, such as at least 35 consecutive amino acid residues, at least 36 consecutive amino acid residues, such as at least 37 consecutive amino acid residues, such as at least 38 consecutive amino acid residues, for example at least 39 consecutive amino acid residues, such as at least 40 consecutive amino acid residues, at least 41 consecutive amino acid residues, for example at least 42 consecutive amino acid residues, such as at least 43 consecutive amino acid residues, at least 44 consecutive amino acid residues, such as at least 45 consecutive amino acid residues, at least 46 consecutive amino acid residues, such as at least 47 consecutive amino acid residues, for example at least 48 consecutive amino acid residues, such as at least 49 consecutive amino acid residues, such as at least 50 consecutive amino acid residues.

Thus the linker peptide of the present invention may comprise at least 2-5 consecutive amino acid residues, at least 6-10 consecutive amino acid residues, such as at least 11-15 consecutive amino acid residues, for example at least 16-20 consecutive amino acid residues, such as at least 21-25 consecutive amino acid residues, at least 26-30 consecutive amino acid residues, for example at least 31-35 consecutive amino acid residues, such as at least 36-40 consecutive amino acid residues, at least 41-45 consecutive amino acid residues, such as at least 46-50 consecutive amino acid residues.

However, in certain embodiments of the present invention the linker polypeptide may comprise at least 55 consecutive amino acid residues, at least 60 consecutive amino acid residues, such as at least 65 consecutive amino acid residues, for example at least 70 consecutive amino acid residues, such as at least 75 consecutive amino acid residues, at least 80 consecutive amino acid residues, for example at least 85 consecutive amino acid residues, such as at least 90 consecutive amino acid residues, for example at least 100 consecutive amino acid residues.

The linker peptide of the present invention comprise amino acids with properties suitable for being a flexible linker, while at the same time comprising a mixture of hydrophobic and hydrophilic amino acids, in a way that the linker will be soluble under different hydrophilic and hydrophobic conditions. The linker peptide of the present invention may comprise any hydrophobic and any hydrophilic amino acid, and it is within the scope of the present invention that the linker comprises hydrophilic and hydrophobic amino acid residues in a ratio of at least 1:1, such as at least 1:1.1, for example at least 1:1.2, such as at least 1:1.3, at least 1:1.4, such as at least 1:1.5 for example at least 1:1.6, such as at least 1:1.7, at least 1:1.8, such as at least 1:1.9, for example at least 1:2, such as at least 1:2.1, at least 1:2.2, such as at least 1:2.3, for example at least 1:2.4, such as at least 1:2.5, at least 1:2.6, such as at least 1:2.7, for example at least 1:2.8, such as at least 1:2.9, at least 1:3, such as at least 1:3.1, for example at least 1:3.2, such as at least 1:3.3, at least 1:3.4, such as at least 1:3.5, for example at least 1:3.6, such as at least 1:3.7, at least 1:3.8, such as at least 1:3.9, for example at least 1:4, such as at least 1:4.1, at least 1:4.2, such as at least 1:4.3, for example at least 1:4.4, such as at least 1:4.5, at least 1:4.6, such as at least 1:4.7, for example at least 1:4.8, such as at least 1:4.9, for example at least 1:5 hydrophilic to hydrophopic amino acid residues.

However, the linker peptide of the present invention may comprise hydrophobic and hydrophilic amino acid residues in a ratio of at least 1:1, such as at least 1:1.1, for example at least 1:1.2, such as at least 1:1.3, at least 1:1.4, such as at least 1:1.5 for example at least 1:1.6, such as at least 1:1.7, at least 1:1.8, such as at least 1:1.9, for example at least 1:2, such as at least 1:2.1, at least 1:2.2, such as at least 1:2.3, for example at least 1:2.4, such as at least 1:2.5, at least 1:2.6, such as at least 1:2.7, for example at least 1:2.8, such as at least 1:2.9, at least 1:3, such as at least 1:3.1, for example at least 1:3.2, such as at least 1:3.3, at least 1:3.4, such as at least 1:3.5, for example at least 1:3.6, such as at least 1:3.7, at least 1:3.8, such as at least 1:3.9, for example at least 1:4, such as at least 1:4.1, at least 1:4.2, such as at least 1:4.3, for example at least 1:4.4, such as at least 1:4.5, at least 1:4.6, such as at least 1:4.7, for example at least 1:4.8, such as at least 1:4.9, for example at least 1:5 hydrophobic to hydrophilic amino acid residues.

In a particular embodiment of the present invention, the linker comprises the amino acids serine (Ser, S) and glycine (Gly, G). The mixture of these two amino acids will provide both flexibility, because of relative small size of these amino acids, and an optimal hydrophilicity owing to the nature of the hydroxylic side chain of serine. Thus, according to the present invention, the linker may in one embodiment comprise at least 20% glycine residues, such as at least 25% glycine residues, for example at least 30% glycine residues, at least 35% glycine residues, such as at least 40% glycine residues, for example at least 45% glycine residues, at least 50% glycine residues, such as at least 55% glycine residues, for example at least 60% glycine residues, at least 65% glycine residues, such as at least 70% glycine residues, for example at least 75% glycine residues, at least 80% glycine residues, such as at least 85% glycine residues, for example at least 90% glycine residues, at least 95% glycine residues, such as at least 100% glycine residues.

However, the linker may in another embodiment comprise at least 20% serine residues, such as at least 25% serine residues, for example at least 30% serine residues, at least 35% serine residues, such as at least 40% serine residues, for example at least 45% serine residues, at least 50% serine residues, such as at least 55% serine residues, for example at least 60% serine residues, at least 65% serine residues, such as at least 70% serine residues, for example at least 75% serine residues, at least 80% serine residues, such as at least 85% serine residues, for example at least 90% serine residues, at least 95% serine residues, such as at least 100% serine residues.

In a preferred embodiment of the present invention, the linker comprise one or more peptides with amino acid sequences consisting of SEQ ID NO: 19, or any part thereof or fragment thereof, or mimic thereof, or functional homologue thereof, or an amino acid sequence at least 80% identical SEQ ID NO: 19, such as at least 81% identity, for example at least 82% identity, at least 83% identity, such as at least 84% identity, for example at least 85% identity, at least 86% identity, such as at least 87% identity, for example at least 88% identity, at least 89% identity, such as at least 90% identity, for example at least 91% identity, at least 92% identity, such as at least 93% identity, for example at least 94% identity, at least 95% identity, such as at least 96% identity, for example at least 97% identity, at least 98% identity, such as at least 99% identity to a peptide with amino acid sequence consisting of any of SEQ ID NO: 19.

In another preferred embodiment of the present invention, the linker comprise one or more peptides with amino acid sequences consisting of SEQ ID NO: 19, or any part thereof or fragment thereof, or mimic thereof, or functional homologue thereof, or an amino acid sequence at least 80% identical SEQ ID NO: 19

### The Bivalent Molecule

It is with in the scope if the present invention that any first part of the bivalent molecules as described above may be combined with any second part of the bivalent molecule as described above, and the first part and the second part of the bivalent molecules may be separated, and hence joined by any one or more linkers as described above. Thus the present invention pertains in one aspect to bivalent molecules that are molecules that comprise:
i) any first part as defined herein above, and
ii) optionally any one or more linkers as defined herein above, and
iii) any second part as defined herein above

It is preferred that the first part of the bivalent molecules is located N-terminally (N′) relative to the second part of the bivalent molecules, which comprises one or more. However, it is also within the scope of the present invention that the first part of the bivalent molecules is located C-terminally (C′) relative to the second part of the bivalent molecules, which comprise one or more peptides. In either case it is preferred that the first part and second part is separated, and hence joined by one or more linkers.

Thus the relative orientation of the different parts of the bivalent molecules comprise in one embodiment the following structures:

First Part-Linker-N′-Second Part Peptide-C′ and/or

### N′-Second Part Peptide-C′-Linker-First Part

In another embodiment of the present invention, when the first part of the bivalent molecules also is a peptide, the relative orientation of the different parts of the bivalent molecules comprise the following structures:

N′-First Part Peptide-C′-Linker-N′-Second Part Peptide and/or
N′-First Part Peptide-C′-Linker-C″-Second Part Peptide-N′ and/or
C′-First Part Peptide-N′-Linker-N′-Second Part Peptide-C′ and/or

### C′-First Part Peptide-N′-Linker-C′-Second Part Peptide-N′

In a particular embodiment of the present invention, the linker is also a peptide, and hence the orientation of the different parts of the bivalent molecules comprises the following structures:

N′-First Part Peptide-Linker Peptide-Second Part Peptide-C′ and/or

### C′-First Part Peptide-Linker Peptide-Second Part Peptide-N′

In a preferred embodiment of the present invention the orientation of the different parts of the bivalent molecules is of the following structure:

### N′-First Part Peptide-Linker Peptide-Second Part Peptide-C′

It is also within the scope of the present invention that the bivalent molecules comprise more than one first part as described above and/or optionally more than one linker as described above and/or more than one second part as described above. As described elsewhere herein, any first part may be combined with any other first part (or first parts), and any second part may be combined with any other second part (or second parts), wherein said first part (or first parts) and second part (or second parts) may be separated by any linker combined with any other linker (or linkers).

Thus, in one embodiment of the present invention the bivalent molecules comprise one or more polypeptides selected from the group of polypeptides with amino acid sequences consisting of SEQ ID NOS: 1-8: or any part thereof or fragment thereof, or mimic thereof, or functional homologue thereof, or an amino acid sequence at least 80% identical to any of SEQ ID NOS: 1-8, such as at least 81% identity, for example at least 82% identity, at least 83% identity, such as at least 84% identity, for example at least 85% identity, at least 86% identity, such as at least 87% identity, for example at least 88% identity, at least 89% identity, such as at least 90% identity, for example at least 91% identity, at least 92% identity, such as at least 93% identity, for example at least 94% identity, at least 95% identity, such as at least 96% identity, for example at least 97% identity, at least 98% identity, such as at least 99% identity to a peptide with amino acid sequence consisting of any of SEQ ID NOS: 1-8.

In another embodiment of the present invention the bivalent molecules comprise one or more polypeptides selected from the group of polypeptides with amino acid sequences consisting of SEQ ID NOS: 2-5: or any part thereof or fragment thereof, or mimic thereof, or functional homologue thereof, or an amino acid sequence at least 80% identical to any of SEQ ID NOS: 2-5.

In a further embodiment of the present invention bivalent molecules comprise one or more polypeptides selected from the group of polypeptides with amino acid sequences consisting of SEQ ID NOS: 1, 6-8 or any part thereof or fragment thereof, or mimic thereof, or functional homologue thereof, or an amino acid sequence at least 80% identical to any of SEQ ID NOS: 1, 6-8.

In a preferred embodiment of the present invention the bivalent molecules comprise one or more polypeptides with amino acid sequences consisting of SEQ ID NO: 1, or any part thereof or fragment thereof, or mimic thereof, or functional homologue thereof, or an amino acid sequence at least 80% identical to any of SEQ ID NO: 1.

SEQ ID NO: 1
MNRGVPFRHLLLVLQLALLPAATQGKKVVLGKKGDTVELTCTASQKKSIQ
FHWKNSNQIKILGNQGSFLTKGPSKLNDRADSRRSLWDQGNFPLIIKNLK
IEDSDTYICEVEDQKEEVQLLVFGLTANSDTHLLQGQSLTLTLESPPGSS
PSVQCRSPRGKNIQGGKTLSVSQLELQDSGTWTCTVLQNQKKVEFKIDIV
VLAFQKASSIVYKKEGEQVEFSFPLAFTVEKLTGSGELWWQAERASSSKS
WITFDLKNKEVSVKRVTQDPKLQMGKKLPLHLTLPQALPQYAGSGNLTLA
LEAKTGKLHQEVNLVVMRATQLQKNLTCEVWGPTSPKLMLSLKLENKEAK
VSKREKAVWVLNPEAGMWQCLLSDSGQVLLESNIKVLPTWSTPVSSGGSG
GSGGSGGSGGSSGEWDREINNYTSLIHSLIEESQNQQEKNEQELLELDKW
ASLWNWF

In another preferred embodiment of the present invention the bivalent molecules comprise one or more polypeptides with amino acid sequences consisting of SEQ ID NO: 6, or any part thereof or fragment thereof, or mimic thereof, or functional homologue thereof, or an amino acid sequence at least 80% identical to any of SEQ ID NO: 6.

SEQ ID NO: 6
MNRGVPFRHLLLVLQLALLPAATQGKKVVLGKKGDTVELTCTASQKKSIQ
FHWKNSNQIKILGNQGSFLTKGPSKLNDRADSRRSLWDQGNFPLIIKNLK
IEDSDTYICEVEDQKEEVQLLVFGLTANSDTHLLQGQSLTLTLESPPGSS
PSVQCRSPRGKNIQGGKTLSVSQLELQDSGTWTCTVLQNQKKVEFKIDIV
VLAFQKASSIVYKKEGEQVEFSFPLAFTVEKLTGSGELWWQAERASSSKS
WITFDLKNKEVSVKRVTQDPKLQMGKKLPLHLTLPQALPQYAGSGNLTLA
LEAKTGKLHQEVNLVVMRATQLQKNLTCEVWGPTSPKLMLSLKLENKEAK
VSKREKAVWVLNPEAGMWQCLLSDSGQVLLESNIKVLPTWSTPVSSGGSG
GSGGSGGSGGSSGTTAVPWNASWSNKSLEQIWNHTT

In yet another preferred embodiment of the present invention the bivalent molecules comprise one or more polypeptides with amino acid sequences consisting of SEQ ID NO: 7, or any part thereof or fragment thereof, or mimic thereof, or functional homologue thereof, or an amino acid sequence at least 80% identical to any of SEQ ID NO: 7.

SEQ ID NO: 7
MNRGVPFRHLLLVLQLALLPAATQGKKVHHHHHHKVVLGKKGDTVELTCT
ASQKKSIQFHWKNSNQIKILGNQGSFLTKGPSKLNDRADSRRSLWDQGNF
PLIIKNLKIEDSDTYICEVEDQKEEVQLLVFGLTANSDTHLLQGQSLTLT
LESPPGSSPSVQCRSPRGKNIQGGKTLSVSQLELQDSGTWTCTVLQNQKK
VEFKIDIVVLAFQKASSIVYKKEGEQVEFSFPLAFTVEKLTGSGELWWQA
ERASSSKSWITFDLKNKEVSVKRVTQDPKLQMGKKLPLHLTLPQALPQYA
GSGNLTLALEAKTGKLHQEVNLVVMRATQLQKNLTCEVWGPTSPKLMLSL
KLENKEAKVSKREKAVWVLNPEAGMWQCLLSDSGQVLLESNIKVLPTWST
PVSSGGSGGSGGSGGSGGSSGTTAVPWNASWSNKSLEQIWNHTT

In another preferred embodiment of the present invention the bivalent molecules comprise one or more peptides with amino acid sequences consisting of SEQ ID NO: 8, or any part thereof or fragment thereof, or mimic thereof, or functional homologue thereof, or an amino acid sequence at least 80% identical to any of SEQ ID NO: 8.

SEQ ID NO 8
MNRGVPFRHLLLVLQLALLPAATQGKKVHHHHHHKVVLGKKGDTVELTCT
ASQKKSIQFHWKNSNQIKILGNQGSFLTKGPSKLNDRADSRRSLWDQGNF
PLIIKNLKIEDSDTYICEVEDQKEEVQLLVFGLTANSDTHLLQGQSLTLT
LESPPGSSPSVQCRSPRGKNIQGGKTLSVSQLELQDSGTWTCTVLQNQKK
VEFKIDIVVLAFQKASSIVYKKEGEQVEFSFPLAFTVEKLTGSGELWWQA
ERASSSKSWITFDLKNKEVSVKRVTQDPKLQMGKKLPLHLTLPQALPQYA
GSGNLTLALEAKTGKLHQEVNLVVMRATQLQKNLTCEVWGPTSPKLMLSL
KLENKEAKVSKREKAVWVLNPEAGMWQCLLSDSGQVLLESNIKVLPTWST
PVSSGGSGGSGGSGGSGGSSGEWDREINNYTSLIHSLIEESQNQQEKNEQ
ELLELDKWASLWNWF

It is within the scope of the present invention that the bivalent molecules are monomers and/or dimers and/or trimers. The dimers and/or trimers of the bivalent molecules of the present invention may be homodimers and/or heterodimers and/or homotrimers and/or heterotrimers. However, the bivalent molecules of the present invention may in certain embodiments also comprise polymers of more than two (dimers) or three (trimers) bivalent molecules. Thus, polymers of the bivalent molecules of the present invention may in certain embodiments comprise at least 4 bivalent molecules, such as at least 5, for example at least 6, at least 7, such as at least 8, for example at least 9, such as least 10 bivalent molecules.

In another embodiment of the present invention, polymers of the bivalent molecules of the present invention may comprise at least 12 bivalent molecules, such as at least 14, for example at least 16, at least 18, such as at least 20, for example at least 22, such as least 25 bivalent molecules.

In a further embodiment of the present invention, polymers of the bivalent molecules of the present invention may comprise at least 30 bivalent molecules, such as at least 35, for example at least 40, at least 50, such as at least 75, for example at least 100, such as least 200 bivalent molecules.

It is also within the scope of the present invention that the polymers of the bivalent molecules of the invention may be homo-polymers or hetero-polymers.

### Function of the Bivalent Molecules of the Present Invention

Another aspect of the present invention pertains to the function of the bivalent molecules of the present invention, namely the function as virus entry and/or fusion inhibitors, and in particular HIV entry/fusion inhibitor. The bivalent molecules of the present invention define, as described herein above, a new class of entry/fusion inhibitors, herein termed pre-fusion inhibitors, because the bivalent molecules of the present invention are able to neutralize the virus particle, and render it harmless, even before the fusion process (entry process) has started. Viral envelopes mediate fusion by undergoing several sequential conformational changes. The envelope protein (ENV) is kinetically arrested in a meta-stable conformation upon synthesis in the producer cells. It is this meta-stable protein that finds its way into virions. In other words, the envelope protein on the surface of the viral particles is not in its thermodynamically most stable conformation. This is necessary, since fusion between the cellular and viral membranes involves overcoming a large activation-energy barrier. The events that lead to membrane fusion benefit from the latent energy stored in the envelope protein. This energy is released when the ENV protein undergoes conformational changes. The release of this latent energy involves several stepwise conformational changes, the most important of which is binding to the target cell receptor and formation and folding of the extended triple helix. The bivalent molecules of the present invention work by lowering the activation energies of at least two of the conformational these changes, and thus stabilizing the intermediates/transition states. The first (“Receptor binding”, which is the conformational change that occurs when ENV binds to the CD4 receptor protein) is through binding of the first part of the bivalent molecules, that mimics receptor binding, to the ENV, and the second (“Triple-helix formation”) is by stabilizing the coiled coil structures that are formed in the gp41 protein (in the case of HIV) during fusion, through interaction of second part of the bivalent molecules with the alpha-helices of this protein. One other consequence of the large difference between the free energy of the pre-fusion conformation and the post-fusion conformation in the envelope protein is that there is no equilibrium between the two forms: Once the conformational changes occur, the post-fusion form of the ENV protein can never go back to its meta-stable conformation. This means that the bivalent molecules of the present invention triggers the envelope proteins on the viral surface to undergo the conformational changes towards the thermodynamically stable form of the protein (post-fusion conformation), while not in the vicinity of the target cell membrane, the stored energy that was meant for mediating membrane fusion is thus wasted and the envelope protein is neutralized as far as fusion activity is concerned, and rendered harmless as a result of the effect of the bivalent molecules of the present invention.

The bivalent molecules of the present invention are particularly effective against viruses that mediate fusion via the type 1 envelope fusion mechanism belonging to the groups of Orthomyxoviridae, Paramyxoviridae, Retroviridae, Filoviridae and Coronaviridae. The bivalent molecules of the present invention are effective to a wide variety of viruses, such as HTLV-1, HTLV-2, HERV, BLV, ELV, FeLV, PuLV, O/CLV, visna/maedi, PrLV, HIV-1, HIV-2, SIV, MLV, JSRV, FeLV A, Influenza HA, and ebola. It is however within the scope of the present invention that the bivalent molecules of the present invention are effective against HTLV-1, HTLV-2, HERV, HIV-1, HIV-2, SIV, MLV, BLV, JSRV and FeLV A. Even so, it is within the scope of the present invention that the bivalent molecules of the present invention are effective HIV-1, HIV-2 and SIV, and in particular HIV-1 and HIV-2.

Thus, the bivalent molecules of the present invention are able to inhibit virus particles, and in particular HIV virus particles. It is within the scope of the present invention that the bivalent molecules are able to inhibit the HIV particles as measured by different assays suitable for detecting and quantifying the spreading of the HIV particles, three of which are described herein below.

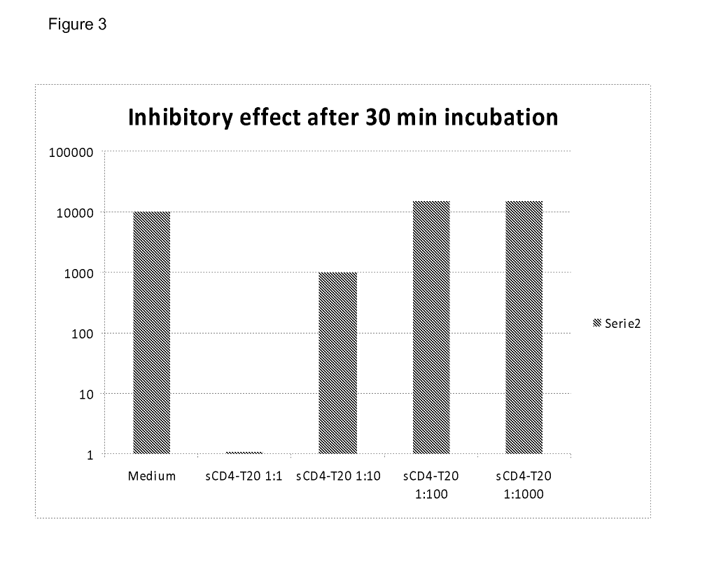

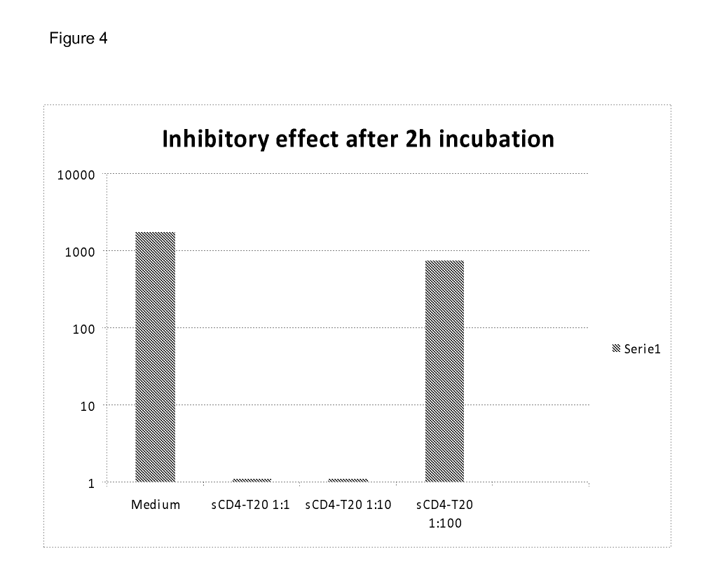

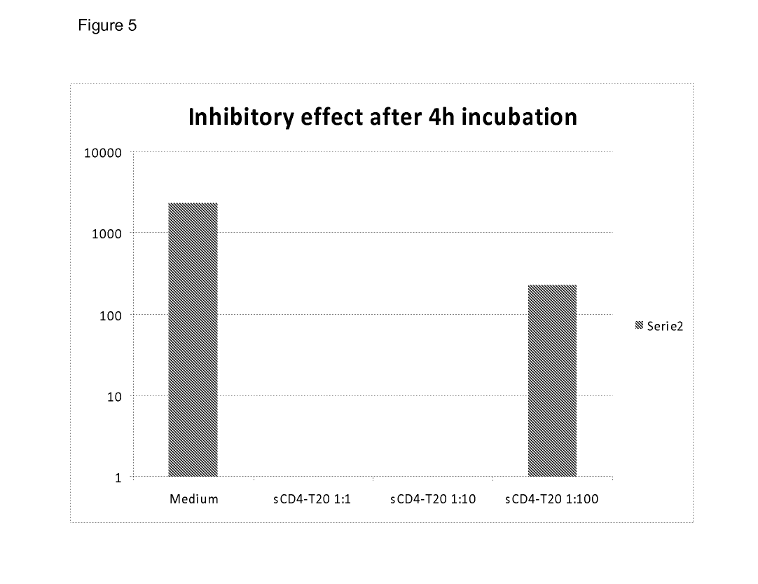

### The cfu Assay (Example 1, FIGS. 3-5)

Pseudotyped viral particles containing MLV core (gagpol and containing a neo expressing retroviral vector) and truncated HIV envelope protein are incubated with supernatant containing the bivalent molecules of the present invention for 30 minutes, 2 hours and 4 hours respectively at 37 degrees Celsius. Subsequently, the infectivity (titer) of the virus is measured on D17 cells that stably express HIV receptor and co-receptor, through serial dilutions. After 10 days of selection with G418, colonies are counted and the titer (cfu/ml) is calculatedThe protocol of the cfu assay is described in Example 1 herein below.

The bivalent molecules of the present invention are able to inhibit the infection by HIV particles measured as the titer (cfu/ml) according to the cfu assay as described above. In one embodiment the bivalent molecules of the present invention are able to reduce the titer with a factor of 100-15000 as measured by the cfu assay as described here above, such as a factor of 100-12500, for example a factor of 100-10000, or a factor of 100-8000, such as a factor of 100-6000, for example a factor of 100-4000, or a factor of 100-2000, such as a factor of 100-1000, for example a factor of 100-800, such as a factor of 100-500.

In another embodiment the bivalent molecules of the present invention are able to reduce the titer with a factor of 1000-15000 as measured by the cfu assay as described here above, such as a factor of 1000-12500, for example a factor of 1000-10000, or a factor of 100-8000, such as a factor of 1000-6000, for example a factor of 1000-4000, such as a factor of 1000-2000.

In a further embodiment the bivalent molecules of the present invention are able to reduce the titer after 30 minutes incubations time with a factor of 1000-10000 as measured by the cfu assay as described here above, such as a factor of 1000-9000, for example a factor of 1000-8000, or a factor of 1000-7000, such as a factor of 1000-6000, for example a factor of 1000-5000, or a factor of 1000-4000, such as a factor of 1000-3000, for example a factor of 1000-2000, such as a factor of 1000-1500.

In an even further embodiment the bivalent molecules of the present invention are able to reduce the titer after 2-4 hours incubations time with a factor of 500-3500 as measured by the cfu assay as described here above, such as 500-3250, for example 500-300, such as a factor of 500-2750, for example a factor of 500-2500, or a factor of 500-2250, such as a factor of 500-2000, for example a factor of 500-1750, or a factor of 500-1500, such as a factor of 500-1000.

In another embodiment the bivalent molecules of the present invention are able to reduce the titer after 4 hours incubations time with a factor of 1000-10000 as measured by the cfu assay as described here above, such as a factor of 1000-9000, for example a factor of 1000-8000, or a factor of 1000-7000, such as a factor of 1000-6000, for example a factor of 1000-5000, or a factor of 1000-4000, such as a factor of 1000-3000, for example a factor of 1000-2000, such as a factor of 1000-1500.

The bivalent molecule of the present invention with amino acid sequence consisting of SEQ ID NO: 1 is in one embodiment able to reduce the titer after 30 minutes of incubation time with a factor of about 10000 as measured by the cfu assay as described above.

In another embodiment the bivalent molecule of the present invention with amino acid sequence consisting of SEQ ID NO: 1 is able to reduce the titer after 2 hours of incubation time with a factor of about 1750 as measured by the cfu assay as described above.

In a further embodiment the bivalent molecule of the present invention with amino acid sequence consisting of SEQ ID NO: 1 is able to reduce the titer after 4 hours of incubation time with a factor of about 2300 as measured by the cfu assay as described above

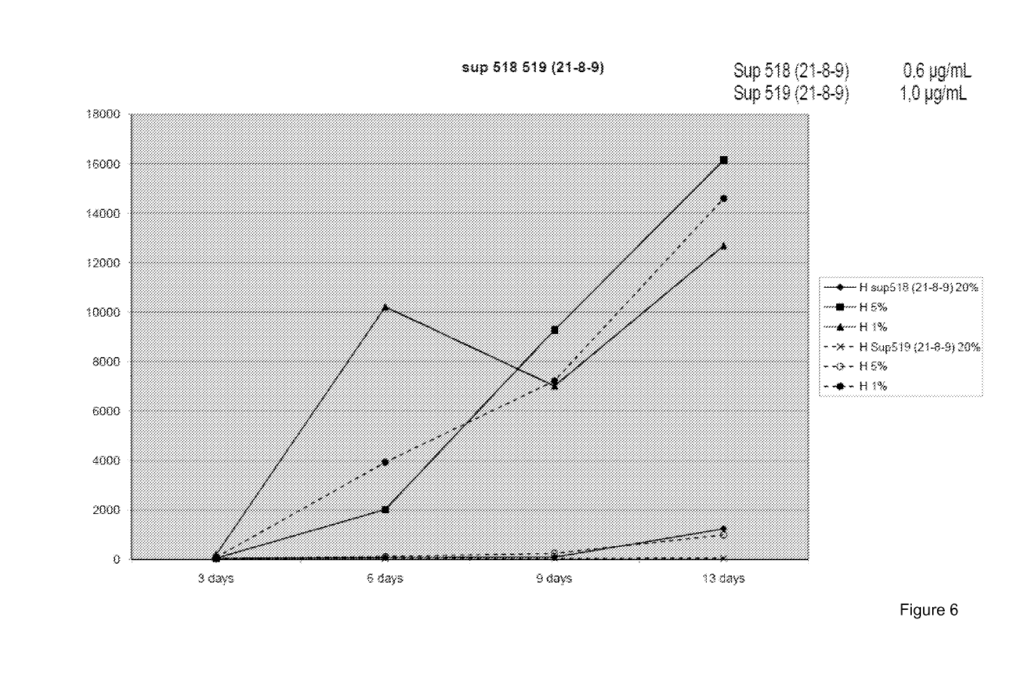

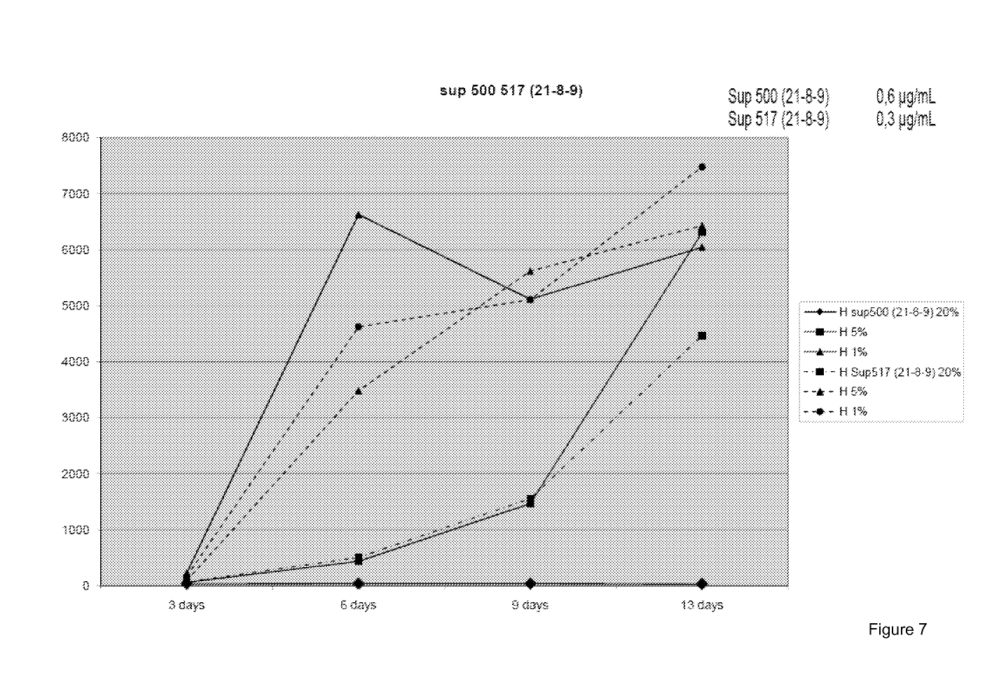

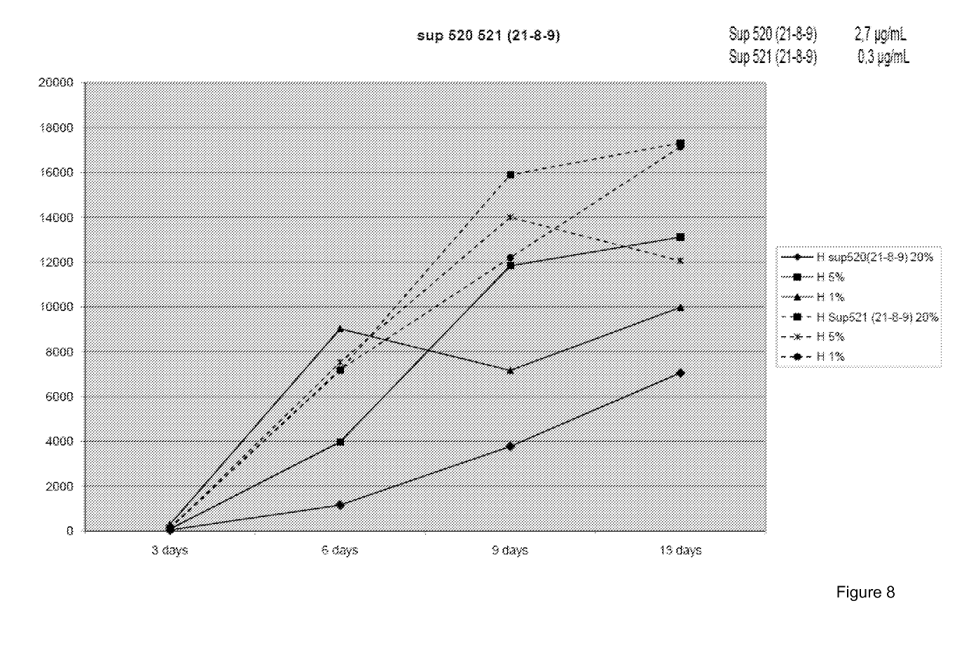

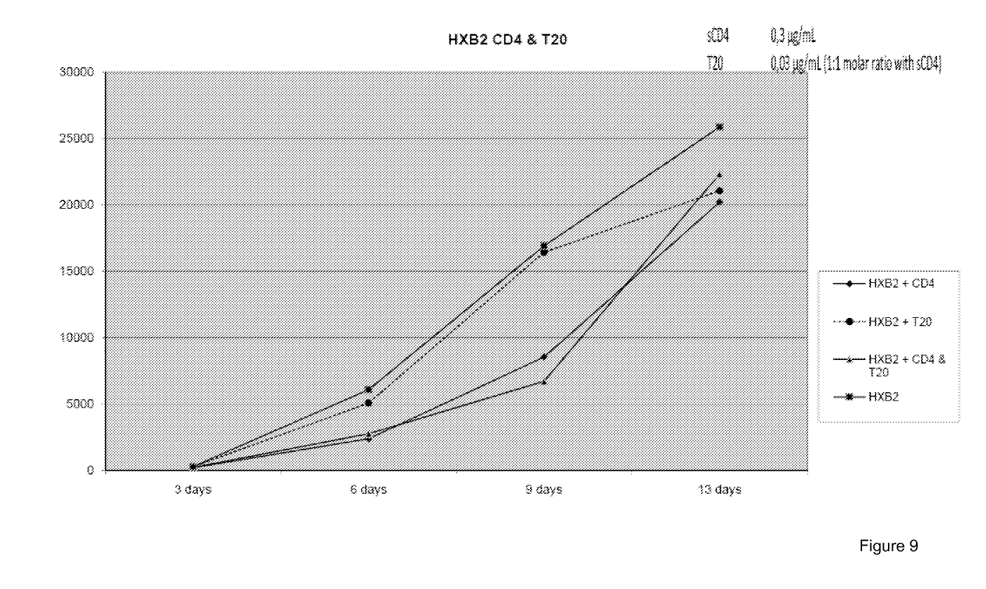

The p24gag Assay (Examples 2+3, FIGS. 6-9)

Day 0: HXB2 strain of HIV is used for the following experiment. Supernatant containing replication competent HIV (HXB2) is incubated with supernatants that contain the bivalent molecules of the present invention or medium containing known amounts of recombinant sCD4 (R & D) and/or T20 peptide (Roche) (controls) or just plain medium (control) at 37 degrees Celsius for 30 minutes. Subsequently the inactivated virus is added to Jurkat cells.

Day 1: The cells are centrifuged at after which the supernatant is removed. The cells are resuspended in RPMI 1640 containing 10% FCS. The cell suspension is subsequently mixed with medium containing the same amount of the bivalent molecules of the present invention (or the controls sCD4/T20 controls) as used on day 0. The cells are divided into wells of a 24 well plate and incubated at 37 degrees Celsius and left for the virus to replicate. Triplicates for each sample is set-up

Day 3, 6, 9 and 13: Supernatants from 3 wells are centrifuged and the amount of HIV p24gag is determined by ELISA. The amount of HIV p24gag in the medium is proportional to the extent of spreading of the HIV particles.

The bivalent molecules of the present invention are in one embodiment able to inhibit the spreading of HIV particles measured as the amount of the HIV p24gag protein (pg/ml) according to the p24gag assay as described above. In one embodiment the bivalent molecules of the present invention are able to reduce the amount of p24gag present in the medium with as much as about 50000 pg/ml as measured by the p24gag assay as described here above, such as about 45000 pg/ml, for example about 40000 pg/ml, as much as about 35000 pg/ml, for example about 30000 pg/ml, such as about 25000 pg/ml, for example about 20000 pg/ml, such as about 15000 pg/ml, as much as about 10000 pg/ml, for example about 8000 pg/ml, such as about 6000 pg/ml, for example about 4000 pg/ml, as much as about 2000 pg/ml, for example about 1000 pg/ml as measured by the p24gag assay as described above.

In another embodiment the bivalent molecules of the present invention are able to reduce the amount of p24gag present in the medium after 6 days of incubation time with as much as about 10000 pg/ml as measured by the p24gag assay as described here above, such as about 9000 pg/ml, for example about 8000 pg/ml, as much as about 7000 pg/ml, for example about 6000 pg/ml, such as about 5000 pg/ml, for example about 4000 pg/ml, such as about 3000 pg/ml, as much as about 2000 pg/ml, for example about 1000 pg/ml, such as about 800 pg/ml, for example about 600 pg/ml, as much as about 400 pg/ml, for example about 200 pg/ml, such as about 100 pg/ml as measured by the p24gag assay as described above.

In another embodiment the bivalent molecules of the present invention are able to reduce the amount of p24gag present in the medium after 9 days of incubation time with as much as about 20000 pg/ml as measured by the p24gag assay as described here above, such as about 18000 pg/ml, for example about 16000 pg/ml, as much as about 14000 pg/ml, for example about 12000 pg/ml, such as about 10000 pg/ml, for example about 8000 pg/ml, such as about 6000 pg/ml, as much as about 4000 pg/ml, for example about 2000 pg/ml, such as about 1000 pg/ml, for example about 800 pg/ml, as much as about 600 pg/ml, for example about 400 pg/ml, such as about 200 pg/ml as measured by the p24gag assay as described above.

In yet another embodiment the bivalent molecules of the present invention are able to reduce the amount of p24gag present in the medium after 13 days of incubation time with as much as about 30000 pg/ml as measured by the p24gag assay as described here above, such as about 28000 pg/ml, for example about 26000 pg/ml, as much as about 24000 pg/ml, for example about 22000 pg/ml, such as about 20000 pg/ml, for example about 18000 pg/ml, such as about 16000 pg/ml, as much as about 14000 pg/ml, for example about 12000 pg/ml, such as about 10000 pg/ml, for example about 8000 pg/ml, as much as about 6000 pg/ml, for example about 4000 pg/ml, such as about 2000 pg/ml, for example about 1000 pg/ml as measured by the p24gag assay as described above.

In a particular embodiment the bivalent molecule of the present invention with amino acid sequence consisting of SEQ ID NO: 1 is able to reduce the amount of p24gag present in the medium after 6 days of incubation time with as much as about 7000 pg/ml as measured by the p24gag assay as described here above.

In a another particular embodiment the bivalent molecule of the present invention with amino acid sequence consisting of SEQ ID NO: 1 is able to reduce the amount of p24gag present in the medium after 6 days of incubation time with as much as about 17000 pg/ml as measured by the p24gag assay as described here above.

In a further particular embodiment the bivalent molecule of the present invention with amino acid sequence consisting of SEQ ID NO: 1 is able to reduce the amount of p24gag present in the medium after 13 days of incubation time with as much as about 26000 pg/ml as measured by the p24gag assay as described here above.

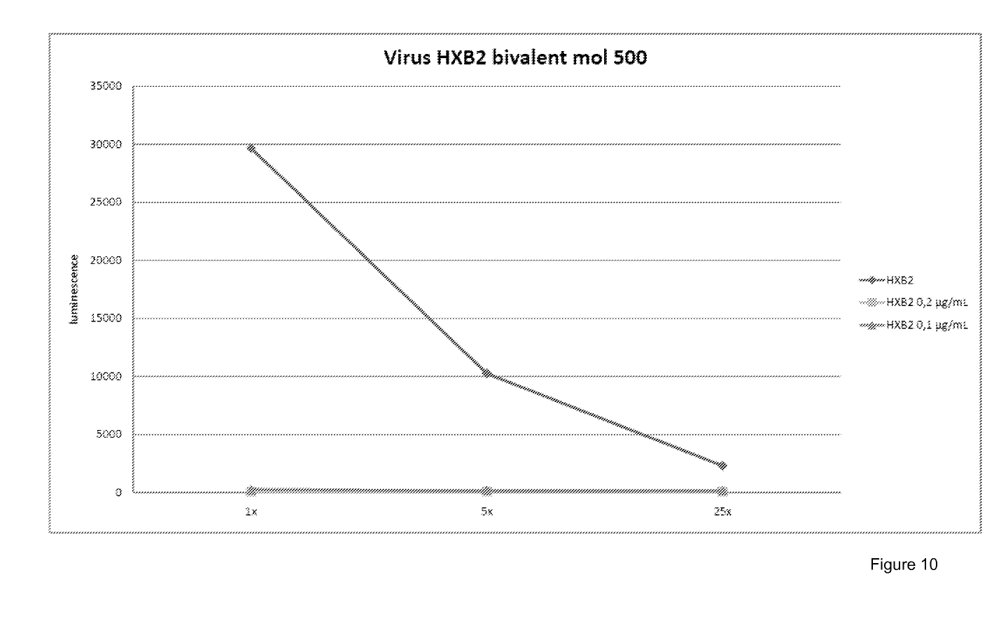

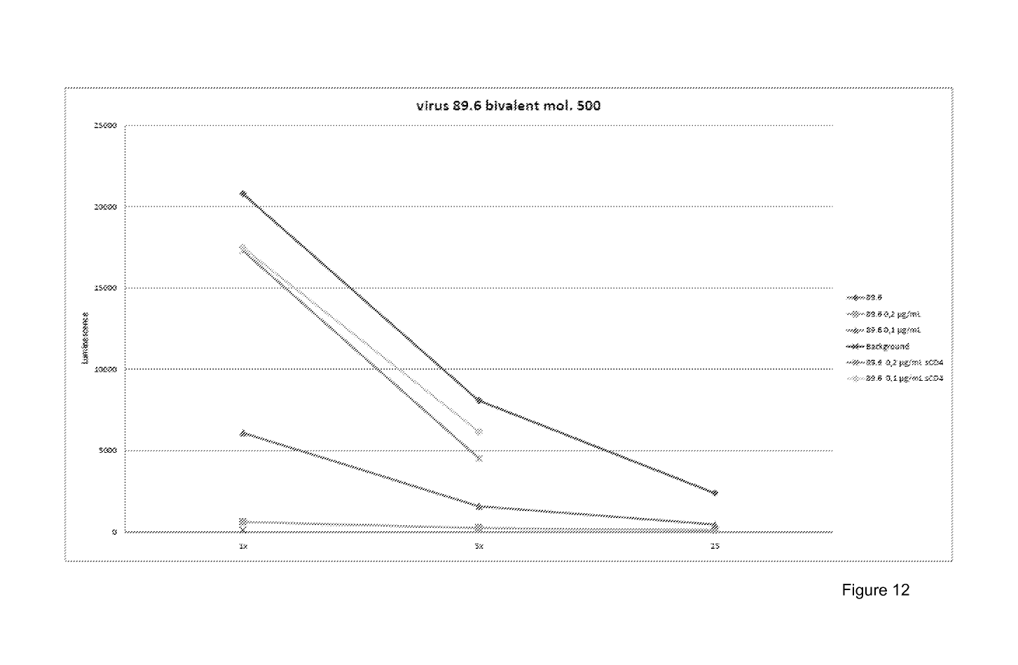

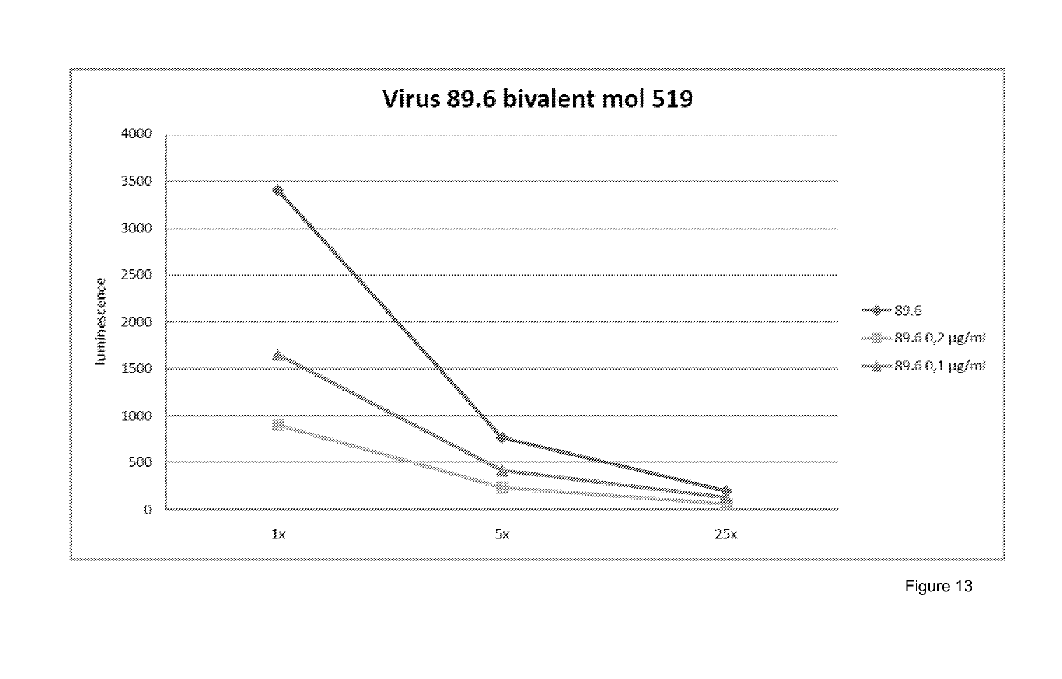

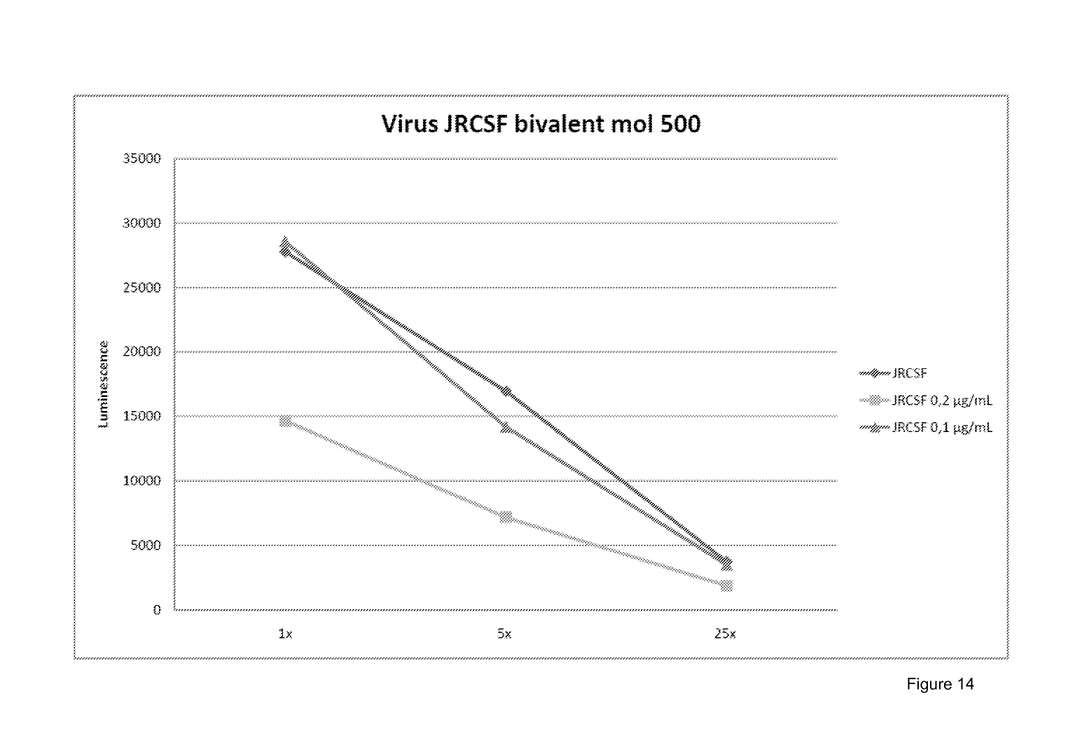

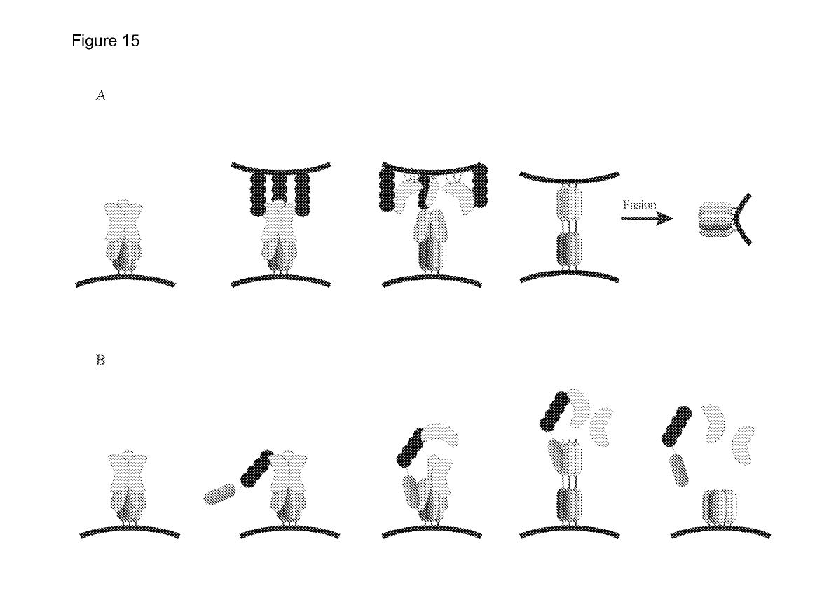

### The Luciferase Assay (Example 4, FIGS. 10-14)

The experiment is based on the activation of the luciferase gene upon infection of TZM-bl cells with 3 different virus strains (HXB2 (CRCX4-tropic), virus 89.6 (Dual-tropic) or JRCSF (CCR5-tropic)). The more luminescence measured, the greater activation of the luciferase gene has occurred, and the more inefficient the inhibition of infection has been by the molecule of the present invention

The bivalent molecules of the present invention are able to inhibit the infection of HIV particles as measured by the amount of decreased luminescence detected in TZM-bl cells according to the luciferase assay as described above. In one embodiment the bivalent molecules of the present invention are able to reduce the amount of luminescence in TMZ-bl cells incubated with HXB2 with as much as about 35000 as measured by the luciferase assay as described here above, such as a decrease of about 30000, such as a decrease of about 25000, for example a decrease of about 20000, such as a decrease of about 15000, for example a decrease of about 10000, such as a decrease of about 5000, for example a decrease of about 2500, such as a decrease of about 2000, for example a decrease of about 1000, such as a decrease of about 500 as measured by the luciferase assay as described here above.

In a particular embodiment the bivalent molecule of the present invention with amino acid sequence consisting of SEQ ID NO: 1 is able to decrease luminescence in TMZ-bl cells incubated with HXB2 with as much as about 30000 as measured by the luciferase assay as described here above.

In another particular embodiment the bivalent molecule of the present invention with amino acid sequence consisting of SEQ ID NO: 6 is able to decrease luminescence in TMZ-bl cells incubated with HXB2 with as much as about 17800 as measured by the luciferase assay as described here above.

The bivalent molecules of the present invention are also able to reduce the amount of luminescence in TMZ-bl cells incubated with Virus 89.6 with as much as about 25000 as measured by the luciferase assay as described here above, such as a decrease of about, for example a decrease of about 20000, such as a decrease of about 15000, for example a decrease of about 10000, such as a decrease of about 5000, for example a decrease of about 2500, such as a decrease of about 2000, for example a decrease of about 1000, such as a decrease of about 500 as measured by the luciferase assay as described here above.

In a particular embodiment the bivalent molecule of the present invention with amino acid sequence consisting of SEQ ID NO: 1 is able to decrease luminescence in TMZ-bI cells incubated with Virus 89.6 with as much as about 22000 as measured by the luciferase assay as described here above.

In another particular embodiment the bivalent molecule of the present invention with amino acid sequence consisting of SEQ ID NO: 6 is able to decrease luminescence in TMZ-bl cells incubated with Virus 89.6 with as much as about 2500 as measured by the luciferase assay as described here above.

The bivalent molecules of the present invention are also able to reduce the amount of luminescence in TMZ-bl cells incubated with JRCSF with as much about 15000, for example a decrease of about 10000, such as a decrease of about 5000, for example a decrease of about 2500, such as a decrease of about 2000, for example a decrease of about 1000, such as a decrease of about 500 as measured by the luciferase assay as described here above.

In a particular embodiment the bivalent molecule of the present invention with amino acid sequence consisting of SEQ ID NO: 1 is able to decrease luminescence in TMZ-bl cells incubated with JRCSF with as much as about 12500 as measured by the luciferase assay as described here above.

### Polynucleotides and Expression Vectors

Another aspect of the present invention pertains to polynucleotides comprising and/or consisting of one or more nucleic acid sequences encoding at least one of the bivalent molecules of the present invention as described herein above, or any part thereof, or fragment thereof, or mimic thereof, or functional homologue of said molecules, or a polynucleotide with at least 80% identity to said nucleic acid sequence or part thereof, such as 81% identity for example at least 82% identity, at least 83% identity, such as at least 84% identity, for example at least 85% identity, at least 86% identity, such as at least 87% identity, for example at least 88% identity, at least 89% identity, such as at least 90% identity, for example at least 91% identity, at least 92% identity, such as at least 93% identity, for example at least 94% identity, at least 95% identity, such as at least 96% identity, for example at least 97% identity, at least 98% identity, such as at least 99% identity to said nucleic acid sequence, or any polynucleotide that have been modified by codon optimization, encoding at least one of the bivalent molecules of the present invention.

Thus, in one embodiment of the present invention the polynucleotides as described above comprise and/or consist of nucleic acid sequences selected from the group of nucleic acid sequences consisting of SEQ ID NOS: 205-212 or 226-235 or any part thereof or fragment thereof, or any polynucleotide that have been modified by codon optimization, encoding any of the bivalent molecules of the present invention with SEQ ID NOS: 1-8 or 216-225.

In another embodiment of the present invention the polynucleotides as described above comprise and/or consist of nucleic acid sequences selected from the group of nucleic acid sequences consisting of SEQ ID NOS: 206-209, or any polynucleotide that have been modified by codon optimization, encoding any of the bivalent molecules of the present invention with SEQ ID NOS: 2-5.

In a further embodiment of the present invention the polynucleotides as described above comprise and/or consist of nucleic acid sequences selected from the group of nucleic acid sequences consisting of SEQ ID NOS: 205, 210-212, or any polynucleotide that have been modified by codon optimization, encoding any of the bivalent molecules of the present invention with SEQ ID NOS: 1, 6-8.

In a preferred embodiment of the present invention the polynucleotides as described above comprise and/or consist of a nucleic acid sequences consisting of SEQ ID NO: 205 or any part thereof or fragment thereof, or an nucleic acid sequence at least 80% identical to SEQ ID NO: 205, or any polynucleotide that have been modified by codon optimization, encoding any of the bivalent molecules of the present invention with SEQ ID NO: 1

In another preferred embodiment of the present invention the polynucleotides as described above comprise and/or consist of a nucleic acid sequences consisting of SEQ ID NO: 210 or any part thereof or fragment thereof, or an nucleic acid sequence at least 80% identical to SEQ ID NO: 210, or any polynucleotide that have been modified by codon optimization, encoding any of the bivalent molecules of the present invention with SEQ ID NOS: 6

In another preferred embodiment of the present invention the polynucleotides as described above comprise and/or consist of a nucleic acid sequences consisting of SEQ ID NO: 211 or any part thereof or fragment thereof, or an nucleic acid sequence at least 80% identical to SEQ ID NO: 211, or any polynucleotide that have been modified by codon optimization, encoding any of the bivalent molecules of the present invention with SEQ ID NO: 7.

In yet another preferred embodiment of the present invention the polynucleotides as described above comprise and/or consist of a nucleic acid sequences consisting of SEQ ID NO: 212 or any part thereof or fragment thereof, or an nucleic acid sequence at least 80% identical to SEQ ID NO: 212, or any polynucleotide that have been modified by codon optimization, encoding any of the bivalent molecules of the present invention with SEQ ID NOS: 8.

In a further aspect, the present invention also relates to an isolated expression vector comprising at least one nucleic acid sequence according to the present invention or a functional homolog or a fragment thereof, or a nucleic acid encoding a polypeptide with at least 80% identity thereto, or any polynucleotide that have been modified by codon optimization, encoding any of the bivalent molecules of the present invention. The vector of the present invention is a prokaryotic expression vector or a eukaryotic expression vector, preferably a mammalian expression vector. Thus, in one embodiment, the present invention relates to an isolated eukaryotic expression vector comprising at least one nucleic acid sequence encoding at least of the bivalent molecules of the present invention, or a fragment thereof and/or a nucleic acid sequence encoding at least one antigen as defined herein.

Numerous vectors are available and the skilled person will be able to select a useful vector for the specific purpose. The vector may, for example, be in the form of a plasmid, cosmid, viral particle or artificial chromosome. The appropriate nucleic acid sequence may be inserted into the vector by a variety of procedures, for example, DNA may be inserted into an appropriate restriction endonuclease site(s) using techniques well known in the art. Apart from the nucleic acid sequence according to the invention, the vector may furthermore comprise one or more of a signal sequence, an origin of replication, one or more marker genes, an enhancer element, a promoter, and a transcription termination sequence. The vector may also comprise additional sequences, such as enhancers, poly-A tails, linkers, polylinkers, operative linkers, multiple cloning sites (MCS), STOP codons, internal ribosomal entry sites (IRES) and host homologous sequences for integration or other defined elements. Methods for engineering nucleic acid constructs are well known in the art (see, e.g., Molecular Cloning: A Laboratory Manual, Sambrook et al., eds., Cold Spring Harbor Laboratory, 2nd Edition, Cold Spring Harbor, N.Y., 1989).

In one preferred embodiment the vector is a viral vector. The vector may also be a bacterial vector, such as an attenuated bacterial vector. Attenuated bacterial vectors may be used in order to induce lasting mucosal immune responses at the sites of infection and persistence. Different recombinant bacteria may be used as vectors, for example the bacterial vector may be selected from the group consisting of Salmonella, Lactococcus), and Listeria.

The vector of the present invention may be any eukaryotic expression vector, for example a mammalian expression vector, or a yeast vector. The vector may comprise at least one intron, which will facilitate the transport from the nucleus to the cytoplasma of the vector encoded RNA, for example in packaging cells. In another embodiment, the vector is capable of expressing RNA in the cytoplasm by cytoplasmic transcription, which can be translated into envelope polypeptide. The vector is also, in one embodiment, capable of expressing high levels of vector encoded RNA, which is transported to the cytoplasma to be translated into envelope polypeptide as encoded in the vector. Thus, in one embodiment the vector of the present invention is transcribed in the nucleus, thereby producing high levels of transcript, which after transport to the cytoplasm can be translated into envelope polypeptide. The vector of the present invention may be transfected into a packaging cell which is capable of producing viral particles comprising said lentiviral envelope polypeptide.

In one embodiment, the vector is a retroviral vector. The retroviral vector may be either replication deficient or replication competent.

### Pharmaceutical Compositions, Formulations, Administration- and Dosage Forms

Another aspect of the present invention pertains to pharmaceutical compositions comprising one or more bivalent molecules as described herein above.

Any suitable route of administration of the pharmaceutical composition of the present invention comprising one or more bivalent molecules of the invention may be employed for providing a mammal, especially a human, with an effective dose of a compound of the present invention. For example, oral, rectal, vaginal, topical, parenteral, ocular, pulmonary, nasal, and the like may be employed. Other examples of administration include sublingually, intravenously, intramuscularly, intrathecally, subcutaneously, cutaneously and transdermally administration. In one preferred embodiment the administration comprises injection or release from any type of implant. The administration of the compound according to the present invention can result in a local (topical) effect or a bodywide (systemic) effect.

Pharmaceutical compositions containing the bivalent molecules of the present invention may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example suspensions or as a solution, lubricant, gel, cream, lotion, shake lotion, ointment, foam, shampoo, mask or similar forms.

Whilst it is possible for the compositions or salts of the present invention to be administered as the raw chemical, it is preferred to present them in the form of a pharmaceutical formulation. Accordingly, the present invention further provides a pharmaceutical formulation, for medicinal application, which comprises a composition of the present invention or a pharmaceutically acceptable salt thereof, as herein defined, and a pharmaceutically acceptable carrier therefore.

The pharmaceutical compositions and dosage forms may comprise the compositions of the invention or its pharmaceutically acceptable salt or a crystal form thereof as the active component. The pharmaceutically acceptable carriers can be either solid, semi-solid or liquid. Emulsions may be prepared in solutions in aqueous propylene glycol solutions or may contain emulsifying agents such as lecithin, sorbitan monooleate, or acacia. Aqueous solutions can be prepared by suspending or mixing the active component in water and adding suitable colorants, flavors, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include suspensions and emulsions, and may contain, in addition to the active component, colorants, stabilizers, buffers, artificial and natural dispersants, thickeners, and the like.

The compositions of the present invention may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Oils useful in formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in formulations include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The formulations typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, immediately prior to use.

The pharmaceutical composition may include a pharmaceutically acceptable carrier adapted for topical administration. Thus, the composition may take the form of a suspension, solution, ointment, lotion, lubricant, cream, foam, aerosol, spray, suppository, tablet, capsule, dry powder, syrup, or balm. Methods for preparing such compositions are well known in the pharmaceutical industry.

The compositions of the present invention may be formulated as lubricants, ointments, creams or lotions, or as a transdermal patch. Ointments and creams may, for example, be formulated with an aqueous or oily base with the addition of suitable thickening or gelling agents. Lubricants and lotions may be formulated with an aqueous or oily base and will in general also containing one or more emulsifying agents, stabilizing agents, dispersing agents, suspending agents, thickening agents, or colouring agents. Formulations suitable for topical administration in the mouth include lozenges comprising active agents in a flavoured base, usually sucrose and acacia or tragacanth; pastilles comprising the active ingredient in an inert base such as gelatine and glycerin or sucrose and acacia; and mouthwashes comprising the active ingredient in a suitable liquid carrier.

Lubricants, creams, ointments, gels, balms, or pastes according to the present invention are semi-solid formulations of the active ingredient for external and/or internal application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included. Suitable permeable membrane materials may be selected based on the desired degree of permeability, the nature of the complex, and the mechanical considerations related to constructing the device. Exemplary permeable membrane materials include a wide variety of natural and synthetic polymers, such as polydimethylsiloxanes (silicone rubbers), ethylenevinylacetate copolymer (EVA), polyurethanes, polyurethane-polyether copolymers, polyethylenes, polyamides, polyvinylchlorides (PVC), polypropylenes, polycarbonates, polytetrafluoroethylenes (PTFE), cellulosic materials, e.g., cellulose triacetate and cellulose nitrate/acetate, and hydrogels, e.g., 2-hydroxyethylmethacrylate (HEMA).

Other items may be contained in the device, such as other conventional components of therapeutic products, depending upon the desired device characteristics. For example, the compositions according to this invention may also include one or more preservatives or bacteriostatic agents, e.g., methyl hydroxybenzoate, propyl hydroxybenzoate, chlorocresol, benzalkonium chlorides, and the like. These pharmaceutical compositions also can contain other active ingredients such as antimicrobial agents, particularly antibiotics, anesthetics, analgesics, and antipruritic agents.

The compositions of the present invention may be formulated for administration as suppositories, for example as rectal and/or vaginal suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active composition may be formulated into a suppository comprising, for example, about 0.5% to about 50% of a composition of the invention, disposed in a polyethylene glycol (PEG) carrier (e.g., PEG 1000 [96%] and PEG 4000 [4%].

The compositions of the present invention may be formulated for aerosol administration, particularly for spraying on the site for topical application. The composition will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the composition in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatine or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

In one embodiment the pharmaceutical composition of the present invention is a composition comprising one or more bivalent molecules of the present invention.

In a preferred embodiment the pharmaceutical composition of the present invention is a composition comprising one or more bivalent molecules of the present invention with amino acid sequence selected from the group of amino acid sequences consisting of SEQ ID NOS: 1, 6-8.

A certain aspect of the present invention relates to pharmaceutical compositions comprising one or more of the bivalent molecules of the invention for use as a medicament.

### Coating Composition, Contraceptive Devices and Medico-Technological Devices

Another aspect of the present invention relates to a coating composition and the use of such coating composition comprising one or more bivalent molecules of the present invention. Such a coating composition may be used to coat for example contraceptive devices or any microdevice or medico-technological device used under conditions where a potential risk of virus infection exist. Contraceptive devices, which are also a type of medico-technological devises according to the present invention, may be any device or any means used in contraception, such as condoms, female condoms, sponges, diaphragms, vaginal rings, cervical caps, coils, spermicides, contraceptive lubricants and/or any other intrauterine devices.

The medico-technological device to be coated with the coating composition comprising the bivalent molecules of the present invention include all devices, instruments, structures, etc. intended to be in contact with at least one mammalian body fluid and/or at least one mammalian tissue. A “medico-technological device”, as used herein, thus refers to a device having surfaces that contact tissue, blood, or other bodily fluids of a mammal, in particular humans, in the course of their operation or utility.

Medico-technological devices can be prepared by coating the exposed surface in part or completely with the coating composition of the present invention. For example, this can be done by submersing the device into the coating composition of the present invention and then allowing excess coating composition to drain from the device. Alternately, the coating may be applied by spraying techniques, dipping techniques and other techniques that allow the coating composition to come into contact with the device or device surface. The coating may then be dried in an appropriate atmosphere (low humidity, temperature-controlled, dust-free, and sterile if aseptic processing is required).

The contraceptive devices and medico-technological devices may be made of a variety of metals, including stainless steel and platinum. However, the medico-technological devices may also be made of plastic.

The present invention also relates to contraceptive devices and medico-technological devices containing biological entities, such as cells or single-cell organisms, that produce the bivalent molecules of the present invention. Such devices may be transplanted in an individual so as to achieve a continuously production of the bivalent molecules of the present invention.

Bone plates and bone plating systems are also within the scope of the present invention as medico-technological devices. Biodegradable fixation systems consisting of plates, plates and mesh, and mesh, in varying configurations and length, can be attached to bone for reconstruction. Such uses include the fixation of bones of the craniofacial and midfacial skeleton affected by trauma, fixation of zygomatic fractures, or for reconstruction. The plates may also be contoured by molding. Examples of such state of the art devices include the Howmedica LEIBINGER™ Resorbable Fixation System (Howmedica, Rutherford, N.J.),

Similarly, repair patches are examples of medico-technological devices of the present invention. Biodegradable repair patches are often used in general surgery. Patches may be used for pericardial closures, the repair of abdominal and thoracic wall defects, inguinal, paracolostomy, ventral, paraumbilical, scrotal, femoral, and other hernias, urethral slings, muscle flap reinforcement, to reinforce staple lines and long incisions, reconstruction of pelvic floor, repair of rectal and vaginal prolapse, suture and staple bolsters, urinary and bladder repair, pledgets and slings, and other soft tissue repair, reinforcement, and reconstruction. Examples of such state of the art patches include the TISSUEGUARD™ product (Bio-Vascular Inc., St. Paul, Minn., USA). In analogy, cardiovascular patches such as biodegradable cardiovascular patches used for vascular patch grafting, (pulmonary artery augmentation), for intracardiac patching, and for patch closure after endarterectomy are examples within the scope of the present invention.

Examples of similar state of the art (non-degradable) patch materials include Sulzer Vascutek FLUOROPASSIC™ patches and fabrics (Sulzer Carbomedics Inc., Austin Tex., USA)

Other useful devices to be coated with the composition of the present invention include sutures, suture fasteners, meniscus repair devices, rivets, tacks, staples, screws (including interference screws), bone plates and bone plating systems, surgical mesh, repair patches, slings, cardiovascular patches, orthopedic pins, heart valves and vascular grafts, adhesion barriers, stents, guided tissue repair/regeneration devices, articular cartilage repair devices, nerve guides, tendon repair devices, atrial septal defect repair devices, pericardial patches, bulking and filling agents, vein valves, bone marrow scaffolds, meniscus regeneration devices, ligament and tendon grafts, ocular cell implants, spinal fusion cages, skin substitutes, dural substitutes, bone graft substitutes, bone dowels, wound dressings, tubings, catheters and hemostats. Particular embodiments of medico-technological device of the present invention are catheters, tubings and guide wires.

In one embodiment the coating composition of the present invention is a composition comprising one or more bivalent molecules of the present invention.

In a preferred embodiment the coating composition of the present invention is a composition comprising one or more bivalent molecules of the present invention with amino acid sequence selected from the group of amino acid sequences consisting of SEQ ID NOS: 1, 6-8.

### Uses

Yet another aspect of the present invention pertains to the use of the bivalent molecules of the invention. A major aspect of the present invention is the use of the bivalent molecules of the invention for virus inhibition, and particularly inhibition of viruses belonging to the groups of Orthomyxoviridae, Paramyxoviridae, Retroviridae, Filoviridae and Coronaviridae. The virus to be inhibited by the bivalent molecules of the present invention may be any virus as disclosed herein, However, a certain aspect of the present invention relates to the use of the bivalent molecules of the invention for inhibition of Human Immunodeficiency Virus (HIV).

Thus, the present invention relates to the use of the bivalent molecules of the invention, and compositions comprising the bivalent molecules of the invention, for use as a virus fusion inhibitor and/or entry inhibitor, preferably a pre-fusion inhibitor.

In one embodiment the present invention relates to the use of the bivalent molecules, or compositions comprising the bivalent molecules of the invention with amino acid sequence selected from the group of amino acid sequences consisting of SEQ ID NOS: 1-8 as a virus fusion inhibitor and/or entry inhibitor, preferably a pre-fusion inhibitor.

In a preferred embodiment the present invention relates to the use of the bivalent molecules, or compositions comprising the bivalent molecules of the invention with amino acid sequence selected from the group of amino acid sequences consisting of SEQ ID NOS: 1, 6-8 as HIV fusion inhibitor and/or entry inhibitor, preferably a pre-fusion inhibitor.

In yet another preferred embodiment the present invention relates to the use of the bivalent molecules, or compositions comprising the bivalent molecules of the invention with amino acid sequence selected from the group of amino acid sequences consisting of SEQ ID NOS: 1, 6-8 as virus fusion inhibitor and/or entry inhibitor, preferably a pre-fusion inhibitor, wherein said inhibitor is able to destabilize the virus envelope structure by triggering conformational changes in said virus envelope structure.

In a further preferred embodiment the present invention relates to the use of the bivalent molecules, or compositions comprising the bivalent molecules of the invention with amino acid sequence selected from the group of amino acid sequences consisting of SEQ ID NOS: 1, 6-8 as HIV fusion inhibitor and/or entry inhibitor, preferably a pre-fusion inhibitor, wherein said inhibitor is capable transforming the virus envelope structure (ENV) from the pre-fusion state to the post-fusion state, or any intermediate transition state.

The present invention also relates to pharmaceutical compositions comprising one or more of the bivalent molecules of the invention for use as a medicament.

The present invention further relates to the use of one or more bivalent molecules of the present invention for the manufacture of a medicament for the treatment and/or amelioration and/or prevention of diseases and/or clinical conditions. It is appreciated that the diseases and/or clinical conditions arise from infections, in particular virus infections, and preferably infections caused by HIV.

The present invention also relates to the use of the bivalent molecules of the invention, and compositions comprising the bivalent molecules of the invention, for use in gene therapy, including gene therapy where genes encoding the bivalent molecules of the present invention are inserted into cells and/or tissues of the individual wherein the bivalent molecules are to be expressed and utilized, as well as transgenic cells expressing bivalent molecules of the present invention that have been transplanted in the individual. Transplanted cells may originate from the same organism or a different organism.

Further, the present invention relates to the use of the bivalent molecules of the invention, and compositions comprising the bivalent molecules of the invention, for use in gene therapy, including gene therapy where genes encoding the bivalent molecules of the present invention are used for continuous and stable production of the bivalent molecules of the present invention by single-cell organisms, including bacteria, protozoa, amoebae, viruses, moulds, yeast, fungus, and the like, so that the bivalent molecules may be constantly supplied to the individual wherein the bivalent molecules are to be utilized.

Another aspect of the present invention relates to the use of the bivalent molecules, or compositions comprising the bivalent molecules of the invention for use as a microbicide, in particular a microbicide for sexually transmitted diseases. The microbicide may any kind of antibiotic, fungicide, bactericide, in particular any kind of microbicide effective against viruses. The microbicide is useful for application to or coating of any type of implant, medico-technical device or contraceptive device as described elsewhere herein

### Compound and Method of Treatment

Another aspect of the present invention relates to a compound comprising one or more bivalent molecules of the invention and/or amelioration and/or treatment of a disease and/or clinical condition belonging to the group of diseases and/or clinical conditions arising from virus infections, in particular retroviral infections and preferably infections by HIV. In a certain aspect the disease is AIDS or ARC.

The present invention also relates to a method of treating, preventing and/or ameliorating a disease and/or clinical condition, said method comprising administering to an individual suffering from said disease and/or clinical condition an effective amount of one or more bivalent molecules of the invention, wherein said disease and/or clinical condition belongs to the group of diseases and/or clinical condition arising from virus infections, in particular retroviral infections and preferably infections by HIV. The disease is in one embodiment any diseases caused by a virus belonging to the groups of Orthomyxoviridae, Paramyxoviridae, Retroviridae, Filoviridae and Coronaviridae. In a preferred embodiment the disease is AIDS and/or ARC.

Further, the present invention relates to a method of treating, preventing and/or ameliorating a disease and/or clinical condition, said method comprising administering to an individual suffering from said disease and/or clinical condition an effective amount of one or more bivalent molecules of the present invention selected from the group consisting of SEQ ID NOS: 1, 6-8, wherein said disease and/or clinical condition is AIDS and/or ARC arising from infections by HIV.

### Method of Preparation of the Compositions

Another aspect relates to a method of preparation of the compositions of the present invention
a. providing one or more bivalent molecules as defined herein
b. optionally providing a salt and/or a carrier
c. providing a substance
d. mixing the molecules of step a. or b. with the substance of step c.
e. obtaining the compositions of the present invention.

The compositions of the present invention as defined herein above may also be prepared by following the steps of
a. providing one or more bivalent molecules selected from the group consisting of SEQ ID NOS: 1-8
b. optionally providing a salt and/or a carrier
c. providing a substance
d. mixing the molecules of step a. or b. with the substance of step c.
e. obtaining the compositions of the present invention

The compositions of the present invention as defined herein above may preferably be prepared by following the steps of
a. providing one or more bivalent molecules selected from the group consisting of SEQ ID NOS: 1, 6-8
b. optionally providing a salt and/or a carrier
c. providing a substance
d. mixing the molecules of step a. or b. with the substance of step c.
e. obtaining the compositions of the present invention

### EXAMPLES

### Example 1

### The cfu Assay—Protocol

Pseudotyped viral particles containing MLV core (gagpol and a neo-containing retroviral vector) and truncated HIV envelope protein were incubated with supernatant containing sCD4-T20 for the indicated period of time at 37 C. Subsequently, the infectivity (titer) of the virus was measured on D17 cells that stably express HIV receptor and co-receptor, through serial dilutions. After 10 days of selection with G418, colonies were counted and the titer calculated.

### Example 2

### The p24gag Assay—Protocol

### Day 0:

HXB2 strain of HIV is used for the following experiment.

200 uL of supernatant containing replication competent HIV (HXB2) is incubated with 200 ul supernatants that contain the bivalent inhibitors of interest or 200 μl of medium containing known amounts of recombinant sCD4 (R & D) and/or T20 peptide (Roche) (controls) at 37° C. for 30 minutes.

Subsequently the inactivated virus is added to 10⁶ Jurkat cells are seeded in a total volume of 2 ml (RPMI 1640 containing 10% Foetal Calf Serum, 1% Pen/strep all from (Invitrogen) in a 12 well plate (Nunc) for the inhibition set-up).

### Day 1:

The cells are centrifuged at 1250 rpm in a Hermle Z 300K centrifuge for 5 minutes after which the supernatant is removed.

The cells are resuspended in 1 ml of RPMI 1640 containing 10% FCS.

The cell suspension is subsequently mixed with medium containing the same amount of the bivalent inhibitor (or the controls) as used on day 0.

The cells are divided into wells of a 24 well plate (Nunc) (50000 cells in 1.5 ml medium/well) and incubated at 37° C. and left for the virus to replicate. Triplicates for each sample is set-up

### Day 4, 7, 10 and 13:

Supernatants from 3 wells are centrifuged (1250 rpm 5 minutes).

A sample of each supernatant is diluted 1:1 with 2% Empigen (Sigma cat no: 45165) and frozen at −20° C. until it is used to determine the presence and amount of p24gag by ELISA.

### Example 3

### Elisa p24gag

### Day 1:

1×96 well plate (Nunc) is coated with 5 pg/ml Anti H1 antibody (Aalto Bio Reagents Cat no:07320)

100 μl/well incubate overnight at 4° C.

### Day 2:

Wash the plate 1 time with PBS.

Block non-specifik binding by adding 300 μl/well “full well” of blocking buffer: 0.5% BSA (Sigma A 8022) in PBS.

Incubate at room temperature for 1-2 hours.

Wash the plate 2 times with PBS containing 0.05% Tween-20 (Sigma p1379).

Load samples & standards 100 μl/well. Incubate overnight at 4° C.

Samples: dilute the samples 1:1 with 2% Empigen (Sigma Cat no: 45165) in PBS for 45 minutes at room temperature to inactivate the HIV-virus.

Standards: Rec H1 P24 (Aalto Bio Reagents Code AG 6054)

Dilute the standards 1000 times in PBS containing 0.1% Empigen to 50 ng/ml and then make serial 3 fold dilutions to 0.85 ng/ml.

### Day 3:

Wash the plate 3 times in PBS 0.05% Tween-20.

The secondary antibody (Biotinylated Conjugate of Anti-HIV-1-p24 Mouse Monoclonal (Aalto Bio Reagents Code: BC 1071-BIOT)) is diluted 1000× in PBS containing 0.25% BSA, 10% lamb serum, 0.05% Tween-20 and 0.05% Empigen and added to the wells (100 μl/well).

Incubate at least 2 hours at room temperature.

Wash the plate 5 times in PBS 0.05% Tween-20.

Add 100 μl/well of Streptavidin HRP cytoset in PBS (Biosource Cat no:CHC 2203 part no: 41.000.03)

which is diluted 1:1000 in PBS 0.25% BSA 0.05% Tween-20.

Incubate 30 minutes at room temperature.

The plate is washed 4 times in PBS 0.05% Tween-20.

Add 100 μl/well TMB X-tra Ready to use substrate (KEM EN TEC Diagnostics Cat no: 4800L)

Incubate 5-10 minute—kept in dark! (until it turns blue)

Stop colour development by adding 100 μl/well 0.2M H₂SO₄.

Read the optical density (OD) for each well with 405 nm and 650 nm as reference subtract blank values. Using FLUO star Omega (BMG LABTECH GmbH).

### Example 4

### The Luciferase Assay—Protokol

### Day 0:

10000 TZM-bl cells are seeded in a total volume of 200 μl media (DMEM containing 10% FCS, 1% pen/strep all from Invitrogen) in a 96 well plate (Nunc).

### Day 1:

The cell medium is removed and replaced with either 100 μl fresh media or media containing different amounts of supernatants containing the bivalent inhibitor or recombinant sCD4 (R&D systems).

Different amounts of replication competent HIV virus are incubated with various amounts of supernatants containing the bivalent inhibitors or recombinant sCD4 (to the indicated final concentrations and the volume of 100 μl) for 30 min at 37° C.

The inactivated virus is subsequently added to the cells to the final volume of 200 μl.

### Day 4:

The medium is removed from the wells and 90 μl of DMEM+0.5% NP40 is added to each well and incubated for 45 min at room temperature in order to lyse the cells and inactivate any remaining virus.

90 μl of Luciferin (britelite plus Perkin Elmer cat no: 6016761) is added to each well in order to start the chemo-luminescence reaction.

150 μl of the liquid is removed from each well and the luminescence is measured in a FLUO star Omega (BMG LABYECH Gmbh).

### Example 5

### Detection of Human CD4 Elisa

### Day 1:

1×96 well plate (Nunc) is coated with 5 pg/ml Monoclonal Anti human antibody (R & D Systems Cat no: MAB 3791) in PBS (Lonza BE17-516F).

100 μl/well incubate overnight at 4° C.

### Day 2:

Wash the plate 1 time with PBS.

Block non-specific binding by adding 300 μl/well “full well” of blocking buffer: 0.5% BSA (Sigma A 8022) in PBS.

Incubate at room temperature for 1-2 hours.

Wash the plate 2 times with PBS containing 0.05% Tween-20 (Sigma p1379).

Load samples & standards 100 μl/well. Incubate overnight at 4° C.

Samples: dilutions of 5, 25 and 100 times of the CD4 containing supernatants in PBS.

Standards: Recombinant Human CD4 (R & D systems Cat no: 514CD, Stock 50 μg/ml in sterile PBS 0.1% BSA).

Dilute the standard to 1000000 pg/ml (50×) in PBS 0.1% BSA and then make serial 2 fold dilutions to 976,56 pg/ml.

### Day 3:

Wash the plate 3 times in PBS 0.05% Tween-20.

Add secondary antibody solution which 100× dilution of: R & D Systems Cat no: BAF 379 Stock 50 pg/ml in sterile Tris buffered saline pH 7.3 (20 mM Trizma base 150 mM NaCl) containing 0.1% BSA in PBS containing 0.25% BSA 10% lamb serum 0.05% Tween-20. The secondary antibody is added (100 μl/well).

Incubate at least 2 hours at room temperature.

Wash the plate 5 times in PBS 0.05% Tween-20.

Dilute the Streptavidin HRP 1:1000 in PBS 0.25% BSA 0.05% Tween-20 Add 100 μl/well of diluted Streptavidin HRP cytoset in PBS (Biosource Cat no:CHC 2203 part no: 41.000.03). Incubate 30 minutes at room temperature.

The plate is washed 4 times in PBS 0.05% Tween-20.

Add 100 μl/well TMB X-tra Ready to use substrate (KEM EN TEC Diagnostics Cat no: 4800L)

Incubate 5-10 minute—keep in dark! (until it turns blue)

Stop colour development by adding 100μ/well 0.2M H₂SO₄.

Read the optical density (OD) for each well with 405 nm and 650 nm as reference subtract blank values. Using FLUO star Omega (BMG LABTECH GmbH).

### Example 6

### Protocol for Obtaining the Bivalent Molecules of the Present Invention

293T cells are seeded (7×10⁴ cells/cm2) in a T80 bottle. The cells are transfected with 9 ug of the expression plasmid for the bivalent molecules and 1 ug of an egfp expression plasmid in order to facilitate visual estimation of the transfection efficiency. 24 h later the medium is renewed (DMEM containing 10% FCS). 48 h posttransfection the supernatant is collected and filtered using 0.22μ filters, aliquoted and frozen for later use. The concentration of the bivalent molecule is measured using ELISA.

### Example 7

### Viruses

It is within the scope of the present invention that the bivalent molecules of the present invention may effective against any virus that have a type 1 fusion mechanism, belonging to the main groups of Orthomyxoviridae, Paramyxoviridae, Retroviridae, Filoviridae and Coronaviridae.

Below is a non-limiting list of specific viruses which the bivalent molecules of the present invention may be effective against. Further it is within the scope of the present invention that the second part of the bivalent molecules may derived from any virus as listed here below.

### Orthomyxoviridae

### Paramyxoviridae

### Retroviridae

### Filoviridae

### Coronaviridae

### Lentivirus

Bovine lentivirus group
   Bovine immunodeficiency virus
      Bovine immunodeficiency virus FL112
      Bovine immunodeficiency virus FL491
      Bovine immunodeficiency virus OK
      Bovine immunodeficiency virus R29
      Jembrana disease virus
Equine lentivirus group
   Equine infectious anemia virus
      Equine infectious anemia virus (CLONE 1369)
      Equine infectious anemia virus (clone CL22)
      Equine infectious anemia virus (CLONE P3.2-1)
      Equine infectious anemia virus (CLONE P3.2-2)
      Equine infectious anemia virus (CLONE P3.2-3)
      Equine infectious anemia virus (CLONE P3.2-5)
      Equine infectious anemia virus (ISOLATE WYOMING)
      Equine infectious anemia virus (STRAIN WSU5)
Feline lentivirus group
   Feline immunodeficiency virus
      Feline immunodeficiency virus (isolate Petaluma)
      Feline immunodeficiency virus (isolate San Diego)
      Feline immunodeficiency virus (isolate TM2)
      Feline immunodeficiency virus (isolate wo)
      Feline immunodeficiency virus (strain UK2)
      Feline immunodeficiency virus (strain UK8)
      Feline immunodeficiency virus (strain UT-113)
      Lion lentivirus
      Panther lentivirus
   Puma lentivirus
      Puma lentivirus 14
      Puma lentivirus 21
Ovine/caprine lentivirus group
   Caprine arthritis encephalitis virus G63
   Caprine arthritis encephalitis virus Ov496
   Caprine arthritis encephalitis virus Roccaverano
   Caprine arthritis encephalitis virus strain Cork
   Visna/Maedi virus
      Small ruminant Lentivirus CA-Ireland
      Visna/maedi virus 1514
      Visna/maedi virus EV1
      Visna/maedi virus EV1 KV1772
      Visna/maedi virus SA-OMVV
   unclassified Ovinelcaprine lentivirus
      Caprine lentivirus
      Ovine progressive pneumonia virus
      Small ruminant lentivirus
Primate lentivirus group
   Human immunodeficiency virus
   Human immunodeficiency virus 1
      HIV-1 circulating recombinant forms
      HIV-1 group M
      HIV-1 group N
      HIV-1 group O
      HIV-1 unknown group
      Human immunodeficiency virus 3
   Human immunodeficiency virus 2
      HIV-2 subtype A
      HIV-2 subtype B
      Human immunodeficiency virus type 2 (isolate 7312A)
      Human immunodeficiency virus type 2 (ISOLATE CAM2)
      Human immunodeficiency virus type 2 (ISOLATE D194)
      Human immunodeficiency virus type 2 (ISOLATE GHANA-1)
      Human immunodeficiency virus type 2 (isolate KR)
      Human immunodeficiency virus type 2 (ISOLATE NIH-Z)
      Human immunodeficiency virus type 2 (ISOLATE SBLISY)
   Simian immunodeficiency virus
      Human T-cell lymphotropic virus type 4
      Simian immunodeficiency virus—agm
      Simian immunodeficiency virus—cpz
      Simian immunodeficiency virus—mac
      Simian immunodeficiency virus—mnd
      Simian immunodeficiency virus—mon
      Simian immunodeficiency virus—olc
      Simian immunodeficiency virus—sm
      Simian immunodeficiency virus—stm
      Simian immunodeficiency virus—wrc
      Simian immunodeficiency virus Qu
      Simian immunodeficiency virus SIVsun
   Simian-Human immunodeficiency virus
unclassified Lentivirus
   Brazilian caprine lentivirus
   Grey mouse lemur immunodeficiency virus 1
   HIV-like human cancer virus
   Ovine lentivirus

### Example 8

### Comparative Analyses of Inhibitor Properties

### Introduction

The bivalent inhibitor has been designed using extensive knowledge of the HIV envelope fusion mechanism and the structure of the envelope protein. We have fused the extracellular domain of CD4 (the primary receptor for HIV) to a region from HIV gp41 including the sequence of a licensed fusion inhibitor −T20. This composite design creates a molecule that can bind to and inactivate the envelope protein of HIV in an active fashion. Without being bound by theory, FIG. 15 depicts the likely mode of action of the bivalent inhibitor.

The bivalent inhibitor used in the listed experiments is produced in 293T cells by transient transfection. Supernatant from the transfected cells is harvested and the concentration of the active protein is measured using ELISA. Supernatant from mock transfected 293T cells is used as control.

We have established the superior potency of the bivalent inhibitors using HIV subtypes HXB2, JR-CSF and 89.6 in both single round infection assays as well as inhibition of replication in both T-cell cultures and human PBMCs. In all cases it is evident that the bivalent inhibitors are significantly more potent than other anti-HIV drugs at low concentrations.

### Dose Dependent Effect on Proliferation of HIV in T-Cell Cultures

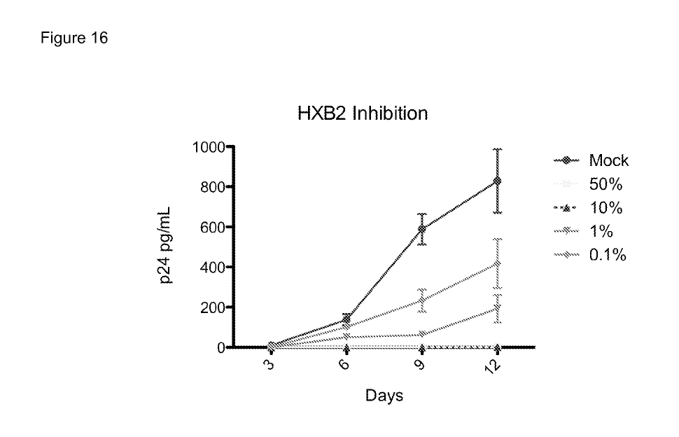

FIG. 16 shows the dose dependency of the effect of the bivalent inhibitor on proliferation of HIV-1 strain HXB2 in a T cell line (Jurkat). Briefly, HXB2 virus is incubated with different amounts of supernatants from transfected 293T cells 30 min prior to addition to Jurkat cells in a 96 well format. The cells grow in medium containing the bivalent inhibitor at the given percentages. Samples are taken out at the given time post infection and p24 amounts are measured by ELISA.

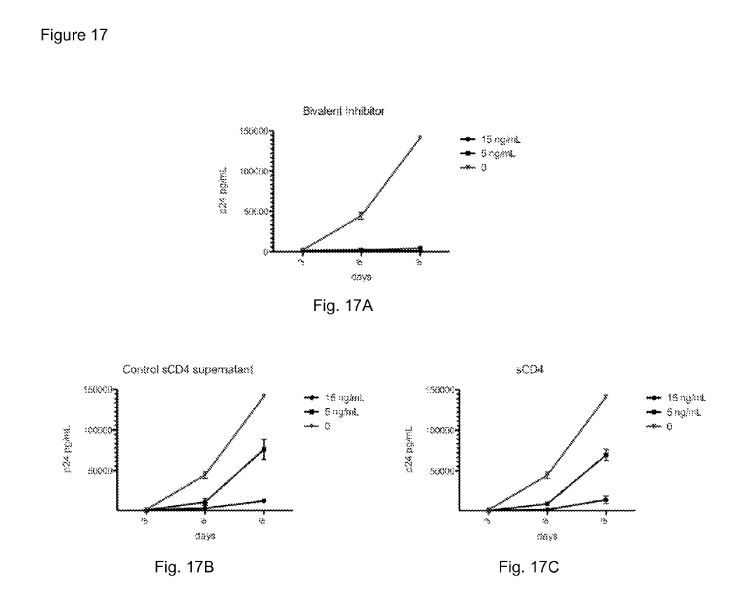

FIG. 17 shows the comparison between the different doses of the bivalent inhibitor and soluble CD4 on proliferation of the HXB2 virus in Jurkat cells. Two different sCD4 preparations are used as controls. “Control sCD4 supernatant” is produced under the same conditions as the bivalent inhibitor, while sCD4 is a commercially available preparation. It is clear that the bivalent inhibitor is a significantly more potent anti HIV compound. The experiment was performed as described above. Please notice that the molecular weight of the bivalent inhibitor is slightly more than that of sCD4.

Effect on Proliferation of Virus in Human Peripheral Blood Mononuclear Cells (PBMCs)

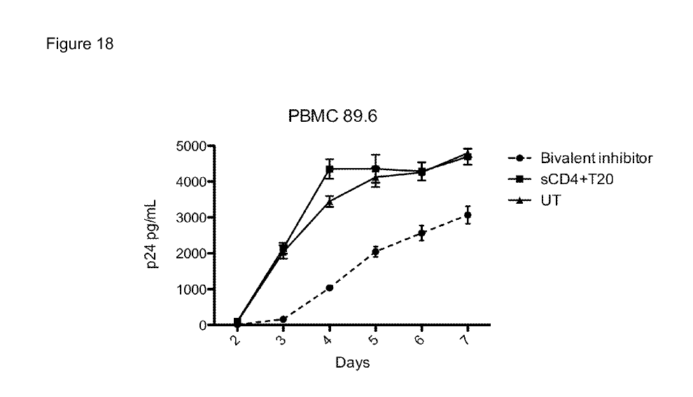

FIG. 18 shows a direct comparison of the bivalent inhibitor to the combined effect of sCD4 and T20, the only compounds that inhibit HIV by acting on the Envelope protein. As evident, at comparable concentrations, only the bivalent inhibitor can significantly inhibit HIV spreading in the culture.

A single round infection assay using TZM-bl cells, which are HeLa derivatives containing a TAT dependent luciferase cassette is used. Infection with HIV and the subsequent expression of TAT initiates high levels of the luciferase reporter gene. Thus, within the linear curve of the viral dosage the relative light intensity, produced by the luciferase enzyme, is proportional to viral titers.

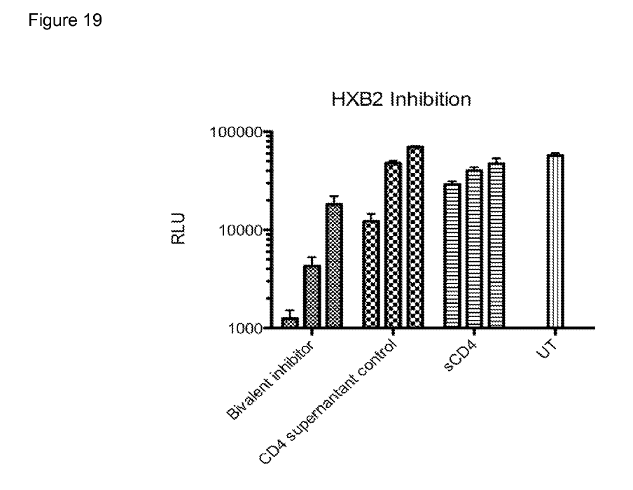

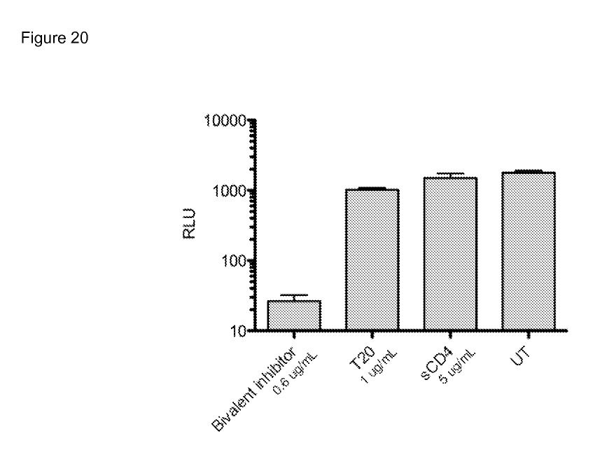

In direct comparison with several different classes anti HIV drugs, we find that the bivalent inhibitor is significantly more potent at low concentrations. The data presented in FIGS. 19 and 20 indicates that the bivalent inhibitor is much more potent in neutralizing the different HIV strains in direct comparison with sCD4 (and T20 in FIG. 20), the only known compounds targeting viral entry through interaction with the Envelope protein.

The two viral strains used in these experiments represent the extremes in susceptibility to neutralization by sCD4. HXB2 is one of the most sensitive strains, while JR-CSF is completely impervious to neutralization by sCD4.

The viral strain JR-CSF is a strain isolated from the cerebrospinal fluids and it displays limited infection dependency on CD4. Thus, it is very hard to neutralize by addition of soluble sCD4. FIG. 20 shows the effect of the bivalent inhibitor on this virus as measured in the aforementioned TZM-bl assay.

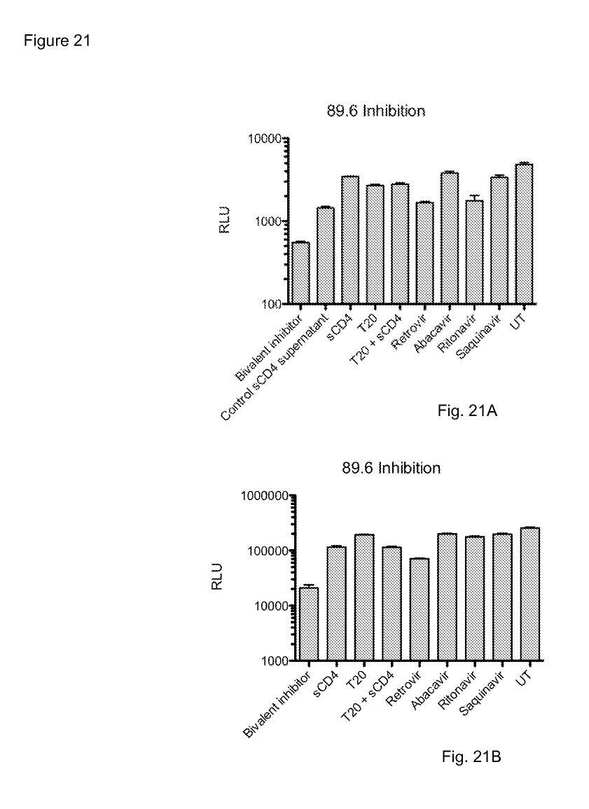

A direct comparison of the bivalent inhibitor with several anti-HIV drugs indicates its high potency in very low concentrations. FIG. 21 shows the results of two independent experiments using HIV subtype 89.6.

### Mechanism of Action

The envelope protein on viral particles are in a meta-stable, high energy conformation. Fusion of membranes is only possible using the energy stored in this protein. If the conformational changes that lead to the stable post fusion conformation are triggered prematurely, the potential energy of the envelope protein is wasted and it becomes inactivated. We believe that the bivalent inhibitor neutralizes the virus prior to its interaction with the target cell by facilitating these conformational changes and make the envelope protein “fire” before it is close to the target membrane, thus neutralizing its fusion potential (see FIG. 15).

This unique mechanism of action predicts that the longer the virus is incubated with the inhibitor, the more potent the inhibitor will seem to be. In comparison, other known anti-HIV drugs interfere with a step in the life cycle subsequent to the binding of the virus to its target cells, and thus incubation of the virus stock with these drugs, in absence of target cells, is not expected to affect their potency. sCD4 has been reported to induce shedding of the gp160 in some strains, but we do not find it to show more potency upon incubation with 89.6 virions.

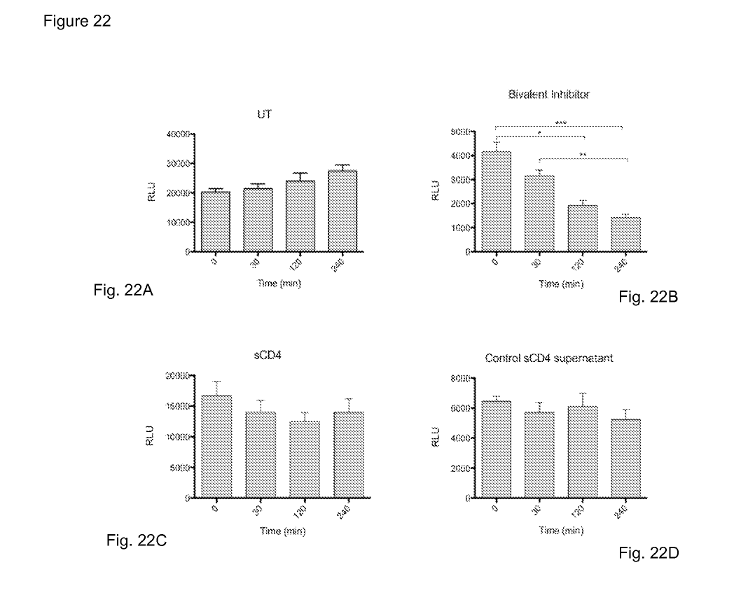

To investigate, whether the bivalent inhibitors show an active enzymatic inactivation of HIV virions, we tested the time-dependency of the neutralization by the bivalent inhibitor and compared it to sCD4 and T20 using the TZM-bl assay (FIG. 22). Virus and the drugs were mixed together and incubated for up to four hours prior to the seeding on target cells. The infectivity was measured as the activity of the luciferase enzyme in the cell lysates. The bivalent inhibitor clearly shows a significant time dependent increase in potency unlike sCD4.

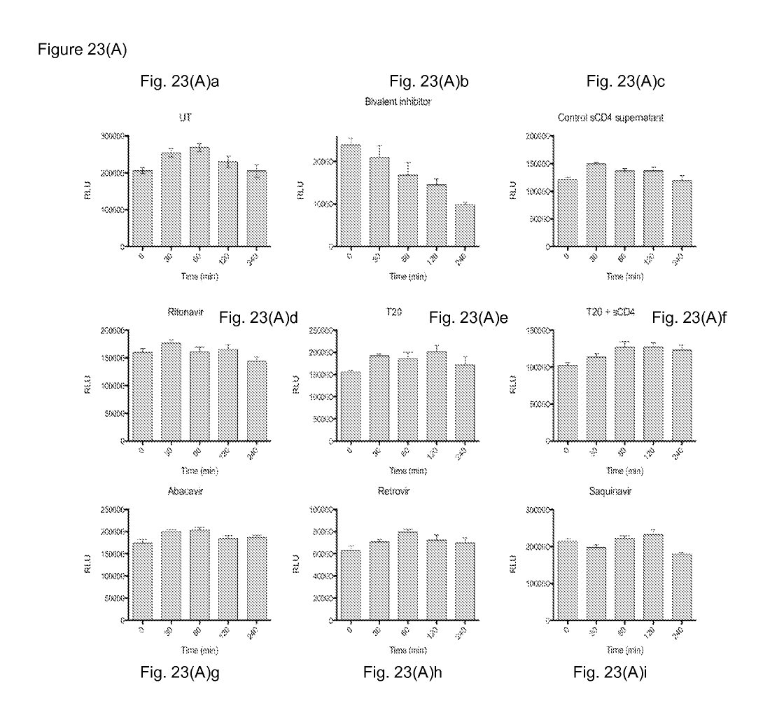

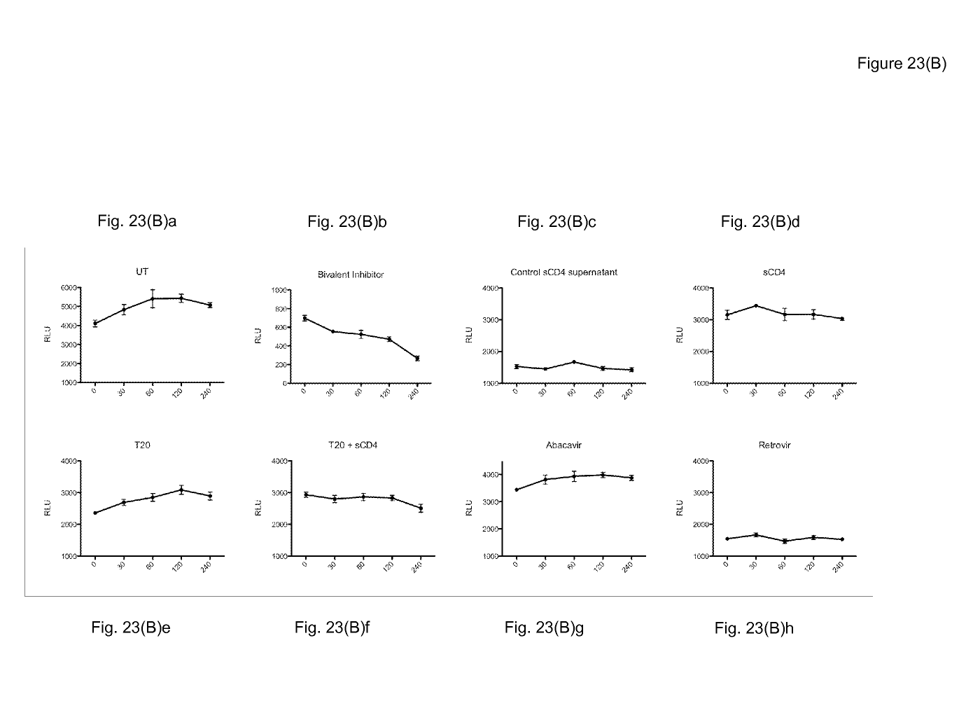

The set of data in FIG. 23 has been expanded to include the time dependency of the anti viral effect of different drug classes. FIG. 23 shows the results of two of the four independent experiments performed.

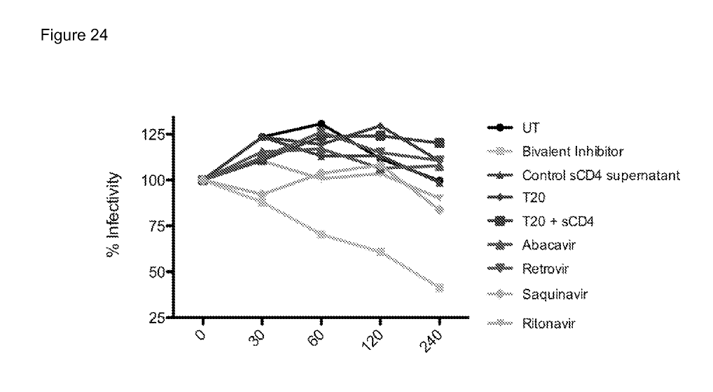

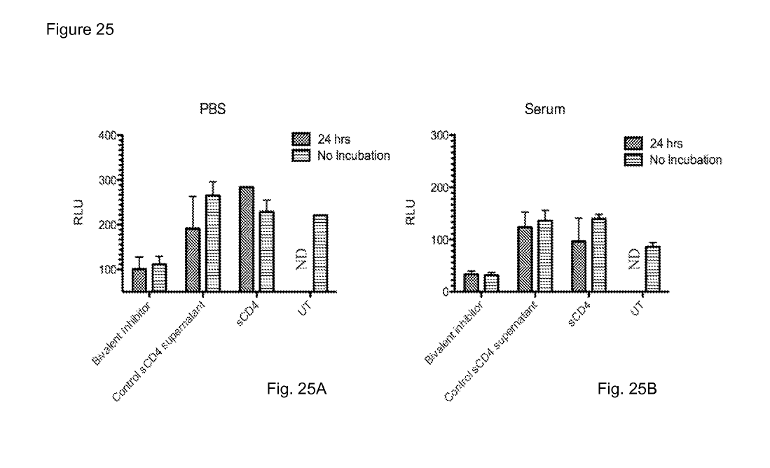

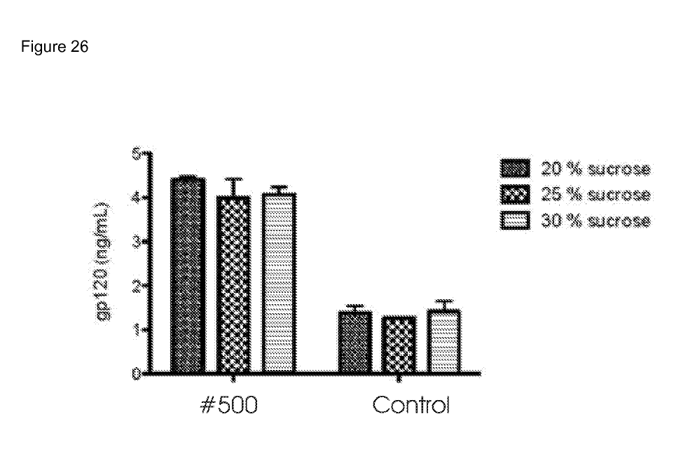

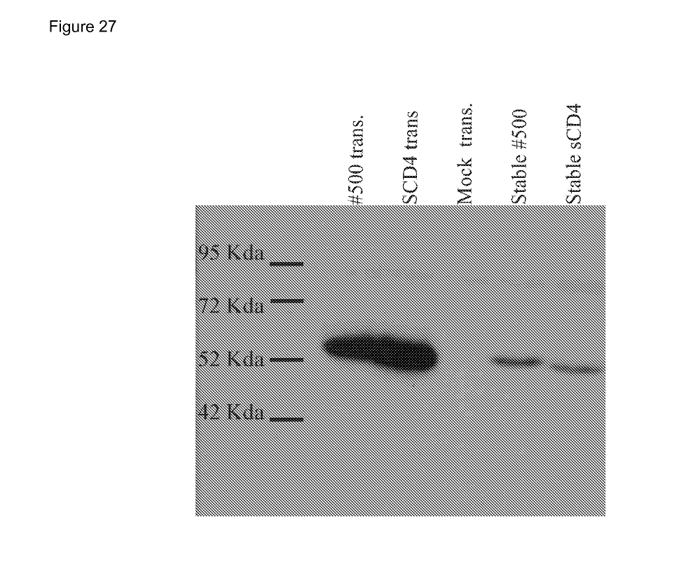

The time dependency data from FIG. 23 is represented in as the ratio of the infectivity at a given time to time zero in FIG. 24, thus illustrating the unique time dependency of the bivalent inhibitor confirming an active mechanism of neutralization rather than a simple passive binding to virions.

### Stability

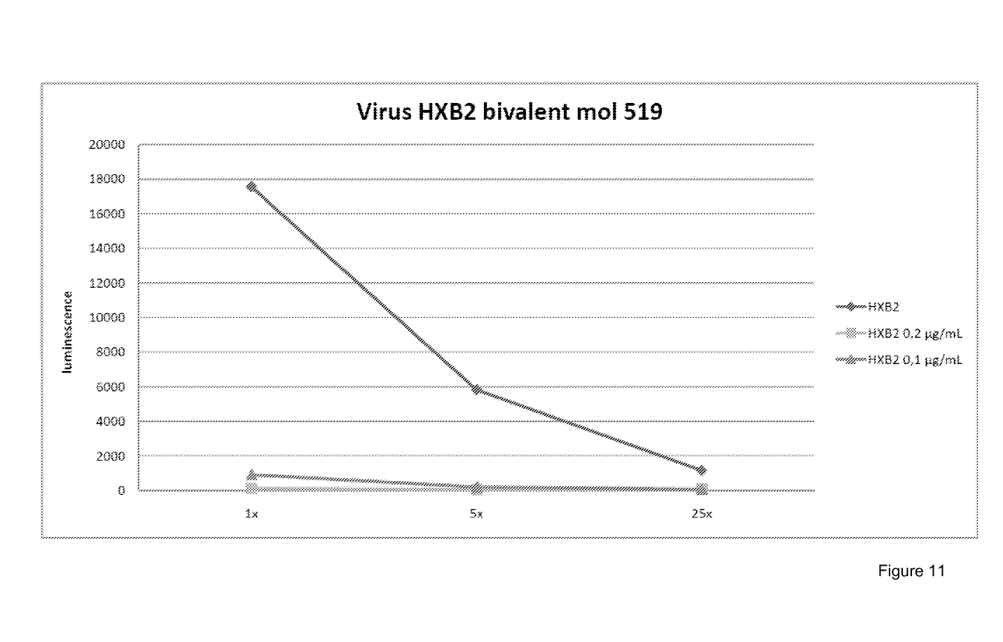

In order to evaluate the stability of the bivalent inhibitor, we measured the anti-viral activity of the bivalent inhibitor after 24 h of incubation in either human serum or PBS at 37° C. As seen in FIG. 11, incubation in neither serum nor PBS has any significant effect on the anti-viral activity of the compound.

### CONCLUSIONS

The data presented here establishes the bivalent inhibitor as a very potent anti-HIV compound. Although derived from sCD4, the bivalent molecule can inactivate HIV isolates that are completely resistant to neutralization by sCD4.

Furthermore, the data suggest that the bivalent molecule is the first representative of a new class of molecules that inactivate the virus independently of (and prior to) its interaction with the target cells, in an active fashion reminiscent of an enzyme. Furthermore the compound is stable at least for 24 h at 37° C. in human serum, which suggests its suitability for use as an anti-HIV medicine in humans.

### Sequences

Bivalent molecules and fragments, amino acid sequences
SEQ ID NO: 1
(#500)
MNRGVPFRHLLLVLQLALLPAATQGKKVVLGKKGDTVELTCTASQKKSIQFHWKNSNQIKILGN
QGSFLTKGPSKLNDRADSRRSLWDQGNFPLIIKNLKIEDSDTYICEVEDQKEEVQLLVFGLTAN
SDTHLLQGQSLTLTLESPPGSSPSVQCRSPRGKNIQGGKTLSVSQLELQDSGTWTCTVLQNQKK
VEFKIDIVVLAFQKASSIVYKKEGEQVEFSFPLAFTVEKLTGSGELWWQAERASSSKSWITFDL
KNKEVSVKRVTQDPKLQMGKKLPLHLTLPQALPQYAGSGNLTLALEAKTGKLHQEVNLVVMRAT
QLQKNLTCEVWGPTSPKLMLSLKLENKEAKVSKREKAVWVLNPEAGMWQCLLSDSGQVLLESNI
KVLPTWSTPVSSGGSGGSGGSGGSGGSSGEWDREINNYTSLIHSLIEESQNQQEKNEQELLELD
KWASLWNWF
SEQ ID NO: 2
(#521)
MNRGVPFRHLLLVLQLALLPAATQGKKVVLGKKGDTVELTCTASQKKSIQFHWKNSNQIKILGN
QGSFLTKGPSKLNDRADSRRSLWDQGNFPLIIKNLKIEDSDTYICEVEDQKEEVQLLVFGLTAN
SDTHLLQGQSLTLTLESPPGSSPSVQCRSPRGKNIQGGKTLSVSQLELQDSGTWTCTVLQNQKK
VEFKIDIVVLAFQKASSIVYKKEGEQVEFSFPLAFTVEKLTGSGELWWQAERASSSKSWITFDL
KNKEVSVKRVTQDPKLQMGKKLPLHLTLPQALPQYAGSGNLTLALEAKTGKLHQEVNLVVMRAT
QLQKNLTCEVWGPTSPKLMLSLKLENKEAKVSKREKAVWVLNPEAGMWQCLLSDSGQVLLESNI
KVLPTWSTPVSSGGSGGSGGSGGSGGSSGSGIVQQQNNLLRAIEAQQHLLQLTVWGIKQLQARI
L
SEQ ID NO: 3
(#517)
MNRGVPFRHLLLVLQLALLPAATQGKKVVLGKKGDTVELTCTASQKKSIQFHWKNSNQIKILGN
QGSFLTKGPSKLNDRADSRRSLWDQGNFPLIIKNLKIEDSDTYICEVEDQKEEVQLLVFGLTAN
SDTHLLQGQSLTLTLESPPGSSPSVQCRSPRGKNIQGGKTLSVSQLELQDSGTWTCTVLQNQKK
VEFKIDIVVLAFQKASSIVYKKEGEQVEFSFPLAFTVEKLTGSGELWWQAERASSSKSWITFDL
KNKEVSVKRVTQDPKLQMGKKLPLHLTLPQALPQYAGSGNLTLALEAKTGKLHQEVNLVVMRAT
QLQKNLTCEVWGPTSPKLMLSLKLENKEAKVSKREKAVWVLNPEAGMWQCLLSDSGQVLLESNI
KVLPTWSTPVSSGGSGGSGGSGGSGGSSGLQARILAVERYLKDQQLLGIWG
SEQ ID NO: 4
(#520)
MNRGVPFRHLLLVLQLALLPAATQGKKVVLGKKGDTVELTCTASQKKSIQFHWKNSNQIKILGN
QGSFLTKGPSKLNDRADSRRSLWDQGNFPLIIKNLKIEDSDTYICEVEDQKEEVQLLVFGLTAN
SDTHLLQGQSLTLTLESPPGSSPSVQCRSPRGKNIQGGKTLSVSQLELQDSGTWTCTVLQNQKK
VEFKIDIVVLAFQKASSIVYKKEGEQVEFSFPLAFTVEKLTGSGELWWQAERASSSKSWITFDL
KNKEVSVKRVTQDPKLQMGKKLPLHLTLPQALPQYAGSGNLTLALEAKTGKLHQEVNLVVMRAT
QLQKNLTCEVWGPTSPKLMLSLKLENKEAKVSKREKAVWVLNPEAGMWQCLLSDSGQVLLESNI
KVLPTWSTPVSSGGSGGSGGSGGSGGSSGLQARILAVERYLKDQQLLGIWGCSGKLICTTAVPW
NASWSNKSLEQIWNHTT
SEQ ID NO: 5
(#518)
MNRGVPFRHLLLVLQLALLPAATQGKKVVLGKKGDTVELTCTASQKKSIQFHWKNSNQIKILGN
QGSFLTKGPSKLNDRADSRRSLWDQGNFPLIIKNLKIEDSDTYICEVEDQKEEVQLLVFGLTAN
SDTHLLQGQSLTLTLESPPGSSPSVQCRSPRGKNIQGGKTLSVSQLELQDSGTWTCTVLQNQKK
VEFKIDIVVLAFQKASSIVYKKEGEQVEFSFPLAFTVEKLTGSGELWWQAERASSSKSWITFDL
KNKEVSVKRVTQDPKLQMGKKLPLHLTLPQALPQYAGSGNLTLALEAKTGKLHQEVNLVVMRAT
QLQKNLTCEVWGPTSPKLMLSLKLENKEAKVSKREKAVWVLNPEAGMWQCLLSDSGQVLLESNI
KVLPTWSTPVSSGGSGGSGGSGGSGGSSGLQARILAVERYLKDQQLLGIWGSSGKLISTTAVPW
NASWSNKSLEQIWNHTT
SEQ ID NO: 6
(#519)
MNRGVPFRHLLLVLQLALLPAATQGKKVVLGKKGDTVELTCTASQKKSIQFHWKNSNQIKILGN
QGSFLTKGPSKLNDRADSRRSLWDQGNFPLIIKNLKIEDSDTYICEVEDQKEEVQLLVFGLTAN
SDTHLLQGQSLTLTLESPPGSSPSVQCRSPRGKNIQGGKTLSVSQLELQDSGTWTCTVLQNQKK
VEFKIDIVVLAFQKASSIVYKKEGEQVEFSFPLAFTVEKLTGSGELWWQAERASSSKSWITFDL
KNKEVSVKRVTQDPKLQMGKKLPLHLTLPQALPQYAGSGNLTLALEAKTGKLHQEVNLVVMRAT
QLQKNLTCEVWGPTSPKLMLSLKLENKEAKVSKREKAVWVLNPEAGMWQCLLSDSGQVLLESNI
KVLPTWSTPVSSGGSGGSGGSGGSGGSSGTTAVPWNASWSNKSLEQIWNHTT
SEQ ID NO: 7
(#538)
MNRGVPFRHLLLVLQLALLPAATQGKKVHHHHHHKVVLGKKGDTVELTCTASQKKSIQFHWKNS
NQIKILGNQGSFLTKGPSKLNDRADSRRSLWDQGNFPLIIKNLKIEDSDTYICEVEDQKEEVQL
LVFGLTANSDTHLLQGQSLTLTLESPPGSSPSVQCRSPRGKNIQGGKTLSVSQLELQDSGTWTC
TVLQNQKKVEFKIDIVVLAFQKASSIVYKKEGEQVEFSFPLAFTVEKLTGSGELWWQAERASSS
KSWITFDLKNKEVSVKRVTQDPKLQMGKKLPLHLTLPQALPQYAGSGNLTLALEAKTGKLHQEV
NLVVMRATQLQKNLTCEVWGPTSPKLMLSLKLENKEAKVSKREKAVWVLNPEAGMWQCLLSDSG
QVLLESNIKVLPTWSTPVSSGGSGGSGGSGGSGGSSGTTAVPWNASWSNKSLEQIWNHTT
SEQ ID NO: 8
(#539)
MNRGVPFRHLLLVLQLALLPAATQGKKVHHHHHHKVVLGKKGDTVELTCTASQKKSIQFHWKNS
NQIKILGNQGSFLTKGPSKLNDRADSRRSLWDQGNFPLIIKNLKIEDSDTYICEVEDQKEEVQL
LVFGLTANSDTHLLQGQSLTLTLESPPGSSPSVQCRSPRGKNIQGGKTLSVSQLELQDSGTWTC
TVLQNQKKVEFKIDIVVLAFQKASSIVYKKEGEQVEFSFPLAFTVEKLTGSGELWWQAERASSS
KSWITFDLKNKEVSVKRVTQDPKLQMGKKLPLHLTLPQALPQYAGSGNLTLALEAKTGKLHQEV
NLVVMRATQLQKNLTCEVWGPTSPKLMLSLKLENKEAKVSKREKAVWVLNPEAGMWQCLLSDSG
QVLLESNIKVLPTWSTPVSSGGSGGSGGSGGSGGSSGEWDREINNYTSLIHSLIEESQNQQEKN
EQELLELDKWASLWNWF
SEQ ID NO: 9
(sCD4)
MNRGVPFRHLLLVLQLALLPAATQGKKVVLGKKGDTVELTCTASQKKSIQFHWKNSNQIKILGN
QGSFLTKGPSKLNDRADSRRSLWDQGNFPLIIKNLKIEDSDTYICEVEDQKEEVQLLVFGLTAN
SDTHLLQGQSLTLTLESPPGSSPSVQCRSPRGKNIQGGKTLSVSQLELQDSGTWTCTVLQNQKK
VEFKIDIVVLAFQKASSIVYKKEGEQVEFSFPLAFTVEKLTGSGELWWQAERASSSKSWITFDL
KNKEVSVKRVTQDPKLQMGKKLPLHLTLPQALPQYAGSGNLTLALEAKTGKLHQEVNLVVMRAT
QLQKNLTCEVWGPTSPKLMLSLKLENKEAKVSKREKAVWVLNPEAGMWQCLLSDSGQVLLESNI
KVLPTWSTPV
SEQ ID NO: 10
(sCD4-6H)
MNRGVPFRHLLLVLQLALLPAATQGKKVHHHHHHKVVLGKKGDTVELTCTASQKKSIQFHWKNS
NQIKILGNQGSFLTKGPSKLNDRADSRRSLWDQGNFPLIIKNLKIEDSDTYICEVEDQKEEVQL
LVFGLTANSDTHLLQGQSLTLTLESPPGSSPSVQCRSPRGKNIQGGKTLSVSQLELQDSGTWTC
TVLQNQKKVEFKIDIVVLAFQKASSIVYKKEGEQVEFSFPLAFTVEKLTGSGELWWQAERASSS
KSWITFDLKNKEVSVKRVTQDPKLQMGKKLPLHLTLPQALPQYAGSGNLTLALEAKTGKLHQEV
NLVVMRATQLQKNLTCEVWGPTSPKLMLSLKLENKEAKVSKREKAVWVLNPEAGMWQCLLSDSG
QVLLESNIKVLPTWSTPV
SEQ ID NO: 215
(first part, Chimpanzee CD4)
MNRGVPFRHLLLVLQLALLPAATQGKKVVLGKKGDTVELTCTASQKKSIQFHWKNSNQTKILGN
QGSFLTKGPSKLNDRVDSRRSLWDQGNFTLIIKNLKIEDSDTYICEVGDQKEEVQLLVFGLTAN
SDTHLLQGQSLTLTLESPPGSSPSVQCRSPRGKNIQGGKTLSVSQLELQDSGTWTCTVLQNQKK
VEFKIDIVVLAFQKASSIVYKKEGEQVEFSFPLAFTVEKLTGSGELWWQAERASSSKSWITFDL
KNKEVSVKRVTQDPKLQMGKKLPLHLTLPQALPQYAGSGNLTLALEAKTGKLHQEVNLVVMRAT
QLQKNLTCEVWGPTSPKLMLSLKLENKEAKVSKREKAVWVLNPEAGMWQCLLSDSGQVLLESNI
KVLPTWSTPV
SEQ ID NO: 216
(Chim-sCD4-T20, comprising cCD4 with point mutation found in
Chimpanzee(underlined))
MNRGVPFRHLLLVLQLALLPAATQGKKVVLGKKGDTVELTCTASQKKSIQFHWKNSNQIKILGN
QGSFLTKGPSKLNDRADSRRSLWDQGNFPLIIKNLKIEDSDTYICEVGDQKEEVQLLVFGLTAS
DTHLLQGQSLTLTLESPPGSSPSVQCRSPRGKNIQGGKTLSVSQLELQDSGTWTCTVLQNQKKV
EFKIDIVVLAFQKASSIVYKKEGEQVEFSFPLAFTVEKLTGSGELWWQAERASSSKSWITFDKN
KEVSVKRVTQDPKLQMGKKLPLHLTLPQALPQYAGSGNLTLALEAKTGKLHQEVNLVVMRATQL
QKNLTCEVWGPTSPKLMLSLKLENKEAKVSKREKAVWVLNPEAGMWQCLLSDSGQVLLESNKVL
PTWSTPVSSGGSGGSGGSGGSGGSSGEWDREINNYTSLIHSLIEESQNQQEKNEQELLELDKWA
SLWNWF
SEQ ID NO: 217
(cMyc-His6-TEV sCD4-T20-IRES-Puro)
MRAWIFFLLCLAGRALAASEQKLISEEDLNMHTGHHHHHHGENLYFQGKKVVLGKKGDTVELTCTASQKK
SIQFHWKNSNQIKILGNQGSFLTKGPSKLNDRADSRRSLWDQGNFPLIIKNLKIEDSYICEVEDQKEEVQ
LLVFGLTANSDTHLLQGQSLTLTLESPPGSSPSVQCRSPRGKNIQGGKTLSVSQLELQDSGTWTCTVLQN
QKKVEFKIDIVVLAFQKASSIVYKKEGEQVEFSFPLAFTVEKLGGELWWQAERASSSKSWITFDLKNKEV
SVKRVTQDPKLQMGKKLPLHLTLPQALPQYAGSGNLTLALEAKTGKLHQEVNLVVMRATQLQKNLTCEVW
GPTSPKLMLSLKLENKEAKVSKREKAVWVNPAGMWQCLLSDSGQVLLESNIKVLPTWSTPVSSGGSGGSG
GSGGSGGSSGEWDREINNYTSLIHSLIEESQNQQEKNEQELLELDKWASLWNWF
SEQ ID NO: 218
(sCD4 T20 Sifurvitide (Sifurvitide underlined))
MNRGVPFRHLLLVLQLALLPAATQGKKVVLGKKGDTVELTCTASQKKSIQFHWKNSNQI
KILGNQGSFLTKGPSKLNDRADSRRSLWDQGNFPLIIKNLKIEDSDTYICEVEDQKEEV
QLLVFGLTADTHLLQGQSLTLTLESPPGSSPSVQCRSPRGKNIQGGKTLSVSQLELQDS
GTWTCTVLQNQKKVEFKIDIVVLAFQKASSIVYKKEGEQVEFSFPLAFTVEKLTGSGEL
WWQAERASSSKSWITFDKKEVSVKRVTQDPKLQMGKKLPLHLTLPQALPQYAGSGNLTL
ALEAKTGKLHQEVNLVVMRATQLQKNLTCEVWGPTSPKLMLSLKLENKEAKVSKREKAV
WVLNPEAGMWQCLLSDSGQVLLESNKVLPTWSTPVSSGGSGGSGGSGGSGGSSGSWETW
EREIENYTRQIYRILEESQEQQDRNERDLLE
SEQ ID NO: 219
(sCD4-DSL20 (Alternative sequence of the helix
that interacts with gp120))
MNRGVPFRHLLLVLQLALLPAATQGKKVVLGKKGDTVELTCTASQKKSIQFHWKNSNQI
KILGNQGSFLTKGPSKLNDRADSRRSLWDQGNFPLIIKNLKIEDSDTYICEVEDQKEEV
QLLVFGLTADTHLLQGQSLTLTLESPPGSSPSVQCRSPRGKNIQGGKTLSVSQLELQDS
GTWTCTVLQNQKKVEFKIDIVVLAFQKASSIVYKKEGEQVEFSFPLAFTVEKLTGSGEL
WWQAERASSSKSWITFDKKEVSVKRVTQDPKLQMGKKLPLHLTLPQALPQYAGSGNLTL
ALEAKTGKLHQEVNLVVMRATQLQKNLTCEVWGPTSPKLMLSLKLENKEAKVSKREKAV
WVLNPEAGMWQCLLSDSGQVLLESNKVLPTWSTPVSSGGSGGSGGSGGSGGSSGERYLK
DQQLLGIWGCSGKLICTTAVPWNASWSNKSLEQIWNHTTWMEWDREIN
SEQ ID NO: 220
(sCD4-DSL20ss (Alternative sequence of the helix
that interacts with gp120))
MNRGVPFRHLLLVLQLALLPAATQGKKVVLGKKGDTVELTCTASQKKSIQFHWKNSNQI
KILGNQGSFLTKGPSKLNDRADSRRSLWDQGNFPLIIKNLKIEDSDTYICEVEDQKEEV
QLLVFGLTADTHLLQGQSLTLTLESPPGSSPSVQCRSPRGKNIQGGKTLSVSQLELQDS
GTWTCTVLQNQKKVEFKIDIVVLAFQKASSIVYKKEGEQVEFSFPLAFTVEKLTGSGEL
WWQAERASSSKSWITFDKKEVSVKRVTQDPKLQMGKKLPLHLTLPQALPQYAGSGNLTL
ALEAKTGKLHQEVNLVVMRATQLQKNLTCEVWGPTSPKLMLSLKLENKEAKVSKREKAV
WVLNPEAGMWQCLLSDSGQVLLESNKVLPTWSTPVSSGGSGGSGGSGGSGGSSGERYLK
DQQLLGIWGSSGKLISTTAVPWNASWSNKSLEQIWNHTTWMEWDREIN
SEQ ID NO: 221
(sCD4-DSL49 (alternative helix sequence thought
to interact with gp120))
MNRGVPFRHLLLVLQLALLPAATQGKKVVLGKKGDTVELTCTASQKKSIQFHWKNSNQI
KILGNQGSFLTKGPSKLNDRADSRRSLWDQGNFPLIIKNLKIEDSDTYICEVEDQKEEV
QLLVFGLTADTHLLQGQSLTLTLESPPGSSPSVQCRSPRGKNIQGGKTLSVSQLELQDS
GTWTCTVLQNQKKVEFKIDIVVLAFQKASSIVYKKEGEQVEFSFPLAFTVEKLTGSGEL
WWQAERASSSKSWITFDKKEVSVKRVTQDPKLQMGKKLPLHLTLPQALPQYAGSGNLTL
ALEAKTGKLHQEVNLVVMRATQLQKNLTCEVWGPTSPKLMLSLKLENKEAKVSKREKAV
WVLNPEAGMWQCLLSDSGQVLLESNKVLPTWSTPVSSGGSGGSGGSGGSGGSSGERYLK
DQQLLGIWGCSGKLICTTAVPWNASWSNKSLEQIWNHTTWMEWDREINNYTSLIHSLIE
ESQNQQEKNEQELLELDK
SEQ ID NO: 222
(sCD4-sgg3-T20 (shorter linker))
MNRGVPFRHLLLVLQLALLPAATQGKKVVLGKKGDTVELTCTASQKKSIQFHWKNSNQI
KILGNQGSFLTKGPSKLNDRADSRRSLWDQGNFPLIIKNLKIEDSDTYICEVEDQKEEV
QLLVFGLTADTHLLQGQSLTLTLESPPGSSPSVQCRSPRGKNIQGGKTLSVSQLELQDS
GTWTCTVLQNQKKVEFKIDIVVLAFQKASSIVYKKEGEQVEFSFPLAFTVEKLTGSGEL
WWQAERASSSKSWITFDKKEVSVKRVTQDPKLQMGKKLPLHLTLPQALPQYAGSGNLTL
ALEAKTGKLHQEVNLVVMRATQLQKNLTCEVWGPTSPKLMLSLKLENKEAKVSKREKAV
WVLNPEAGMWQCLLSDSGQVLLESNKVLPTWSTPVSSGGSGGSGGSSGEWDREINNYTS
LIHSLIEESQNQQEKNEQELLELDKWASLWNWF
SEQ ID NO: 223
(sCD4-sgg7-T20 (longer linker))
MNRGVPFRHLLLVLQLALLPAATQGKKVVLGKKGDTVELTCTASQKKSIQFHWKNSNQI
KILGNQGSFLTKGPSKLNDRADSRRSLWDQGNFPLIIKNLKIEDSDTYICEVEDQKEEV
QLLVFGLTADTHLLQGQSLTLTLESPPGSSPSVQCRSPRGKNIQGGKTLSVSQLELQDS
GTWTCTVLQNQKKVEFKIDIVVLAFQKASSIVYKKEGEQVEFSFPLAFTVEKLTGSGEL
WWQAERASSSKSWITFDKKEVSVKRVTQDPKLQMGKKLPLHLTLPQALPQYAGSGNLTL
ALEAKTGKLHQEVNLVVMRATQLQKNLTCEVWGPTSPKLMLSLKLENKEAKVSKREKAV
WVLNPEAGMWQCLLSDSGQVLLESNKVLPTWSTPVSSGGSGGSGGSGGSGGSGGSGGSS
GEWDREINNYTSLIHSLIEESQNQQEKNEQELLELDKWASLWNWF
SEQ ID NO: 224
(Short sCD4-link-T20 (sCD4 containing the first
two immunoglobulin like domains))
MNRGVPFRHLLLVLQLALLPAATQGKKVVLGKKGDTVELTCTASQKKSIQFHWKNSNQI
KILGNQGSFLTKGPSKLNDRADSRRSLWDQGNFPLIIKNLKIEDSDTYICEVEDQKEEV
QLLVFGLTANSDTHLLQGQSLTLTLESPPGSSPSVQCRSPRGKNIQGGKTLSVSQLELQ
DSGTWTCTVLQNQKKVEFKIDIVVLAFQKASSGGSGGSGGSGGSGGSSGEWDREINNYT
SLIHSLIEESQNQQEKNEQELLELDKWASLWNWF
SEQ ID NO: 225
(Short sCD4-sgg7-T20 (sCD4 containing the first
two immunoglobulin like domains))
MNRGVPFRHLLLVLQLALLPAATQGKKVVLGKKGDTVELTCTASQKKSIQFHWKNSNQIKILGN
QGSFLTKGPSKLNDRADSRRSLWDQGNFPLIIKNLKIEDSDTYICEVEDQKEEVQLLVFGLTAN
SDTHLLQGQSLTLTLESPPGSSPSVQCRSPRGKNIQGGKTLSVSQLELQDSGTWTCTVLQNQKK
VEFKIDIVVLAFQKASSGGSGGSGGSGGSGGSSGEWDREINNYTSLIHSLIEESQNQQEKNEQE
LLELDKWASLWNWF
SEQ ID NO: 11
(#500, second part)
EWDREINNYTSLIHSLIEESQNQQEKNEQELLELDKWASLWNWF
SEQ ID NO: 12
(#521, second part)
SGIVQQQNNLLRAIEAQQHLLQLTVWGIKQLQARIL
SEQ ID NO: 13
(#517, second part)
LQARILAVERYLKDQQLLGIWG
SEQ ID NO: 14
(#520, second part)
LQARILAVERYLKDQQLLGIWGCSGKLICTTAVPWNASWSNKSLEQIWNHTT
SEQ ID NO: 15
(#518, second part)
LQARILAVERYLKDQQLLGIWGSSGKLISTTAVPWNASWSNKSLEQIWNHTT
SEQ ID NO: 16
(#519, second part)
TTAVPWNASWSNKSLEQIWNHTT
SEQ ID NO: 17
(#538, second part)
TTAVPWNASWSNKSLEQIWNHTT
SEQ ID NO: 18
(#539, second part)
EWDREINNYTSLIHSLIEESQNQQEKNEQELLELDKWASLWNWF
SEQ ID NO: 19
(Linker)
SSGGSGGSGGSGGSGGSSG
HIV-1 envelope derived triple-helices
SEQ ID NO: 20
A1.AU.x.PS1044_Day0.DQ676872
STMGAASITLTVQARQLLSGIVQQQSNLLRAIEAQQHMLKLTVWGIKQLQARVLALERYLKDQQ
LLGIWGCSGKLICTTNVPWNNTWSNKNKSEIWDKMTWLQWDKEISNYTQIIYNLIEESQTQQEI
NEQELLALDKWANLWNWFDISQWLWYIK
SEQ ID NO: 21
A1.KE.94.Q23_17.AF004885
STMGATSITLTVQARQLLSGIVQQQNNLLRAIEAQQHLLKLTVWGIKQLQARVLAVERYLRDQQ
LLGIWGCSGKLICTTNVPWNSSWSNKSLDEIWNNMTWLQWDKEINNYTQLIYRLIEESQNQQEK
NEKELLELDKWANLWSWFDISNWLWYIK
SEQ ID NO: 22
A1.RW.92.92RW008.AB253421
STMGAASMTLTVQARQLLSGIVQQQSNLLRAIEAQQHLLKLTVWGIKQLQARVLAVESYLRDQQ
LLGIWGCSGKLICTTTVPWNASWSNKSYSEIWENMTWLQWDKEISNYTNLIYGLIEESQNQQEK
NEQDLLALDKWANLWSWFEISNWLWYIK
SEQ ID NO: 23
A1.UG.92.92UG037.AB253429
STMGAASITLTVQARKLLSGIVQQQSNLLRAIEAQQHLLKLTVWGIKQLQARVLAVERYLRDQQ
LLGIWGCSGKLICPTNVPWNSSWSNKSLDEIWENMTWLQWDKEISNYTIKIYELIEESQIQQER
NEKDLLELDKWASLWNWFDISKWLWYIK
SEQ ID NO: 24
A2.CD.97.97CDKS10.AF286241
STMGAASITLTVQARQLLSGIVQQQSNLLKAIEAQQHLLKLTVWGIKQLQARVLALERYLQDQQ
LLGIWGCSGKLICTTTVPWNSSWSNKTYEEIWNNMTWLQWDREIDNYTNIIYNLLEESQNQQEK
NEQDLLALDKWASLWNWFSITNWLWYIR
SEQ ID NO: 25
A2.CD.97.97CDKTB48.AF286238
STMGAASITLTVQARQLLTGIVQQQSNLLKAIEAQQQMLRLTVWGIKQLQARVLALERYLQDQQ
LLGIWGCSGKLICATDVRWNSSWSNKTQEQIWKNMTWLQWDKEISTYTDIIYMLLEESQNQQEK
NEQDLLALDKWANLWNWFDITRWLWYIK
SEQ ID NO: 26
A2.CY.94.94CY017_41.AF286237
STMGAASLTLTVQARQLLSGIVQQQSNLLQAIEAQQHLLKLTVWGIKQLQARVLAVERYLKDQQ
LLGIWGCSGKLICATTVPWNTSWSNKSQDEIWDNMTWLQWDKEISNYTNIIYRLLEESQNQQEK
NEQDLLALDKWADLWSWFNISHWLWYIR
SEQ ID NO: 27
B.FR.83.HXB2_LAI_IIIB_BRU.K03455
STMGAASMTLTVQARQLLSGIVQQQNNLLRAIEAQQHLLQLTVWGIKQLQARILAVERYLKDQQ
LLGIWGCSGKLICTTAVPWNASWSNKSLEQIWNHTTWMEWDREINNYTSLIHSLIEESQNQQEK
NEQELLELDKWASLWNWFNITNWLWYIK
SEQ ID NO: 28
B.NL.00.671_00T36.AY423387
STMGAASMALTVQARQLLSGIVQQQNNLLRAIEAQQHLLQLTVWGIKQLQARVLAIERYLQDQQ
LLGIWGCSGKLICTTTVPWNASWSNKSLDQIWENMTWMQWEREIDNYTSLIYTLIEDSQKQQEK
NEQELLALDTWASLWNWFSITNWLWYIK
SEQ ID NO: 29
B.TH.90.BK132.AY173951
STMGAASVTLTVQARLLLSGIVQQQNNLLRAIEAQQHLLQLTVWGIKQLQARVLAVERYLKDQQ
LLGIWGCSGKLICTTAVPWNASWSNKSLDEIWNNMTWMQWEREINNYTGLIYTLIEESQNQQEK
NELDLLQLDKWASLWNWFDITNWLWYIK
SEQ ID NO: 30
B.US.98.1058_11.AY331295
STMGAASMTLTVQARLLLSGIVQQQNNLLRAIEAQQHLLQLTVWGIKQLQARVLAVERYLEDQQ
LLGIWGCSGKLICTTAVPWNASWSNKSRSEIWNNMTWMQWDKEIHNYTNLIYTLIGESQIQQEK
NEQELLGLDKWASLWNWFDITKWLWYIK
SEQ ID NO: 31
B.US.98.15384_1.DQ853463
STMGAASIALTVQTRHLLSGIVQQQNNLLRAIEAQQHLLQLTVWGIKQLQARILAVERYLRDQQ
LLGIWGCSGKLICPTAVPWNASWSNKSLEEIWENMTWREWEREIDNYTGKIYDLLAKSQNQREM
NEQELLKLDKWADLWNWFDITQWLWYIK
SEQ ID NO: 32
C.BR.92.BR025_d.U52953
STMGAASITLTVQVRQLLSGIVQQQSNLLRAIEAQQHMLQLTVWGIKQLQTRVLAIERYLRDQQ
LLGIWGCSGKLICTTAVPWNSSWSNRSQEDIWNNMTWMQWDREISNYTNTIYRLLEDSQNQQEK
NEQDLLALDKWQNLWTWFGITNWLWYIK
SEQ ID NO: 33
C.ET.86.ETH2220.U46016
STMGAASITLTVQARQLLSGIVQQQSNLLKAIEAQQHMLQLTVWGIKQLQTRVLAIERHLRDQQ
LLGIWGCSGKLICTTAVPWNSSWSNKSQEEIWDNMTWMQWDREISNYTDIIYNLLEVSQNQQDK
NEKDLLALDKWENLWNWFNITNWLWYIK
SEQ ID NO: 34
C.IN.95.95IN21068.AF067155
STMGAASITLTVQARQLLSGIVQQQSNLLRAIEAQQHLLQLTVWGIKQLQTRVLAIERYLKDQQ
LLGIWGCSGKLICTTAVPWNSSWSNRTQKEIWDNMTWMQWDREINNYTNTIYRLLEESQNQQEE
NEKDLLALDSWKNLWNWFDITKWLWYIK
SEQ ID NO: 35
C.ZA.04.SK164B1.AY772699
STMGAASITLTVQARQLLSGIVQQQSNLLRAIEAQQHMLQLTVWGIKQLQARVLAIERYLKDQQ
LLGLWGCSGKLICTTAVHWNSSWSNKSQDYIWGNMTWMQWDREINNYTDIIYTLLEESQSQQEK
NEKDLLALDSWNNLWNWFSITKWLWYIKIFIMIVGGLIGLRIILGVLSIV
SEQ ID NO: 36
D.CD.83.ELI.K03454
STMGARSVTLTVQARQLMSGIVQQQNNLLRAIEAQQHLLQLTVWGIKQLQARILAVERYLKDQQ
LLGIWGCSGKHICTTNVPWNSSWSNRSLNEIWQNMTWMEWEREIDNYTGLIYSLIEESQTQQEK
NEKELLELDKWASLWNWFSITQWLWYIK
SEQ ID NO: 37
D.CM.01.01CM_4412HAL.AY371157
STMGAASVTLTVQARQLLSGIVQQQNNLERAIEAQQHLLQLTVWGIKQLQARVLAVERYLKDQQ
LLGIWGCSGKHICTTNVPWNSSWSNRSLDDIWQNMTWMQWEREIENYTGVIYSLIEESQIQQEK
NEKELLELDKWASLWNWFSISNWLWYIR
SEQ ID NO: 38
D.TZ.01.A280.AY253311
STMGAASLTLTVQARQLLSGIVQQQNNLLRAIEAQQHLLQLTVWGIKQLQARVLAVESYLKDQQ
LLGIWGCSGKHICTTAVPWNSSWSNKSLDDIWNNMTWMEWEKEIDNYTGVIYSLIEESQVQQEK
NEKELLELDKWASLWNWFSITKWLWYIK
SEQ ID NO: 39
D.UG.94.94UG114.U88824
STMGAVSLTLTVQARQVLSGIVQQQNNLLRAIEAQQHLLQLTVWGIKQLQARILAVESYLKDQQ
LLGIWGCSGKHICTTNVPWNSSWSNRSVDEIWNNMTWMEWEREIDNYTELVYSLLEVSQIQQEK
NEQELLKLDTWASLWNWFSITQWLWYIK
SEQ ID NO: 40
F1.BE.93.VI850.AF077336
EHMGAASITLTVQARQLLSGIVQQQNNLLRAIEAQQHLLQLTVWGIKQLQARVLAVERYLKDQQ
LLGIWGCSGKLICTTNVPWNSSWSNKSQEEIWNNMTWMEWEKEISNYSNIIYKLIEESQNQQEK
NEQELLALDKWASLWNWFDISNWLWYIK
SEQ ID NO: 41
F1.BR.93.93BR020_1.AF005494
STMGAASITLTVQARQLLSGIVQQQSNLLRAIEAQQHLLQLTVWGIKQLQARVLAVERYLKDQQ
LLGLWGCSGKLICTTNVPWNSSWSNKSLEEIWGNMTWMEWEKEVSNYSKEIYRLIEDSQNQQEK
NEQELLALDKWASLWNWFDITQWLWYIK
SEQ ID NO: 42
F1.FI.93.FIN9363.AF075703
STMGAASLTLTVQARQLLSGIVQQQNNLLQAIEAQQHMLQLTVWGIKQLQARVLAVERYLKDQQ
LLGLWGCSGKLICTTNVPWNSSWSNKSQDEIWNNMTWMQWEKEISNYSKTIYMLIEKSQSQQER
NEQELLELDKWDSLWSWFDITNWLWYIK
SEQ ID NO: 43
F1.FR.96.MP411.AJ249238
SNIGAASITLTVQARQLLSGIVQQQNNLLRAIEAQQHLLQLTVWGIKQLQARVLAVERYLKDQQ
LLGIWGCSGKLICTTNVPWNTSWSNKSHDEIWNNMTWMQWEKEINNYSNTIYRLIEESQNQQEK
NEQELLALDKWASLWSWFDISNWLWYIK
SEQ ID NO: 44
F2.CM.02.02CM_0016BBY.AY371158
STMGAASITLTVQARQLLSGIVQQQNNLLKAIEAQQHLLQLTVWGIKQLQARILAVERYLKDQQ
LLGIWGCSGKLICTTNVPWNSSWSNKSQEEIWGNMTWMQWEKEIDNYTDTIYRLIEEAQNQQEK
NEQDLLALDKWDSLWSWFTITNWLWYIR
SEQ ID NO: 99
SEQ ID NO: 45
F2.CM.95.MP255.AJ249236
STMGAAAITLTAQARQLLSGIVQQQSNLLKAIEAQQHLLQLTVWGIKQLQARILAVERYLKDQQ
LLGIWGCSGKLICTTNVRWNSSWSNKSYDDIWDNMTWMQWEKEIDNYTKTIYSLIEDAQNQQER
NEQELLALDKWDSLWSWFSITNWLWYIK
SEQ ID NO: 46
F2.CM.95.MP257.AJ249237
STMGAASITLTVQARNLLSGIVQQQSNLLKAIEAQQHLLQLTVWGIKQLQARILAVERYLKDQQ
LLGIWGCSGKLICPTTVPWNLSWSNKSQDEIWGNMTWMEWEKEIGNYTDTIYRLIESAQNQQEK
NEQDLLALDKWDNLWNWFSITRWLWYIE
SEQ ID NO: 47
F2.CM.97.CM53657.AF377956
STMGAASMTLTVQARQLLSGIVQQQNNLLKAIEAQQHLLQLTVWGIKQLQARILAVERYLKDQQ
LLGIWGCSGKLICTTNVPWNSSWSNKSQNEIWENMTWMQWEKEISNYTGTIYKLIENAQNQQEK
NEQDLLALDKWDNLWSWFTITNWLWYIK
SEQ ID NO: 48
G.BE.96.DRCBL.AF084936
STMGAASITLTVQVRQLLSGIVQQQSNLLRAIEAQQHLLQLTVWGIKQLRARVLALERYLKDQQ
LLGIWGCSGKLICTTNVPWNTSWSNKSYNEIWENMTWIEWEREIDNYTYHIYSLIEQSQIQQEK
NEQDLLALDQWASLWSWFSISNWLWYIR
SEQ ID NO: 49
G.KE.93.HH8793_12_1.AF061641
STMGAASITLTVQVRQLLSGIVQQQSNLLRAIEAQQHLLQLTVWGIKQLQARVLALERYLRDQQ
LLGIWGCSGKLICTTNVPWNASWSNKTYNDIWDNMTWIQWDREISNYTQQIYSLIEESQNQQEK
NEQDLLALDNWASLWTWFDITKWLWYIK
SEQ ID NO: 50
G.NG.92.92NG083.U88826
STMGAASITLTAQVRQLLSGIVQQQSNLLRAIEAQQHLLQLTVWGIKQLQSRVLAIERYLKDQQ
LLGIWGCSGKLICTTNVPWNTSWSNKSYNEIWDNMTWLEWEREIHNYTQHIYSLIEESQNQQEK
NEQDLLALDKWASLWNWFDISNWLWYIR
SEQ ID NO: 51
G.PT.x.PT2695.AY612637
STMGAASITLTVQVRQLLSGIVQQQSNLLRAIEAQQHLLQLTVWGIKQLQARVLAVERYLKDQQ
LLGIWGCSGRLICTTNVPWNASWSNKSYNQIWDNLTWVQWEREISNYTQQIYTLLEESQNQQEK
NEQDLLALDKWADLWNWFDISRWLWYIK
SEQ ID NO: 52
H.BE.93.VI991.AF190127
STMGAASITLTVQARQLLSGIVQQQSNLLRAIQAQQHMLQLTVWGIKQLQARVLAVERYLKDQQ
LLGIWGCSGKLICTTNVPWNSSWSNKSLDEIWDNMTWMEWDKQINNYTDEIYRLLEVSQNQQEK
NEQDLLALDKWANLWNWFSITNWLWYIR
SEQ ID NO: 53
H.BE.93.VI997.AF190128
STMGAASITLTVQARQLLSGIVQQQSNLLRAIQAQQHMLQLTVWGVKQLQARVLAVERYLKDQQ
LLGIWGCSGKLICTTNVPWNSTWSNKSLAEIWDNMTWMEWDRQIDNYTEVIYRLLELSQTQQEQ
NEQDLLALDKWDSLWNWFSITNWLWYIK
SEQ ID NO: 54
H.CF.90.056.AF005496
STMGAASITLTVQARQLLSGIVQQQSNLLRAIQARQHMLQLTVWGIKQLQARVLAVERYLRDQQ
LLGIWGCSGKLICTTNVPWNSSWSNKSQSEIWDNMTWMEWDKQISNYTEEIYRLLEVSQTQQEK
NEQDLLALDKWASLWTWFDISHWLWYIK
SEQ ID NO: 55
J.CD.97.J_97DC_KTB147.EF614151
STMGAASIALTVQARQLLSGIVQQQSNLLKAIEAQQHLLRLTVWGIKQLQARILAVERYLKDQQ
LLGIWGCSGKLICTTNVPWNSSWSNKSHDEIWNNMTWVEWEREIDNYTRIIYNLIEVSQNQQEK
NEQDLLALDKWTSLWSWFKISNWLWYIR
SEQ ID NO: 56
J.SE.93.SE7887.AF082394
STMGAASITLTVQVRQLLSGIVQQQSNLLKAIEAQQHLLKLTVWGIKQLQARVLAVERYLKDQQ
LLGIWGCSGKLICTTNVPWNASWSNKSYEDIWENMTWIQWEREINNYTGIIYSLIEEAQNQQEN
NEKDLLALDKWTNLWNWFNISNWLWYIK
SEQ ID NO: 57
J.SE.94.SE7022.AF082395
STMGAASITLTVQVRQLLSGIVQQQSNLLKAIXAQQHLLKLTVWGIKQLQARVLAVERYLKDQQ
LLGIWGCSGKLICTTNVPWNASWSNKSYEDIWENMTWIQWEREINNYTGIIYSLIEEAQNQQET
NEKDLLALDKWTNLWNWFNISNWLWYIK
SEQ ID NO: 58
K.CD.97.EQTB11C.AJ249235
STMGAASITLTVQARQLLSGIVQQQNNLLRAIEAQQQMLQLTVWGIKQLRARVLAVERYLRDW
LLGIWGCSGKLICTTNVPWNSSWSNKSQSEIWENMTWMQWEKEISNHTSTIYRLIEESQIQQEK
NEQDLLALDKWASLWNWFDISNWLWYIK
SEQ ID NO: 59
K.CM.96.MP535.AJ249239
STMGAASITLTVQARQLLSGIVQQQSNLLRAIEAQQHLLQLTVWGIKQLRARILAVERYLKDQQ
LLGIWGCSGKLICTTNVPWNSSWSNKSWEEIWNNMTWMEWEKEIGNYSDTIYKLIEESQTQQEK
NEQDLLALDKWASLWNWFDITKWLWYIK
SEQ ID NO: 114
SEQ ID NO: 60
N.CM.02.DJO0131.AY532635
STMGAASITLTVQARTLLSGIVQQQNNLVRAIEAQQHLLQLSIWGIKQLRAKVLAIERYLRDQQ
ILSLWGCSGKTICYTTVPWNETWSNNTSYDXIWGNLTWQQWDRKVRNYSGVIFELIXKAQEQQN
TNEKSLLELDQWASLWNWFSITNWLWYIK
SEQ ID NO: 61
N.CM.95.YBF30.AJ006022
STMGAASITLTVQARTLLSGIVQQQNILLRAIEAQQHLLQLSIWGIKQLQAKVLAIERYLRDQQ
ILSLWGCSGKTICYTTVPWNETWSNNTSYDTIWNNLTWQQWDEKVRNYSGVIFGLIEQAQEQQN
TNEKSLLELDQWDSLWSWFGITKWLWYIK
SEQ ID NO: 62
N.CM.97.YBF106.AJ271370
STMGAASITLTVQARTLLSGIVQQQNNLLRAIEAQQHLLQLSIWGIKQLRAKVLAIERYLRDQQ
ILSLWGCSGKTICYTTVPWNDXWSSNTSYDTIWXNLTWQQWDRKVRNYSGVIFDLIEQAQEQQN
TNEKALLELDQWASLWNWFDITKWLWYIK
SEQ ID NO: 63
O.BE.87.ANT70.L20587
STMGAAATTLAVQTHTLLKGIVQQQDNLLRAIQAQQQLLRLSXWGIRQLRARLLALETLLQNQQ
LLSLWGCKGKLVCYTSVKWNRTWIGNESIWDILTWQEWDRQISNISSTIYEEIQKAQVQQEQNE
KKLLELDEWASIWNWLDITKWLWYIK
SEQ ID NO: 64
O.CM.91.MVP5180.L20571
STMGAAATALTVRTHSVLKGIVQQQDNLLRAIQAQQHLLRLSVWGIRQLRARLQALETLIQNQQ
RLNLWGCKGKLICYTSVKWNTSWSGRYNDDSIWDNLTWQQWDQHINNVSSIIYDEIQAAQDQQE
KNVKALLELDEWASLWNWFDITKWLWYIK
SEQ ID NO: 65
O.SN.99.SEMP1300.AJ302647
STMGAAATTLAVQTHTLMKGIVQQQDNLLRAIQAQQQLLRLSVWGIRQLRARLLALETLIQNQQ
LLNLWGCKGRLVCYTSVKWNRTWTNNNTDLDTIWGNLTWQEWDQQISNISATIYDEIQKAQVQQ
EHNEKKLLELDEWASIWNWLDITKWLWYIK
HIV-2 envelope derived triple-helices
SEQ ID NO: 66
H2A.GW.x.ALI.AF082339
SAMGTAALTLSAQSRTLLAGIVQQQQQLLDVVKRQQEMLRLTVWGTKNLQARVTAIEKYLKDQA
RLNSWGCAFRQVCHTTVPWVNNSLKPDWDNMTWQEWEQQVRYLEANISEQLERAQIQQEKNTYE
LQKLNSWDVFTNWLDLTAWVKYIQYGVYIIVGIVALRIV
H2A.GM.x.MCN13.AY509259
SAMGAASLTLSAQSRTLLAGIVQQQQQLLDVVKRQQEMLRLTVWGTKNLQARVTAIEKYLKDQA
QLNSWGCAFRQVCHTTVPWVNDTLTPEWNNMTWQEWEGKIRDLEANISQQLEQAQIQQEKNMYE
LQKLNSWDVFGNWFDLTSWIKYIQYGVYIIIGIVVLRIVIYIV
SEQ ID NO: 67
H2A.GM.x.MCR35.AY509260
SAMGAASLTLSAQSRTLLAGIVQQQQQLLDVVKRQQEMLRLTVWGTKNLQARVTAIEKYLKDQA
QLNSWGCAFRQVCHTTVPWVNDTLTPEWNNMTWQEWEGKIRDLEANISQQLEQAQIQQEKNMYE
LQKLNSWDVFSNWFDLTSWIKYIQYGVYIIIGIV
SEQ ID NO: 68
H2A.GW.87.CAM2CG.D00835
VAMGTASLTLSAQSRTLLAGIVQQQQQLLDVVKRQQELLRLTVWGTKILQARVTAIEKYLKDQA
QLNSWGCAFRQVCHTTVPWANESLTPDWNNMTWQEWEQKVRYLEANISQSLEEAQLQQEKNMYE
LQKLNNWDVFTNWFDLTSWISYIQYGVYIV
SEQ ID NO: 69
H2A.GW.86.FG.J03654
SAMGAASLTLSAQSRTLLAGIVQQQQQLLDVVKRQQEMLRLTVWGTKNLQARVTAIEKYLKDQA
QLNSWGCAFRQVCHTSVPWVNDTLTPDWNNMTWQEWEQKVRYLEANISQSLEQAQIQQEKNMYE
LQKLNSWDVFTNWLDFTSWVRYIQYGVYVVVGIV
SEQ ID NO: 70
H2A.SN.85.ROD.M15390
SAMGAASLTVSAQSRTLLAGIVQQQQQLLDVVKRQQELLRLTVWGTKNLQARVTAIEKYLQDQA
RLNSWGCAFRQVCHTTVPWVNDSLAPDWDNMTWQEWEKQVRYLEANISKSLEQAQIQQEKNMYE
LQKLNSWDIFGNWFDLTSWVKYIQYGVLIIVAVIALRIV
SEQ ID NO: 71
H2A.GM.x.ISY.J04498
AAMGAASLTLSAQSRTLFRGIVQQQQQLLDVVKRQQEMLRLTVWGTKNLQARVTAIEKYLADQA
RLNSWGCAFRQVCHTTVPWVNDTLTPEWNNMTWQEWEHKIRFLEANISESLEQAQIQQEKNMYE
LQKLNSWDVFGNWFDLTSWIKYIQYGVMIVVGIV
SEQ ID NO: 72
H2A.DE.x.PEI2.U22047
SAMGAASLTLSAHPGLYWAGIVQQQQQLLDVVKRQQEMLRLTVWGTKNLQTRVTAIEKYLRDQA
RLNSWGCAFRQVCYTTVLWENNSIVPDWNNMTWQEWEQQTRDLEANISRSLEQAQIQQEKNMYE
LQKLNSWDVFGNWFDLTSWIKYIQYGVYVIIGIIALRIV
SEQ ID NO: 73
H2A.GM.87.D194.J04542
SAMGGASLTLSAQSRTLLAGIVQQQQQLLDVVKRQQEMLRLTVWGTKNLQARVTAIEKYLKDQA
QLNSWGCAFRQVCHTTVPWVNDSLTPOWNNMTWQEWEKRVHYLEANISQSLEQAQIQQEKNMYE
LQKLNSWDVFGNWFDLTSWIKYIQYGVYIVVGIIGLRIAIYIV
SEQ ID NO: 74
H2A.DE.x.BEN.M30502
SAMGARSLTLSAQSRTLLAGIVQQQQQLLDVVKRQQEMLRLTVWGTKNLQARVTAIEKYLKHQA
QLNSWGCAFRQVCHTTVPWVNDSLSPDWKNMTWQEWEKQVRYLEANISQSLEEAQIQQEKNMYE
LQKLNSWDILGNWFDLTSWVKYIQYGVHIVVGIIALRIAIYVV
SEQ ID NO: 75
H2A.GH.x.GH1.M30895
SAMGAASLTLSAQSRTLLAGIVQQQQQLLDVVKRQQEMLRLTVWGTKNLQARVTAIEKYLKDQA
QLNSWGCAFRQVCHTTVPWVNDSLSPDWNNMTWQEWEKQVRYLEANISQSLEQAQIQQEKNMYE
LQKLNSWDVFGNWFDLTSWIKYIQYGVYIVVGVIVLRIAIYIV
SEQ ID NO: 76
H2A.CI.88.UC2.U38293
SAMGAASLTLSAQSRTLLAGIVQQQQQLLDIVKRQQELLRLTVWGTKNLQARVTAIEKYLKDQA
QLNSWGCTFRQVCHTTVPWVNDSLTPRWNNMTWQEWEKQVRYLEANISQSLEEAQIQQEKNMYE
LQKLNSWDVFGNWFDLTSWIKYIQYGVYIVVGIIALRIAIYVV
SEQ ID NO: 77
H2A.GW.x.MDS.Z48731
AAMGAASLTLSAQSRTLLAGIVQQQQQLLDVVKRQQEMLRLTVWGTKNLQARVTAIEKYLKDQA
QLNSWGCAFRQVCHTTVPWVNSSLEPDWENMTWQEWEQKVRYLEANISQKLEEAQIQQEQNMYE
LQKLNSWDIFGNWFDLTSWIKYIQYGVYIVVGIIVLRIVIYVV
SEQ ID NO: 78
H2B.JP.01.KR020.AB100245
SAMGAASLTLSAQSRTLLAGIVQQQQQLLDVVKRQQELLRLTVWGTKNLQARVTAIEKYLKDQA
LLNSWGCAFRQVCHTTVPWPNKNFTPNWDNMTWQQWENQVRFLDENITKLLEVAQIQQEENMYK
LQKLNQWDVFSNWFDFTSWIAYIQIGLYVIVGLVVLRIVIYIL
SEQ ID NO: 79
H2B.CI.88.UC1.L07625
SAMGAASLTLSAQSRTLLAGIVQQQQQLLDVVKRQQELLRLTVWGTKNLQARVTAIEKYLKDQA
LLNSWGCAFRQVCHTTVPWPNKNFTPNWDNMTWQQWENQVRFLDENITKLLEVAQIQQEENMYK
LQKLNQWDVFSNWFDFTSWIAYIQIGLYVIVGLVVLRIVIYIL
SEQ ID NO: 80
H2B.CI.x.EHO.U27200
SAMGAASLTLSAQSRTLLAGIVQQQQQLVDVVKRQQELLRLTVWGTKNLQARVTAIEKYLKDQA
QLNSWGCAFRQVCHTTVPWVNESLKPDWNNMTWQQWERQVRFLDANITKLLEEAQIQQEKNMYE
LQKLNQWDIFSNWFDFTSWMAYIRLGLYIVIGIVVLRIAIYII
SEQ ID NO: 81
H2B.GH.86.D205.X61240
SAMGATSLTLSAQSRTLLAGIVQQQQQPVDVVKRQQELLRLTVWGTKNLQARVTAIEKYLKDQA
QLNSWGCAFRQVCHTTVPWPNETLTPNWNNMTWQQWEKQVHFLDANITALLEEAQIQQEKNMYE
LQKINSWDVFGNWFDLTSWIKYIHLGLYIVAGLVVLRIVVYIV
SEQ ID NO: 82
H2AB.CI.90.7312A.L36874
AAMGAASLTLSAQSRTLLAGIVQQQQQLLDVVKRQQEMLRLTVWGTKNLQARVTAIEKYLKDQA
QLNSWGCAFRQVCHTTVPWVNDSLTPDWDNMTWQQWEKQIRDLEANISESLEQAQIQQEKNMYE
LQKLNSWDVFGNWFDLASWVKYIQYGVYIVVGIVALRVIIYVV
SEQ ID NO: 83
H2U.FR.96.12034.AY530889
SAMGAASLTLSAQSRTLLAGIVQQQQQLLDVVKRQQEMLRLTVWGTKNLQTRVTAIEKYLKDQA
SLNAWGCAFRQVCHTTVPWINDTLTPNWDNMTWQEWEEKVNYLEENITQLLEAAQIQQEKNMYE
LQKLNNWDIFGNWFDLTSWVKYVYLGLYVVAGIIILRIVIYVV
SIV envelope derived triple-helices
SEQ ID NO: 84
MAC.US.x.r90131.AY576481
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWNNETWQEWERKVDFLEENITALLEEAQIQQEKNMYE
LQKLNSWDVFGNWFDLASWIKYIQYGVYIVVGVILLRIVIYIV
SEQ ID NO: 85
MAC.US.x.97074.AY599198
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWNNETWQEWERKVDFLEENITALLEEAQIQQEKNMYE
LQKLNSWDVFGNWFDLASWIKYIQYGVYIVVGVILLRIVIYIV
SEQ ID NO: 86
MAC.US.x.95112.AY588946
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWNNETWQEWERKVDFLEENITALLEEAQIQQEKNMYE
LQKLNSWDVFGNWFDLASWIKYIQYGVYIVVGVILLRIVIYIV
SEQ ID NO: 87
MAC.US.x.96081.AY597209
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWNNETWQEWERKVDFLEENITALLEEAQIQQEKNMYE
LQKLNSWDVFGNWFLASWIKYIQYGVYIVVGVILLRIVIYIV
SEQ ID NO: 88
MAC.US.x.97074.AY599198
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWNNETWQEWERKVDFLEENITALLEEAQIQQEKNMYE
LQKLNSWDVFGNWFDLASWIKYIQYGVYIVVGVILLRIVIYIV
SEQ ID NO: 89
MAC.US.x.97009.AY599199
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWSNETWQEWERKVDFLEENITALLEEAQIQQEKNMYE
LQKLNSWDVFGNWFDLASWIKYIQYGVYIVVGVILLRIVIYIV
SEQ ID NO: 90
MAC.US.x.81035.AY599200
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWNNETWQEWERKVDFLEENITALLEEAQIQQEKNMYE
LQKLNSWDVFGNWFDLASWIKYIQYGVYIVVGVILLRIVIYIV
SEQ ID NO: 91
MAC.US.x.MAC239_87082.AY600249
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWNNETWQEWERKVDFLEENITALLEEAQIQQEKNMYE
LQKLNSWDVFGNWFDLASWIKYIQYGVYIVVGVILLRIVIYIV
SEQ ID NO: 92
MAC.US.x.92050.AY603959
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWNNETWQEWERKVDFLEENITALLEEAQIQQEKNMYE
LQKLNSWDVFGNWFDLASWIKYIQYGVYIVVGVILLRIVIYIV
SEQ ID NO: 93
MAC.US.x.96135.AY607702
SAMGAASLTLTAQSRTLLAGIVQQQQQLLDVVKRQQELLRLTVWGTKNLQTRVTAIEKYLKDQA
QLNAWGCAFRQVCHTTVPWPNANLTPKWNNETWQEWERKVDFLEENITALLEEAQIQQEKNMYE
LQKLNSWDVFGNWFDLASWIKYIQYGVYIVVGVILLRIVIYIV
SEQ ID NO: 94
MAC.US.x.93062.AY607704
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWNNETWQEWERKVDFLEENITALLEEAQIQQEKNMYE
LQKLNSWDVFGNWFDLASWIKYIQYGVYIVVGVILLRIVIYIV
SEQ ID NO: 95
MAC.US.x.80035.AY611486
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWNNETWQEWERKVDFLEENITALLEEAQIQQEKNMYE
LQKLNSWDVFGNWFDLASWIKYIQYGVYIVVGVILLRIVIYIV
SEQ ID NO: 96
MAC.US.x.96020.AY611488
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWNNETWQEWERKVDFLEENITALLEEAQIQQEKNMYE
LQKLNSWDVFGNWFDLASWIKYIQYGVYIVVGVILLRIVIYIV
SEQ ID NO: 97
MAC.US.x.96093.AY611489
SAMGAASLTLTAQSRTLLAGIVQQQQQLLDVVKRQQELLRLTVWGTKNLQTRVTAIEKYLKDQA
QLNAWGCAFRQVCHTTVPWPNASLTPEWNNETWQEWERKVDFLEENITALLEEAQIQQEKNMYE
LQKLNSWDVFGNWFDLTSWIKYIQYGVYIVVGVILLRIVIYIV
SEQ ID NO: 98
MAC.US.x.96072.AY611491
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWDNETWQEWERKVDFLEENITALLEEAQIQQEKNMYE
LQKLNSWDVFGNWFDLTSWIKYIQYGVYIVVGVILLRIVIYIV
SEQ ID NO: 99
MAC.US.x.2065.AY611493
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWDNETWQEWERKVDFLEENITALLEEAQIQQEKNMYE
LQKLNSWDVFGNWFDLXSWIKYIQYGVYIVVGVILLRIVIYIV
SEQ ID NO: 100
MNE.US.82.MNE_8.M32741
SAMGAASLTLTAQSRTLLAGIVQQQQQLLDVVKRQQELLRLTVWGTKNLQTRVTAIEKYLKDQA
QLNAWGCAFRQVCHTTVPWPNANLTPNWNNETWQEWERKVDFLEENITALLEEAQIQQEKNMYE
LQKLNSWDVFGNWFDLASWIRYIQYGVYIVVGVILLRIVIYIV
SEQ ID NO: 101
SMM.SL.92.SL92B.AF334679
SAMGAASLTLTAQSRTLLAGIVQQQQQLLDVVKRQQELLRLTVWGTKNLQTRVTAIEKYLKDQA
QLNSWGCAFRQVCHTTVLWPNDSLVPDWNNMTWQEWEKKVEFLEANITQMLEEARLQQEKNMYE
LQKLNSWDVFGNWFDLTSWVRYIQYGVFLVIGIVLLRIVIYVV
SEQ ID NO: 102
SMM.US.x.SME543.U72748
SAMGAASLTLSAQSRTLLAGIVQQQQQLLDVVKRQHELLRLTVWGTKNLQTRVTAIEKYLKDQA
QLNSWGCAFRQVCHTTVPWPNDSLVPNWDNMTWQEWEGKVDFLEANITQLLEEAQIQQEKNMYE
LQKLNSWDIFGNWFDLTSWIRYIQYGVLIVLGVVGLRIVIYVV
SEQ ID NO: 103
MAC.US.x.17EFR.AY033146
SAMGAASLTLTAQSRTLLAGIVQQQQQLLDVVKRQQELLRLTVWGTKNLQTRVTAIEKYLKDQA
QLNTWGCAFRQVCHTTVPWPNASLIPKWNNETWQEWERKVDFLEENITALLEEAQIQQEKNMYE
LQKLNSWNVFGNWFDLASWIKYIQYGVYIVVGVILLRIVIYIV
SEQ ID NO: 104
MAC.US.x.17EC1.AY033233
SAMGAASLTLTAQSRTLLAGIVQQQQQLLDVVKRQQELLRLTVWGTKNLQTRVTAIEKYLKDQA
QLNTWGCAFRQVCHTTVPWPNASLIPKWNNETWQEWERKVDFLEENITALLEEAQIQQEKNMYE
LQKLNSWNVFGNWFDLASWIKYIQYGVYIVVGVILLRIVIYIV
SEQ ID NO: 105
MAC.US.x.251_32H_PJ5.D01065
SAMGAASLTLTAQSRTLLAGIVQQQQQLLDVVKRQQELLRLTVWGTKNLQTRVTAIEKYLKDQA
QLNAWGCAFRQVCHTTVPWPNASLTPDWNNDTWQEWERKVDFLEENITALLEEAQIQQEKNMYE
LQKLNSWDVFGNWFDLASWIKYIQYGVYIVVGVILLRIVIYIV
SEQ ID NO: 106
MAC.US.x.239.M33262
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWNNETWQEWERKVDFLEENITALLEEAQIQQEKNMYE
LQKLNSWDVFGNWFDLASWIKYIQYGVYIVVGVILLRIVIYIV
SEQ ID NO: 107
MNE.US.x.MNE027.U79412
SAMGAASLTLTAQSRTLLAGIVQQQQQLLDVVKRQQELLRLTVWGTKNLQTRVTAIEKYLKDQA
QLNAWGCAFRQVCHTTVPWPNASLTPNWNNETWQEWERKVDFLEENITALLEEAQIQQEKNMYE
LQKLNSWDVFGNWFDLASWIRYIQYGVYIVVGVILLRIVIYIV
SEQ ID NO: 108
SMM.US.x.PGM53.AF077017
SAMGAASVTRSAQSRTLLAGIVQQQQQLLDVVKRQQELLRLTVWGTKNLQTRVTAIEKYRKDQA
QLNSWGCAFRQVCHTTVPWPNASLVPNWNNMTWQEWERQVDDLEANITQALEEAQIQQEKNMYE
LQKLNSWDIFGNWFDLTSWIKYIQYGVLIVLGVVGLRIVIYVV
SEQ ID NO: 109
SMM.US.x.PBJ14_15.L03295
SAMGAASVTLSAQSRTLLAGIVQQQQQLLDVVKRQQELLRLTVWGAKNLQTRVTAIEKYLKDQA
QLNSWGCAFRQVCHTTVPWPNDTLTPNWNNMTWQEWEKQVNFLEANITQSLEEAQIQQEKNTYE
LQKLNSWDIFGNWFDLTSWIKYIQYGVLIVLGVIGLRIVIYVV
SEQ ID NO: 110
SMM.US.x.PBJ_6P6.L09212
SAMGAASVTLSAQSRTLLAGIVQQQQQLLDVVKRQQELLRLTVWGAKNLQTRVTAIEKYLKDQA
QLNSWGCAFRQVCHTTVPWPNDTLTPNWNNMTWQEWEKQVNFLEANITQSLEEAQIQQEKNTYE
LQKLNSWDIFGNWFDLTSWIKYIQYGVLIVLGVIGLRIVIYVV
SEQ ID NO: 111
SMM.US.x.PBJA.M31325
SAMSAASVTLSAQSRTLLAGIVQQQQQLLDVVKRQQELLRLTVWGAKNLQTRVTAIEKYLKDQA
QLNSWGCAFRQVCHTTVPRPNDTLTPNWNNMTWQEWEKQVNFLEANITQSLEEAQIQQEKNTYE
LQKLNSWDIFGNWFDLTSWIKYIQYGVLIVLGVIGLRIVIYVV
SEQ ID NO: 112
SMM.US.x.F236_H4.X14307
SAMGAASVTLSAQSRTLLAGIVQQQQQLLDVVKRQQELLRLTVWGTKNLQTRVTAIEKYLKDQA
QLNSWGCAFRQVCHTTVPWPNETLVPNWNNMTWQEWERQVDFLEANITQLLEEAQIQQEKNMYE
LQKLNSWDIFGNWFDLTSWIRYIQYGVLIVLGVIGLRIVIYVV
SEQ ID NO: 113
STM.US.x.STM.M83293
SAMGAASLTLTAQSRTLLTGIVQQQQQLLDVVKRQQELLRLTVWGTKNLQTRVTAIEKYLKDQA
QLNSWGCAFRQVCHTTVPWPNDSLVPDWNNMTWQEWERKVDFLEANITQLLEEAQVQQEKNMYE
LQKLNSWDVFGNWFDLTSWVRYIQYGVYLVIGLVMLRVAIYI
SEQ ID NO: 114
CPZ.CM.05.SIVCpzEK505.DQ373065
STMGAASITLTVQARKLLSGIVQQQNNLLRAIEAQQHLLQLSVWGIKQLQARVLAIERYLRDQQ
ILGLWGCSGKSVCYTNVPWNTTWSNNNSYDTIWGNMTWQNWDEQVRNYSGVIFGLLEQAQEQQS
INEKSLLELDQWSSLWNWFDITKWLWYIKIFIMVVAGIVGIRI
SEQ ID NO: 115
CPZ.CM.05.SIVCpzLB7.DQ373064
STMGAASLTLTVQARQLLTGIVQQQSNLLRAIEAQQHLLQLSVWGIKQLQARVLAIERYLKDQQ
LLGIWGCSGKLICTTSVPWNRTWSNKTYNEIWDNMTWMEWDREVRNYTEIIYGLIEQAQDQQEN
NEKKLLELDHWTSLWNWFDISHWLWYIKIFIMIIGGLIVCRIIFAVLAIV
SEQ ID NO: 116
CPZ.CM.01.SIVCpzCAM13.AY169968
STMGAAAVTLTVQARQLLSGIVQQQNNLLRAIEAQQHLLQLTVWGVKQLQARLLAVERYLQDQQ
ILGLWGCSGKSICYTTVPWNKTWSGKSMSDIWNNLTWQQWDKLITNYTGTIFGLLEEAQSQQEK
NEKDLLELDQWASLWNWFDITNWLWYIKIFLMAVGGIIGLRIIMSVVSVIR
SEQ ID NO: 117
CPZ.CM.05.SIVCpzMB66.DQ373063
STMGAASLTLTVQARQLLSGIVQQQSNLLRAIEAQQHLLQLSVWGIKQLQARVLAVERYLKDQQ
LLGLWGCSGKLICTTSVPWNTTWTNKSYDDIWYNMTWMQWDKEVSNYTDVIYNLLEKAQTQQEN
NEKELLELDKWASLWNWFDITSWLWYIKIFIIIVGGLIGLRIVFALLSIV
SEQ ID NO: 118
CPZ.CM.05.SIVCpzMT145.DQ373066
STMGAASVVLTVQARQLLTGIVQQQNNLLRAIEAQQHLLQLSVWGIKQLQARVLAVERYLRDQQ
LLGLWGCTGKTICPTAVRWNKTWGNISDYQVIWNNYTWQQWDREVNNYTGLIYTLLEEANTQQE
KNEKELLELDSWANLWSWFDITNWLWYIKMFLIVVGGIIGLRICFAIGSLI
SEQ ID NO: 119
CPZ.CM.98.CAM3.AF115393
STMGAASVVLTVQARQLLSGIVQQQNNLLRAIEAQQHLLQLSVWGIKQLQARVLAVERYLRDQQ
ILGLWGCSGKAICYTTVPWNNTWSANTSFDEIWNNLTWQDWDKRVKNYSGVIFSLIEQAQEQQN
TNEKSLLELDQWSSLWNWFDITRWLWYIKLFIMIVAGLVGIRIVGAII
SEQ ID NO: 120
CPZ.CM.98.CAM5.AJ7271369
STMGAASVVLTVQARHLLSGIVQQQNNLLRAIEAQQHLLQLSVWGIKQLQARVLAVERYLKDQQ
ILSLWGCSGKAICYTTVPWNATWSANTSYDEIWNNLTWQDWDKKVKNYSGVIFSLIEQAQEQQN
TNEKDLLELDQWSSLWSWFNITQWLWYIKIFLIVVAGLIGFRLIGIV
SEQ ID NO: 121
CPZ.GA.88.GAB1.X52154
STMGAAAVTLTVQARQLLSGIVQQQNNLLKAIEAQQHLLQLSIWGVKQLQARLLAVERYLQDQQ
ILGLWGCSGKAVCYTTVPWNNSWPGSNSTDDIWGNLTWQQWDKLVSNYTGKIFGLLEEAQSQQE
KNERDLLELDQWASLWNWFDITKWLWYIKIFLMAVGGIIGLRII
SEQ ID NO: 122
CPZ.GA.88.GAB2.AF382828
STMGAAAVTLTVQARQLLSGIVQQQNNLLKAIEAQQHLLQLSIWGVKQLQARLLAVERYLQDQQ
ILGLWGCSGKAVCYTTVPWNNSWPGSNSTDDIWGNLTWQQWDKLVSNYTGKIFGLLEEAQSQQE
KNERDLLELDQWASLWNWFDITKWLWYIKIFLMAVGGIIGLRII
SEQ ID NO: 123
CPZ.US.85.CPZUS.AF103818
STMGAASVVLTVQARQLLSGIVQQQNNLLRAIEAQQHLLQLSVWGIKQLQARVLAVERYLKDQQ
ILGLWGCSGKTICYTTVPWNDTWSNNLSYDAIWGNLTWQEWDRKVRNYSGTIFSLIEQAQEQQN
TNEKSLLELDQWSSLWNWFDITNWLWYIKIFLIVVASLVGIRIV
SEQ ID NO: 124
CPZ.CD.90.ANT.U42720
STMGAASIALTAQTRNLXHGIVQQQANLLQAIETQQHLLQLSVWGVKQLQARMLAVEKYLRDQQ
LLSLWGCADKVTCHTTVPWNNSWVNFTQTCAKNSSDIQCIWENMTWQEWDRLVQNSTGQIYNIL
QIAHEQQERNKKELYELDKWSSLWNWFDITQWLWYIKIFIMIVGAIV
SEQ ID NO: 125
CPZ.TZ.01.TAN1.AF447763
STMGAASIALTAQARGLLSGIVQQQQNLLQAIEAQQHLLQLSVWGIKQLQARMLAVEKYIRDQQ
LLSLWGCANKLVCHSSVPWNLTWAEDSTKCNHSDAKYYDCIWNNLTWQEWDRLVENSTGTIYSL
LEKAQTQQEKNKQELLELDKWSSLWDWFDITQWLWYIKIAIIIV
SEQ ID NO: 126
COL.CM.x.CGU1.AF301156
AMGSASVALTIQAQSLNGRASASSNRMLLKLVETQSALLQLTVWGVKNLQVRVATIEGYLEEQA
KLASIGCANMQICRTIVPWNKTWGEEDPWQNMTWKQWHERVRNYTDIIEADLVEAYDLQEENEK
KLAELGDWTNWFSGEGLFNIFKYVLYAAYVVGGLIGLRIIMVVIA
SEQ ID NO: 127
DEB.CM.99.CM40.AY523865
AAMGAASTALTVQSRSLLSGIVQQQQELLKAVEAHGQLLTLTAWGVRNLNTRLTAIEKYLKDQA
KLNEWGCAFKQICHTTVPWNNSLEDPDWDNMTWQEWEMKVANYTDEWEGALQRAQEQQERNVHA
LQSLQDWDSLWNWFDLSRWFWWIRLVVYIIAALILLRIAMFGVNI
SEQ ID NO: 128
DEB.CM.99.CM5.AY523866
AAMGAASTALTVQSRSLLSGIVQQQQELLKAVEAHGHLLSLTAWGVRNLNTRLTAIEKYLKDQS
KLNEWGCAFKQICHTTVPWNHTWGEPDWNNMTWQEWERKVANYTDEWEGALQRAQEQQERNVHA
LQSLTDWDSLWNWFDLSRWFWWIRLVVYIIAALILLRI
SEQ ID NO: 129
GSN.CM.99.CN166.AF468659
TTMGAAATALTEQSRSLLAGIMQQQENLLRAVEAQQSLLQPSVWGIKQLQTRLSSLEKYLRDQT
ILQAWGCANRPICHTIVPWNTSWANGSLPDWENMTWQKWSMLVENDTYTIQQLLEQANQQQASN
LNELMKLSKWDSLWSWFDISDWQRYIKIFVIVVAALIALRIV
SEQ ID NO: 130
GSN.CM.99.CN71.AF468658
ATMGAAATALTVQSRSLLAGIVQQQENLLRAVEAQQSLLQLSVWGIKQLQARLSSLEKYLRDQT
ILQAWGCANQPICHTIVPWNDSWAKNSTPDWEHMTWQEWSKLIENDTYTIQQLLENANHQQSKN
MNDLLKLSKWDSLWSWFDISNWLWYIKIFIMVVAALVALRII
SEQ ID NO: 131
A.GW.x.ALI.AF082339
SAMGTAALTLSAQSRTLLAGIVQQQQQLLDVVKRQQEMLRLTVWGTKNLQARVTAIEKYLKDQA
RLNSWGCAFRQVCHTTVPWVNNSLKPDWDNMTWQEWEQQVRYLEANISEQLERAQIQQEKNTYE
LQKLNSWDVFTNWLDLTAWVKYIQYGVYIIVGIVALRIVIYVV
SEQ ID NO: 132
A.DE.x.BEN.M30502
SAMGARSLTLSAQSRTLLAGIVQQQQQLLDVVKRQQEMLRLTVWGTKNLQARVTAIEKYLKHQA
QLNSWGCAFRQVCHTTVPWVNDSLSPDWKNMTWQEWEKQVRYLEANISQSLEEAQIQQEKNMYE
LQKLNSWDILGNWFDLTSWVKYIQYGVHIVVGIIALRIAIYVV
SEQ ID NO: 133
A.SN.x.ST.M31113
AAMGAASLTLSAQSRTLLAGIVQQQQQLLDVVKRQQEMLRLTVWGTKNLQARVTAIEKYLKDQA
QLNSWGCAFRQVCHTTVPWVNDTLTPDWNNMTWQEWEQRIRNLEANISESLEQAQIQQEKNMYE
LQKLNSWDVFGNWFDLTSWIKYIQYGVYIVVGIIVLRIVIY
SEQ ID NO: 134
B.GH.86.D205.X61240
SAMGATSLTLSAQSRTLLAGIVQQQQQPVDVVKRQQELLRLTVWGTKNLQARVTAIEKYLKDQA
QLNSWGCAFRQVCHTTVPWPNETLTPNWNNMTWQQWEKQVHFLDANITALLEEAQIQQEKNMYE
LQKINSWDVFGNWFDLTSWIKYIHLGLYIVAGLVVLRIVVYIV
SEQ ID NO: 135
B.CI.x.EHO.U27200
SAMGAASLTLSAQSRTLLAGIVQQQQQLVDVVKRQQELLRLTVWGTKNLQARVTAIEKYLKDQA
QLNSWGCAFRQVCHTTVPWVNESLKPDWNNMTWQQWERQVRFLDANITKLLEEAQIQQEKNMYE
LQKLNQWDIFSNWFDFTSWMAYIRLGLYIVIGIVVLRIAIYII
SEQ ID NO: 136
G.CI.x.ABT96.AF208027
SAMGTASLTLSAQSRTLLAGIVQQQQQLLDVVKRQQEMLRLTVWGTKNLQTRVTAIEKYLKDQA
RLNSWGCAFRQVCHTTVPWDALGANKTLEPQWNNMTWQEWEKQINFLEDNITRLLEEAQIQQEK
NMYELQKLNSWDVFGNWFDLTSWVKYVYLGLYVVAGVIVLRIVIYVV
SEQ ID NO: 137
U.FR.96.12034.AY530889
SAMGAASLTLSAQSRTLLAGIVQQQQQLLDVVKRQQEMLRLTVWGTKNLQTRVTAIEKYLKDQA
SLNAWGCAFRQVCHTTVPWINDTLTPNWDNMTWQEWEEKVNYLEENITQLLEAAQIQQEKNMYE
LQKLNNWDIFGNWFDLTSWVKYVYLGLYVVAGIIILRIVIYVV
SEQ ID NO: 138
MAC.US.x.EMBL_3.Y00295
SAMGAASFRLTAQSRTLLAGIVQQQQQLLGVVKRQQELLRLTVWGTKNLQTRVTAIEKYLEDQA
QLNAWGCAFRQVCHTTVPWPNASLTPDWNNDTWQEWERKVDFLEENITALLEEAQIQQEKNMYE
LQKLNSWDVFGNWFDLASWIKYIQYGIYVVVGVILLRIVIYIV
SEQ ID NO: 139
MAC.US.x.239.M33262
SAMGAASLTLTAQSRTLLAGIVQQQQQLLDVVKRQQELLRLTVWGTKNLQTRVTAIEKYLKDQA
QLNAWGCAFRQVCHTTVPWPNASLTPKWNNETWQEWERKVDFLEENITALLEEAQIQQEKNMYE
LQKLNSWDVFGNWFDLASWIKYIQYGVYIVVGVILLRIVIYIV
SEQ ID NO: 140
SMM.US.x.SIVsmH635FC.DQ201174
SAMGAASLTLSAQSRTLLAGIVQQQQQLLDVVKRQHELLRLTVWGTKNLQTRVTAIEKYLKDQA
QLNSWGCAFRQVCHTTVPWPNDSLVPNWDNMTWQEWEGKVDFLEANITQLLEEAQIQQEKNMYE
LQKLNSWDIFGNWFDLTSWIRYIQYGVLIVLGVVGLRIVIYVV
SEQ ID NO: 141
SMM.US.x.H9.M80194
SAMGAASLTLSAQSRTLLAGIVQQQQQLLDVVKRQQELLRLTVWGTKNLQTRVTAIEKYLKDQA
PXNSWGCAFRQVCHTTVPWPNDTLTPXWNNMXWQEWEKQVNFLEANITZXLEEAQIQQEXNMYE
LQKLNXXDXFGNWXDLTXWIKYIQYGVLIVLGVIGLRIVIYVV
SEQ ID NO: 142
STM.US.x.STM.M83293
SAMGAASLTLTAQSRTLLTGIVQQQQQLLDVVKRQQELLRLTVWGTKNLQTRVTAIEKYLKDQA
QLNSWGCAFRQVCHTTVPWPNDSLVPDWNNMTWQEWERKVDFLEANITQLLEEAQVQQEKNMYE
LQKLNSWDVFGNWFDLTSWVRYIQYGVYLVIGLVMLRVAIYI
SEQ ID NO: 143
SMM.SL.92.SL92B.AF334679
SAMGAASLTLTAQSRTLLAGIVQQQQQLLDVVKRQQELLRLTVWGTKNLQTRVTAIEKYLKDQA
QLNSWGCAFRQVCHTTVLWPNDSLVPDWNNMTWQEWEKKVEFLEANITQMLEEARLQQEKNMYE
LQKLNSWDVFGNWFDLTSWVRYIQYGVFLVIGIVLLRIVIYVV
SEQ ID NO: 144
SMM.US.x.PGM53.AF077017
SAMGAASVTRSAQSRTLLAGIVQQQQQLLDVVKRQQELLRLTVWGTKNLQTRVTAIEKYRKDQA
QLNSWGCAFRQVCHTTVPWPNASLVPNWNNMTWQEWERQVDDLEANITQALEEAQIQQEKNMYE
LQKLNSWDIFGNWFDLTSWIKYIQYGVLIVLGVVGLRIVIYVV
SEQ ID NO: 145
MNE.US.x.MNE027.U79412
SAMGAASLTLTAQSRTLLAGIVQQQQQLLDVVKRQQELLRLTVWGTKNLQTRVTAIEKYLKDQA
QLNAWGCAFRQVCHTTVPWPNASLTPNWNNETWQEWERKVDFLEENITALLEEAQIQQEKNMYE
LQKLNSWDVFGNWFDLASWIRYIQYGVYIVVGVILLRIVIYIV
SEQ ID NO: 146
RCM.NG.x.NG411.AF349680
TAMGAAATALTVQSRHLLAGILQQQKKLLDIVEQQQELLKLTVWGTKNLQARVTAIEKYLADQS
LLNTFGCAWRQVCHTTVEWIYSQTPEWNKQTWLEWERNISRLEGNISVALQDAQEQHERNVHDL
EKLNSWGDMLSWLNMDWWLKYIRIGIFIILGIIGLRIIFLLWS
SEQ ID NO: 147
RCM.GA.x.GAB1.AF382829
TAMGAAATALTVQSRHLLAGILQQQKNLLDIVKRQQNLLKLTVWGTKNLQARVTAIEKYLADQS
LLNTFGCAWRQVCHTVVPWTFNKTPEWQKESWLQWERNISYLEANITIALQEAQDQHEKNVHEL
EKLSNWGDAFSWLNLDWWMQYIKIGFFIVIGIIGLRVAWLL
SEQ ID NO: 148
DRL.x.x.FAO.AY159321
SAMCSVATAMTVQSQALLTGMVEQQKQLLRLVEQQQELLKLTIWGVKNLQARLTALEEYIGDQA
MLSLWGCSFAQVCHTNVVWPNESVTPNWTSETWMEWQKRVDSISNNITLDLQKAYEQEQKNIFE
LQKLGDLTSWANWFDFTWWSKYIKIGFFIVMAIIGLRILAAL
SEQ ID NO: 149
MND-1.GA.x.MNDGB1.M27470
SAMGSVSVALTVQSQSLVTGIVEQQKQLLKLIEQQSELLKLTIWGVKNLQTRLTSLENYIKDQA
LLSQWGCSWAQVCHTSVEWTNTSITPNWTSETWKEWETRTDYLQQNITEMLKQAYDREQRNTYE
LQKLGDLTSWASWFDFTWWVQYLKWGVFLVLGIIGLRILLAL
SEQ ID NO: 150
MND-2.x.x.5440.AY159322
SAMGSVAVALTVQSQTLLNGIVEQQKVLLSLIDQHSELLKLTIWGVKNLQVRLTALEEYVADQS
RLSVWGCSFSQVCHTSVKWPNNSIVPNWTSETWLEWDRRVNSIVTNMTIDLQRAYELEQRNIFE
LQKLGDLNFHGLTGFDLTWWLKYVKIGLLVVVVIIGLRMLACL
SEQ ID NO: 151
MND-2.GA.x.M14.AF328295
SAMGSVAVALTVQSQALLNGIVEQQKILLSLIDQHSELLKLTIWGVKNLQARLTALEDYVADQS
RLAVWGCSFSQVCHTNVPWPNESITPNWTSETWLEWDRRVTAITNNMTIDLQRAYELEQKNMYE
LQKLGDLTSWASWFDLTWWLKYVKIGILIIMVVIGLRILAC
SEQ ID NO: 152
MND-2.CM.98.CM16.AF367411
STMGSVAVALTVQSQALLNGIVEQQKVLLSLIDQHSELLKLTIWGVKNLQARLTALEDYVADQA
RLSMWGCSFAQVCHTHVPWPNDSITPNWTSETWLEWDKRVTALTDNMTVNLQKAYELEQKNIYE
LEKLGDWTSWASWFDFTWWLKYVKIGLLIVIVIIVLRILAC
SEQ ID NO: 153
GRV.ET.x.GRI_677.M66437
TAMGAAATTLTVQSRHLLAGILQQQKNLLAAVEQQQQLLKLTIWGVKNLNARVTALEKYLEDQA
RLNSWGCAWKQVCHTTVPWKYNNTPKWDNMTWLEWERQINALEGNITQLLEEAQNQESKNLDLY
QKLDDWSGFWSWFSLSTWLGYVKIGELVIVIILGLRFAWVLWGC
SEQ ID NO: 154
TAN.UG.x.TAN1.U58991
AAMGATATALTVQSQQLLAGILQQQKNLLAAVEDQQQMLKLTIWGVKNLNARVTALEKYLEDQT
RLNLWGCAFKQVCHTTVPWTFNNTPDWDNMTWQEWESQITALEGNISTTLVKAYEQEQKNMDTY
QKLGDWTSWWNIFDVSSWFWWIKWGFYIVIGLILFRMAWLIWGC
SEQ ID NO: 155
LST.CD.88.447.AF188114
TAMGLVSTILTVQAQVVIQGILQQQKQLLVLVEKQQELLRLTIWGVKNLQARLTAIEEYLKDQT
LLASWGCQWKQVCHTNVEWNYNITPNWTRDTWIEWDRQVGVLEANISTLLQEAYTTELENRNAF
KKLQEFNFWNWLDILSWFQYIKYAVLIIIGIIVLRVVSFIVQNIVKMC
SEQ ID NO: 156
LST.CD.88.485.AF188115
TAMGLVSTILTVQAQVVIQGILQQQKQLLVLVEKQQELLRLTIWGVKNLQARLTAIEEYLKDQA
LLASWGCQWKQICHTNVEWNYNITPNWTRDTWIEWDRRVGVLEANISTLLQEAYTTELENRNAF
KKLQEFNFWSWLDILSWFQYIKYAVLIIIGIIVLRIVSFIVQNIVKMC
SEQ ID NO: 157
LST.CD.88.524.AF188116
TAMGLVSTILTVQAQVVIQGILQQQKQLLVLVEKQQELLRLTIWGVKNLQARLTAIEEYLKDQA
LLASWGCQWKQVCHTNVPWNYNVTPNWTRDTWIEWERQVGSLEANITTLLQEAYTTELENRNNF
KKLQDFNFWSWMDLTTWFQYIKYAVLIIIGIIILRILSFIIQSVVKMC
SEQ ID NO: 158
LST.KE.x.1ho7.AF075269
TAMGLVSTILTVQAQAVLQGILQQQKQLLVLVEKQQELLRLTIWGVKNLQARLTALEEYVKHQA
LLASWGCQWKQVCHTNVEWTYNITPNWTKDTWREWESKVAIYDKNITSLLQEAYTTELENQNKF
KKLQEFNFWSWLDISHWFTYVKYAVLIILVIIGLRVLSFIIQNVVKMC
SEQ ID NO: 159
MON.CM.99.L1.AY340701
GTMGAAATALTVQSRSLLAGIVQQQENLLRAVTAQQSLLQLTVWGVKQLQARLTAVEKFIKDQT
LLNAWGCANKAVCHTTVPWNNSWAKGHFPEWDNMTWQQWSELVDNDTMTIQQLLEAAQEQQGKN
QHELMKPGQWDFLWNWFDISKWLWYIKIFIIVVAALIGLRILMFILGVI
SEQ ID NO: 160
MON.NG.x.NG1.AJ549283
GTMGAAATALTVQSRSLLAGIVQQQENLLRAVTAQQSLLQLSVWGIKQLQARLTAVEKFIKDQT
LLNSWGCANRAVCHTQVLWNNTWAKGHFPEWDNMTWQQWSMLVDNDTALIQXLLEEAQEQQGKN
AHELMKLGQWDWLWNWFDISKWLWYIKIFIIVVAALVGLRVLMFILGII
SEQ ID NO: 161
TAL.CM.01.8023.AM182197
TAMGAVATALTVQSRSLLSGIVQQQEHLLRAIEHQQHLLQLTVWGIKNLNARLTALEKYLEDQA
RLNSWGCAWKQICYTSVPWNKTWTNSTNPDWQNMTWQEWEKLVDNASDTITVLLQEAQEQQERN
VHELQKLNDWDSLWSWFNLSAWFRWLRIAVIVVASLILLRIVMYII
SEQ ID NO: 162
TAL.CM.00.266.AY655744
TAMGAVATALTVQSRSLLSGIVQQQEHLLRAIEHQQHLLQLTVWGIKNLNARLTALEKYLEDQA
KLNSWGCAWKQICYTSVPWNKTWSNYTDPQWQNMTWQEWEMKVDNHTGLISQLLQEAQEQQERN
VHELQKLNDWDSLWSWFNLSAWFRWLRIAVIVVASLILLRIVMYIV
SEQ ID NO: 163
MUS-1.CM.01.1085.AY340700
GTMGAAATALTVQSRSLLAGIVQQQANLLRAVEAQQHLLQLSVWGIKQLQARLTALEKFIKDQA
LLNLWGCANRQICHTRVPWNDSWANHTQPGWENMTWQQWSRLVDNDTTTIQELLELAQRQQEEN
QHKLQKLLEWDSLWEWFDISKWLWYIKIFCMVVAGLVLFRLVMFVLGIL
SEQ ID NO: 164
SAB.SN.x.SAB1C.U04005
AAMGAAATALTVQSQQLLAGILQQQKNLLAAVEQQQQMLKLTIWGVKNLNARVTALEKYLEDQA
RLNIWGCAFRQVCHTTVLWKYNNTPDWENMTWQEWERQIEKYEANISRILEQAHEQEQKNLDSY
QKLVSWSDFWSWFDLTKWFGWMKIAIMVIAGIIVARVLLVIIGIL
SEQ ID NO: 165
VER.DE.x.AGM3.M30931
TAMGAAATALTVQSQHLLAGILQQQKNLLAAVEAQQQMLKLTIWGVKNLNARVTALEKYLEDQA
RLNAWGCAWKQVCHTTVPWQWNNRTPDWNNMTWLEWERQISYLEGNITTQLEEARAQEEKNLDA
YQKLSSWSDFWSWFDFSKWLNILKIGFLDVLGIIGLRLLYTVYSC
SEQ ID NO: 166
VER.KE.x.9063.L40990
TAMGAAATALTVQSQHLLAGIMQQQKNLLAAVEAQQQMLKLTIWGVKNLNARVTALEKYLEDQA
RLNVWGCAWKQVCHTTVPWQWQNMTPNWQNMTWLEWERQIGELEGNITEQLVKAREQEEKNLDA
YQRLTSWSNFWSWFDFSKWLNILKIGFLVVVGIIGLRLLYTIYSC
SEQ ID NO: 167
VER.KE.x.AGM155.M29975
TAMGAAATALTVQSQHLLAGILQQQKNLLAAVGAQQQMLKLTIWGVKNLNARVTALEKYLADQA
RLNAWGCAWKQVCHTTVPWTWNNTPEWNNMTWLEWEKQIEGLEGNITKQLEQAREQEEKNLDAY
QKLSDWSSFWSWFDFSKWLNILKIGFLAVIGVIGLRLLYTLYTC
SEQ ID NO: 168
VER.KE.x.TYO1.X07805
TAMGAAASSLTVQSRHLLAGILQQQKNLLAAVEAQQQMLKLTIWGVKNLNARVTALEKYLEDQA
RLNSWGCAWKQVCHTTVEWPWTNRTPDWQNMTWLEWERQIADLESNITGQLVKAREQEEKNLDA
YQKLTSWSDFWSWFDFSKWLNILKMGFLVIVGIIGLRLLYTVYGCIV
SEQ ID NO: 169
SUN.GA.98.L14.AF131870
TAMGLVSTILTVQAQAVLQGILQQQKQLLVLVEKQQELLRLTIWGVKNLQARLTALEEYVQDQS
LLASWGCQWKQVCHTNVPWNYNITPNWTKDTWMEWDRQVKMYDDNITALLQEAYVTELENQNKF
KQLQEFNFWSWLDLSQWFLYIKYAVLIIGIIIAARILSFIIQQIYRMCQGYRVLSPSAYVEQDW
LQETCPKPTDKEEEEETEKERIYINLEQSKKESLPPP
SEQ ID NO: 170
SYK.KE.x.SYK173.L06042
TAMGGAATALTLQSQTLLAGIVQQQQKLLEAVEAQQHLLGLTVWGVKNLNARLTALETYLRDQA
ILSNWGCAFKQICHTAVTWEKACGNNSNFCPKPQWKNMTWHRWEQEVDNLTDHIDGLLREAQEQ
QERNVHDLTKLQEWDSLWSWFDLSKWFFYLKIGFYVIGALV
SEQ ID NO: 171
SYK.KE.x.KE51.AY523867
TAMGSAATALTLQSQTLLAGIVQQQQKLLEAVEAQQHLLGLTVWGVKNLNARLTALETYLRDQA
IMSNWGCAFKQICHTAVTWQQACGNNSRCPTPQWENMTWHTWERQVDNLTDHIDNLLREAQEQQ
EKNVHDLTKLQEWDSLWSWFDLSKWFQYLKIGFFAIAAIV
SEQ ID NO: 172
(MLV triple-helix)
LKEVEKSITNLEKSLTSLSEVVLQNRRGLDLLFLKEGGLCAALKEECCFYADHTGLVRDSMAKL
RERLSQRQKLFESQQGWFEGLFNKSPWFTTLISTIMG
SEQ ID NO: 173
(JSRV triple-helix)
TKVMGTQEDIDKKIEDRLSALYDVVRVLGEQVQSINFRMKIQCHANYKWICVTKKPYNTSDFPW
DKVKKHLQGIWFNTNLSLDLLQLHNEILDIENSPKATLNIADTVDNFLQNLFSNFPSLHSLWKT
LIGLGIF
SEQ ID NO: 174
(FeLV triple-helix)
IQALEESISALEKSLTSLSEVVLQNRRGLDILFLQEGGLCAALKEECCFYADHTGLVRDNMAKL
RERLKQRQQLFDSQQGWFEGWFNKSPWFTTLISSIMG
SEQ ID NO: 175
(BLV triple-helix)
LEQDQQRLITAINQTHYNLLNVASVVAQNRRGLDWLYIRLGFQSLCPTINEPCCFLRIQNDSII
RLGDLQPLSQRVSTDWQWPWNWDLGLTAWVRETIHSVLS
SEQ ID NO: 176
(Influenza HA triple-helix)
STQKAIDGVTNKVNSIIDKMNTQFEAVGREFNNLERRIENLNKQMEDGFLDVWTYNAELLVLME
NEHDSNVKNLYDKVRLQLRDNAKELGNGCFEFYHKCDNECMESVKNGTYDYPQYSEEARLNREE
ISGVKLE
Influenza A derived triple-helices
SEQ ID NO: 177
>gi|221342|dbj|BAA02768.1| hemagglutinin
[Influenza A virus (A/Suita/1/89(H1N1))
STQNAINGITNKVNSVIEKMNTQFTAVGKEFNKLERRMEYLNKKVDDGFLDIWTYNAELLVLLE
NERTLDFHDSNVKNLYEKVKSQLKNNAKEIGYGCFEFYHKCNNECMESVKNGTYDYPKYSEESK
LNREKIDGVKLE
SEQ ID NO: 178
>gi|407004|gb|AAA16880.1| hemagglutinin
[Influenza A virus (A/duck/WI/259/80(H1N1))
STQNAIDGITNKVNSVIEKMNTKFTAVGKEFNNLERRIENLNKKVDDGFLDVWTYNAELLVLLE
NERTLDFHDSNVRNLYEKVKSQLRNNAKELGNGCFEFYHKCDDECIESVKNGTYDYPKYSEESK
LNREEIDGVKLE
SEQ ID NO: 179
>gi|206236519|gb|AC106177.1| hemagglutinin
[Influenza A virus (A/HongKong/HK12MA21-3/2008(H3N2))
STQAAIDQINGKLNRVIEKTNEKFHQIEKEFSEVEGRIQDLEKYVEDTKIDLWSYNAELLVALE
NQHTIDLTDSEMNKLFEKTRRQLRENAEDMGNGCFKIYHKCDNACIESIRNGTYDHNVYRDEAL
NNRFQIKGVELK
SEQ ID NO: 180
>gi|305171|gb|AAA64366.1| hemagglutinin
[Influenza A virus (A/Singapore/1/1957(H2N2))
STQKAFDGITNKVNSVIEKMNTQFEAVGKEFSNLERRLENLNKKMEDGFLDVWTYNAELLVLME
NERTLDFHDSNVKNLYDKVRMQLRDNVKELGNGCFEFYHKCDDECMNSVKNGTYDYPKYEEESK
LNRNEIKGVKLS
SEQ ID NO: 181
>gi|134044971|gb|ABO51994.1|hemagglutinin
[Influenza A virus (A/mallard/Ohio/653/2002(H6N2))
STQKAIDGITNKVNSIIDKMNTQFEAVEHEFSNLERRIGNLNKRMEDGFLDVWTYNAELLVLLE
NERTLDMHDANVKNLHEKVKSQLKDNAKDLGNGCFEFWHKCDNDCIKSVKNGTYDYPKYQEESR
LNRQEIKSVMLE
SEQ ID NO: 182
>gi|158604880|gb|ABW74711.1| hemagglutinin
[Influenza A virus (A/Indonesia/TLL011/2006(H5N1))
STQKAIDGVTNKVNSIIDKMNTQFEAVGREFNNLERRIENLNKKMEDGFLDVWTYNAELLVLME
NERTLDFHDSNVKNLYDKVRLQLRDNAKELGNGCFEFYHKCDNECMESIRNGTYNYPQYSEEAR
LKREEISGVKLE
SEQ ID NO: 183
>gi|145280564|gb|ABP49542.1| hemagglutinin
[Influenza A virus (A/mallard/Ohio/81/1986(Mixed))
STQKAIDGITNKVNSIIDKMNTQFEAVEHEFSSLERRIDNLNKRMEDGFLDVWTYNAELLVLLE
NERTLDMHDANVKNLHEKVKSQLKDNAKDLGNGCFEFWHKCDDECINSVKNGTYDYPKYQEESR
LNRQEIKSVMLE
SEQ ID NO: 184
>gi|122900|sp|P26099.1|HEMA_I73A4 RecName:
Full = Hemagglutinin; Contains: RecName: Full = Hemagglutinin HA1
chain; Contains: RecName: Full = Hemagglutinin HA2 chain; Flags:
Precursor
STQSAINQITGKLNRLIEKTNQQFELIDNEFNEIEKQIGNVINWTRDSIIEVWSYNAEFLVAVE
NQHTIDLTDSEMNKLYEKVRRQLRENAEEDGNGCFEIFHQCDNDCMASIRNNTYDHKKYRKEAI
QNRIQIDAVKLS
SEQ ID NO: 185
>gi|229598493|gb|ACQ83087.1| hemagglutinin
[Influenza A virus (A/northern
shoveler/California/HKWF1204/2007(H8N4))
STQEAIDKITNKVNNIVDKMNREFEVVNHEFSEVEKRINMINDKIDDQIEGLWAYNAELLVLLE
NQKTLDEHDSNVKNLFDEVKRRLSTNAMDAGNGCFDILHKCNNECMETIKNGTYNHKEYEEEAK
LERSKINGVKLE
SEQ ID NO: 186
>gi|61661418|gb|AAX51299.1| hemagglutinin
[Influenza A virus (A/chicken/Shandong/2/02(H9N?))
STQRAIDKITSKVNNIVDKMNKQYEIIDHEFSEVETRLNMINNKIDDQIQDIWAYNAELLVLLE
NQKTLDEHDANVNNLYNKVKRALGSNAVEDGKGCFELYHKCDDQCMETIRNGTYNKRKYKEESR
LERQKIEGVKLE
SEQ ID NO: 187
>gi|156992297|gb|ABU99135.1| hemagglutinin
[Influenza A virus (A/Duck/Indonesia/Jakarta Utara1631-
29/2006(H10))
STQTAIDQITGKLNRLIEKTNTEFESIESEFSQIEHQIGNVINWTKDSITDIWTYQAELLVAME
NQHTIDMADSEMLNLYERVRKQLRQNAEEDGKGCFEIYHTCDDSCMESIRNNTYDHSQYREEAL
LNRLNINPVELS
SEQ ID NO: 188
>gi|90186540|gb|ABD91535.1| hemagglutinin
[Influenza A virus (A/ruddy turnstone/NJ/650678/2002(H11N4))
STQKAIDQITSKVNNIVDRMNTNFESVQHEFSEIEERINQLSAHVDDSLIDIWSYNAQLLVLLE
NEKTLDLHDSNVRNLHEKVRRMLKDNAKDEGNGCFTFYHKCDNECIEKVRNGTYDHKEFEEESK
LNRQEIEGVKLD
SEQ ID NO: 189
>gi|254952420|gb|ACT97061.1| hemagglutinin
[Influenza A virus (A/mallard/Switzerland/WV4060166/2006(H12N2))
STQKAIDNMQNKLNNVIDKMNKQFEVVKHEFSEVESRINMINSKIDDQITDIWAYNAELLVLLE
NQKTLDEHDANVRNLHDRVRRVLKENAIDTGDGCFEILHKCDDGCMDTIKNGTYNHQDYEEESK
LERQRINGVKLE
SEQ ID NO: 190
>gi|82653632|gb|ABB87811.1| hemagglutinin
[Influenza A virus (A/laughing gull/DE/2838/1987(H13N2))
STQKAIDQITTKINNIIDKMNGNYDSIRGEFSQVERRINMLADRIDDAVTDVWSYNAKLLVLLE
NDKTLDMHDANVRNLHEQVRRTLKANAINEGNGCFELLHKCNDSCMETIRNGTYNHAEYAEESK
LKRQEIEGIKLK
SEQ ID NO: 191
>gi|419003|pir||A46339 hemagglutinin precursor
- influenza A virus (strain A/ Mallard/Gurjev/263/82 [H14N5])
STQAAIDQINGKLNRLIEKTNEKYHQIEKEFEQVEGRIQDLEKYVEDTKIDLWSYNAELLVALE
NQHTIDVTDSEMNKLFERVRRQLRENAEDQGNGCFEIFHQCDNNCIESIRNGTYDHNIYRDEAI
NNRIKINPVTLT
SEQ ID NO: 192
>gi|1226071|gb|AAA96134.1| hemagglutinin
[Influenza A virus (A/shearwater/West Australia/2576/79(H15N9))
STQAAIDQITGKLNRLIEKTNKQFELIDNEFTEVEQQIGNVINWTRDSLTEIWSYNAELLVAME
NQHTIDLADSEMNKLYERVRRQLRENAEEDGTGCFEIFHRCDDQCMESIRNNTYNHTEYRQEAL
QNRIMINPVKLS
SEQ ID NO: 193
>gi|56425021|gb|AAV91217.1| hemagglutinin
[Influenza A virus (A/black-headed gull/Sweden/5/99(H16N3))
STQKAIDEITTKINNIIEKMNGNYDSIRGEFNQVEKRINMLADRVDDAVTDIWSYNAKLLVLLE
NGRTLDLHDANVRNLHDQVYRILKSNAIDEGDGCFNLLHKCNDSCMETIRNGTYNHEDYREESQ
LKRQEIEGIKLK
SEQ ID NO: 194
>gi|324231|gb|AAA43222.1| hemagglutinin
[Influenza A virus (A/ruddy turnstone/NJ/47/1985(H4N6))
STQAAIDQINGKLNRLIEKTNEKYHQIEKEFEQVEGRIQDLEKYVEDTKIDLWSYNAELLVALE
NQHTIDVTDSEMNKLFERVRRQLRENAEDKGNGCFEIFHQCDNNCIESIRNGTYDHDIYRDEAI
NNRFQIQGVKLT
SEQ ID NO: 195
>gi|56291612|emb|CAE48276.1| hemagglutinin
[Influenza A virus (A/Chicken/Italy/1067/99(H7N1))
STQSAIDQVTGKLNRLIEKTNQQFKLIDNEFTEVEKQIGNVINWTRDSMTEVWSYNAELLVAME
NQHTIDLADSEMNKLYERVKRQLRENAEEDGTGCFEIFHKCDDDCMASIRNNTYDHSKYREEAM
QNRIQIDPVKLS
SEQ ID NO: 196
>gi|82020763|sp|Q67333.1|HEMA_I57A5 RecName:
Full = Hemagglutinin; Contains: RecName: Full = Hemagglutinin HA1
chain; Contains: RecName: Full = Hemagglutinin HA2 chain; Flags:
Precursor
STQKAFDGITNKVNSVIEKMNTQFEAVGKEFSNLERRLENLNKKMEDGFLDVWTYNAELLVLME
NERTLDFHDSNVKNLYDKVRMQLRDNVKELGNGCFEFYHKCDDECMNSVKNGTYDYPKYEEESK
LNRNEIKGVKLS
SEQ ID NO: 197
>gi|122853|sp|P03437.1|HEMA_I68A0 RecName:
Full = Hemagglutinin; Contains: RecName: Full = Hemagglutinin HA1
chain; Contains: RecName: Full = Hemagglutinin HA2 chain; Flags:
Precursor
STQAAIDQINGKLNRVIEKTNEKFHQIEKEFSEVEGRIQDLEKYVEDTKIDLWSYNAELLVALE
NQHTIDLTDSEMNKLFEKTRRQLRENAEEMGNGCFKIYHKCDNACIESIRNGTYDHDVYRDEAL
NNRFQIKGVELK
SEQ ID NO: 198
>gi|160395568|sp|Q9WFX3.2|HEMA_I18A0 RecName:
Full = Hemagglutinin; Contains: RecName: Full = Hemagglutinin HA1
chain; Contains: RecName: Full = Hemagglutinin HA2 chain; Flags:
Precursor
STQNAIDGITNKVNSVIEKMNTQFTAVGKEFNNLERRIENLNKKVDDGFLDIWTYNAELLVLLE
NERTLDFHDSNVRNLYEKVKSQLKNNAKEIGNGCFEFYHKCDDACMESVRNGTYDYPKYSEESK
LNREEIDGVKLE
SEQ ID NO: 199
>gi|9518852|gb|AAB29091.2| hemagglutinin [H1N1
swine influenza virus
STQNAIDGITNKVNSVIEKMNTQFTAVGKEFNHLEKRIENLNKKVDDGFLDVWTYNAELLVLLE
NERTLDFHDSNVKNLYEKVRSQLRNNAKEIGNGCFEFYHKCDDTCMESVKNGTYDYSKYSEESK
LNREVIDGVKLD
SEQ ID NO: 200
>gi|238623304|gb|ACR47014.1| hemagglutinin
[Influenza A virus (A/reassortant/NYMC X-179A(California/07/2009
x NYMC X-157)(H1N1))
STQNAIDEITNKVNSVIEKMNTQFTAVGKEFNHLEKRIENLNKKVDDGFLDIWTYNAELLVLLE
NERTLDYHDSNVKNLYEKVRSQLKNNAKEIGNGCFEFYHKCDNTCMESVKNGTYDYPKYSEEAK
LNREEIDGVKLE
Influenza B derived triple-helices
SEQ ID NO: 201
>gi|122961|sp|P03460.1|HEMA_INBLE RecName:
Full = Hemagglutinin; Contains: RecName: Full = Hemagglutinin HA1
chain; Contains: RecName: Full = Hemagglutinin HA2 chain; Flags:
Precursor
STQEAINKITKNLNYLSELEVKNLQRLSGAMNELHDEILELDEKVDDLRADTISSQIELAVLLS
NEGIINSEDEHLLALERKLKKMLGPSAVEIGNGCFETKHKCNQTCLDRIAAGTFNAGDFSLPTF
DSLNITAASLND
SEQ ID NO: 202
>gi|122955|sp|P10757.1|HEMA_INBEN RecName:
Full = Hemagglutinin; Contains: RecName: Full = Hemagglutinin HA1
chain; Contains: RecName: Full = Hemagglutinin HA2 chain; Flags:
Precursor
STQEAINKITKNLNSLSELEVKNLQRLSGAMDELHNEILELDEKVDDLRADTISSQIELAVLLS
NEGIINSEDEHLLALERKLKKMLGPSAVDIGNGCFETKHKCNQTCLDRIAAGTFNAGEFSLPTF
DSLNITAASLND
Influenza C derived triple-helices
SEQ ID NO: 203
>gi|122976|sp|P07975.1|HEMA_INCJH RecName:
Full = Hemagglutinin-esterase-fusion glycoprotein; Short = HEF;
Contains: RecName: Full = Hemagglutinin-esterase-fusion
glycoprotein chain 1; Short = HEF1; Contains: RecName:
Full = Hemagglutinin-esterase-fusion glycoprotein chain 2;
Short = HEF2; Flags: Precursor
SAEKGFEKIGNDIQILKSSINIAIEKLNDRISHDEQAIRDLTLEIENARSEALLGELGIIRALL
VGNISIGLQESLWELASEITNRAGDLAVEVSPGCWIIDNNICDQSCQNFIFKFNETAPVPTIPP
LDTKIDLQSDPFYW
SEQ ID NO: 204
>gi|119364590|sp|P68762.2|HEMA_INCAA RecName:
Full = Hemagglutinin-esterase-fusion glycoprotein; Short = HEF;
Contains: RecName: Full = Hemagglutinin-esterase-fusion
glycoprotein chain 1; Short = HEF1; Contains: RecName:
Full = Hemagglutinin-esterase-fusion glycoprotein chain 2;
Short = HEF2; Flags: Precursor
SAEKGFEKIGNDIQILRSSTNIAIEKLNDRISHDEQAIRDLTLEIENARSEALLGELGIIRALL
VGNISIGLQESLWELASEITNRAGDLAVEVSPGCWVIDNNICDQSCQNFIFKFNETAPVPTIPP
LDTKIDLQSDPFYW
Bivalent molecules, nucleotide sequences
SEQ ID NO: 205
(#500)
atgaaccggggagtcccttttaggcacttgcttctggtgctgcaactggcgctcctcccagcag
ccactcagggaaagaaagtggtgctgggcaaaaaaggggatacagtggaactgacctgtacagc
ttcccagaagaagagcatacaattccactggaaaaactccaaccagataaagattctgggaaat
cagggctccttcttaactaaaggtccatccaagctgaatgatcgcgctgactcaagaagaagcc
tttgggaccaaggaaactttcccctgatcatcaagaatcttaagatagaagactcagatactta
catctgtgaagtggaggaccagaaggaggaggtgcaattgctagtgttcggattgactgccaac
tctgacacccacctgcttcaggggcagagcctgaccctgaccttggagagcccccctggtagta
gcccctcagtgcaatgtaggagtccaaggggtaaaaacatacagggggggaagaccctctccgt
gtctcagctggagctacaggatagtggcacctggacatgcactgtcttgcagaaccagaagaag
gtggagttcaaaatagacatcgtggtgctagctttccagaaggcctccagcatagtctataaga
aagagggggaacaggtggagttctccttcccactcgcctttacagttgaaaagctgacgggcag
tggcgagctgtggtggcaggcggagagggcttcctcctccaagtcttggatcacctttgacctg
aagaacaaggaagtgtctgtaaaacgggttacccaggaccctaagctccagatgggcaagaagc
tcccgctccacctcaccctgccccaggccttgcctcagtatgctggctctggaaacctcaccct
ggcccttgaagcgaaaacaggaaagttgcatcaggaagtgaacctggtggtgatgagagccact
cagctccagaaaaatttgacctgtgaggtgtggggacccacctcccctaagctgatgctgagtt
tgaaactggagaacaaggaggcaaaggtctcgaagcgggagaaggcggtgtgggtgctgaaccc
agaagcggggatgtggcagtgtctgctgagtgactcgggacaggtcctgctggaatccaacatc
aaggttctgcccacatggtccaccccggtctcgagtgggggatccggaggttcaggtgggtctg
gaggctcggggggctcctcaggtgaatgggatagagaaattaataactatacttctctgatcca
cagccttatagaggaatcgcaaaaccaacaggagaagaacgaacaggagcttctggaactggat
aaatgggcatcgctttggaattggttc
SEQ ID NO: 206
(#521)
atgaaccggggagtcccttttaggcacttgcttctggtgctgcaactggcgctcctcccagcag
ccactcagggaaagaaagtggtgctgggcaaaaaaggggatacagtggaactgacctgtacagc
ttcccagaagaagagcatacaattccactggaaaaactccaaccagataaagattctgggaaat
cagggctccctcttaactaaaggtccatccaagctgaatgatcgcgctgactcaagaagaagcc
tttgggaccaaggaaactttcccctgatcatcaagaatcttaagatagaagactcagatactta
catctgtgaagtggaggaccagaaggaggaggtgcaattgctagtgttcggattgactgccaac
tctgacacccacctgcttcaggggcagagcctgaccctgaccttggagagcccccccggtagta
gcccctcagtgcaatgtaggagtccaaggggtaaaaacatacagggggggaagaccctctccgt
gtctcagctggagctacaggatagtggcacctggacatgcactgtcttgcagaaccagaagaag
gtggagttcaaaatagacatcgtggtgctagctttccagaaggcctccagcatagtctataaga
aagagggggaacaggtggagttctccttcccactcgcctttacagttgaaaagctgacgggcag
tggcgagctgtggtggcaggcggagagggcttcctcctccaagtcttggatcacctttgacctg
aagaacaaggaagtgtctgtaaaacgggttacccaggaccctaagctccagatgggcaagaagc
tcccgctccacctcaccctgccccaggccttgcctcagtatgctggctctggaaacctcaccct
ggcccttgaagcgaaaacaggaaagttgcatcaggaagtgaacctggtggtgatgagagccact
cagctccagaaaaatttgacctgtgaggtgtggggacccacctcccctaagctgatgctgagtt
tgaaactggagaacaaggaggcaaaggtctcgaagcgggagaaggcggtgtgggtgctgaaccc
agaagcggggatgtggcagtgtctgctgagtgactcgggacaggtcctgctggaatccaacatc
aaggttctgcccacatggtccaccccggtctcgagtgggggatccggaggttcaggtgggtctg
gaggctcggggggctcctcaggttctggtatagtgcagcagcagaacaatttgctgagggctat
tgaggcgcaacagcatctgttgcaactcacagtctggggcatcaagcagctccaggcaagaatc
ctgtaa
SEQ ID NO: 207
(#517)
atgaaccggggagtcccttttaggcacttgcttctggtgctgcaactggcgctcctcccagcag
ccactcagggaaagaaagtggtgctgggcaaaaaaggggatacagtggaactgacctgtacagc
ttcccagaagaagagcatacaattccactggaaaaactccaaccagataaagattctgggaaat
cagggctccttcttaactaaaggtccatccaagctgaatgatcgcgctgactcaagaagaagcc
tttgggaccaaggaaactttcccctgatcatcaagaatcttaagatagaagactcagatactta
catctgtgaagtggaggaccagaaggaggaggtgcaattgctagtgttcggattgactgccaac
tctgacacccacctgcttcaggggcagagcctgaccctgaccttggagagcccccctggtagta
gcccctcagtgcaatgtaggagtccaaggggtaaaaacatacagggggggaagaccctctccgt
gtctcagctggagctacaggatagtggcacctggacatgcactgtcttgcagaaccagaagaag
gtggagttcaaaatagacatcgtggtgccagctttccagaaggcctccagcatagtctataaga
aagagggggaacaggtggagttctccttcccactcgcctttacagttgaaaagctgacgggcag
tggcgagctgtggtggcaggcggagagggcttcctcctccaagtcttggatcacctttgacctg
aagaacaaggaagtgtctgtaaaacgggttacccaggaccctaagctccagatgggcaagaagc
tcccgctccacctcaccctgccccaggccttgcctcagtatgctggctccggaaacctcaccct
ggcccttgaagccaaaacaggaaagttgcatcaggaagtgaacctggtggtgatgagagccact
cagctccagaaaaatttgacctgtgaggtgtggggacccacctcccctaagctgatgctgagtt
tgaaactggagaacaaggaggcaaaggtctcgaagcgggagaaggcggtgtgggtgctgaaccc
agaagcggggatgtggcagtgtctgctgagtgactcgggacaggtcctgctggaatccaacatc
aaggttctgcccacatggtccaccccggtctcgagtgggggatccggaggttcaggtgggtctg
gaggctcggggggctcctcaggtctccaggcaagaatcctggctgtggaaagatacctaaagga
tcaacagctcctggggatttggggttaa
SEQ ID NO: 208
(#520)
atgaaccggggagtcccttttaggcacttgcttctggtgctgcaactggcgctcctcccagcag
ccactcagggaaagaaagtggtgctgggcaaaaaaggggatacagtggaactgacctgtacagc
ttcccagaagaagagcatacaattccactggaaaaactccaaccagataaagattctgggaaat
cagggctccttcttaactaaaggtccatccaagctgaatgatcgcgctgactcaagaagaagcc
tttgggaccaaggaaactttcccctgatcatcaagaatcttaagatagaagactcagatactta
catctgtgaagtggaggaccagaaggaggaggtgcaattgctagtgttcggattgactgccaac
tctgacacccacctgcttcaggggcagagcctgaccctgaccttggagagcccccctggtagta
gcccctcagtgcaatgtaggagtccaaggggtaaaaacatacagggggggaagaccctctccgt
gtctcagctggagctacaggatagtggcacctggacatgcactgtcttgcagaaccagaagaag
gtggagttcaaaatagacatcgtggtgctagctttccagaaggcctccagcatagtctataaga
aagagggggaacaggtggagttctccttcccactcgcctttacagttgaaaagctgacgggcag
tggcgagctgtggtggcaggcggagagggcttcctcctccaagtcttggatcacctttgacctg
aagaacaaggaagtgtctgtaaaacgggttacccaggaccctaagctccagatgggcaagaagc
tcccgctccacctcaccctgccccaggccttgcctcagtatgctggctctggaaacctcaccct
ggcccttgaagcgaaaacaggaaagttgcatcaggaagtgaacctggtggtgatgagagccact
cagctccagaaaaatttgacctgtgaggtgtggggacccacctcccctaagctgatgctgagtt
tgaaactggagaacaaggaggcaaaggtctcgaagcgggagaaggcggtgtgggtgctgaaccc
agaagcggggatgtggcagtgtctgctgagtgactcgggacaggtcctgctggaatccaacatc
aaggttctgcccacatggtccaccccggtctcgagtgggggatccggaggttcaggtgggtctg
gaggctcggggggctcctcaggtctccaggcaagaatcctggctgtggaaagatacctaaagga
tcaacagctcctggggatttggggttgctccggaaaactcatttgcaccactgctgtgccttgg
aatgctagttggagtaataaatctctggaacagattcggaatcacacgacctaa
SEQ ID NO: 209
(#518)
atgaaccggggagtcccttttaggcacttgcttctggtgctgcaactggcgctcctcccagcag
ccactcagggaaagaaagtggtgctgggcaaaaaaggggatacagtggaactgacctgtacagc
ttcccagaagaagagcatacaattccactggaaaaactccaaccagataaagattctgggaaat
cagggctccttcttaactaaaggtccatccaagctgaatgatcgcgctgactcaagaagaagcc
tttgggaccaaggaaactttcccctgatcatcaagaatcttaagatagaagactcagatactta
catctgtgaagtggaggaccagaaggaggaggtgcaattgctagtgttcggattgactgccaac
tctgacacccacctgcttcaggggcagagcctgaccctgaccttggagagcccccctggtagta
gcccctcagtgcaacgtaggagtccaaggggtaaaaacatacagggggggaagaccctctccgt
gtctcagctggagctacaggatagtggcacctggacatgcactgtcttgcagaaccagaagaag
gtggagttcaaaatagacatcgtggtgctagctttccagaaggcctccagcatagtctataaga
aagagggggaacaggtggagttctccttcccactcgcctttacagttgaaaagctgacgggcag
tggcgagctgtggtggcaggcggagagggcttcctcctccaagtcttggatcacctttgacctg
aagaacaaggaagtgtctgtaaaacgggttacccaggaccctaagctccagatgggcaagaagc
tcccgctccacctcaccctgccccaggccttgcctcagtatgctggctctggaaacctcaccct
ggcccttgaagcgaaaacaggaaagttgcatcaggaagtgaacctggtggtgatgagagccact
cagctccagaaaaatttgacctgtgaggtgtggggacccacctcccctaagctgatgctgagtt
tgaaactggagaacaaggaggcaaaggtctcgaagcgggagaaggcggtgtgggtgctgaaccc
agaagcggggatgtggcagtgtctgctgagtgactcgggacaggtcctgctggaatccaacatc
aaggttctgcccacatggtccaccccggtctcgagtgggggatccggaggttcaggtgggtctg
gaggctcggggggctcctcaggtctccaggcaagaatcctggctgtggaaagatacctaaagga
tcaacagctcctggggatttggggttcctctggaaaactcatttccaccactgctgtgccttgg
aatgctagttggagtaataaatctctggaacagatttggaatcacacgacctaa
SEQ ID NO: 210
(#519)
atgaaccggggagtcccttttaggcacttgcttctggtgctgcaactggcgctcctcccagcag
ccactcagggaaagaaagtggtgctgggcaaaaaaggggatacagtggaactgacctgtacagc
ttcccagaagaagagcatacaattccactggaaaaactccaaccagataaagattctgggaaat
cagggctccttcttaactaaaggtccatccaagctgaatgatcgcgctgactcaagaagaagcc
tttgggaccaaggaaactttcccctgatcatcaagaatcttaagatagaagactcagatactta
catctgtgaagtggaggaccagaaggaggaggtgcaattgctagtgttcggattgactgccaac
tctgacacccacctgcttcaggggcagagcctgaccctgaccttggagagcccccctggtagta
gcccctcagtgcaatgtaggagtccaaggggtaaaaacatacagggggggaagaccctctccgt
gtctcagctggagctacaggatagtggcacctggacatgcactgtcttgcagaaccagaagaag
gtggagttcaaaatagacatcgtggtgctagctttccagaaggcctccagcatagtctataaga
aagagggggaacaggtggagttctccttcccactcgcctttacagttgaaaagctgacgggcag
tggcgagctgtggtggcaggcggagagggcttcctcctccaagtcttggatcacctttgacctg
aagaacaaggaagtgtctgtaaaacgggttacccaggaccctaagctccagatgggcaagaagc
tcccgctccacctcaccctgccccaggccttgcctcagtatgctggctctggaaacctcaccct
ggcccttgaagcgaaaacaggaaagttgcatcaggaagtgaacctggtggtgatgagagccact
cagctccagaaaaatttgacctgtgaggtgtggggacccacctcccctaagctgatgctgagtt
tgaaactggagaacaaggaggcaaaggtctcgaagcgggagaaggcggtgtgggtgctgaaccc
agaagcggggatgtggcagtgtctgctgagtgactcgggacaggtcctgctggaatccaacatc
aaggttctgcccacatggtccaccccggtctcgagtgggggatccggaggttcaggtgggtctg
gaggctcggggggctcctcaggtaccactgctgtgccttggaatgctagttggagtaataaatc
tctggaacagatttggaatcacacgacctaa
SEQ ID NO: 211
(#538)
atgaaccggggagtcccttttaggcacttgcttctggtgctgcaactggcgctcctcccagcag
ccactcagggaaagaaagtgcatcatcaccatcaccacaaagttgtgctgggcaaaaaagggga
tacagtggaactgacctgtacagcttcccagaagaagagcatacaattccactggaaaaactcc
aaccagataaagattctgggaaatcagggctccttcttaactaaaggtccatccaagctgaatg
atcgcgctgactcaagaagaagcctttgggaccaaggaaactttcccctgatcatcaagaatct
taagatagaagactcagatacttacatctgtgaagtggaggaccagaaggaggaggtgcaattg
ctagtgttcggattgactgccaactctgacacccacctgcttcaggggcagagcctgaccctga
ccttggagagcccccctggtagtagcccctcagtgcaatgtaggagtccaaggggtaaaaacat
acagggggggaagaccctctccgtgtctcagctggagctacaggatagtggcacctggacatgc
actgtcttgcagaaccagaagaaggtggagttcaaaatagacatcgtggtgctagctttccaga
aggcctccagcatagtctataagaaagagggggaacaggtggagttctccttcccactcgcctt
tacagttgaaaagctgacgggcagtggcgagctgtggtggcaggcggagagggcttcctcctcc
aagtcttggatcacctttgacctgaagaacaaggaagtgtctgtaaaacgggttacccaggacc
ctaagctccagatgggcaagaagctcccgctccacctcaccctgccccaggccttgcctcagta
tgctggctctggaaacctcaccctggcccttgaagcgaaaacaggaaagttgcatcaggaagtg
aacctggtggtgatgagagccactcagctccagaaaaatttgacctgtgaggtgtggggaccca
cctcccctaagctgatgctgagtttgaaactggagaacaaggaggcaaaggtctcgaagcggga
gaaggcggtgtgggtgctgaacccagaagcggggatgtggcagtgtctgctgagtgactcggga
caggtcctgctggaatccaacatcaaggttctgcccacatggtccaccccggtctcgagtgggg
gatccggaggttcaggtgggtctggaggctcggggggctcctcaggtaccactgctgtgccttg
gaatgctagttggagtaataaatctctggaacagatttggaatcacacgacctaa
(#539)
acgaaccggggagtcccttttaggcacttgcttctggtgctgcaactggcgctcctcccagcagccactc
agggaaagaaagtgcatcatcaccatcaccacaaagttgtgctgggcaaaaaaggggatacagtggaact
gacctgcacagcttcccagaagaagagcatacaattccactggaaaaactccaaccagataaagattctg
ggaaatcagggctccttcttaactaaaggtccatccaagctgaatgatcgcgctgactcaagaagaagcc
tttgggaccaaggaaactttcccctgatcatcaagaatcttaagatagaagactcagatacttacatctg
tgaagtggaggaccagaaggaggaggtgcaattgctagtgttcggattgactgccaactctgacacccac
ctgcttcaggggcagagcctgaccctgaccttggagagcccccctggtagtagcccctcagtgcaatgta
ggagtccaaggggtaaaaacatacagggggggaagaccctctccgtgtctcagctggagctacaggatag
tggcacctggacatgcactgtcttgcagaaccagaagaaggtggagttcaaaatagacatcgtggtgcta
gctttccagaaggcctccagcatagtctataagaaagagggggaacaggtggagttctccttcccactcg
cctttacagttgaaaagctgacgggcagtggcgagctgtggtggcaggcggagagggcttcctcctccaa
gtcttggatcacctttgacctgaagaacaaggaagtgtctgtaaaacgggttacccaggaccctaagctc
cagatgggcaagaagctcccgctccacctcaccctgccccaggccttgcctcagtatgctggctctggaa
acctcaccctggcccttgaagcgaaaacaggaaagttgcatcaggaagtgaacctggtggtgatgagagc
cactcagctccagaaaaatttgacctgtgaggtgtggggacccacctcccctaagctgatgctgagtttg
aaactggagaacaaggaggcaaaggtctcgaagcgggagaaggcggtgtgggtgctgaacccagaagcgg
ggatgtggcagtgtctgctgagtgactcgggacaggtcctgctggaatccaacatcaaggttctgcccac
atggtccaccccggtctcgagtgggggatccggaggttcaggtgggtctggaggctcggggggctcctca
ggtgaatgggatagagaaattaataactatacttctctgatccacagccttatagaggaatcgcaaaacc
aacaggagaagaacgaacaggagcttctggaactggataaatgggcatcgctttggaattggttc
SEQ ID NO: 226
(Chim-sCD4-T20, comprising cCD4 with point
mutation found in Chimpanzee(underlined))
atgaaccggggagtcccttttaggcacttgcttctggtgctgcaactggcgctcctcccagcagccactc
agggaaagaaagtggtgctgggcaaaaaaggggatacagtggaactgacctgtacagtcccagaagaaga
gcatacaattccactggaaaaactccaaccagataaagattctgggaaatcagggctccttcttaactaa
aggtccatccaagctgaatgatcgcgctgactcaagaagaagcttgggaccaaggaaactttcccctgat
catcaagaatcttaagatagaagactcagatacttacatctgtgGagtggaggaccagaaggaggaggtg
caattgctagtgttcggattgactgccaatctgacacccacctgcttcaggggcagagcctgaccctgac
cttggagagcccccctggtagtagcccctcaggcaatgtaggagtccaaggggtaaaaacatacaggggg
ggaagaccctctccggtctcagctggagctacaggatagtggcacctggacatgcactgtcttgcagaac
cagaagaaggtggagttcaaatagacatcgtggtgctagctttccagaaggcctccagcatagtctataa
gaagagggggaacaggtggagttctccttcccactcgcctttacagttgaaaagctgacgggcagtggcg
agctgtgtggcaggcggagagggcttcctcctccaagtcttggatcacctttgacctaagaacaaggaag
tgtctgtaaaacgggttacccaggaccctaagctccagatgggcaagaagctcccgctccacccaccctg
ccccaggccttgcctcagtatgctggctctggaaacctcacccggcccttgaagcgaaaacaggaaagtt
gcatcaggaagtgaacctggtggtgatgagagccactcagctccagaaaatttgacctgtgaggtgtggg
gacccacctcccctaagctgatgctgagttgaaactggagaacaaggaggcaaaggtctcgaagcgggag
aaggcggtgtgggtgctgaacccagaagcggggatgtgcagtgtctgctgagtgactcgggacaggtcct
gctggaatccaacataaggttctgcccacatggtccaccccggtctcgagtgggggatccggaggttcag
gtgggtctggaggctcggggggctctcaggtgaatgggatagagaaattaataactatacttctctgatc
ccagccttatagaggaatcgcaaaaccaacaggagaagaacgaacaggagcttctggaactggataaatg
ggcatcgctttgaattggttctaa
SEQ ID NO: 227
(cMyc-His6-TEV SCD4 T20-IRES-Puro)
atgagggcctggatcttctttctcctttgcctggccgggagggctctggcagctagcgaacaaaaactca
tctcagaagaggatctgaatatgcataccggtcatcatcaccatcaccatggtgagatctttattttcag
ggtaagaaagtggtgctgggcaaaaaaggggatacagtggaactgacctgtacagcttcccagaaaagag
catacaattccactggaaaaactccaaccagataaagattctggaaatcagggctccttcttaactaaag
gtccatccaagctgaatgatcgcgctgactcaagaagaagcctttgggaccaggaaactttcccctgatc
atcaagaatcttaagatagaagactcagaacttacatctgtgaagtggaggaccagaaggaggaggtgca
attgctagtgttcggattgactgccaactctgacacccactgcttcaggggcagagcctgaccctgacct
tggagagcccccctgtagtagcccctcagtgcaatgtaggagtccaaggggtaaaaacatacaggggggg
aagaccctctccgtgtctcagctggactacaggatagtggcacctggacatgcactgtcttgcagaacca
gagaaggtggagttcaaaatagacatcgtggtgctagctttccagaaggcctccagcatagtctataaga
aagagggggaacagtggagttctccttcccactcgcctttacagttgaaaagctgacggcagtggcgagc
tgtggtggcaggcggagagggcttcctcctccaagtcttggatcacctttgacctgaagaacaaggaagt
tctgtaaaacgggttacccaggaccctaagctccagatgggcagaagctcccgctccacctcaccctgcc
ccaggccttgcctcagtatgctggctctggaaacctcaccctggcccttgaagcgaaacaggaaagttgc
atcaggaagtgaacctggtggtgatgagaccactcagctccagaaaaatttgacctgtgaggtgtgggga
cccacctcccctaagctgatgctgagtttgaaactggagaacaagaggcaaaggtctcgaagcgggagaa
ggcggtgtgggtgctaacccagaagcggggatgtggcagtgtctgctgagtgactcgggacaggtcctgc
tggaatccaacatcaaggttctgcccacatgtccaccccggtctcgagtgggggatccggaggttcaggt
ggtctggaggctcggggggctcctcaggtgaatgggatagagaaattaataactacacttctctgatcca
cagccttatagaggaatccaaaaccaacaggacaagaacgaacaggagcttctggaatggataaatgggc
atcgctttggaattggttc
SEQ ID NO: 228
(sCD4 T20 Sifurvitide (Sifurvitide underlined))
atgaaccggggagtcccttttaggcacttgcttctggtgctgcaactggcgctcctccc
agcagccactcagggaaagaaagtggtgctgggcaaaaaaggggatacagtggaactga
cctgtacagtcccagaagaagagcatacaattccactggaaaaactccaaccagataaa
gattctgggaaatcagggctccttcttaactaaaggtccatccaagctgaatgatcgcg
ctgactcaagaagaagcttgggaccaaggaaactttcccctgatcatcaagaatcttaa
gatagaagactcagatacttacatctgtgaagtggaggaccagaaggaggaggtgcaat
tgctagtgttcggattgactgccaatcgacacccacctgcttcaggggcagagcctgac
cctgaccttggagagcccccctggtagtagcccctcagtgcaatgtaggagtccaaggg
gtaaaaacatacagggggggaagaccctctccggtccagctggagctacaggatagtgg
cacctggacatgcactgtcttgcagaaccagaagaaggtggagttcaaaatagacatcg
tggtgctagctttccagaaggcctccagcatagtctataagaagaggggaacaggtgga
gttctccttcccactcgcctttacagttgaaaagctgacgggcagtggcgagctgtggt
ggcaggcggagagggcttcctcctccaagtcttggatcacctttgacctaagaaaagga
agtgtctgtaaaacgggttacccaggaccctaagctccagatgggcaagaagctcccgc
tccacctcaccctgccccaggccttgcctcagtatgctggctctggaaacctcacccgg
ccctgaagcgaaaacaggaaagttgcatcaggaagtgaacctggtggtgatgagagcca
ctcagctccagaaaaatttgacctgtgaggtgtggggacccacctcccctaagctgatg
ctgagttgaaactgagaacaaggaggcaaaggtctcgaagcgggagaaggcggtgtggg
tgctgaacccagaagcggggatgtggcagtgtctgctgagtgactcgggacaggtcctg
ctggaatccaacataaggttctgcccacatggtccaccccggtctcgagtgggggatcc
ggaggttcaggtgggtctggaggctcggggggctcctcaggttct tgg gaa act
tgg gagcgg gaa attgaa aac tatacc cgt caa att tac cggata cta
gaa gag agc cag gaa caacaa gat cgg aac gag aga gat ctgctc
gaa
SEQ ID NO: 229
(sCD4-DSL20 (Alternative sequence of the helix
that interacts with gp120))
atgaaccggggagtcccctttaggcacttgcttctggtgctgcaactggcgctcctccc
agcagccactcagggaaagaaagtggtgctgggcaaaaaaggggatacagtggaactga
cctgtacagtcccagaagaagagcatacaattccactggaaaaactccaaccagataaa
gattctgggaaatcagggctccttcttaactaaaggtccatccaagctgaatgatcgcg
ctgactcaagaagaagcttgggaccaaggaaactttcccctgatcatcaagaatcttaa
gatagaagactcagatacttacatctgtgaagtggaggaccagaaggaggaggtgcaat
tgctagtgttcggattgactgccaatcgacacccacctgcttcaggggcagagcctgac
cctgaccttggagagcccccctggtagtagcccctcagtgcaatgtaggagtccaaggg
gtaaaaacatacagggggggaagaccctctccggtccagctggagctacaggatagtgg
cacctggacatgcactgtcttgcagaaccagaagaaggtggagttcaaaatagacatcg
tggtgctagctttccagaaggcctccagcatagtctataagaagaggggaacaggtgga
gttctccttcccactcgcctttacagttgaaaagctgacgggcagtggcgagctgtggt
ggcaggcggagagggcttcctcctccaagtcttggatcacctttgacctaagaaaagga
agtgtctgtaaaacgggttacccaggaccctaagctccagatgggcaagaagctcccgc
tccacctcaccctgccccaggccttgcctcagtatgctggctctggaaacctcacccgg
ccctgaagcgaaaacaggaaagttgcatcaggaagtgaacctggtggtgatgagagcca
ctcagctccagaaaaatttgacctgtgaggtgtggggacccacctcccctaagctgatg
ctgagttgaaactgagaacaaggaggcaaaggtctcgaagcgggagaaggcggtgtggg
tgctgaacccagaagcggggatgtggcagtgtctgctgagtgactcgggacaggtcctg
ctggaatccaacataaggttctgcccacatggtccaccccggtcccgagtgggggatcc
ggaggttcaggtgggtctggaggctcggggggctcctcaggtgaacgctacctgaaaga
tcagcaactgctcggcatctgggccgttctgggaagctgatccgtaccaccgcggtccc
ctggaacgcttcttggtcaaacaaatctctagagcagatctggaaccacacaacttgga
tggaatgggaccgggagatcaac
SEQ ID NO: 230
(sCD4-DSL20ss (alternative sequence of the helix
that interacts with gp120))
atgaaccggggagtcccttttaggcacttgcttctggtgctgcaactggcgctcctccc
agcagccactcagggaaagaaagtggtgctgggcaaaaaaggggatacagtggaactga
cctgtacagtcccagaagaagagcatacaattccactggaaaaactccaaccagataaa
gattctgggaaatcagggctccttcttaactaaaggtccatccaagctgaatgatcgcg
ctgactcaagaagaagcttgggaccaaggaaactttcccctgatcatcaagaatcttaa
gatagaagactcagatacttacatctgtgaagtggaggaccagaaggaggaggtgcaat
tgctagtgttcggattgactgccaatcgacacccacctgcttcaggggcagagcctgac
cctgaccttggagagcccccctggtagtagcccctcagtgcaatgtaggagtccaaggg
gtaaaaacatacagggggggaagaccctctccggtccagctggagctacaggatagtgg
cacctggacatgcactgtcttgcagaaccagaagaaggtggagttcaaaatagacatcg
tggtgctagctttccagaaggcctccagcatagtctataagaagaggggaacaggtgga
gttctccttcccactcgcctttacagttgaaaagctgacgggcagtggcgagctgtggt
ggcaggcggagagggcttcctcctccaagtcttggatcacctttgacctaagaaaagga
agtgtctgtaaaacgggttacccaggaccctaagctccagatgggcaagaagctcccgc
tccacctcaccctgccccaggccttgcctcagtatgctggctctggaaacctcacccgg
ccctgaagcgaaaacaggaaagttgcatcaggaagtgaacctggtggtgatgagagcca
ctcagctccagaaaaatttgacctgtgaggtgtggggacccacctcccctaagctgatg
ctgagttgaaactgagaacaaggaggcaaaggtctcgaagcgggagaaggcggtgtggg
tgctgaacccagaagcggggatgtggcagtgtctgctgagtgactcgggacaggtcctg
ctggaatccaacataaggttctgcccacatggtccaccccggtctcgagtgggggatcc
ggaggttcaggtgggtctggaggctcggggggctcctcaggtgaacgctacctgaaaga
tcagcaactgctcggcatctgggctcttctgggaagctgatctctaccaccgcggtccc
ctggaacgcttcttggtcaaacaaatctctagagcagatctggaaccacacaacttgga
tggaatgggaccgggagatcaac
SEQ ID NO: 231
(sCD4-DSL49 (alternative helix sequence thought
to interact with gp120))
atgaaccggggagtcccttttaggcacttgcttctggtgctgcaactggcgctcctccc
agcagccactcagggaaagaaagtggtgctgggcaaaaaaggggatacagtggaactga
cctgtacagtcccagaagaagagcatacaattccactggaaaaactccaaccagataaa
gattctgggaaatcagggctccttcttaactaaaggtccatccaagctgaatgatcgcg
ctgactcaagaagaagcttgggaccaaggaaactttcccctgatcatcaagaatcttaa
gatagaagactcagatacttacatctgtgaagtggaggaccagaaggaggaggtgcaat
tgctagtgttcggattgactgccaatcgacacccacctgcttcaggggcagagcctgac
cctgaccttggagagcccccctggtagtagcccctcagtgcaatgtaggagtccaaggg
gtaaaaacatacagggggggaagaccctctccggtccagctggagctacaggatagtgg
cacctggacatgcactgtcttgcagaaccagaagaaggtggagttcaaaatagacatcg
tggtgctagctttccagaaggcctccagcatagtctataagaagaggggaacaggtgga
gttctccttcccactcgcctttacagttgaaaagctgacgggcagtggcgagctgtggt
ggcaggcggagagggcttcctcctccaagtcttggatcacctttgacctaagaaaagga
agtgtctgtaaaacgggttacccaggaccctaagctccagatgggcaagaagctcccgc
tccacctcaccctgccccaggccttgcctcagtatgctggctctggaaacctcacccgg
ccctgaagcgaaaacaggaaagttgcatcaggaagtgaacctggtggtgatgagagcca
ctcagctccagaaaaatttgacctgtgaggtgtggggacccacctcccctaagctgatg
ctgagttgaaactgagaacaaggaggcaaaggtctcgaagcgggagaaggcggtgtggg
tgctgaacccagaagcggggatgtggcagtgtctgctgagtgactcgggacaggtcctg
ctggaatccaacataaggttctgcccacatggtccaccccggtctcgagtgggggatcc
ggaggttcaggtgggtctggaggctcggggggctcctcaggtgaacgctacctgaaaga
tcagcaactgctcggcatctgggctgttctgggaagctgatctgtaccaccgcggtccc
ctggaacgcttcttggtcaaacaaatctctagagcagatctggaaccacacaacttgga
tggaatgggaccgggagatcaacaactacaccccctcatccattccctcatcgaggaat
cccagaatcaacaggagaaaaacgagcaggaactcctggaactcgataag
SEQ ID NO:232
(sCD4-sgg3-T20 (shorter linker))
atgaaccggggagtcccttttaggcacttgcttctggtgctgcaactggcgctcctccc
agcagccactcagggaaagaaagtggtgctgggcaaaaaaggggatacagtggaactga
cctgtacagtcccagaagaagagcatacaattccactggaaaaactccaaccagataaa
gattctgggaaatcagggctccttcttaactaaaggtccatccaagctgaatgatcgcg
ctgactcaagaagaagcttgggaccaaggaaactttcccctgatcatcaagaatcttaa
gatagaagactcagatacttacatctgtgaagtggaggaccagaaggaggaggtgcaat
tgctagtgttcggattgactgccaatcgacacccacctgcttcaggggcagagcctgac
cctgaccttggagagcccccctggtagtagcccctcagtgcaatgtaggagtccaaggg
gtaaaaacatacagggggggaagaccctctccggtccagctggagctacaggatagtgg
cacctggacatgcactgtcttgcagaaccagaagaaggtggagttcaaaatagacatcg
tggtgctagctttccagaaggcctccagcatagtctataagaagaggggaacaggtgga
gttctccttcccactcgcctttacagttgaaaagctgacgggcagtggcgagctgtggt
ggcaggcggagagggcttcctcctccaagtcttggatcacctttgacctaagaaaagga
agtgtctgtaaaacgggttacccaggaccctaagctccagatgggcaagaagctcccgc
tccacctcaccctgccccaggccttgcctcagtatgctggctctggaaacctcacccgg
ccctgaagcgaaaacaggaaagttgcatcaggaagtgaacctggtggtgatgagagcca
ctcagctccagaaaaatttgacctgtgaggtgtggggacccacctcccctaagctgatg
ctgagttgaaactgagaacaaggaggcaaaggtctcgaagcgggagaaggcggtgtggg
tgctgaacccagaagcggggatgtggcagtgtctgctgagtgactcgggacaggtcctg
ctggaatccaacataaggttctgcccacatggtccaccccggtctcgagtgggggatcc
ggaggttcggggggctcctcaggtgaatgggatagagaaattaataactatacttctct
gatccacagccttatagaggaatgcaaaaccaacaggagaagaacgaacaggagcttct
ggaactggataaatgggcatcgctttggaattggtcc
SEQ ID NO: 233
sCD4-sgg7-T20 (longer linker))
atgaaccggggagtcccttttaggcacttgcttctggtgctgcaactggcgctcctccc
agcagccactcagggaaagaaagtggtgctgggcaaaaaaggggatacagtggaactga
cctgtacagtcccagaagaagagcatacaattccactggaaaaactccaaccagataaa
gattctgggaaatcagggctccttcttaactaaaggtccatccaagctgaatgatcgcg
ctgactcaagaagaagcttgggaccaaggaaactttcccctgatcatcaagaatcttaa
gatagaagactcagatacttacatctgtgaagtggaggaccagaaggaggaggtgcaat
tgctagtgttcggattgactgccaatcgacacccacctgcttcaggggcagagcctgac
cctgaccttggagagcccccctqgtagtagcccctcagtgcaatgtaggagtccaaggg
gtaaaaacatacagggggggaagaccctctccggtccagctggagctacaggatagtgg
cacctggacatgcactgtcttgcagaaccagaagaaggtggagttcaaaatagacatcg
tggtgctagctttccagaaggcctccagcatagtctataagaagaggggaacaggtgga
gttctccttcccactcgcctttacagttgaaaagctgacgggcagtggcgagctgtggt
ggcaggcggagagggcttcctcctccaagtcttggatcacctttgacctaagaaaagga
agtgtctgtaaaacgggttacccaggaccctaagctccagatgggcaagaagctcccgc
tccacctcaccctgccccaggccttgcctcagtatgctggctctggaaacctcacccgg
ccctgaagcgaaaacaggaaagttgcatcaggaagtgaacctggtggtgatgagagcca
ctcagctccagaaaaatttgacctgtgaggtgtggggacccacctcccctaagctgatg
ctgagttgaaactgagaacaaggaggcaaaggtctcgaagcgggagaaggcggtgtggg
tgctgaacccagaagcggggatgtggcagtgtctgctgagtgactcgggacaggtcctg
ctggaatccaacataaggttctgcccacatggtccaccccggtctcgagtgggggatcc
gqaggttcaggtgggAGTggcgggTCAggtggctctggaggctcggggggctcctcagg
tgaatgggatagagaaattaatactatacttctctgatccacagccttatagaggaatc
gcaaaaccaacaggagaagaacgaacaggagcttctggaactggataaatgggcatcgc
tttggaattggttc
SEQ ID NO: 234
(Short sCD4-link-T20 (sCD4 containing the first
two immunoglobulin like domains))
atgaaccggggagtcccttttaggcacttgcttctggtgctgcaactggcgctcctccc
agcagccactcagggaaagaaagtggtgctgggcaaaaaaggggatacagtggaactga
cctgtacagtcccagaagaagagcatacaattccactggaaaaactccaaccagataaa
gattctgggaaatcagggctccttcttaactaaaggtccatccaagctgaatgatcgcg
ctgactcaagaagaagcttgggaccaaggaaactttcccctgatcatcaagaatcttaa
gatagaagactcagatacttacatctgtgaagtggaggaccagaaggaggaggtgcaat
tgctagtgttcggattgactgccaatcgacacccacctgcttcaggggcagagcctgac
cctgaccttggagagcccccctggtagtagcccctcagtgcaatgtaggagtccaaggg
gtaaaaacatacagggggggaagaccctctccggtccagctggagctacaggatagtgg
cacctggacatgcactgtcttgcagaaccagaagaaggtggagttcaaaatagacatcg
tggtgctagctttccagaaggcctccagtgggggatccggagttcaggtgggtctggag
gctcggggggctcctcaggtgaatgggatagagaaattaataactatacttctctgatc
cacagccttatagaggaatcgcaaaaccaacaggagaagaacgaacaggacttctggaa
ctggataaatgggcatcgctttggaattggttc
SEQ ID NO: 235
(Short sCD4-sgg7-T20 (sCD4 containing the first
two immunoglobulin like domains))
atgaaccggggagtcccttttaggcacttgcttctggtgctgcaactggcgctcctcccagcag
ccactcagggaaagaaagtggtgctgggcaaaaaaggggatacagtggaactgacctgtacagt
cccagaagaagagcatacaattccactggaaaaactccaaccagataaagattctgggaaatca
gggctccttcttaactaaaggtccatccaagctgaatgatcgcgctgactcaagaagaagcttg
ggaccaaggaaactttcccctgatcatcaagaatcttaagatagaagactcagatacttacatc
tgtgaagtggaggaccagaaggaggaggtgcaattgctagtgttcggattgactgccaatcgac
acccacctgcttcaggggcagagcctgaccctgaccttggagagcccccctggtagtagcccct
cagtgcaatgtaggagtccaaggggtaaaaacatacagggggggaagaccctctccggtccagc
tggagctacaggatagtggcacctggacatgcactgtcttgcagaaccagaagaaggtggagtt
caaaatagacatcgtggtgctagctttccagaaggcctccagtgggggatccggagttcaggtg
ggagtggcgggtcaggtggctctggaggctcggggggctcctcaggtgaatgggatagagaaat
taataactatacttctctgatccacagccttatagaggaatcgcaaaaccaacagagaagaacg
aacaggagcttctggaactggataaatgggcatcgctttggaattggttc
Peptide tag amino acid and nucleotide sequence
SEQ ID NO: 212
(cMyc-His6-TEV)
MRAWIFFLLCLAGRALAASEQKLISEEDLNMHTGHHHHHHGENLYFQG
SEQ ID NO: 213
(cMyc-His6-TEV)
atgagggcctggatcttctttctcctttgcctggccgggagggctctggcagctagcgaacaaa
aactcatctcagaagaggatctgaatatgcataccggtcatcatcaccatcaccatggtgagaa
tctttattttcagggt
Miscellaneous
SEQ ID NO: 236
(Expression vector for the bivalent inhibitor #500-underlined
sequence corresponds to the coding region of #500
the rest is the vector)
gggctgggctgagacccgcagaggaagacgctctagggatttgtcccggactagcgagatggcaagg
ctgaggacgggaggctgattgagaggcgaaggtacaccctaatctcaatacaacctttggagctaag
ccagcaatggtagagggaagattctgcacgtcccttccaggcggcctccccgtcaccacccccccca
acccgccccgaccggagctgagagtaattcatacaaaaggactcgcccctgccttggggaatcccag
ggaccgtcgttaaactcccactaacgtagaacccagagatcgctgcgttcccgccccctcacccgcc
cgctctcgtcatcactgaggtggagaagagcatgcgtgaggctccggtgcccgtcagtgggcagagc
gcacatcgcccacagtccccgagaagttggggggaggggtcggcaattgaaccggtgcctagagaag
gtggcgcggggtaaactgggaaagtgatgtcgtgtactggctccgcctttttcccgagggtggggga
gaaccgtatataagtgcagtagtcgccgtgaacgttctttttcgcaacgggtttgccgccagaacac
aggtaagtgccgtgtgtggttcccgcgggcctggcctctttacgggttatggcccttgcgtgccttg
aattacttccacgcccctggctgcagtacgtgattcttgatcccgagcttcgggttggaagtgggtg
ggagagttcgaggccttgcgcttaaggagccccttcgcctcgtgcttgagttgaggcctggcctggg
cgctggggccgccgcgtgcgaatctggtggcaccttcgcgcctgtctcgctgctttcgataagtctc
tagccatttaaaatttttgatgacctgctgcgacgctttttttctggcaagatagtcttgtaaatgc
gggccaagatctgcacactggtatttcggtttttggggccgcgggcggcgacggggcccgtgcgtcc
cagcgcacatgttcggcgaggcggggcctgcgagcgcggccaccgagaatcggacgggggtagtctc
aagctggccggcctgctctggtgcctggcctcgcgccgccgtgtatcgccccgccctgggcggcaag
gctggcccggtcggcaccagttgcgtgagcggaaagatggccgcttcccggccctgctgcagggagc
tcaaaatggaggacgcggcgctcgggagagcgggcgggtgagtcacccacacaaaggaaaagggcct
ttccgtcctcagccgtcgcttcatgtgactccacggagtaccgggcgccgtccaggcacctcgatta
gttctcgagcttttggagtacgtcgtctttaggttggggggaggggttttatgcgatggagtttccc
cacactgagtgggtggagactgaagttaggccagcttggcacttgatgtaattctccttggaatttg
ccctttttgagtttggatcttggttcattctcaagcctcagacagtggttcaaagtttttttcttcc
atttcaggtgtcgtgaggaattcgccgccaccatgaaccggggagtcccttttaggcacttgcttct
ggtgctgcaactagcgctcctcccagcagccactcagggaaagaaagtggtgctgggcaaaaaaggg
gatacagtggaactgacctgtacagcttcccagaagaagagcatacaattccactggaaaaactcca
accagataaagattctgggaaatcagggctccttcttaactaaaggtccatccaagctgaatgatcg
cgctgactcaagaagaagcctttgggaccaaggaaactttcccctgatcatcaagaatcttaagata
gaagactcagatacttacatctgtgaagtggaggaccagaaggaggaggtgcaattgctagtgttcg
gattgactgccaactctgacacccacctgcttcaggggcagagcctgaccctgaccttggagagccc
ccctggtagtagcccctcagtgcaatgtaggagtccaaggggtaaaaacatacagggggggaagacc
ctctccgtgtctcagctggagctacaggatagtggcacctggacatgcactgtcttgcagaaccaga
agaaggtggagttcaaaatagacatcgtggtgctagctttccagaaggcctccagcatagtctataa
gaaagagggggaacaggtggagttctccttcccactcgcctttacagttgaaaagctgacaggcagt
ggcgagctgtggtggcaggcggagagggcttcctcctccaagtcttggatcacctttgacctgaaga
acaaggaagtgtctgtaaaacgggttacccaggaccctaagctccagatgggcaagaagctcccgct
ccacctcaccctgccccaggccttgcctcagtatgctggctctggaaacctcaccctggcccttgaa
gcgaaaacaggaaagttgcatcaggaagtgaacctggtggtgatgagagccactcagctccagaaaa
atttgacctgtgaggtgtggggacccacctcccctaagctgatgctgagtttgaaactggagaacaa
ggaggcaaaggtctcgaagcgggagaaggcggtgtgggtgctgaacccagaagcggggatgtggcag
tgtctgctgagtgactcgggacaggtcctgctggaatccaacatcaaggttctgcccacatggtcca
ccccggtctcgagtgggggatccggaggttcaggtgggtctggaggctcggggggctcctcaggtga
atgggatagagaaattaataactatacttctctgatccacagccttatagaggaatcgcaaaaccaa
caggagaagaacgaacaggagcttctggaactggataaatgggcatcgctttggaattggttctaac
cgcggccgctacgtaaattccgcccctctccctcccccccccctaacgttactggccgaagccgctt
ggaataaggccggtgtgcgtttgtctatatgttattttccaccatattgccgtcttttggcaatgtg
agggcccggaaacctggccctgtcttcttgacgagcattcctaggggtctttcccctctcgccaaag
gaatgcaaggtctgttgaatgtcgtgaaggaagcagttcctctggaagcttcttgaagacaaacaac
gtctgtagcgaccctttgcaggcagcggaaccccccacctggcgacaggtgcctctgcggccaaaag
ccacgtgtataagatacacctgcaaaggcggcacaaccccagtgccacgttgtgagttggatagttg
tggaaagagtcaaatggctctcctcaagcgtattcaacaaggggctgaaggatgcccagaaggtacc
ccattgtatgggatctgatctggggcctcggtgcacatgctttacatgtgtttagtcgaggttaaaa
aacgtctaggccccccgaaccacggggacgtggttttcctttgaaaaacacgatgataagcttgcca
caaccatgaccgagtacaagcccacggtgcgcctcgccacccgcgacgacgtcccccgggccgtacg
caccctcgccgccgcgttcgccgactaccccgccacgcgccacaccgtcgacccggaccgccacatc
gagcgggtcaccgagctgcaagaactcttcctcacgcgcgtcgggctcgacatcggcaaggtgtggg
tcgcggacgacggcgccgcggtggcggtctggaccacgccggagagcgtcgaagcgggggcggtgtt
cgccgagatcggcccgcgcatggccgagttgagcggttcccggetggccgcgcagcaacagatggaa
ggcctcctggcgccgcaccggcccaaggagcccgcgtggttcctggccaccgtcggcgtctcgcccg
accaccagggcaagggtctgggcagcgccgtcgtgctccccggagtggaggcggccgagcgcgccgg
ggtgcccgccttcctggagacctccgcgccccgcaacctccccttctacgagcggctcggcttcacc
gtcaccgccgacgtcgagtgcccgaaggaccgcgcgacctggtgcatgacccgcaagcccggtgcct
gaagatccgggcgcccagcatggaaataaagcacccagcgctgccctgggcccctgcgagactgtga
tggttctttccacgggtcaggccgagtctgaggcctgagtggcatgagatctgatatcatcgatgaa
ttaattcctttgcctaatttaaatgaggacttaacctgtggaaatattttgatgtgggaagctgtta
ctgttaaaactgaggttattggggtaactgctatgttaaacttgcattcagggacacaaaaaactca
tgaaaatggtgctggaaaacccattcaagggtcaaattttcatttttttgctgttggtggggaacct
ttggagctgcagggtgtgttagcaaactacaggaccaaatatcctgctcaaactgtaaccccaaaaa
atgctacagttgacagtcagcagatgaacactgaccacaaggctgttttggataaggataatgctta
tccagtggagtgctgggttcctgatccaagtaaaaatgaaaacactagatattttggaacctacaca
ggtggggaaaatgtgcctcctgttttgcacattactaacacagcaaccacagtgcttcttgatgagc
agggtgttgggcccttgtgcaaagctgacagcttgtatgtttctgctgttgacatttgtgggctgtt
taccaacacttctggaacacagcagtggaagggacttcccagatattttaaaattacccttagaaag
cggtctgtgaaaaacccctacccaatttcctttttgttaagtgacctaattaacaggaggacacaga
gggtggatgggcagcctatgattggaatgtcctctcaagtagaggaggttagggtttatgaggacac
agaggagcttcctggggatcgatccagacatgataagatacattgatgagtttggacaaaccacaac
tagaatgcagtgaaaaaaatgctttatttgtgaaatttgtgatgctattgctttatttgtaaccatt
ataagctgcaataaacaagttaacaacaacaattgcattcattttatgtttcaggttcagggggagg
tgtgggaggttttttaaagcaagtaaaacctctacaaatgtggtatggctgattatgatctctagtc
aaggcactatacatcaaatattccttattaacccctttacaaattaaaaagctaaaggtacacaatt
tttgagcatagttattaatagcagacactctatgcctgtgtggagtaagaaaaaacagtatgttatg
attataactgttatgcctacttataaaggttacagaatatttttccataattttcttgtatagcagt
gcagctttttcctttgtggtgtaaatagcaaagcaagcaagagttctattactaaacacagcatgac
tcaaaaaacttagcaattctgaaggaaagtccttggggtcttctacctttctcttcttttttggagg
agtagaatgttgagagtcagcagtagcctcatcatcactagatggcatttcttctgagcaaaacagg
ttttcctcattaaaggcattccaccactgctcccattcatcagttccataggttggaatctaaaata
cacaaacaattagaatcagtagtttaacacattatacacttaaaaattttatatttaccttagagct
ttaaatctctgtaggtagtttgtccaattatgtcacaccacagaagtaaggttccttcacaagatct
aaagccagcaaaagtcccatggtcttataaaaatgcatagctttaggaggggagcagagaacttgaa
agcatcttcctgttagtctttcttctcgtagacttcaaacttatacttgatgcctttttcctcctgg
acctcagagaggacgcctgggtattctgggagaagtttatatttccccaaatcaatttctgggaaaa
acgtgtcactttcaaattcctgcatgatccttgtcacaaagagtctgaggtggcctggttgattcat
ggcttcctggtaaacagaactgcctccgactatccaaaccatgtctactttacttgccaattccggt
tgttcaataagtcttaaggcatcatccaaacttttggcaagaaaatgagctcctcgtggtggttctt
tgagttctctactgagaactatattaattctgtcctttaaaggtcgattcttctcaggaatggagaa
ccaggttttcctacccataatcaccagattctgtttaccttccactgaagaggttgtggtcattctt
tggaagtacttgaactcgttcctgagcggaggccagggtaggtctccgttcttgccaatccccatat
tttgggacacggcgacgatgcagttcaatggtcgaaccatgatggcagcggggataaaatcctacca
gccttcacgctaggattgccgtcaagtttggcgcgaaatcgcagccctgagctgtcccccccccccc
ccccccaagctttttgcaaaagcctaggcctccaaaaaagcctcctcactacttctggaatagctca
gaggccgaggcggcctcggcctctgcataaataaaaaaaattagtcagccatggggcggagaatggg
cggaactgggcggagttaggggcgggatgggcggagttaggggcgggactatggttgctgactaatt
gagatgcatgctgcctcgcgcgtttcggtgatgacggtgaaaacctctgacacatgcagctcccgga
gacggtcacagcttgtctgtaagcggatgccgggagcagacaagcccgtcagggcgcgtcagcgggt
gttggcgggtgtcggggcgcagccatgacccagtcacgtagcgatagcggagtgtatactggcttaa
ctatgcggcatcagagcagattgtactgagagtgcaccatatgcggtgtgaaataccgcacagatgc
gtaaggagaaaataccgcatcaggcgctcttccgcttcctcgctcactgactcgctgcgctcggtcg
ttcggctgcggcgagcggtatcagctcactcaaaggcggtaatacggttatccacagaatcagggga
taacgcaggaaagaacatgtgagcaaaaggccagcaaaaggccaggaaccgtaaaaaggccgcgttg
ctggcgtttttccataggCtccgcccccctgacgagcatcacaaaaatcgacgctcaagtcagaggt
ggcgaaacccgacaggactataaagataccaggcgtttccccctggaagctccctcgtgcgctctcc
tgttccgaccctgccgcttaccggatacctgtccgcctttctcccttcgggaagcgtggcgctttct
catagctcacgctgtaggtatctcagttcggtgtaggtcgttcgctccaagctgggctgtgtgcacg
aaccccccgttcagcccgaccgctgcgccttatccggtaactatcgtcttgagtccaacccggtaag
acacgacttatcgccactggcagcagccactggtaacaggattagcagagcgaggtatgtaggcggt
gctacagagttcttgaagtggtggcctaactacggctacactagaaggacagtatttggtatctgcg
ctctgctgaagccagttaccttcggaaaaagagttggtagctcttgatccggcaaacaaaccaccgc
tggtagcggtggtttttttgtttgcaagcagcagattacgcgcagaaaaaaaggatctcaagaagat
cctttgatcttttctacggggtctgacgctcagtggaacgaaaactcacgttaagggattttggtca
tgagattatcaaaaaggatcttcacctagatccttttaaattaaaaatgaagttttaaatcaatcta
aagtatatatgagtaaacttggtctgacagttaccaatgcttaatcagtgaggcacctatctcagcg
atctgtctatttcgttcatccatagttgcctgactccccgtcgtgtagataactacgatacgggagg
gcttaccatctggccccagtgctgcaatgataccgcgagacccacgctcaccggctccagatttatc
agcaataaaccagccagccggaagggccgagcgcagaagtggtcctgcaactttatccgcctccatc
cagtctattaattgttgccgggaagctagagtaagtagttcgccagttaatagtttgcgcaacgttg
ttgccattgctgcaggcatcgtggtgtcacgctcgtcgtttggtatggcttcattcagctccggttc
ccaacgatcaaggcgagttacatgatcccccatgttgtgcaaaaaagcggttagctccttcggtcct
ccgatcgttgtcagaagtaagttggccgcagtgttatcactcatggttatggcagcactgcataatt
ctcttactgtcatgccatccgtaagatgcttttctgtgactggtgagtactcaaccaagtcattctg
agaatagtgtatgcggcgaccgagttgctcttgcccggcgtcaacacgggataataccgcgccacat
agcagaactttaaaagtgctcatcattggaaaacgttcttcggggcgaaaactctcaaggatcttac
cgctgttgagatccagttcgatgtaacccactcgtgcacccaactgatcttcagcatcttttacttt
caccagcgtttctgggtgagcaaaaacaggaaggcaaaatgccgcaaaaaagggaataagggcgaca
cggaaatgttgaatactcatactcttcctttttcaatattattgaagcatttatcagggttattgtc
tcatgagcggatacatatttgaatgtatttagaaaaataaacaaataggggttccgcgcacatttcc
ccgaaaagtgccacctgacgtctaagaaaccattattatcatgacattaacctataaaaataggcgt
atcacgaggccctttcgtcttcaagaatt
Peptide Based HIV-1 Fusion Inhibitors
SEQ ID NO: 237
(T20 [ENF/DP-178])
YTSLIHSLIEESQNQQEKNEQELLELDKWASLWNWF
SEQ ID NO: 239
(T649)
WMEWDREINNYTSLIHSLIEESQNQQEKNEQELLEL
SEQ ID NO: 239
(SJ-2176)
EWDREINNYTSLIHSLIEESQNQQEKNEQEGGC
SEQ ID NO: 240
(C34)
WMEWDREINNYTSLIHSLIEESQNQQEKNEQELL
SEQ ID NO: 241
(T1249)
WQEWEQKITALLEQAQIQQEKNEYELQKLDKWASLWEWF
SEQ ID NO: 242
(T1249MUT)
WQEWEQKITALLEQAQIQQEKNEYELRKLDKWASLWEWF
SEQ ID NO: 243
(T20S138A)
YTSLIHSLIEEAQNQQEKNEQELLELDKWASLWNWF
SEQ ID NO: 244
(T2410)
MTWMEWDREINNYTSLIHSLIEESQNQQEKNEQELLEL [71]
SEQ ID NO: 245
(T2635)
TTWEAWDRAIAEYAARIEALIRAAQEQQEKNEAALREL
SEQ ID NO: 246
(T2635MUT)
TTWEAWDRAIAEYAARIEALIRAAQEQQEKNEAALQEL
SEQ ID NO: 247
(Sifuvirtide [SFT])
SWETWEREIENYTRQIYRILEESQEQQDRNERDLLE
SEQ ID NO: 248
(SC34EK)
WZEWDRKIEEYTKKIEELIKKSQEQQEKNEKELK1
SEQ ID NO: 249
(SC35EK)
WEEWDKKIEEYTKKIEELIKKSEEQQKKNEEELKK
SEQ ID NO: 250
(SC29EK)
WEEWDKKIEEYTKKIEELIKKSEEQQKKN
SEQ ID NO: 251
(SC22EK)
WEEWDKKIEEYTKKIEELIKKS
SEQ ID NO: 252
(T20EK)
YTSLIEELIKKWEEQQKKNEEELKKLEEWAKKWNWF
SEQ ID NO: 253
(C52D)
NHTTWMEWDREINNYTSLIHSLIEESQNQQEKNEQELLELDKWASLWNWFNIKIKQIEDKIK
SEQ ID NO: 254
(C52L)
NHTTWMEWDREINNYTSLIHSLIEESQNLQEKNEQELLELDKWASLWNWFNIKIK
SEQ ID NO: 255
(CP32M)
VEWNEMTWMEWEREIENYTKLIYKILEESQEQ
SEQ ID NO: 256
(PBD-3HR-LBD)
WMEWDREIEEYTKKIEEYTKKIEEYTKKIWASLWNWF
SEQ ID NO: 257
(P5)
WMEWDREINNYTSLIHSLIEESQNQQEKNEQELLELDKWASLWNWFNITNWLWYIK
SEQ ID NO: 258
(N36)
SGIVQQQNNLLRAIEAQQHLLQLTVWGIKQLQARIL
SEQ ID NO: 259
(N36Mut)
SGIDQEQNNLTRLIEAQIHELQLTQWKIKQLLARIL
SEQ ID NO: 260
(T-21 [DP-107])
NNLLRAIEAQQHLLQLTVWGIKQLQARILAVERYLKDQ
SEQ ID NO: 261
(IQN17 [IZN17])
GCN4-LLQLTVWGIKQLQARIL
SEQ ID NO: 262
(IQN23)
GCN4-IEAQQHLLQLTVWGIKQLQARIL
SEQ ID NO: 263
(NCCG-gp41)
SGIVQQQNNLLRAIEAQQHLLQLTVWGIKQCCGRI-N34(L6)C28
SEQ ID NO: 264
(RC-100)
GICRCICGRGICRCICGR
SEQ ID NO: 265
(RC-101)
GICRCICGKGICRCICGR
SEQ ID NO: 266
(RC-106)
GICYCICGRGICRCICGR
SEQ ID NO: 267
(RC-115)
GICRCICGRYICRCICGR
SEQ ID NO: 268
(RC-116)
RYICRCICGRGICRCICG
SEQ ID NO: 269
(VIRIP)
LEAIPMSIPPEVKFNKPFVF
SEQ ID NO: 270
(VIR-164)
LEAIPCSIPPCVFFNKPFVF
SEQ ID NO: 271
(VIR-165)
LEAIPCSIPPCFAFNKPFVF
SEQ ID NO: 272
(VIR-175)
LEAIPMSIPPEFLFGKPFVF
SEQ ID NO: 273
(VIR-353)
LEAIPCSIPpCFLFNKPFVF2
SEQ ID NO: 274
(VIR-449)
LEAIPMGIPpEV1FNKPFVF3
SEQ ID NO: 275
(VIR-576)
LEAIPCSIPPEFLFGKPFVF

1-161. (canceled)

162. A pre-fusion inhibitor molecule comprising:
i) a first part that comprises or consists of a first virus binding moiety that binds to a first viral protein; and
ii) a second part that comprises or consists of a second virus binding moiety that binds to a second viral protein;
wherein said first part comprises or consists of an amino acid sequence of a mammalian membrane receptor or a fragment, mimic or functional homologue thereof;
or wherein said first or second part is an antibody or an antigen-binding fragment.

163. The molecule according to claim 162, wherein said fragment is at least 75 amino acids long; and wherein said functional homologue is at least 40 percent homologous with said mammalian membrane receptor.

164. The molecule according to claim 162, wherein said first viral protein is HIV gp120, said second viral protein is HIV gp41, and
said first part is mammalian soluble CD4 (sCD4) or a fragment, or functional homologue thereof, or
an amino acid sequence at least 80% identical to soluble CD4 (sCD4) or a fragment, or functional homologue thereof;
preferably wherein the length of said fragment is at least 25% percent of the length of CD4;
and wherein said functional homologue is at least 40 percent homologous with CD4.

165. The molecule according to claim 162, wherein the first part exhibits the virus binding function of a mammalian membrane receptor or a soluble part thereof, preferably wherein the mammalian membrane receptor is selected from the group consisting of CD4, sCD4, ICAM-1, Coxsackievirus-adenovirus receptor (CAR), Poliovirus receptor (CD155), HAVCr-1, Neural cell adhesion molecule (CD56), MHC class I, MHC class II, Nectin 1 and 2, aV integrins, a2b1, a chemokine receptor, Complement receptor CR2 (CD21), CD46, Decay-accelerating factor (CD55), Low-density lipoprotein receptor, Acetylcholine receptor, Epidermal growth factor receptor, Herpesvirus entry mediator (HVEM), Sialic acid and Heparan sulfate.

166. The molecule according to claim 162, wherein the first part exhibits the virus binding function of a mammalian membrane receptor or a soluble part thereof, wherein the mammalian membrane receptor is a co-receptor, preferably wherein said co-receptor is selected from the group consisting of Claudin-1, Occludin, PILR-α in, mannose-binding lectin, FR-alpha, Integrins, AlphaVbeta5 integrin, Human Hepatocyte Growth Factor, CCR5, CXCR4, CCR2, CCR3, CCR8, CCR9, CXCR6 (Bonzo/STRL33/TYMSTR), CX3CR1, ChemR23, APJ, Bob/GPR15, GPR1 and RDC1, more preferred said co-receptor is CCR5, CRCX4, CCR2, CCR3, CCR8, CCR9, CXCR6 (Bonzo/STRL33/TYMSTR), CX3CR1, ChemR23, APJ, Bob/GPR15, GPR1 or RDC1.

167. The molecule according to claim 162, wherein said first part is selected from the group of compounds consisting of an N-phenyl-N′-piperidine-oxalamide derivative, NBD-556, NBD-557, DN-3186, JRC-II-75 and JRC-II-11.

168. The molecule according to claim 162, wherein the molecule further comprises a purification tag, preferably wherein said purification tag is selected from the group consisting of hexahistidine. cMyc and an amino acid sequence according to SEQ ID NO: 213, more preferred said purification tag is a hexahistidine tag.

169. The molecule according to claim 162, wherein said first part is:
a peptide with amino acid sequence selected from the group of amino acid sequences consisting of SEQ ID NOS: 9 and 10 and fragments, mimics and functional homologues thereof, or
a peptide with at least 80% identity to a peptide with amino acid sequence consisting of any of SEQ ID NOS: 9 or 10.

170. The molecule according to claim 162, wherein said virus is selected from the group of viruses consisting of Othomyxoviridae, Paramyxoviridae, Retroviridae, Filoviridae and Coronaviridae, preferably said virus is selected from the group of viruses consisting of HTLV-1, HTLV-2, HERV, BLV, ELV, FeLV, PuLV, O/CLV, visna/maedi, PrLV, HIV-1, HIV-2, SIV, MLV, JSRV, FeLV A, Influenza HA, and ebola.

171. The molecule according to claim 162, wherein the second viral protein is HIV gp41, or any part, fragment, mimic or functional homologue thereof.

172. The molecule according to claim 162, wherein the first or second part is an antibody or an antigen-binding fragment, and wherein the antibody or antigen-binding fragment is selected from the group consisting of intact antibodies, Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]), preferably a single chain Fv (scFv), or wherein the first and/or second part comprises or consists of an antibody-like binding agent, for example an affibody or aptamer.

173. The molecule according to claim 162, wherein the second part comprises or consists of a peptide capable of forming a coiled coil, or a heptad repeat structural motif, or wherein said second viral protein is capable of forming a triple-helix.

174. The molecule according to claim 162, wherein said second part comprises or consists of:
a peptide having an amino acid sequence according to any one of SEQ ID NOS: 11-18 or SEQ ID NOS: 20-204; or a fragment, mimic, or functional homologue thereof, or
a peptide having an amino acid sequence at least 80% identical to any one of SEQ ID NOS: 11-18 or SEQ ID NOS: 20-204.

175. The molecule according to claim 162, wherein said linker is a polymer, preferably selected from the group of polymers consisting of polyamides, polypeptides, polysaccharides and polynucleotides.

176. The molecule according to claim 162, wherein said molecule comprises or consists of:
a peptide having an amino acid sequence according to any one of SEQ ID NOS: 1-8, 19, or 216-225; or any part, fragment, mimic, or functional homologue thereof, or
a peptide having an amino acid sequence at least 80% identical to any one of SEQ ID NOS: 1-8, 19, or 216-225.

177. A pre-fusion inhibitor molecule comprising:
i) a first part that comprises or consists of a first virus binding moiety that binds to a viral protein; and
ii) a second part that comprises or consists of a second virus binding moiety that binds to the viral protein at a different site to the first binding moiety;
wherein the first part binds to a mammalian membrane receptor-binding domain of the viral protein; and wherein the mammalian membrane receptor binding domain of the viral protein overlaps with the site of the viral protein that interacts with and/or binds to a viral membrane anchored protein (or a subunit thereof); and the site of the viral protein that interacts with and/or binds to the viral membrane anchored protein (or a subunit thereof) is responsible for inducing a conformational change in the membrane anchored protein (or a subunit thereof) when the viral protein binds to a mammalian membrane receptor.

178. A polynucleotide comprising or consisting of a nucleic acid sequence encoding a molecule as defined in claim 177, wherein the polynucleotide comprises or consists of:
a polynucleotide having a nucleic acid sequence according to any one of SEQ ID NOS: 205-212 or a part or fragment thereof, or
a polynucleotide having a nucleic acid sequence at least 80% identical to SEQ ID NOS: 205-212, or
a codon optimised polynucleotide encoding a polypeptide according to any one of SEQ ID NOS: 1-8.

179. A method of inhibiting the growth of a microbe, comprising administering to a subject in need thereof one or more molecules as defined in claim 162.

180. A method of inhibiting the growth of a microbe, comprising administering to a subject in need thereof a gene therapy vector comprising one or more polynucleotides as defined in claim 178.

181. A method of treating, preventing and/or ameliorating a disease and/or clinical condition, said method comprising administering to an individual suffering from said disease and/or clinical condition an effective amount of one or more molecules as defined in claim 162, preferably wherein said disease and/or clinical condition belongs to the group of diseases and/or clinical conditions arising from virus infections, more preferred said virus is Human Immunodeficiency Virus (HIV) or wherein said disease and/or clinical condition is Acquired Immune Deficiency Syndrome (AIDS).
